(19) United States
(12) Patent Application Publication
(10) Pub. No.: US 2024/0216526 A1
(43) Pub. Date: Jul. 4, 2024

(54) PREPARATION METHOD FOR DUAL-DRUG-LINKER OF ADC AND USE THEREOF

(71) Applicant: CHENGDU SCIMOUNT PHARMATECH CO., LTD., Chengdu, Sichuan (CN)

(72) Inventors: Jinkun HUANG, Chengdu, Sichuan (CN); Jiahao GENG, Chengdu, Sichuan (CN); Chaoyang FENG, Chengdu, Sichuan (CN); Junqiang LIU, Chengdu, Sichuan (CN); Yue LU, Chengdu, Sichuan (CN); Lei LI, Chengdu, Sichuan (CN); Chenglong WU, Chengdu, Sichuan (CN); Fang CHEN, Chengdu, Sichuan (CN); Dejian XIE, Chengdu, Sichuan (CN)

(21) Appl. No.: 18/551,702

(22) PCT Filed: Mar. 15, 2022
(86) PCT No.: PCT/CN2022/080942

§ 371 Date: Sep. 21, 2023

(30) Foreign Application Priority Data
Mar. 22, 2021 (CN) 202110303043.7

(51) Int. Cl. A61K 47/68; A61P 35/00; C07K 16/32

(52) U.S. Cl. CPC A61K 47/6889; A61K 47/68031; A61K 47/68033; A61K 47/68037; A61K 47/6849; A61P 35/00; C07K 16/32

(57) ABSTRACT

Provided are a dual-drug link assembly unit represented by formula III, or a stereoisomer thereof, or an optical isomer thereof. The dual-drug link assembly unit can be linked to a targeting linker to obtain a dual-drug targeting linker-drug conjugate represented by formula I. The specific structure can effectively reduce the aggregation of the dual-drug targeting linker-drug conjugate, which facilitates process scale-up, thereby improving the efficiency of the targeting effect on tumor cells, reducing the toxic and side effects on normal cells, and at the same time, effectively overcoming drug resistance and achieving a synergistic anti-tumor effect. Compared with DS-8201 which is already on the market, the ADC provided by the present invention significantly improves the inhibition effect on HER2 positive cell strains N87 and SK—BR-3d.

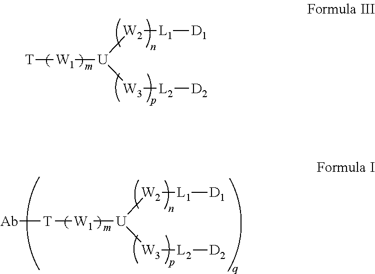

### TECHNICAL FIELD

The present invention belongs to the field of pharmaceuticals, and specifically relates to a preparation method for a dual-drug-linker of ADC and use thereof.

### BACKGROUND TECHNOLOGY

Antibody-Drug Conjugate (ADC) can selectively deliver drugs to cancer cells and kill them, but its impact on normal cells is relatively small, marking the beginning of a new era in tumor therapy. For ADCs, a few drugs have been approved by the FDA for marketing, such as Mylotarg, which is made by linking anti-CD33 antibody to calicheamicin; Adcetris, which is made by linking anti-CD30 antibody to aurestatin E, and used for treating patients with Hodgkin's lymphoma and undifferentiated large cell lymphoma; DS-8201, which is made by linking anti-HER2 antibody to the camptothecin derivative DXd, and used for treating patients with HER2-positive breast cancer; and Sacituzumabgovitecan, which targets antigen TROP-2 (also known as epithelial glycoprotein 1, EGP-1).

ADC that have been approved by the FDA so far mainly targeting TOP isomerase or microtubule proteins. The marketed ADC are prepared by linking antibodies to the medicaments targeting TOP isomerase or tubulin, respectively. Currently, there are no ADC that connect the antibodies and the medicaments targeting TOP isomerase and tubulin simultaneously. It is unclear whether connecting the medicaments targeting TOP isomerase and tubulin simultaneously to the antibodies can kill tumors by two different mechanisms, and whether both of medicaments can effectively play their effects.

As small molecule compounds with anti-tumor properties, camptothecin derivatives such as SN-38, DXd, and DX-8951, known as compounds that inhibit topoisomerase I and thus play anti-tumor actions, have been confirmed to have killing effects on various cancer cells in vivo and in vitro, demonstrating strong anti-tumor effects. Compounds that inhibit microtubule proteins and thus have anti-tumor activities, such as Eribulin, MMAE, MMAF, maytansine, have been confirmed to have killing effects on various cancer cells in vivo and in vitro, indicating significant anti-tumor effects. Linking two anti-tumor drugs with different mechanisms to the same antibody may have a synergistic effect on tumors or an antagonistic effect on tumors, but the actual effect cannot be expected.

Therefore, there is an urgent need to develop an efficient and safe multi-drug ADC with multi-target mechanisms, which is of great significance for the development of anti-tumor drugs with excellent anti-tumor effects and safety.

### SUMMARY OF THE INVENTION

The object of the present invention is to provide a dual-drug link assembly unit and its corresponding dual-drug targeting linker-drug conjugate.

The present invention provides a dual-drug link assembly unit represented by formula III, or a stereoisomer thereof, or an optical isomer thereof, or a deuterated compound thereof:

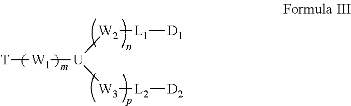

wherein, T is a tether group which can be connected to the targeting linker; the targeting linker is a substance that can target and bind to the lesion site;
U is a Y-shaped linker moiety, with a structure of

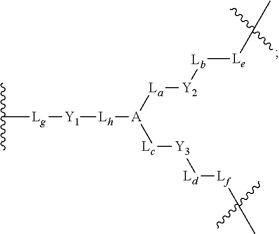

wherein, Y₁, Y₂, and Y₃ are each independently selected from the group consisting of CONH, NHCO, CO, NH, COO, OCO, O, S,

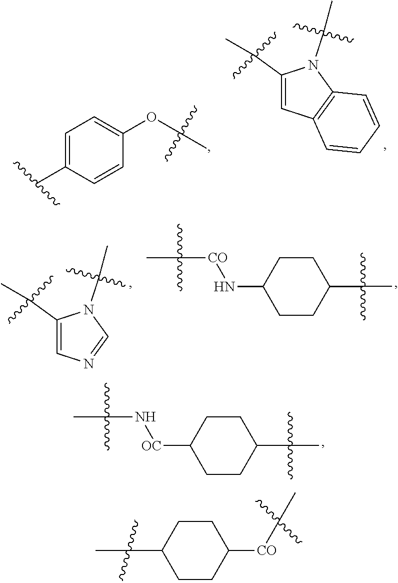

or absence; Lₐ, L_{b}, L_{c}, L_{d}, Lₑ, L_{f}, L_{g}, Lₕ are each independently selected from the group consisting of 0-8 methylenes; A is selected from N, as well as the following substituted or unsubstituted groups: aryl, heteroaryl, chain alkyl, fused cycloalkyl, fused heterocycloalkyl, saturated cycloalkyl or saturated heterocycloalkyl, and the substituent is each independently selected from halogen, cyano, hydroxyl, C₁₋₆ alkyl or C₁₋₆ alkoxy;

m, n, and p are each independently selected from an integer of 0 to 30, as well as n and p are not both 0;

W₁, W₂, and W₃ are each independently selected from the group consisting of methylene,

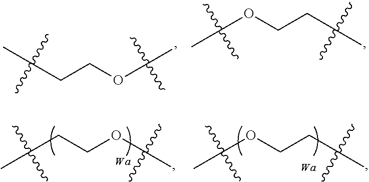

alkenylene, alkynylene, 3-8 membered aryl, 3-8 membered heteroaryl; Wa is selected from an integer of 2 to 8;

L₁ and L₂ are cleavable or non-cleavable linking groups;

D1 and D2 are the first and second drug structural units, respectively, with the same or different structures.

Further, said T can react and connect with thiol or amino groups in the targeting linker.

Further, the structure of said

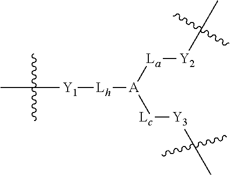

is selected from the group consisting of:

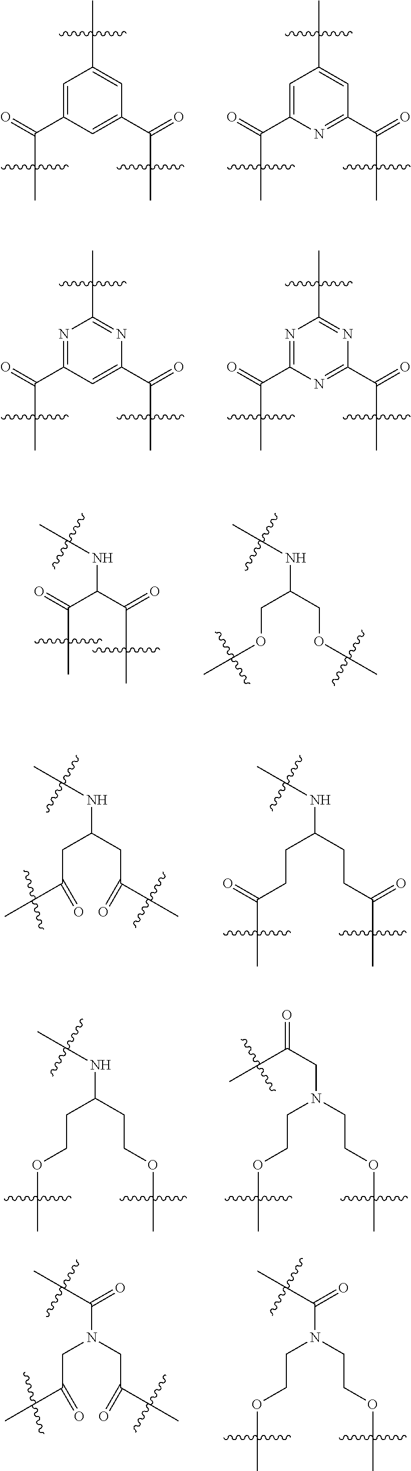

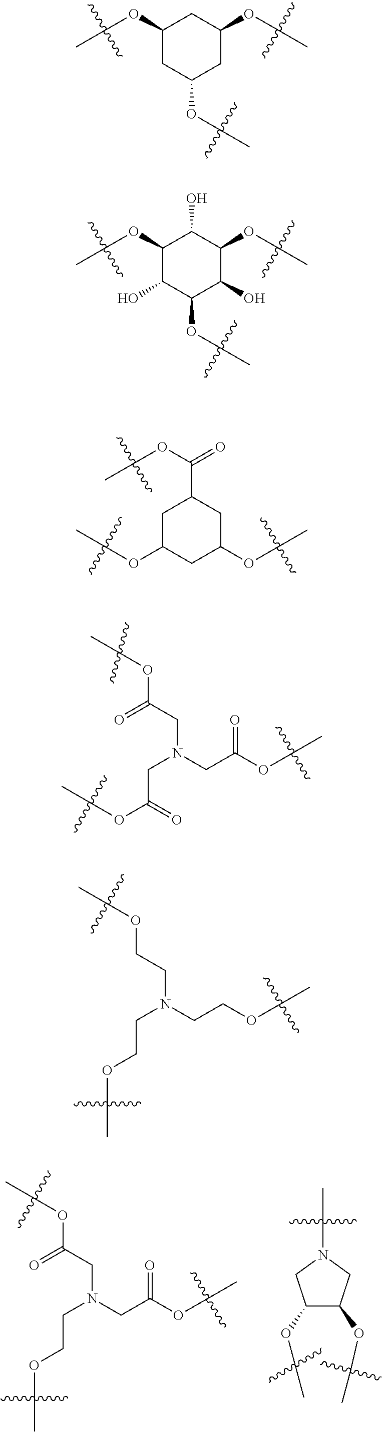

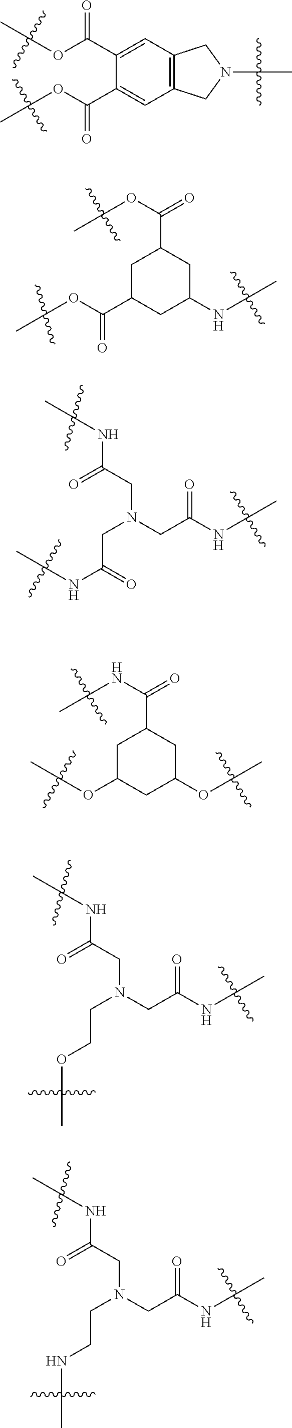 [SMILES: *OC(=O)c1cc2c(cc1C(=O)O*)CN(*)C2 ; *NC1CC(C(=O)O*)CC(C(=O)O*)C1 ; *NC(=O)CN(CC(=O)N*)CC(=O)N* ; *NC(=O)C1CC(O*)CC(O*)C1 ; *NC(=O)CN(CCO*)CC(=O)N* ; *NCCN(CC(=O)N*)CC(=O)N*]

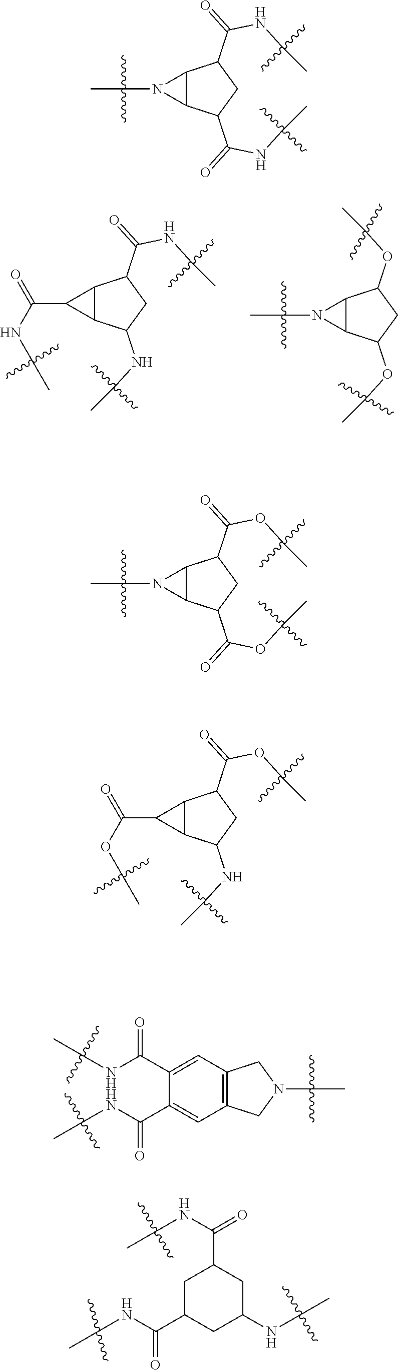

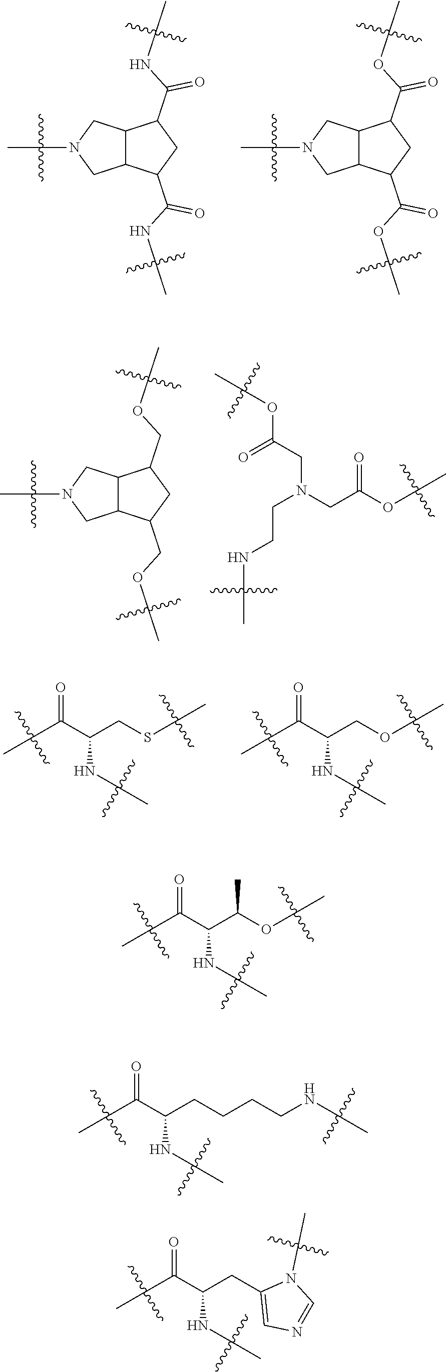

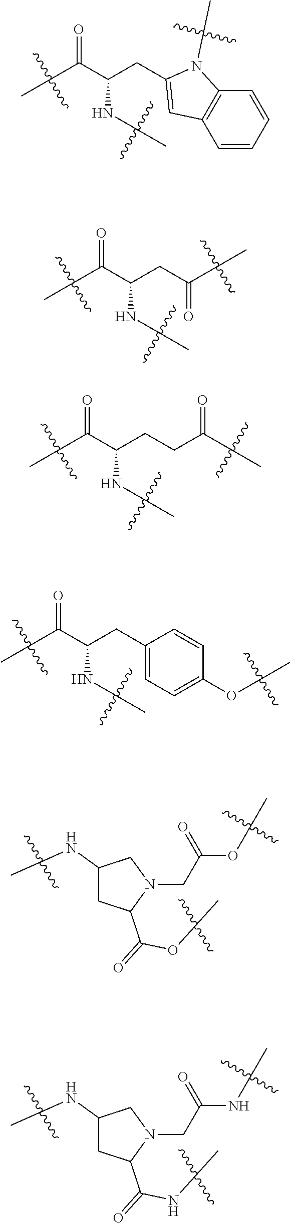 [SMILES: *N[C@@H](Cc1cc2ccccc2n1*)C(*)=O ; *N[C@@H](CC(*)=O)C(*)=O ; *N[C@@H](CCC(*)=O)C(*)=O ; *N[C@@H](Cc1ccc(O*)cc1)C(*)=O ; *NC1CC(C(=O)O*)N(CC(=O)O*)C1 ; *NC(=O)CN1CC(N*)CC1C(=O)N*]

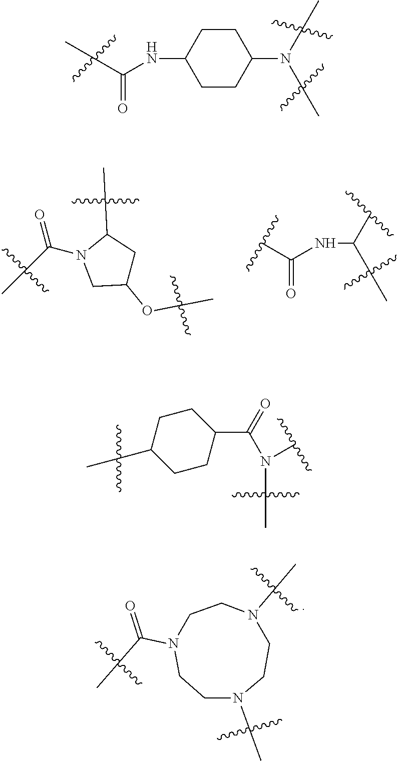 [SMILES: *C(=O)NC1CCC(N(*)*)CC1 ; *OC1CC(*)N(C(*)=O)C1 ; *C(=O)NC(*)* ; *C1CCC(C(=O)N(*)*)CC1 ; *C(=O)N1CCN(*)CCN(*)CC1]

Further, the structure of said dual-drug link assembly unit is as represented by formula IV:

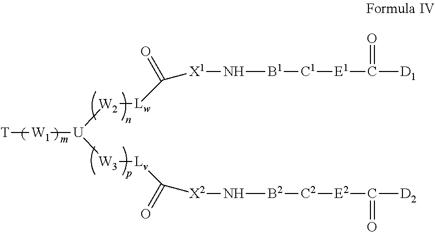

wherein, the structure of U is

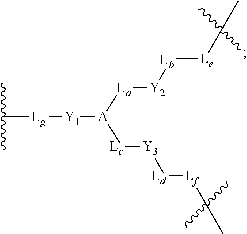

in which Y₁, Y₂, and Y₃ are each independently selected from the group consisting of CONH, CO, NH, O,

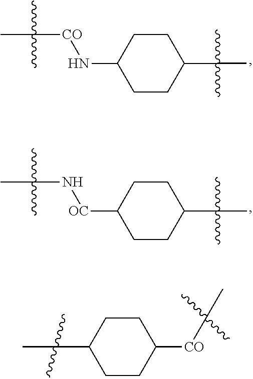

or absence; Lₐ, L_{b}, L_{c}, L_{d}, Lₑ, L_{f}, L_{g}, L_{w}, and Lᵥ, are each independently selected from the group consisting of 0-4 methylenes; A is selected from N, as well as the following substituted or unsubstituted groups: aryl, heteroaryl, chain alkyl, saturated cycloalkyl or saturated heterocycloalkyl, and the substituent is each independently selected from halogen, cyano, hydroxyl, C₁₋₆ alkyl or C₁₋₆ alkoxy;

m, n, and p are each independently selected from an integer of 0 to 30, as well as n and p are not both 0;

W₁, W₂, and W₃ are each independently selected from the group consisting of methylene,

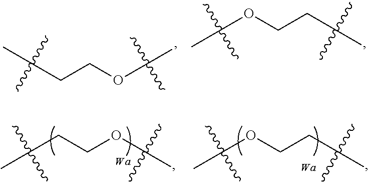

alkenylene, alkynylene, 3-8 membered aryl, 3-8 membered heteroaryl; Wa is selected from an integer of 2 to 4;

X¹ and X² are each independently selected from the group consisting of

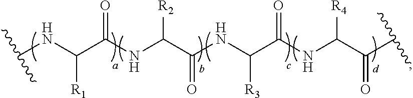

wherein, a, b, c, and d are each independently selected from 0 or 1; R₁, R₂, R₃, and R₄ are each independently selected from the group consisting of H, C₁₋₅ alkyl, substituted or unsubstituted benzyl, and -L₇NHCONH₂; L₇ is 0-3 methylenes;

B¹, B², C¹, C², E¹, and E² are each independently selected from the group consisting of the following substituted or unsubstituted groups:

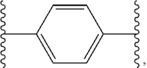 [SMILES: *c1ccc(*)cc1]

L₈NHL₃, L₄OL₅ or absence; the substituent is each independently selected from

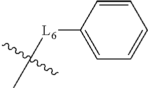

and C_{1˜5} alkyl; wherein L₈, L₃, L₄, L₅, and L₆ are each independently selected from 0˜2 methylenes;

D₁ and D₂ are independently selected from cytotoxic medicaments, medicaments for treating autoimmune diseases, or anti-inflammatory medicaments;

T is as defined in the above.

Further, the structure of said dual-drug link assembly unit is as represented by formula V:

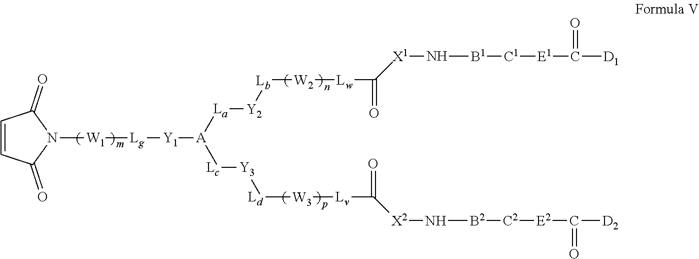

wherein, Y₁, Y₂, and Y₃ are each independently selected from the group consisting of CONH, CO, NH, O,

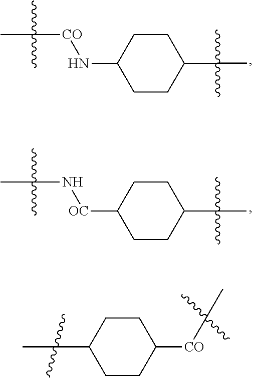

or absence; Lₐ, L_{b}, L_{c}, L_{d}, Lₑ, L_{f}, L_{g}, L_{w}, and Lᵥ, are each independently selected from 0-4 methylenes; A is selected from the group consisting of N, substituted or unsubstituted phenyl, and substituted or unsubstituted

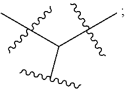 [SMILES: *C(*)*]

and the substituent is each independently selected from halogen, cyano, hydroxyl, C₁₋₆ alkyl or C₁₋₆ alkoxy;

m, n, and p are each independently selected from an integer of 0 to 30, as well as n and p are not both 0;

W₁, W₂, and W₃ are each independently selected from the group consisting of methylene,

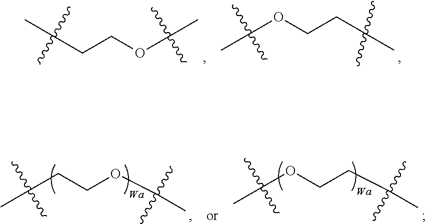

Wa is selected from an integer of 2 to 3; and at least one of W₂ and W₃ is

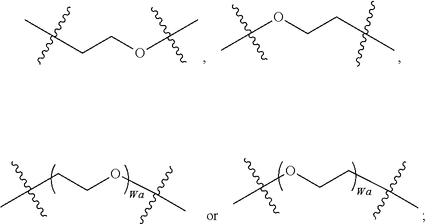

X¹, X², B¹, B², C¹, C², E¹, E², D₁, and D₂ are as defined in the above.

Further, the structure of said dual-drug link assembly unit is as represented by formula VI-1, VI-2, VI-3 or VI-4:

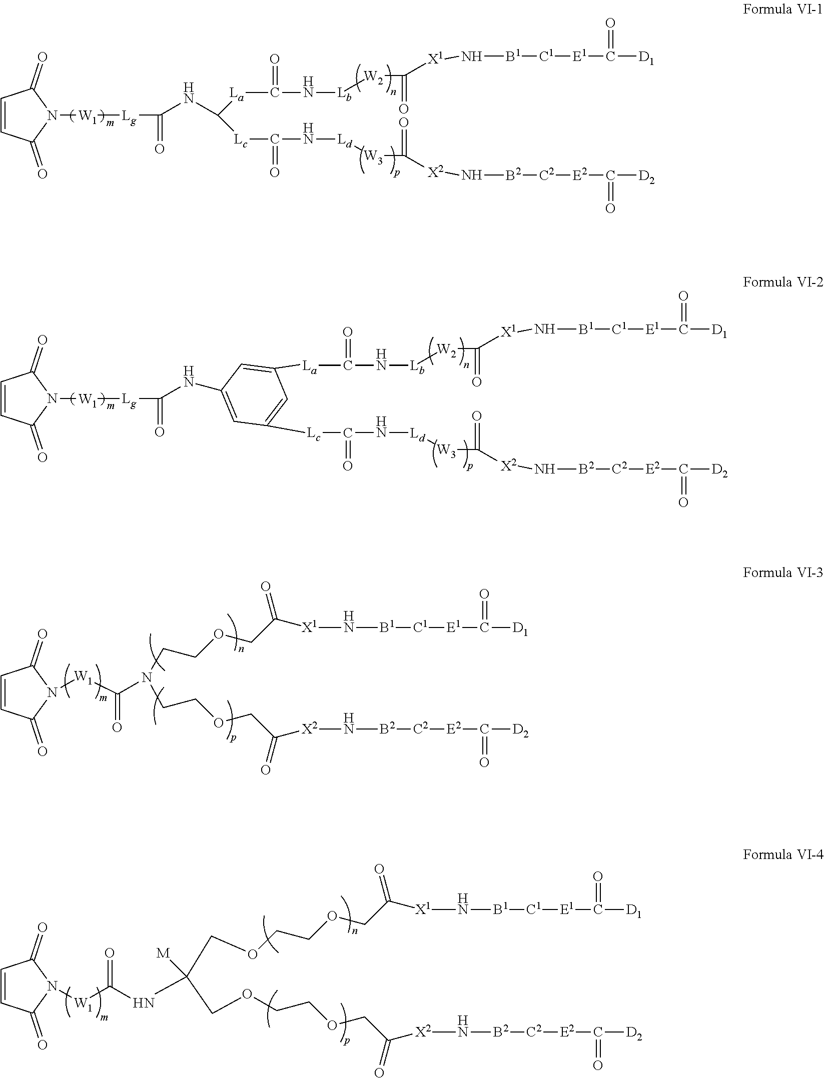

wherein, m, n, and p are each independently selected from an integer of 0 to 30;

W₁, W₂, and W₃ are each independently selected from the group consisting of methylene,

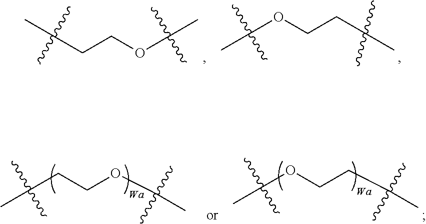

Wa is selected from an integer of 2 to 3; and at least one of W₂ and W₃ is

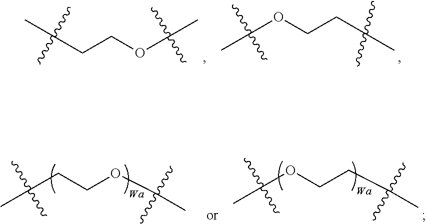

M is selected from the group consisting of halogen, cyano, hydroxyl, C₁₋₆ alkyl or C₁₋₆ alkoxy;

Lₐ, L_{b}, L_{c}, L_{d}, and L_{g} are each independently selected from 0-2 methylenes;

X¹, X², B¹, B², C¹, C², E¹, E², D₁, and D₂ are as defined in the above.

Further, the structure of said dual-drug link assembly unit is selected from one of the following structures:

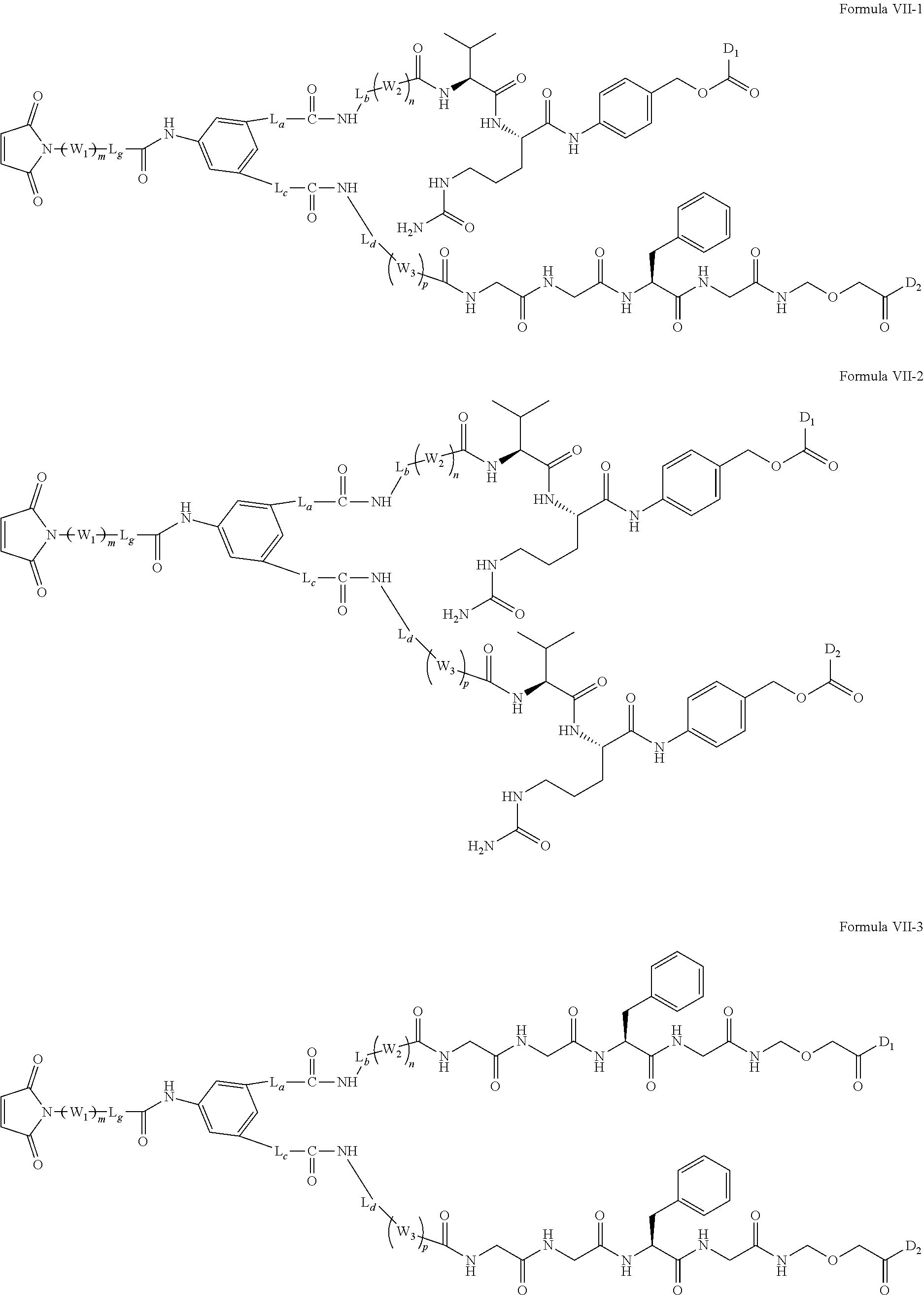

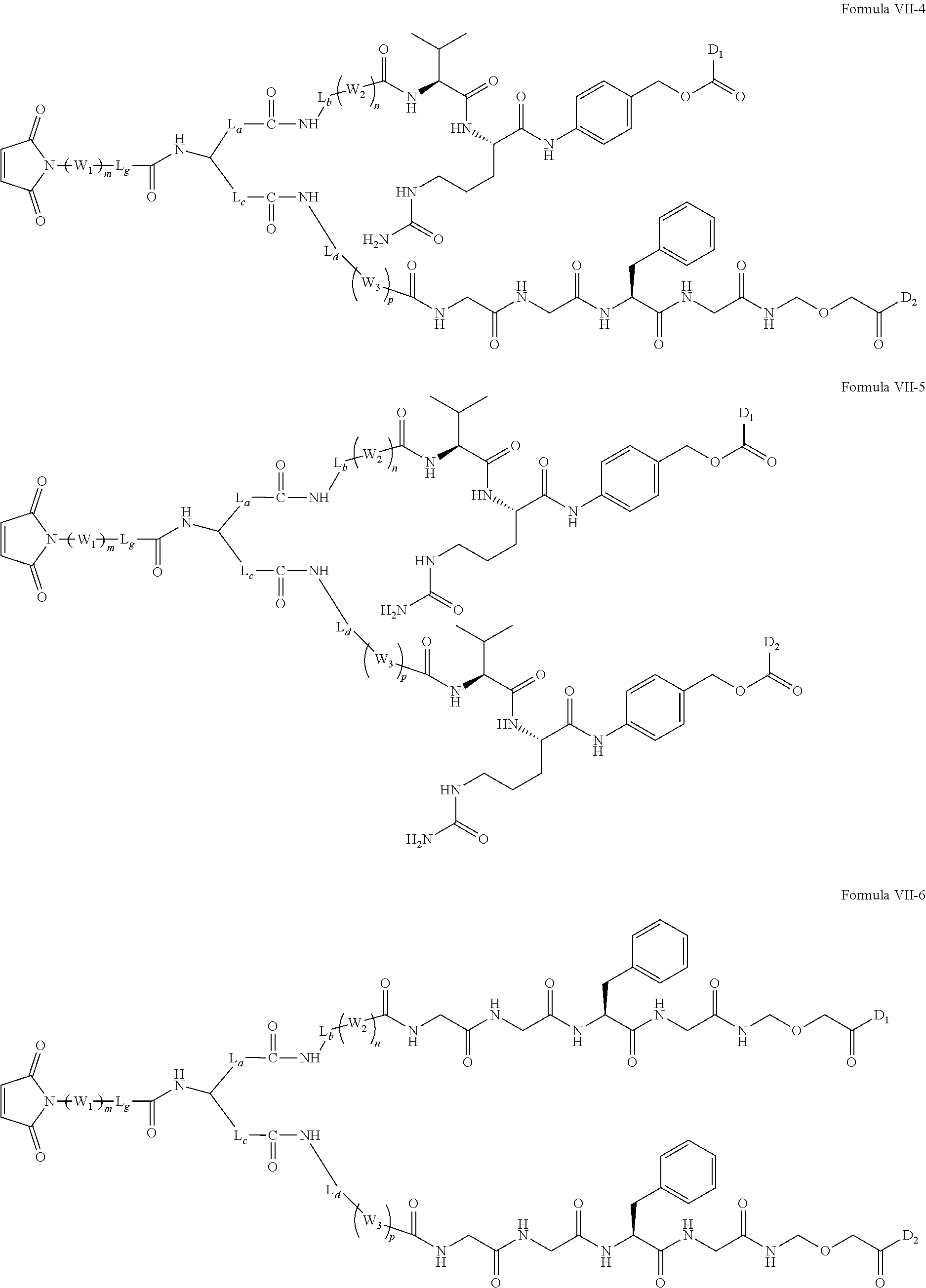

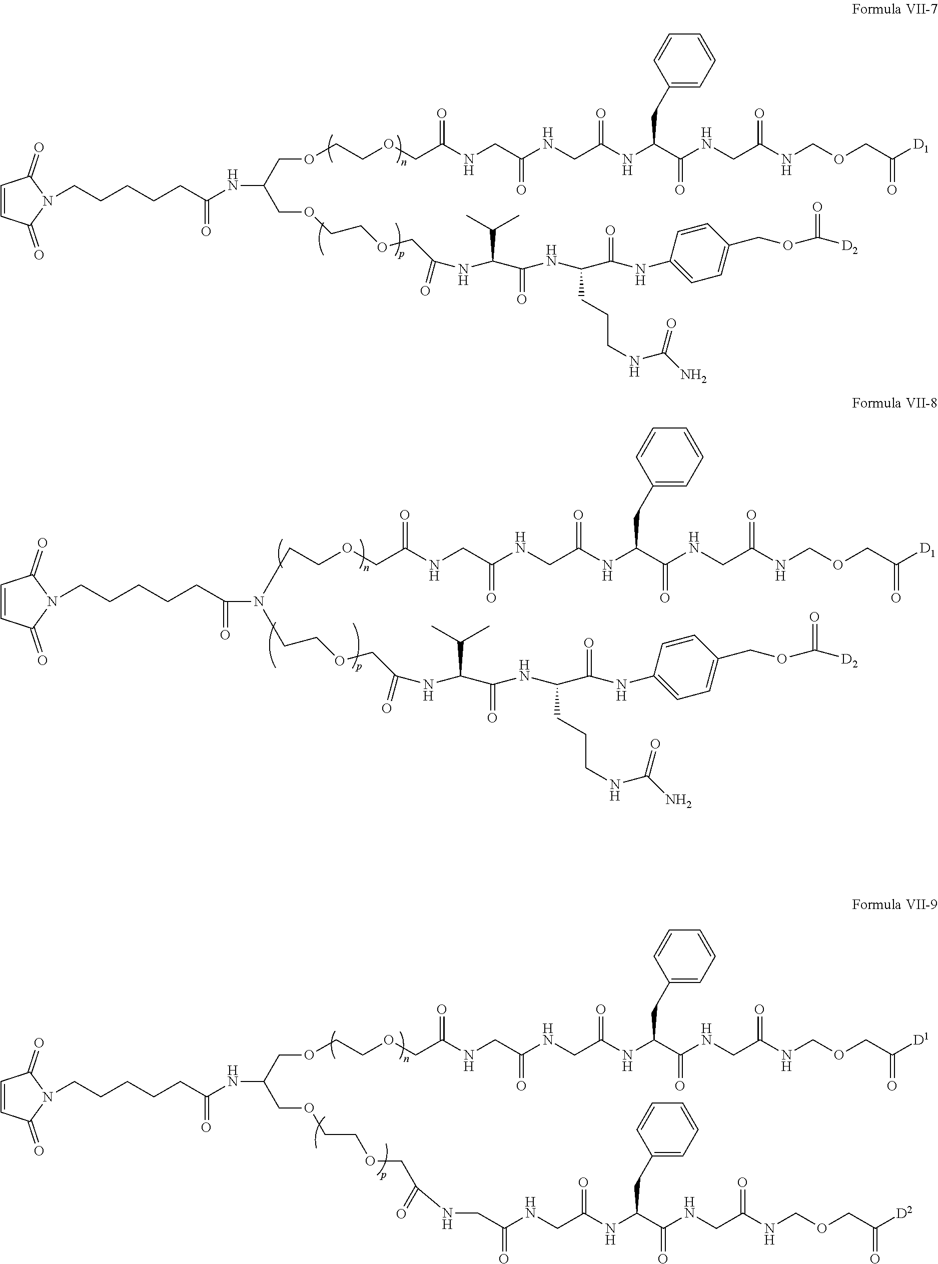

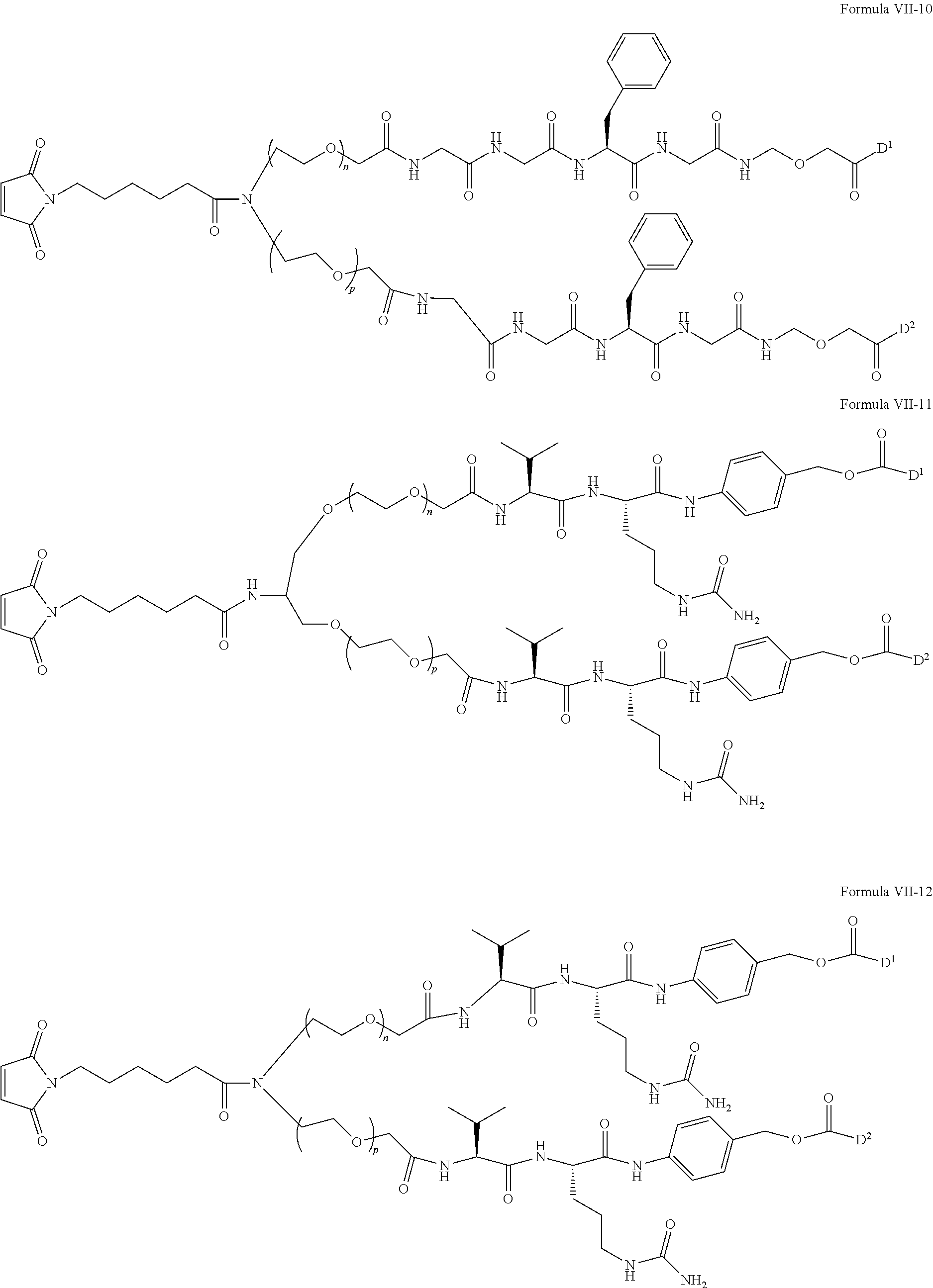

wherein, m is selected from an integer of 0-8;

n and p are each independently selected from an integer of 0-20, as well as n and p are not both 0;

W₁, W₂, and W₃ are each independently selected from the group consisting of methylene,

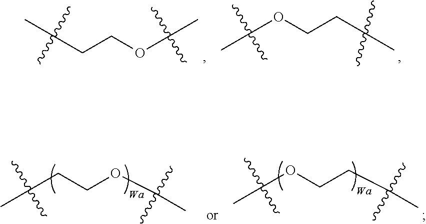

Wa is selected from an integer of 2-3; and at least one of W₂ and W₃ is

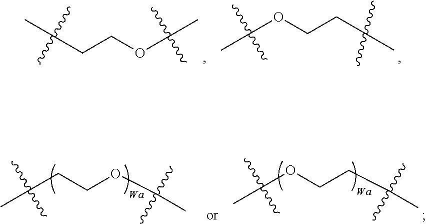

Lₐ, L_{b}, L_{c}, L_{d}, and L_{g} are each independently selected from the group consisting of absence, methylene or ethylene;

D₁ and D₂ are as defined in the above.

Further, the structure of said dual-drug link assembly unit is one of the following structures:

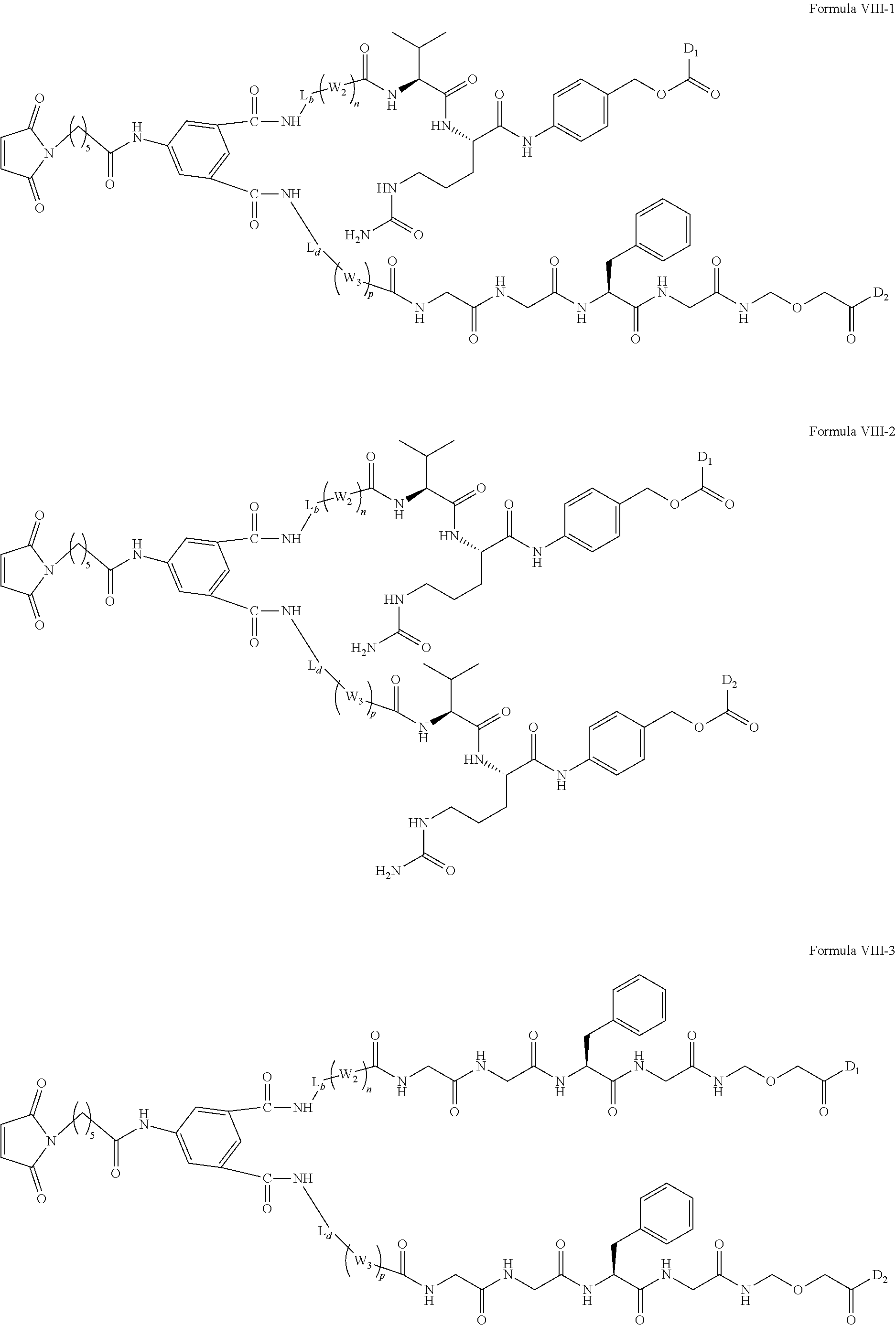

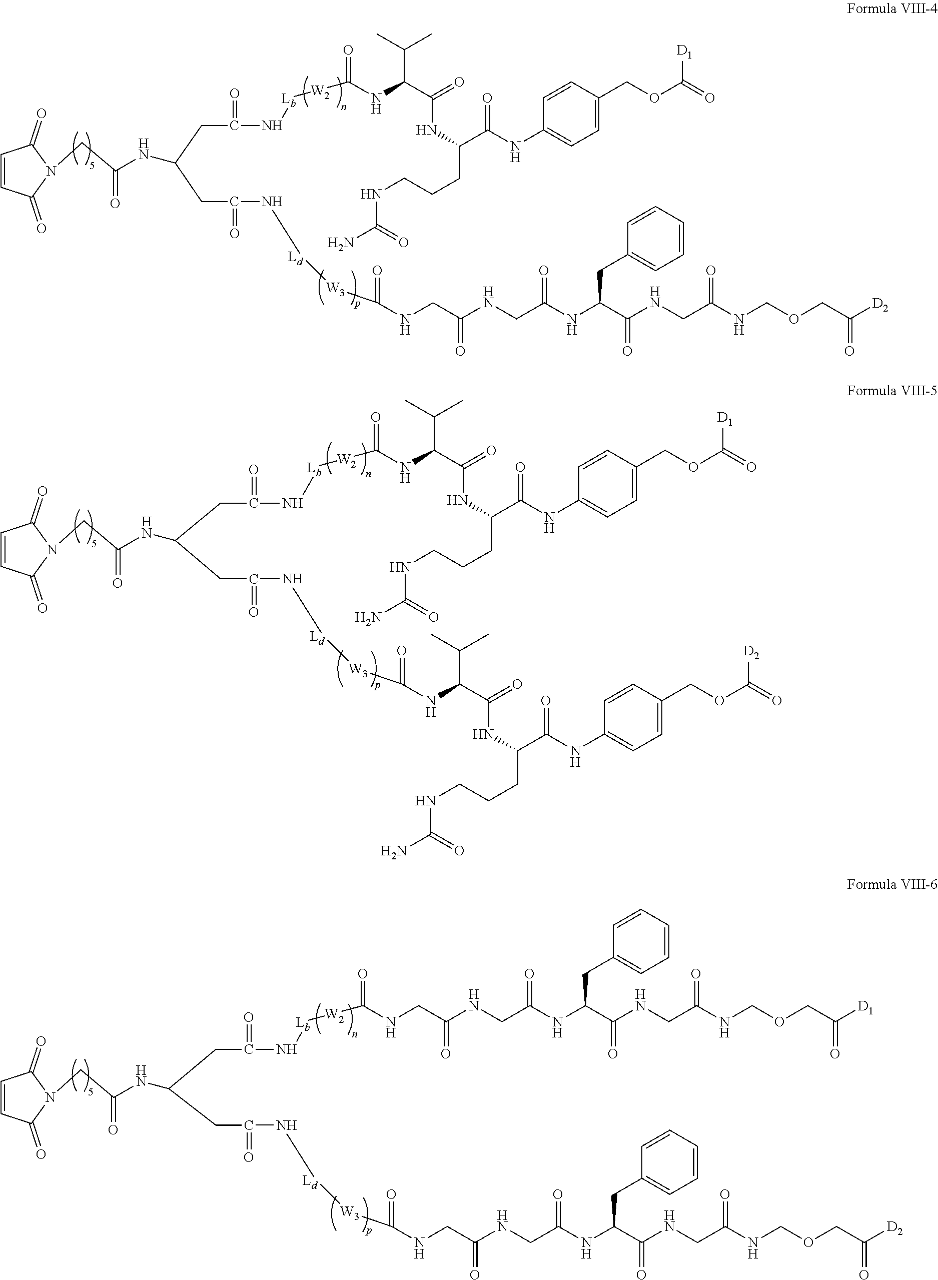

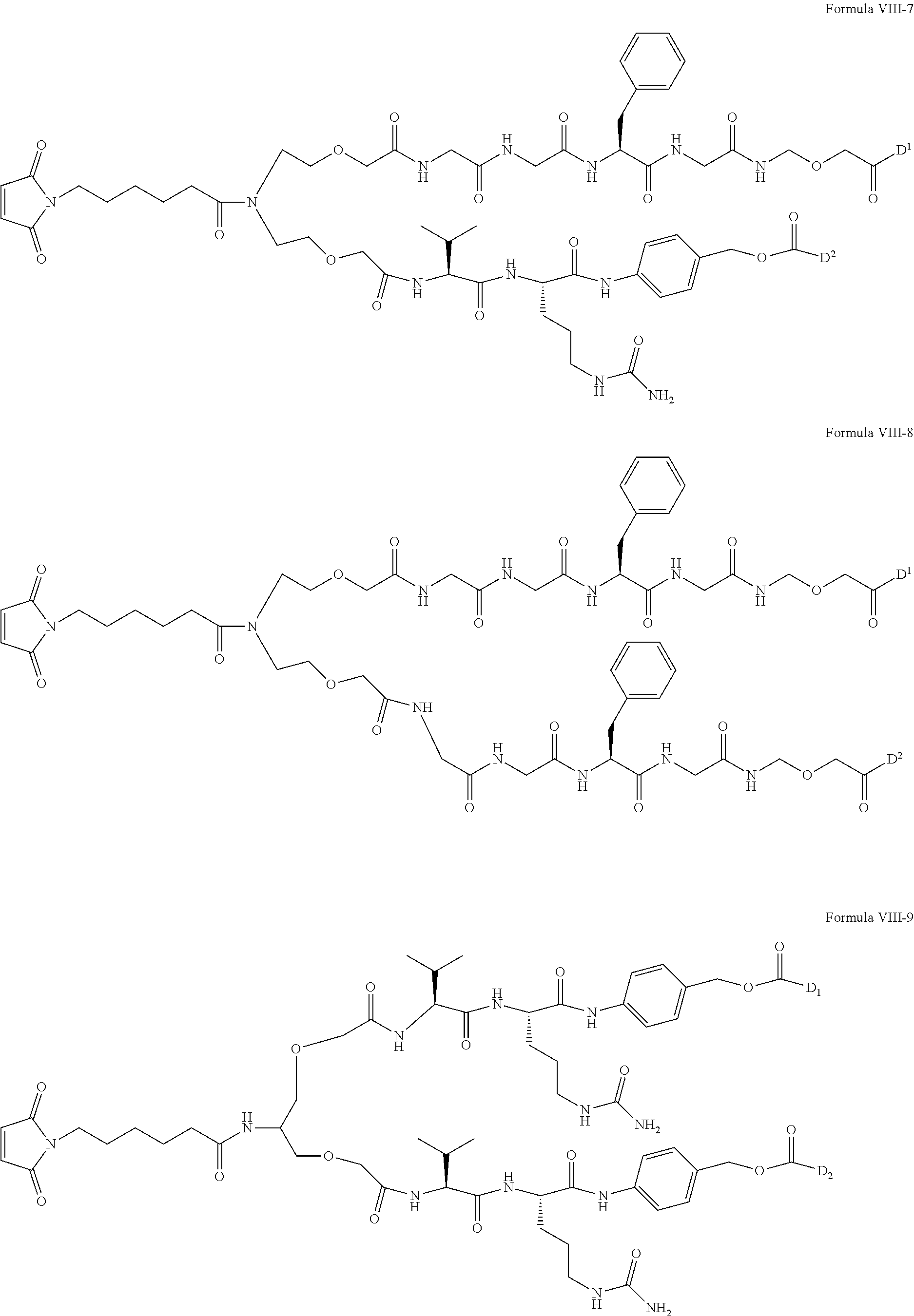

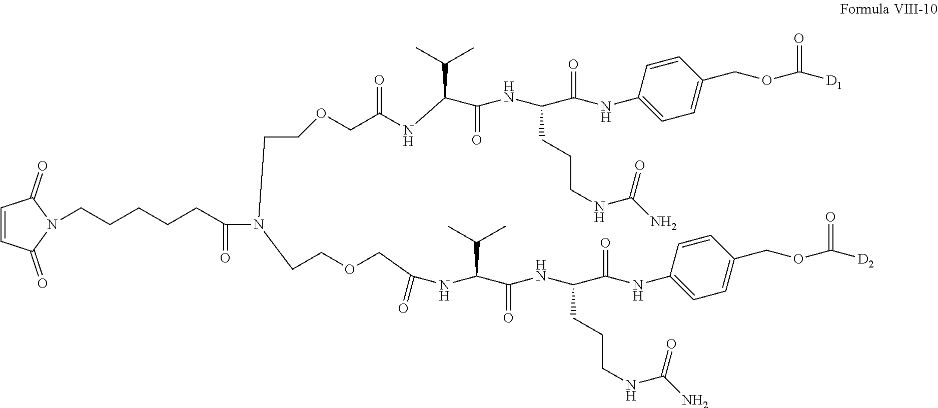

wherein, n and p are each independently selected from an integer of 0-20, and n and p are not both 0; W₂ and W₃ are each independently selected from the group consisting of methylene,

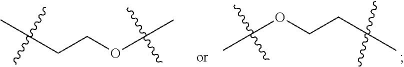 [SMILES: *CCO* ; *CCO*]

and at least one of W₂ and W₃ is

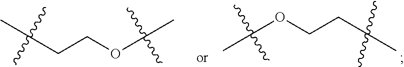 [SMILES: *CCO* ; *CCO*]

L_{b} and L_{d} are each independently selected from absence or ethylene;

D₁ and D₂ are as defined in the above.

Further, said D₁ and D₂ are each independently selected from the drug unit targeting TOP isomerase or the drug unit targeting microtubule proteins; the drug units targeting TOP isomerase are preferably SN-38, DXd, DX-8951 or derivatives thereof, and/or, and the drug units targeting microtubule proteins are preferably Eribulin, MMAE, MMAF, maytansine or derivatives thereof.

Further, the structure of said dual-drug link assembly unit is one of the following structures:

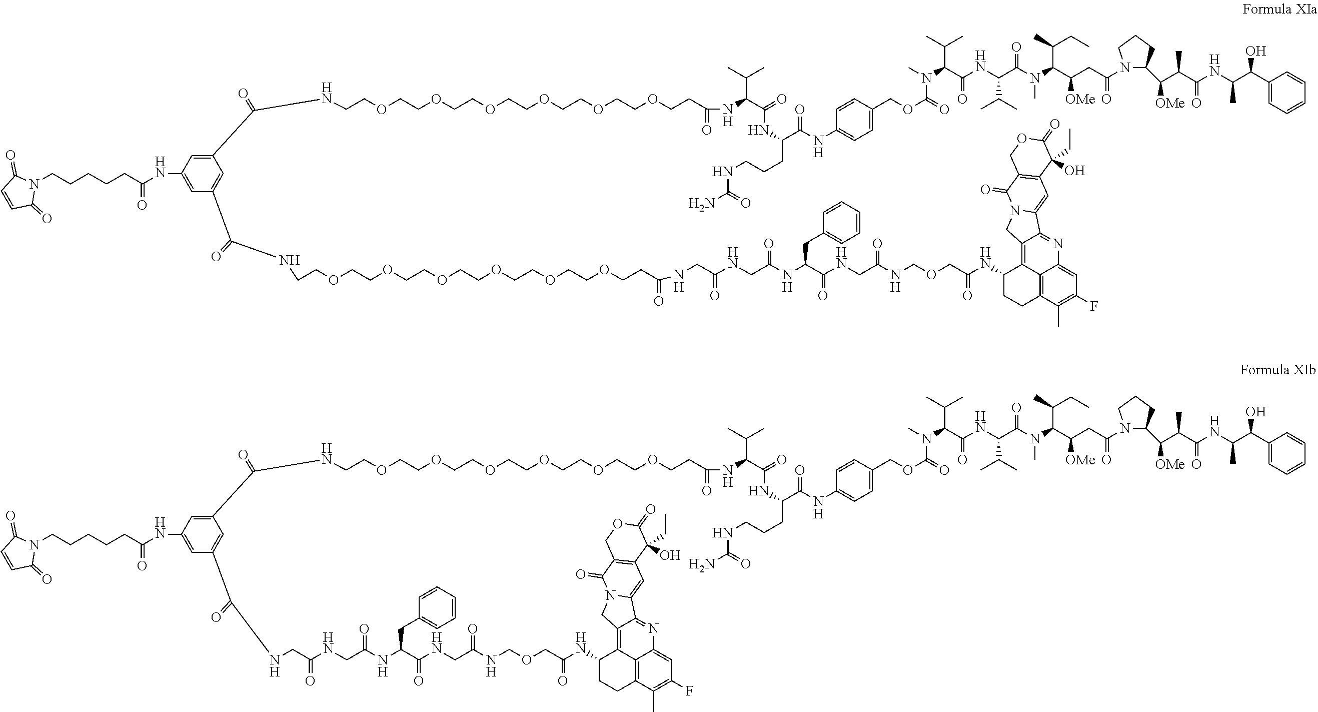

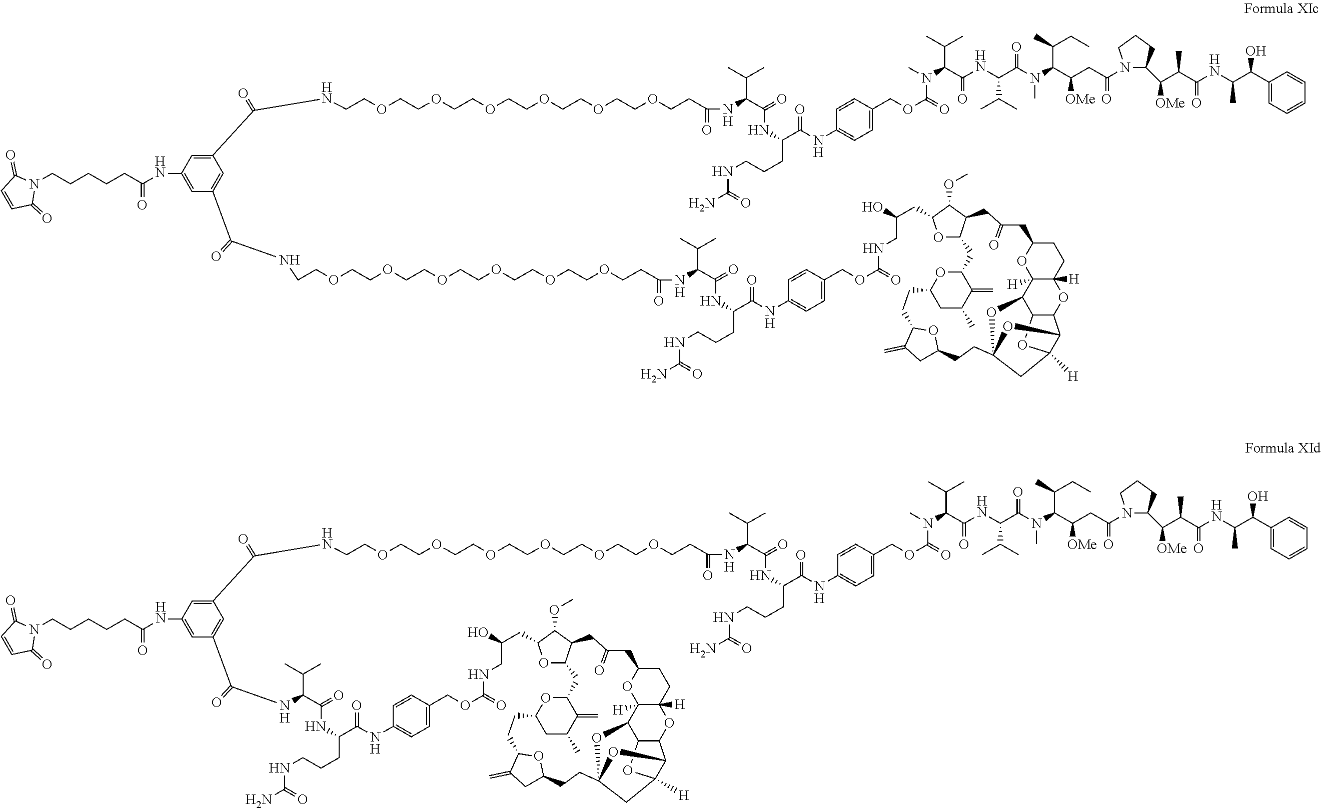

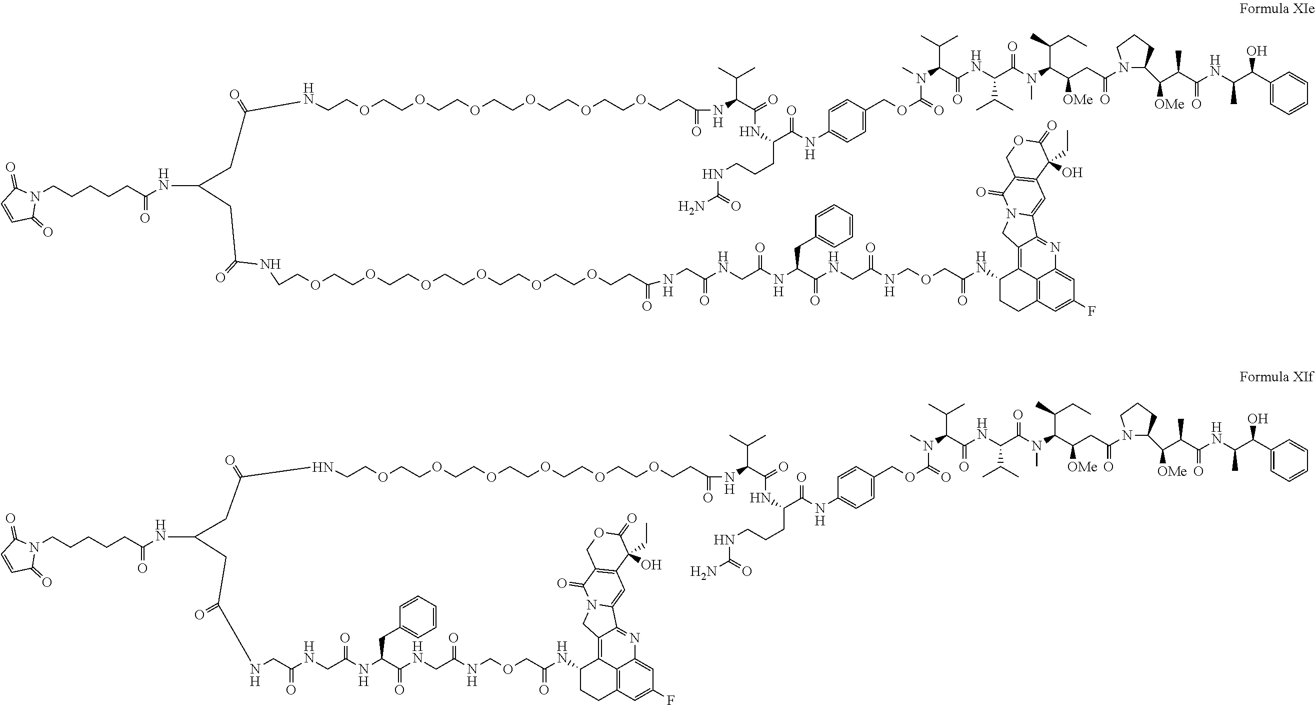

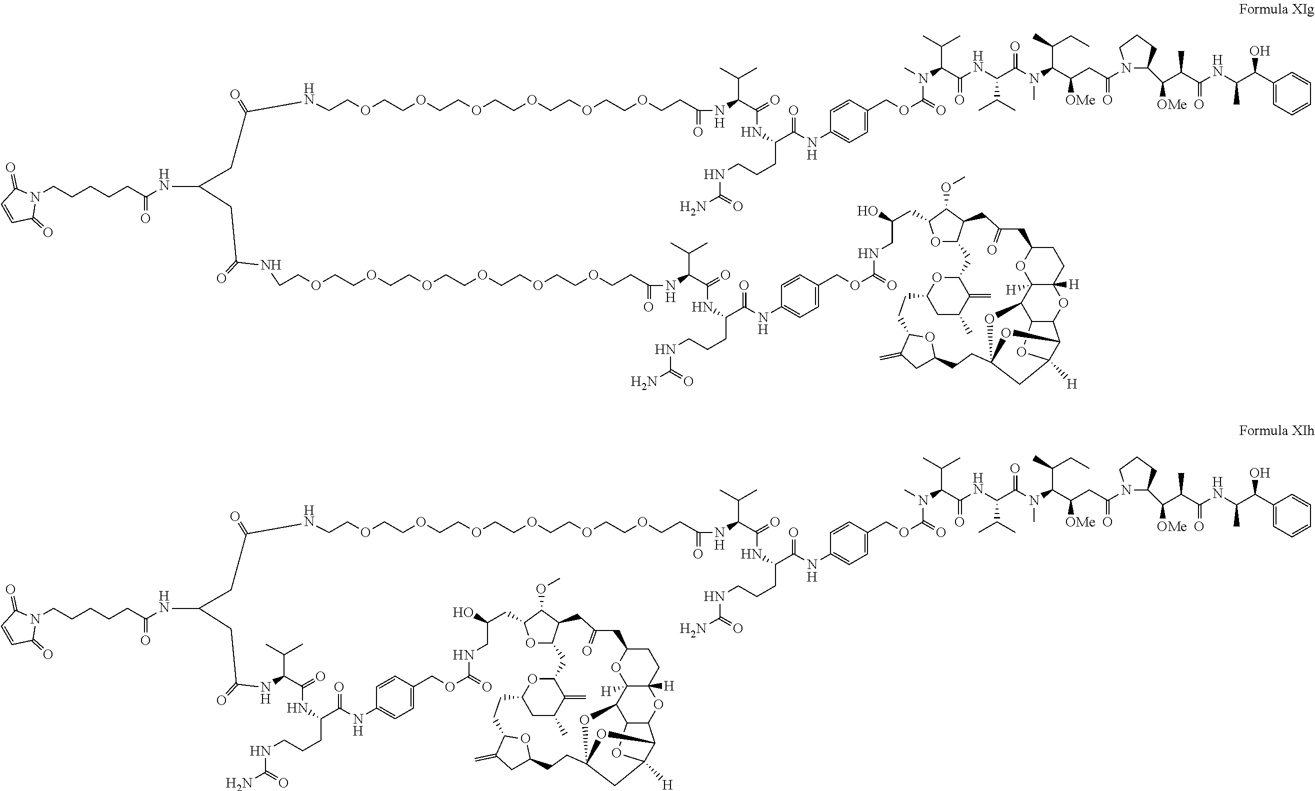

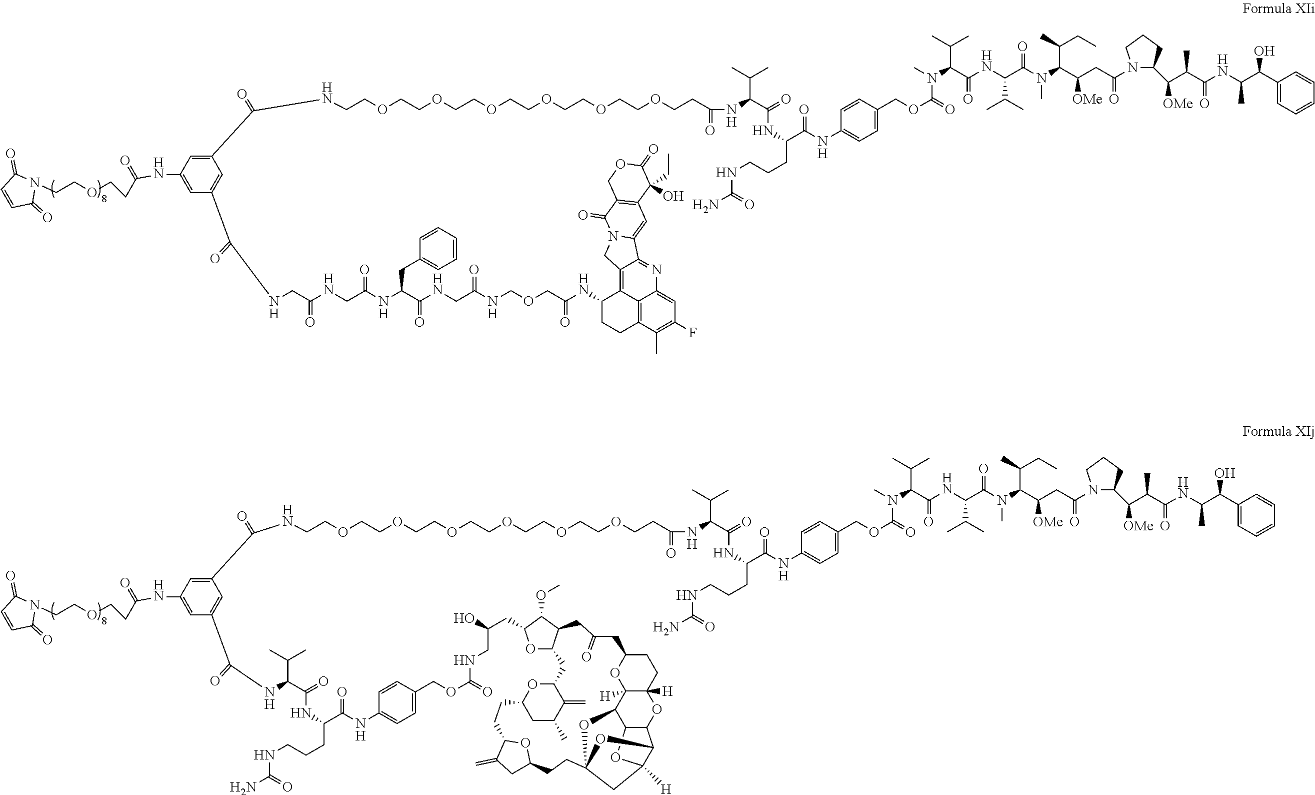

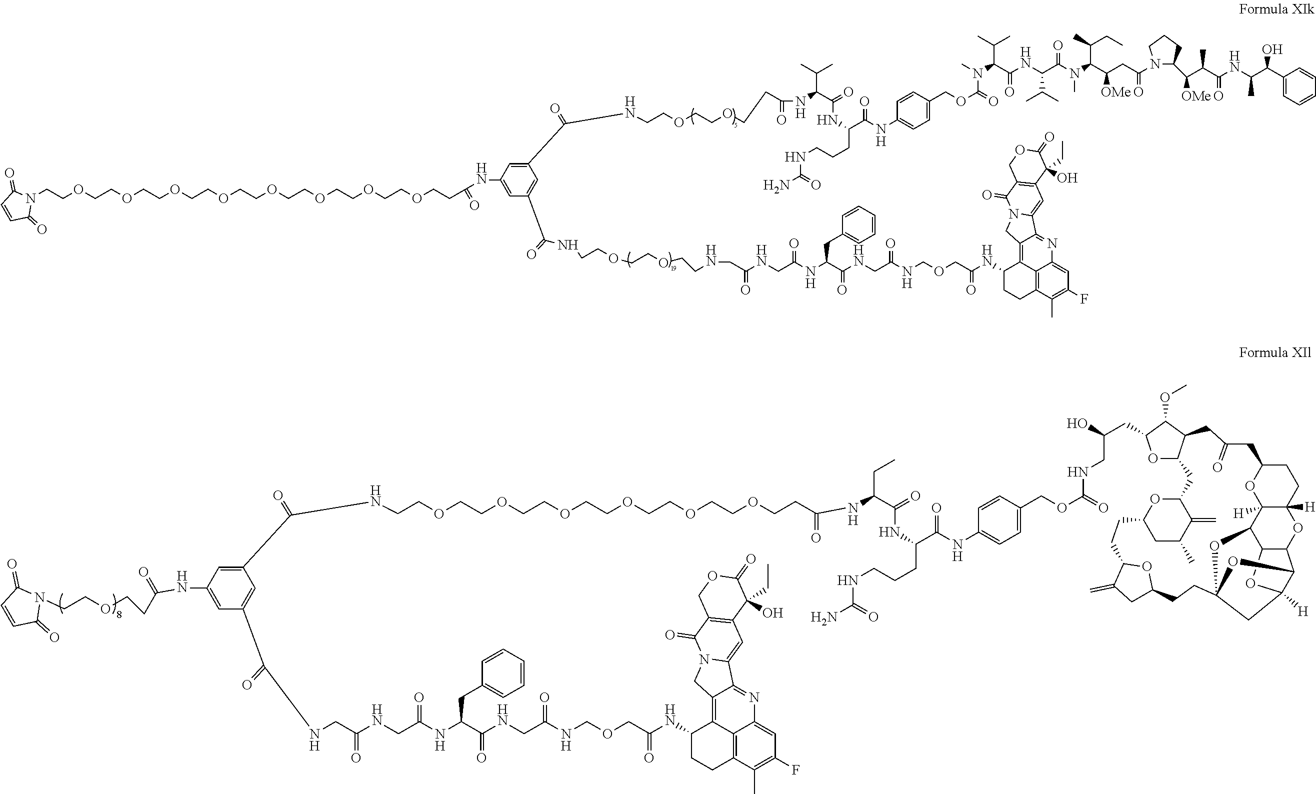

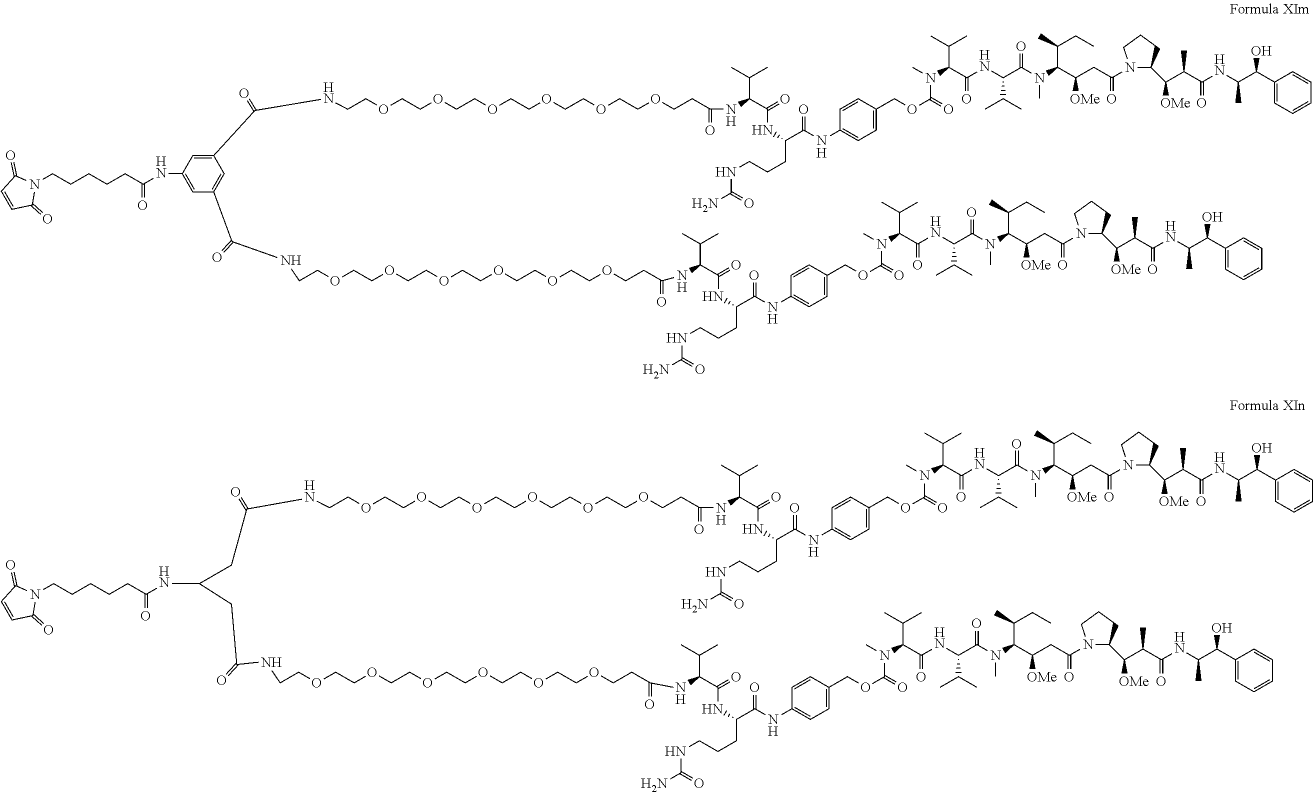

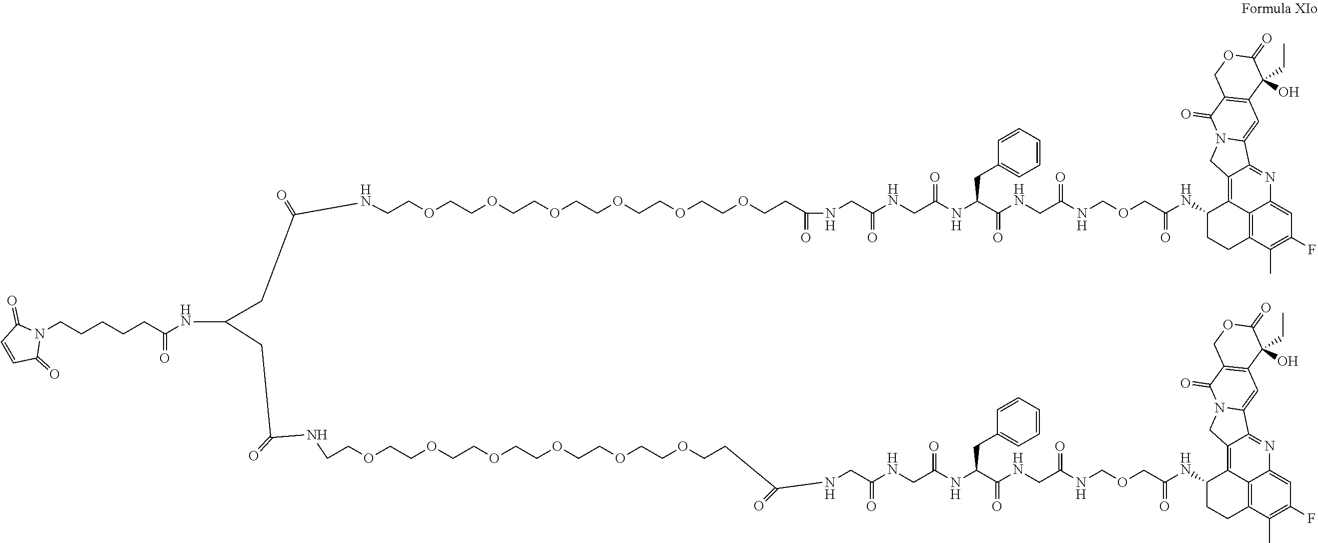 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCOCCOCCOCCOCCNC(=O)CC(CC(=O)NCCOCCOCCOCCOCCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)NC(=O)CCCCCN1C(=O)C=CC1=O)CC3]

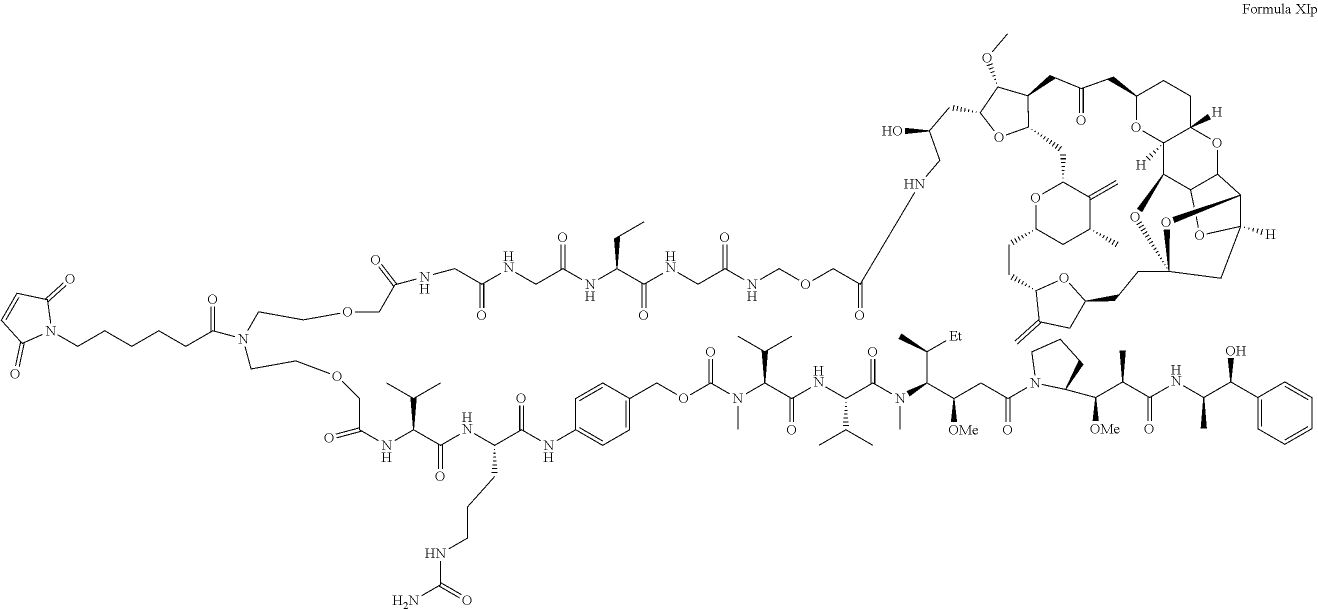 [SMILES: C=C1C[C@@H]2CC[C@@]34C[C@H]5OCC6C(O[C@H]7CC[C@H](CC(=O)C[C@@H]8[C@@H](OC)[C@@H](C[C@H](O)CNC(=O)COCNC(=O)CNC(=O)[C@H](CC)NC(=O)CNC(=O)CNC(=O)COCCN(CCOCC(=O)N[C@H](C(=O)N[C@@H](CCCNC(N)=O)C(=O)Nc9ccc(COC(=O)N(C)[C@H](C(=O)N[C@H](C(=O)N(C)[C@@H]([C@@H](C)CC)[C@@H](CC(=O)N%10CCC[C@H]%10[C@H](OC)[C@@H](C)C(=O)N[C@H](C)[C@@H](O)c%10ccccc%10)OC)C(C)C)C(C)C)cc9)C(C)C)C(=O)CCCCCN9C(=O)C=CC9=O)O[C@H]8C[C@H]8O[C@@H](CC[C@@H]1O2)C[C@@H](C)C8=C)O[C@@H]7[C@@H]6O3)[C@H]5O4]

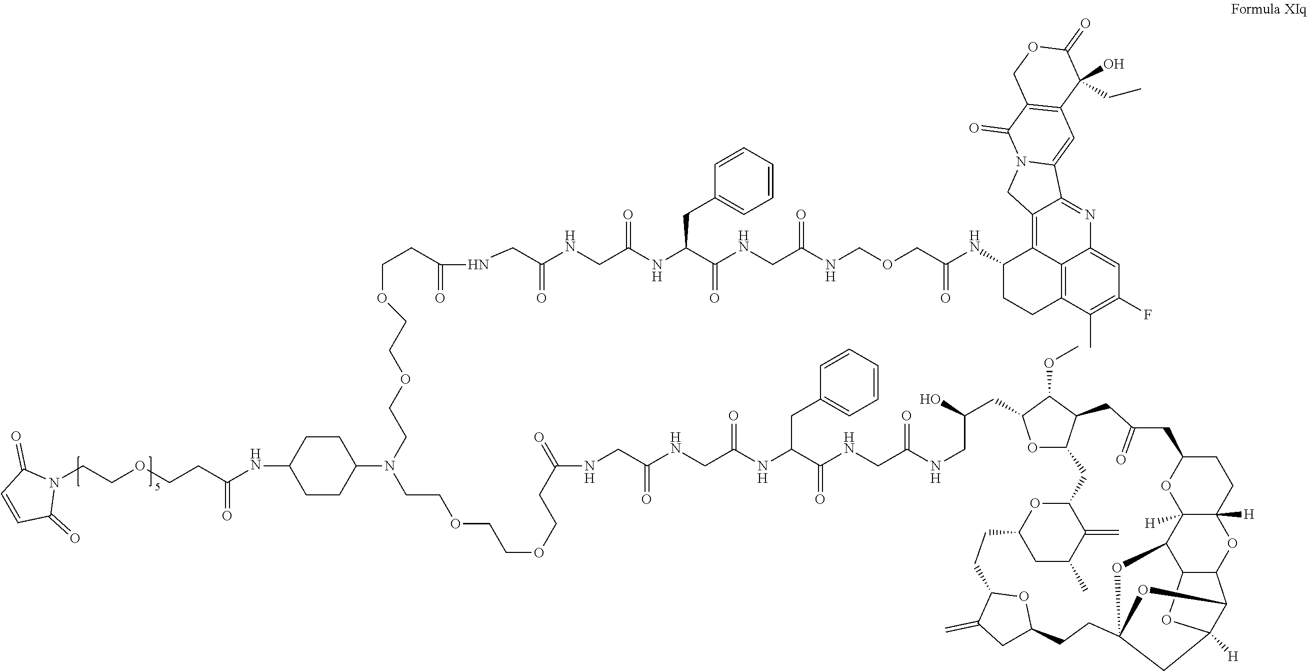

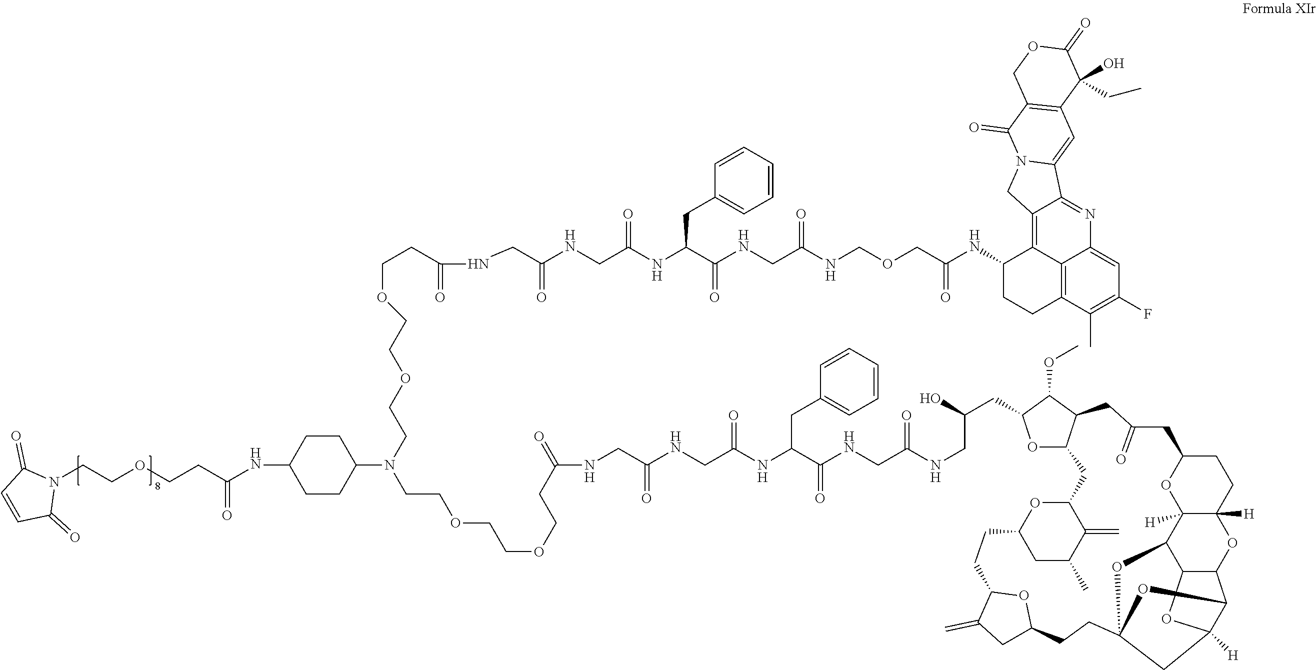

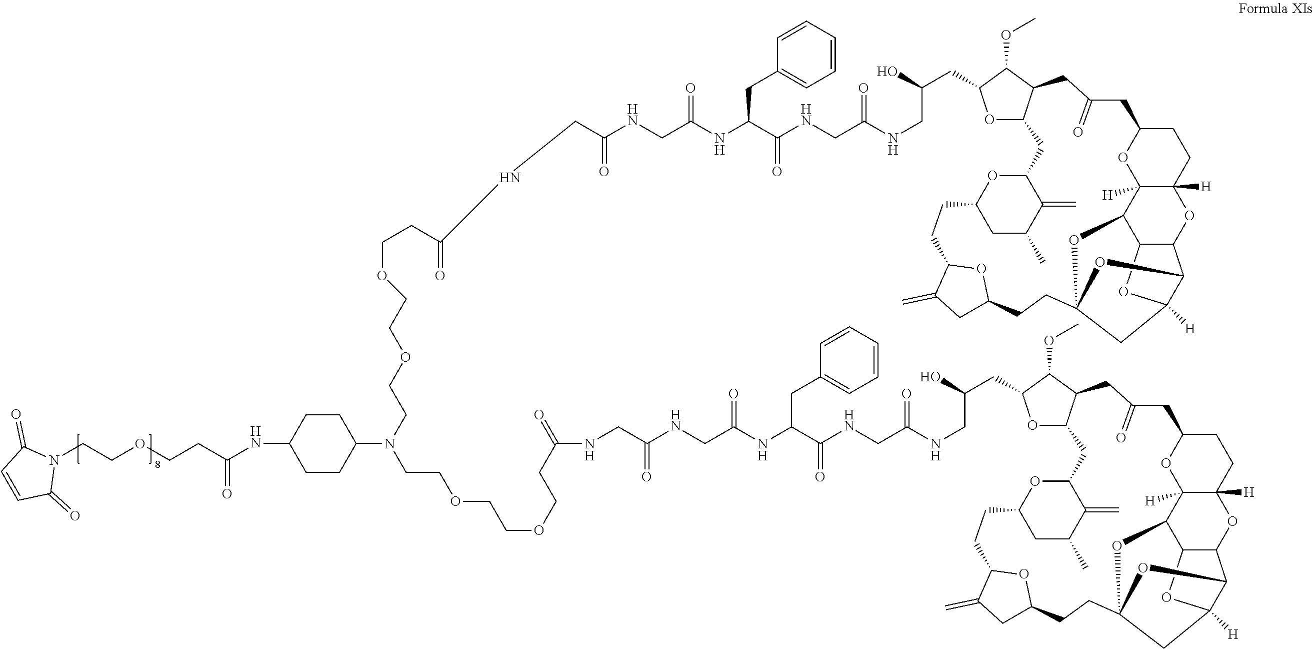

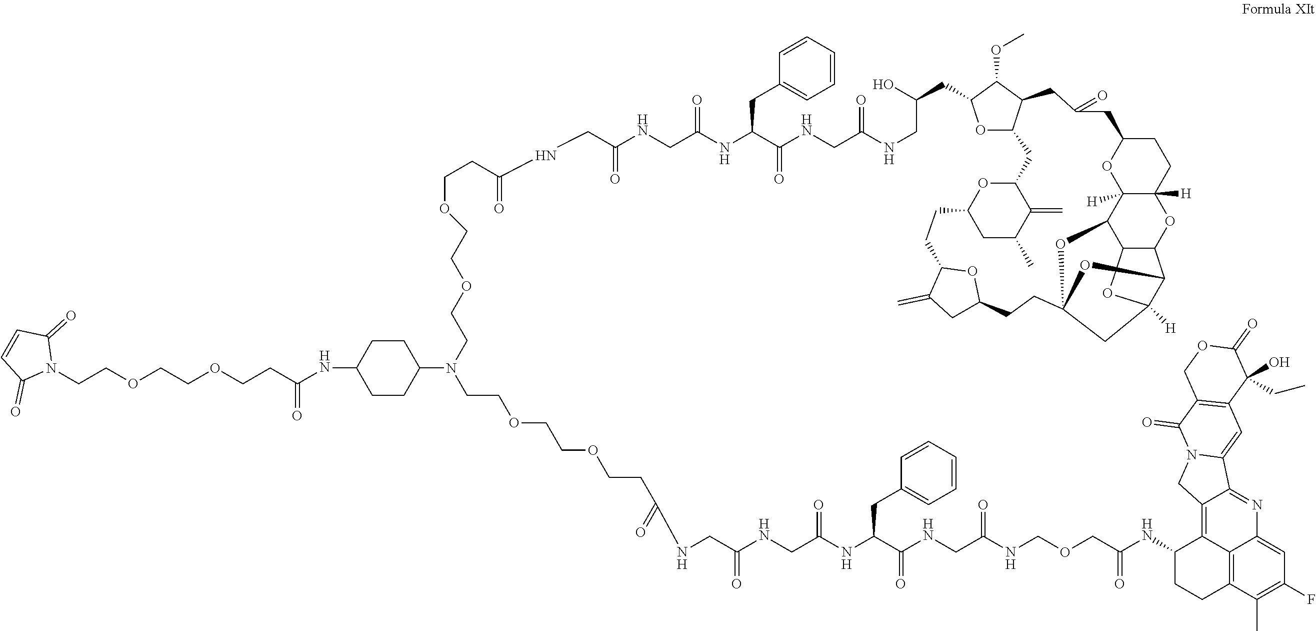 [SMILES: C=C1C[C@@H]2CC[C@@]34C[C@H]5OCC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)CCOCCOCCN(CCOCCOCCC(=O)NCC(=O)NCC(=O)N[C@@H](Cc9ccccc9)C(=O)NCC(=O)NCOCC(=O)N[C@H]9CCc%10c(C)c(F)cc%11nc%12c(c9c%10%11)Cn9c-%12cc%10c(c9=O)COC(=O)[C@]%10(O)CC)C9CCC(NC(=O)CCOCCOCCN%10C(=O)C=CC%10=O)CC9)O[C@H]8C[C@H]8O[C@@H](CC[C@@H]1O2)C[C@@H](C)C8=C)O[C@@H]7[C@@H]6O3)[C@H]5O4]

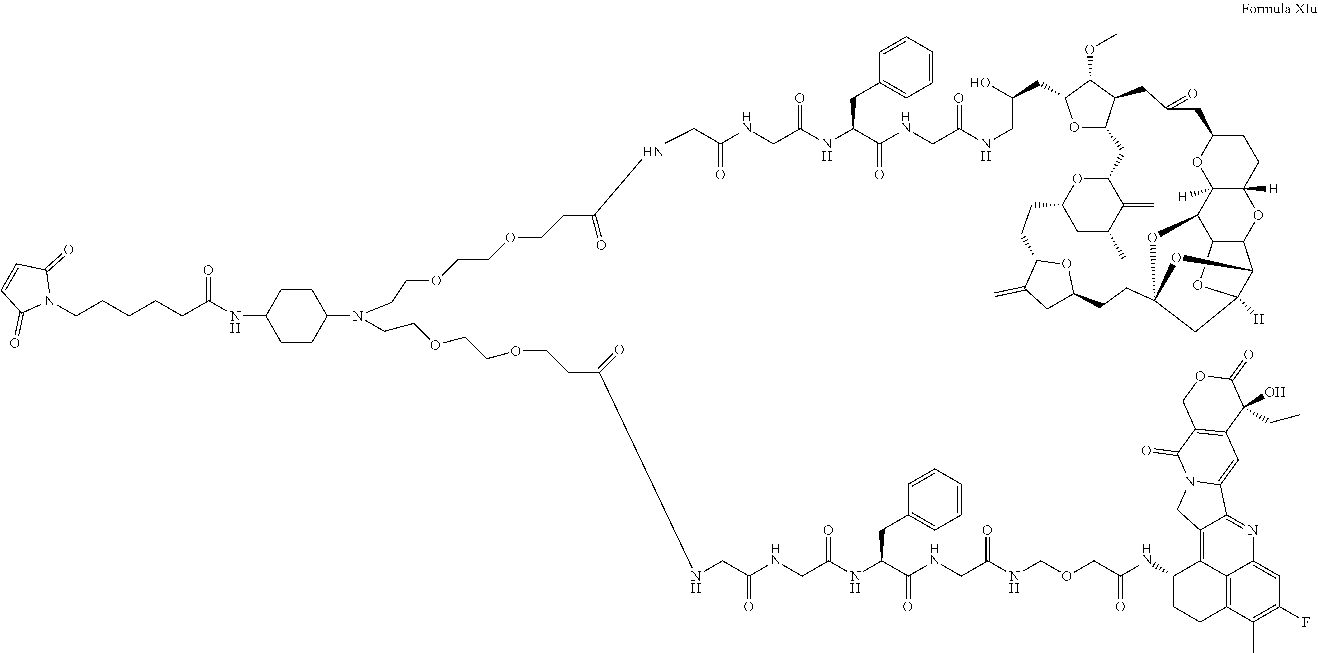 [SMILES: C=C1C[C@@H]2CC[C@@]34C[C@H]5OCC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)CCOCCOCCN(CCOCCOCCC(=O)NCC(=O)NCC(=O)N[C@@H](Cc9ccccc9)C(=O)NCC(=O)NCOCC(=O)N[C@H]9CCc%10c(C)c(F)cc%11nc%12c(c9c%10%11)Cn9c-%12cc%10c(c9=O)COC(=O)[C@]%10(O)CC)C9CCC(NC(=O)CCCCCN%10C(=O)C=CC%10=O)CC9)O[C@H]8C[C@H]8O[C@@H](CC[C@@H]1O2)C[C@@H](C)C8=C)O[C@@H]7[C@@H]6O3)[C@H]5O4]

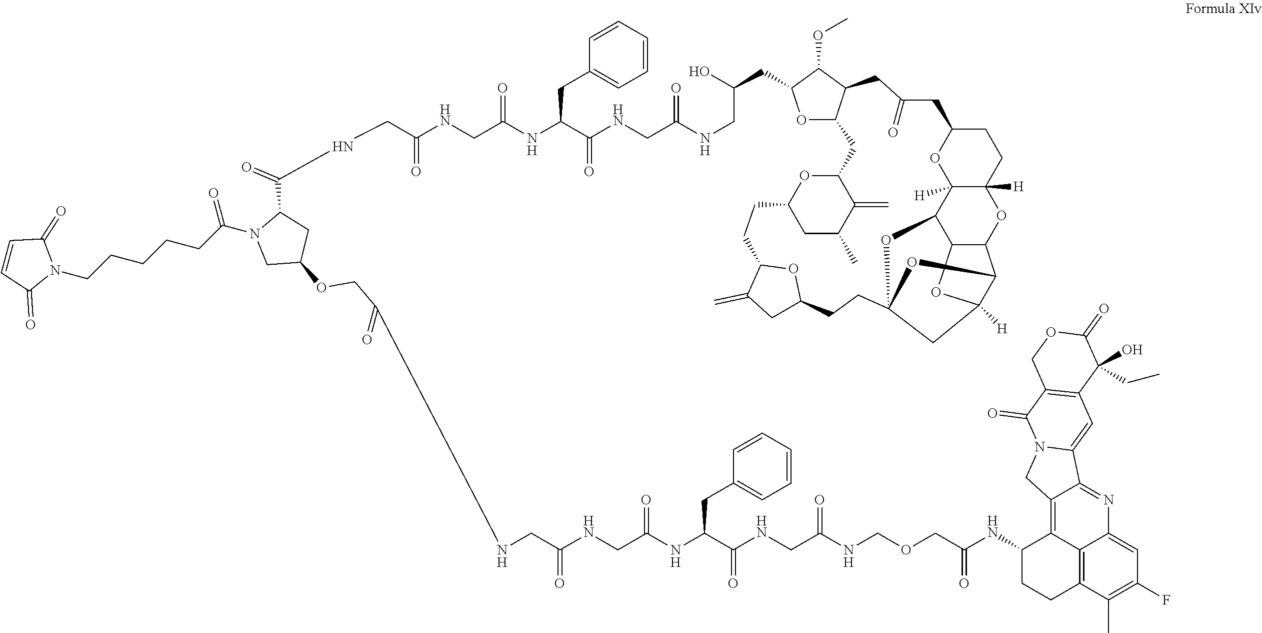 [SMILES: C=C1C[C@@H]2CC[C@@]34C[C@H]5OCC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)[C@@H]9C[C@@H](OCC(=O)NCC(=O)NCC(=O)N[C@@H](Cc%10ccccc%10)C(=O)NCC(=O)NCOCC(=O)N[C@H]%10CCc%11c(C)c(F)cc%12nc%13c(c%10c%11%12)Cn%10c-%13cc%11c(c%10=O)COC(=O)[C@]%11(O)CC)CN9C(=O)CCCCCN9C(=O)C=CC9=O)O[C@H]8C[C@H]8O[C@@H](CC[C@@H]1O2)C[C@@H](C)C8=C)O[C@@H]7[C@@H]6O3)[C@H]5O4]

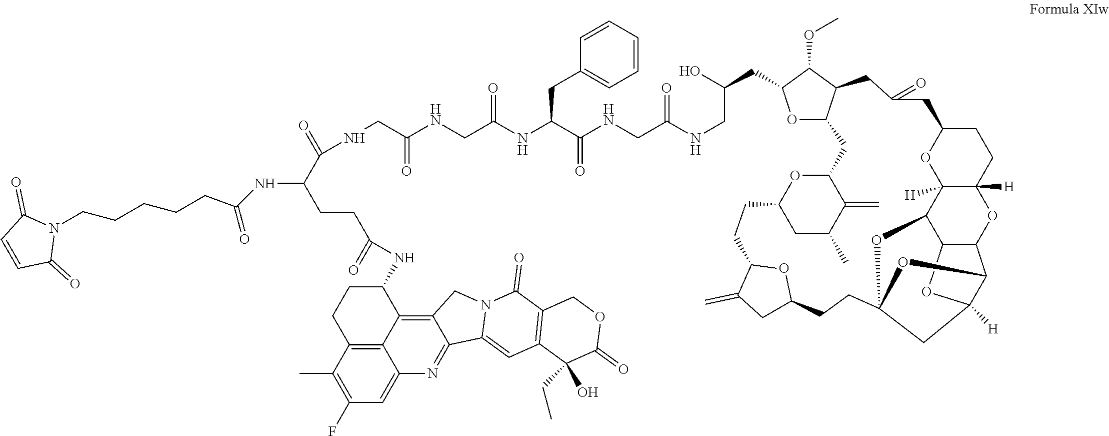 [SMILES: C=C1C[C@@H]2CC[C@@]34C[C@H]5OCC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)C(CCC(=O)N[C@H]9CCc%10c(C)c(F)cc%11nc%12c(c9c%10%11)Cn9c-%12cc%10c(c9=O)COC(=O)[C@]%10(O)CC)NC(=O)CCCCCN9C(=O)C=CC9=O)O[C@H]8C[C@H]8O[C@@H](CC[C@@H]1O2)C[C@@H](C)C8=C)O[C@@H]7[C@@H]6O3)[C@H]5O4]

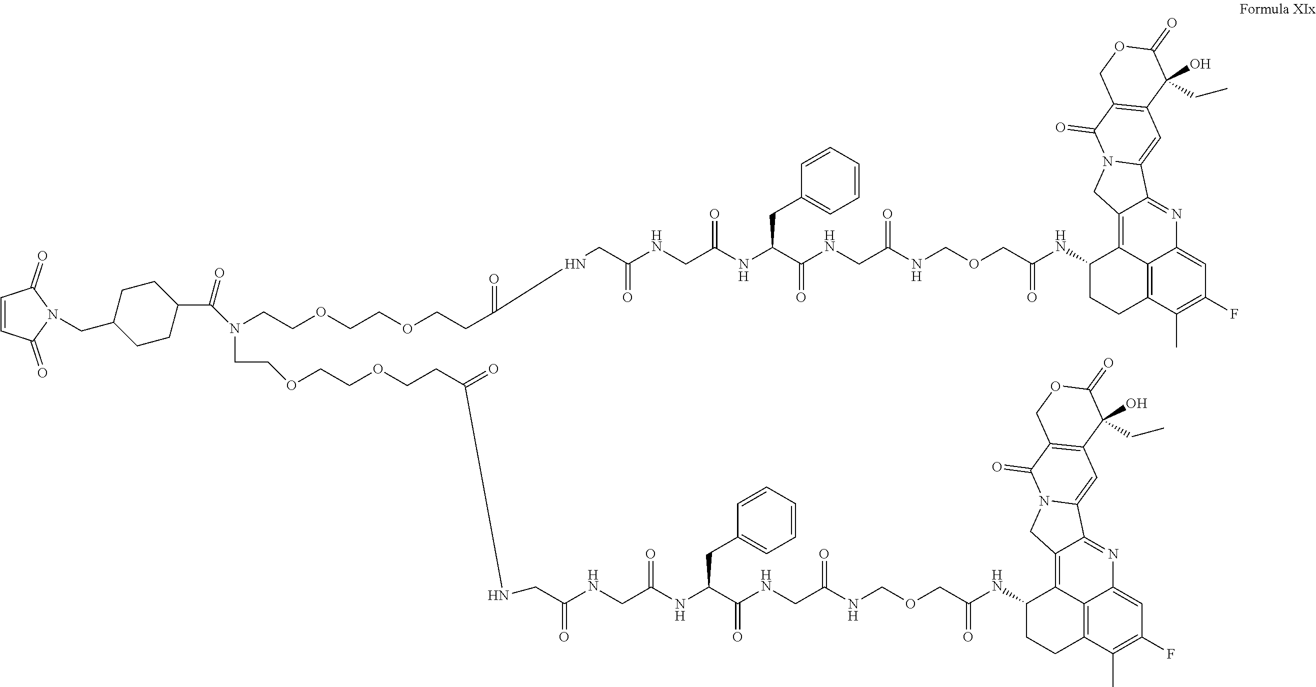 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCN(CCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)C(=O)C1CCC(CN2C(=O)C=CC2=O)CC1)CC3]

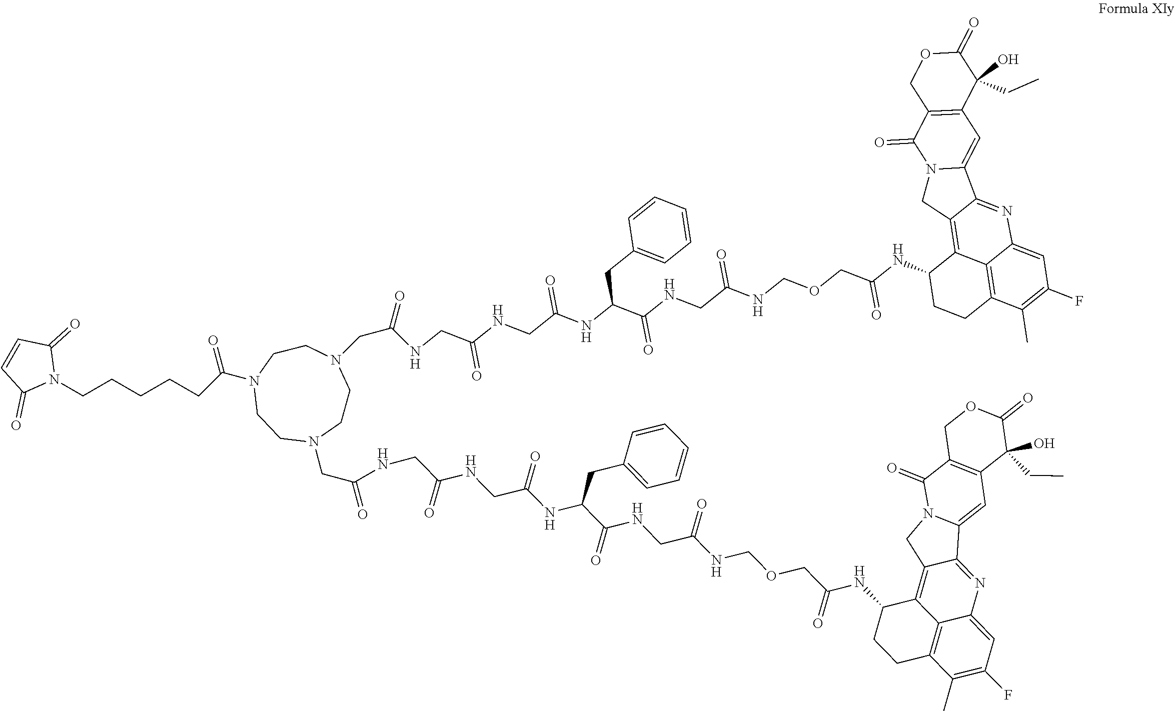 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CN1CCN(CC(=O)NCC(=O)NCC(=O)N[C@@H](Cc2ccccc2)C(=O)NCC(=O)NCOCC(=O)N[C@H]2CCc4c(C)c(F)cc5nc6c(c2c45)Cn2c-6cc4c(c2=O)COC(=O)[C@]4(O)CC)CCN(C(=O)CCCCCN2C(=O)C=CC2=O)CC1)CC3]

The present invention also provides a dual-drug targeting linker-drug conjugate molecule, or a stereoisomer thereof, or an optical isomer thereof, or a deuterated compound thereof, and the dual-drug targeting linker-drug conjugate is obtained by connecting the targeting linker and q dual-drug link assembly unit(s) mentioned above; the targeting linker is a substance that can target and bind to the lesion site; the structure of the dual-drug targeting linker-drug coujugate is as represented by Formula I:

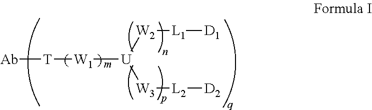

wherein, Ab is a targeting linker; 1≤q≤8; T, W₁, W₂, W₃, m, n, p, U, L₁, L₂, D₁, and D₂ are as defined in the above.

Further, the targeting linker is an antibody, an antibody fragment, a protein, a peptide or an aptamer, and the antibody is preferably an antibody targeting cell surface receptors and tumor-related antigens.

Further, the structure of said dual-drug targeting linker-drug conjugate is selected from one of the following structures:

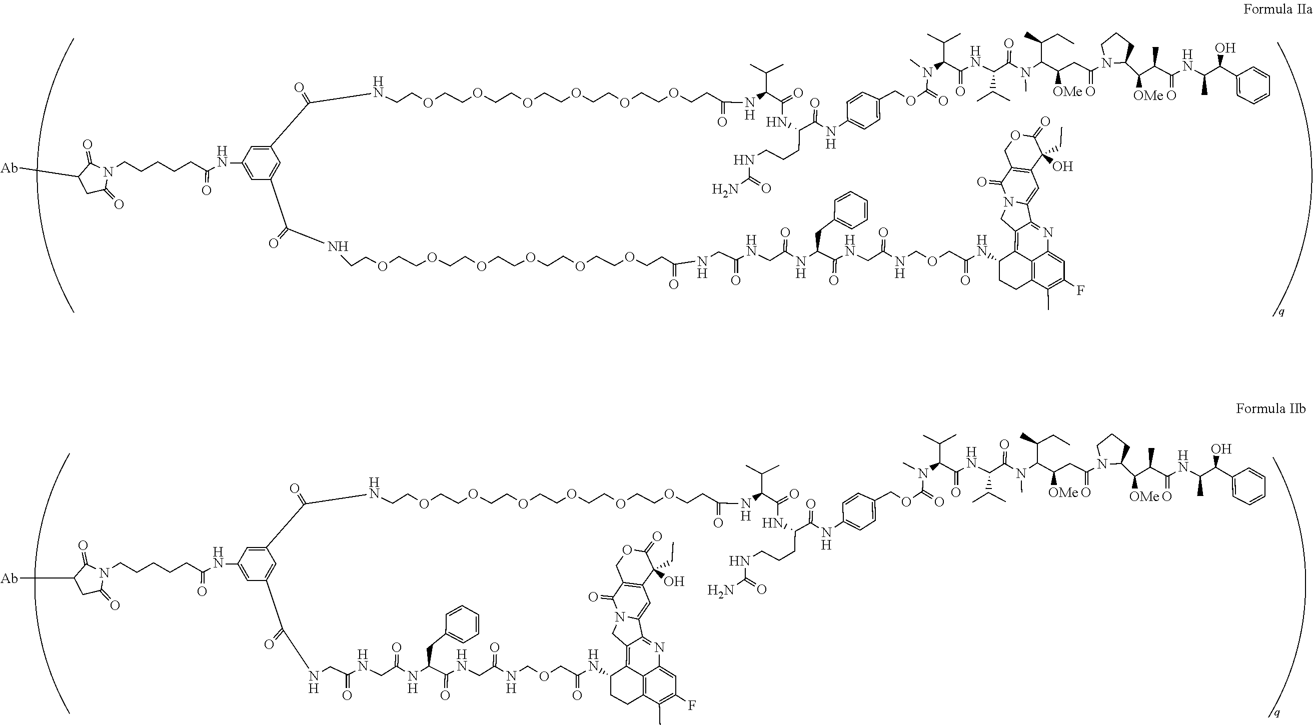

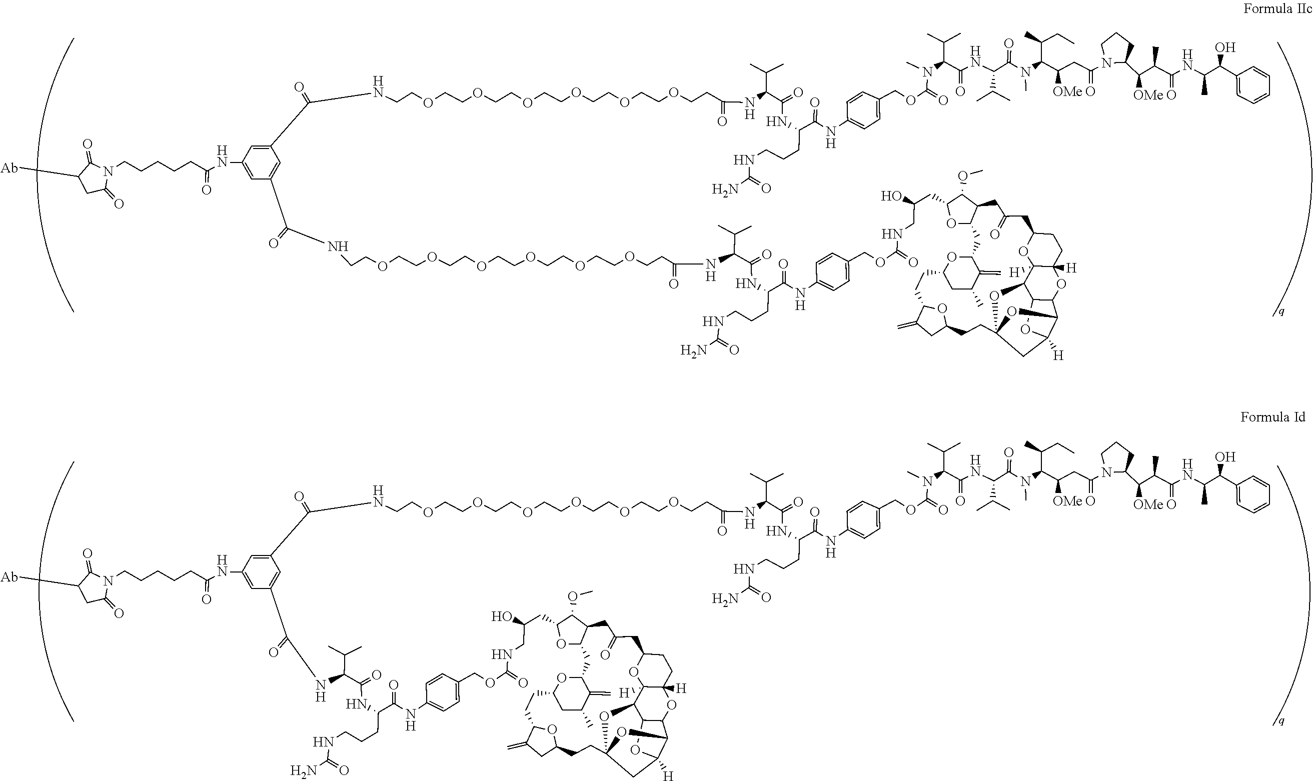

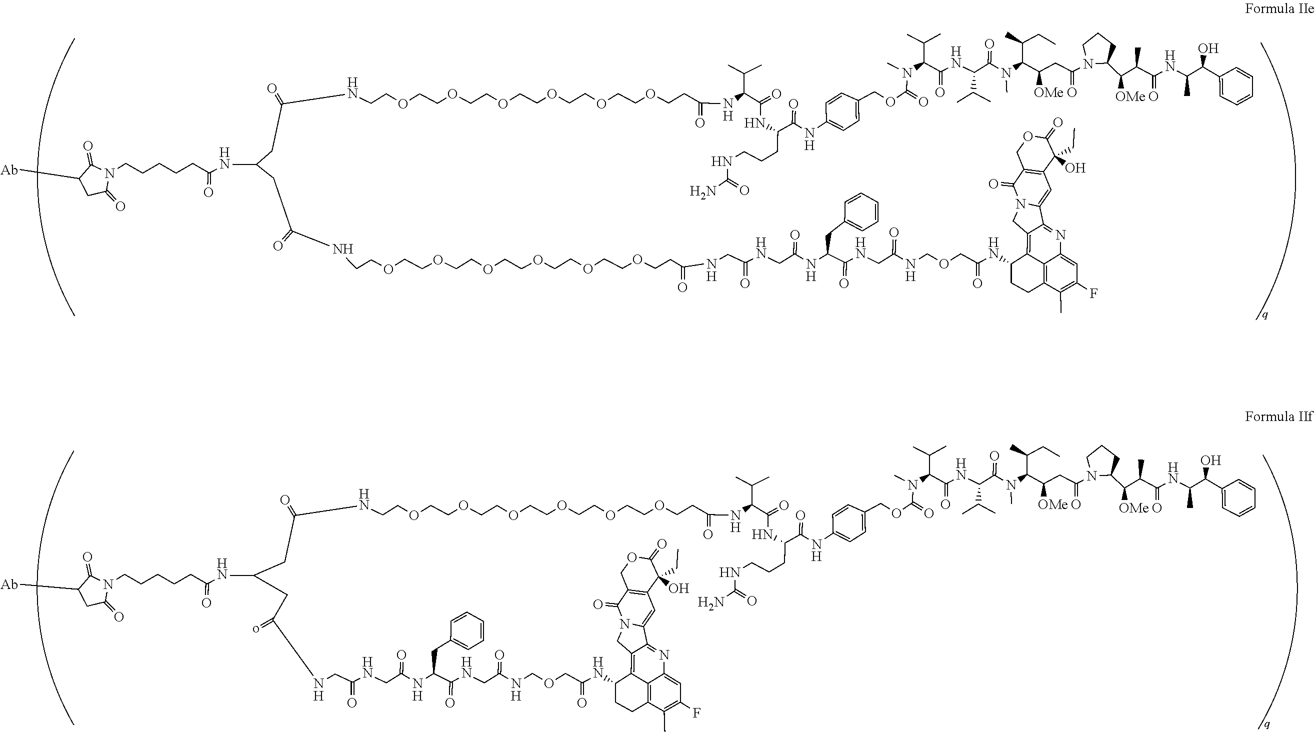

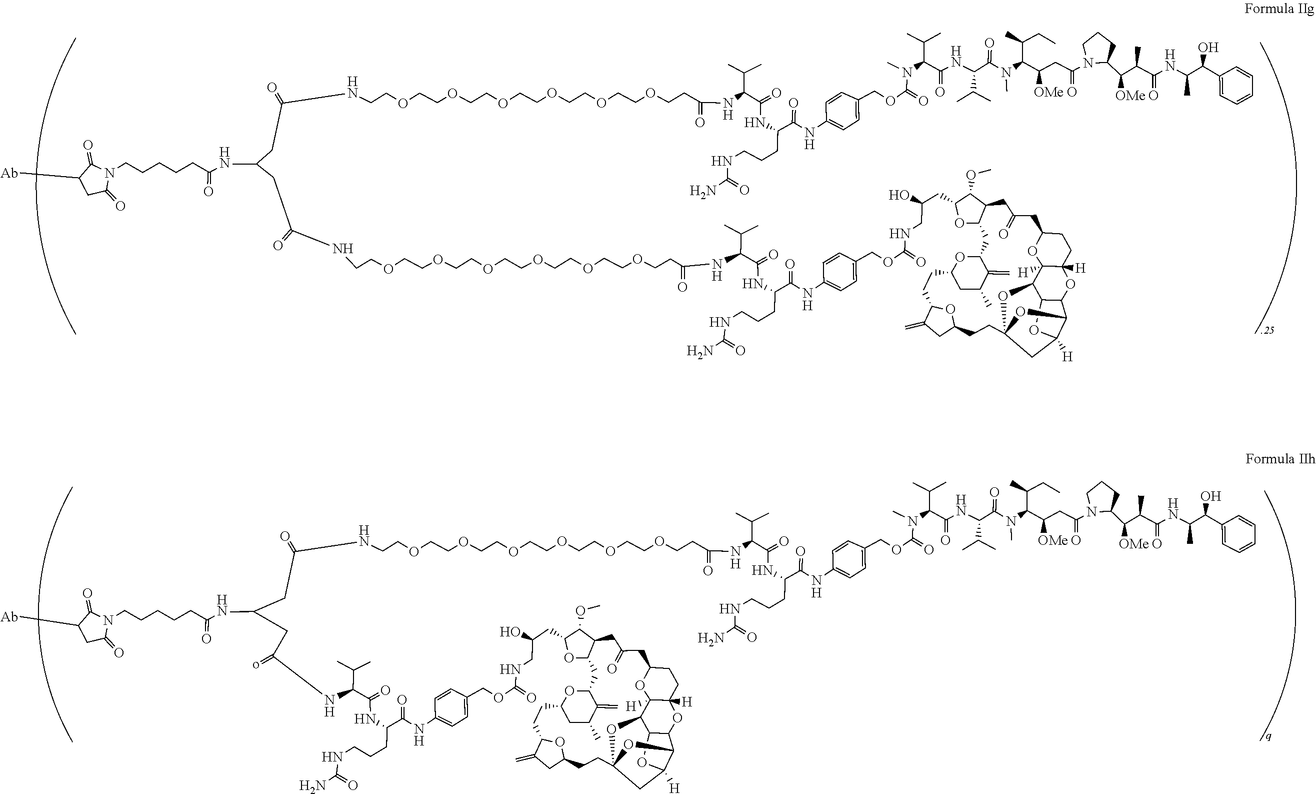

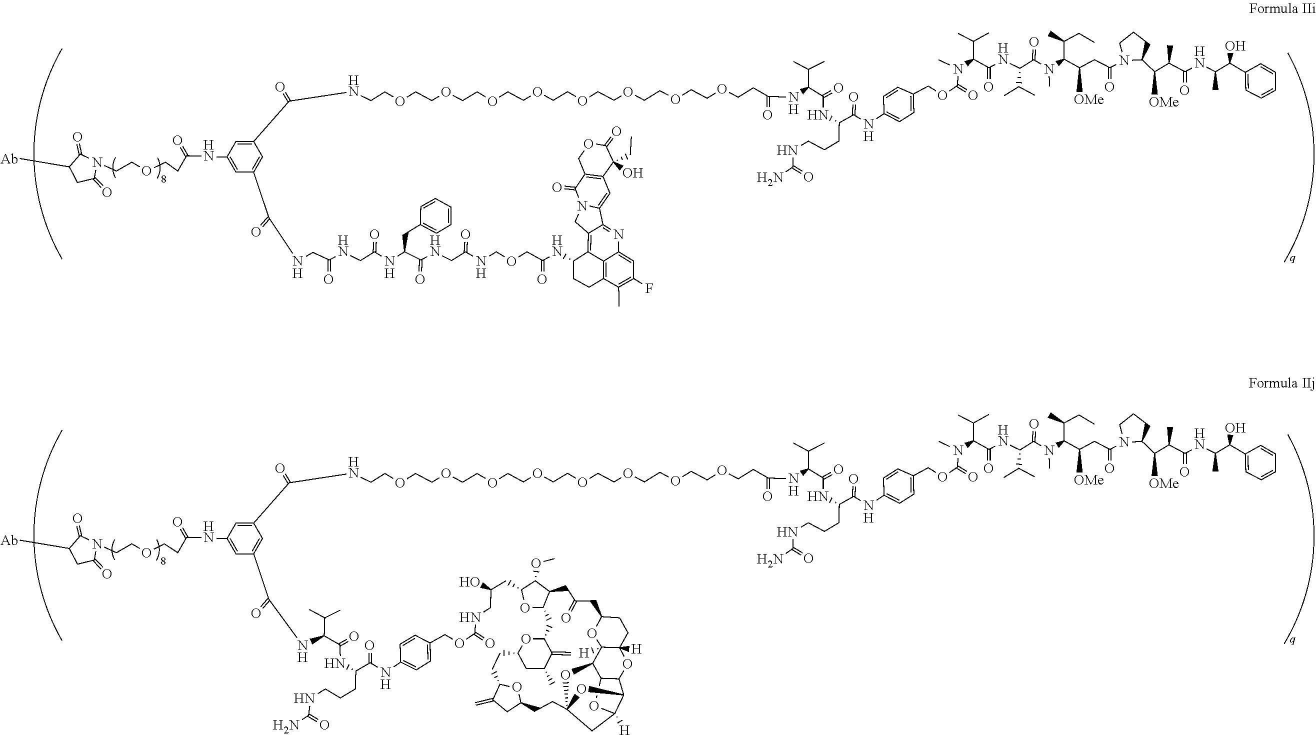

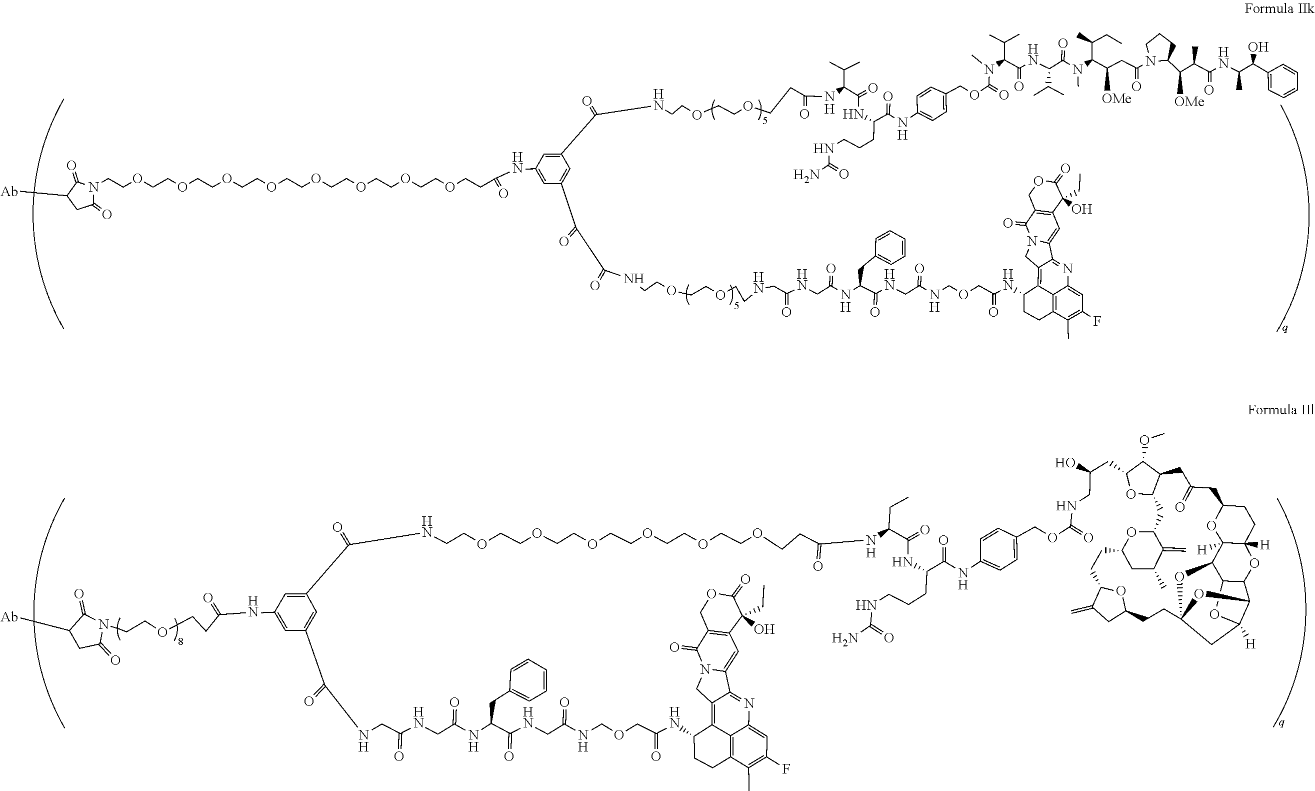

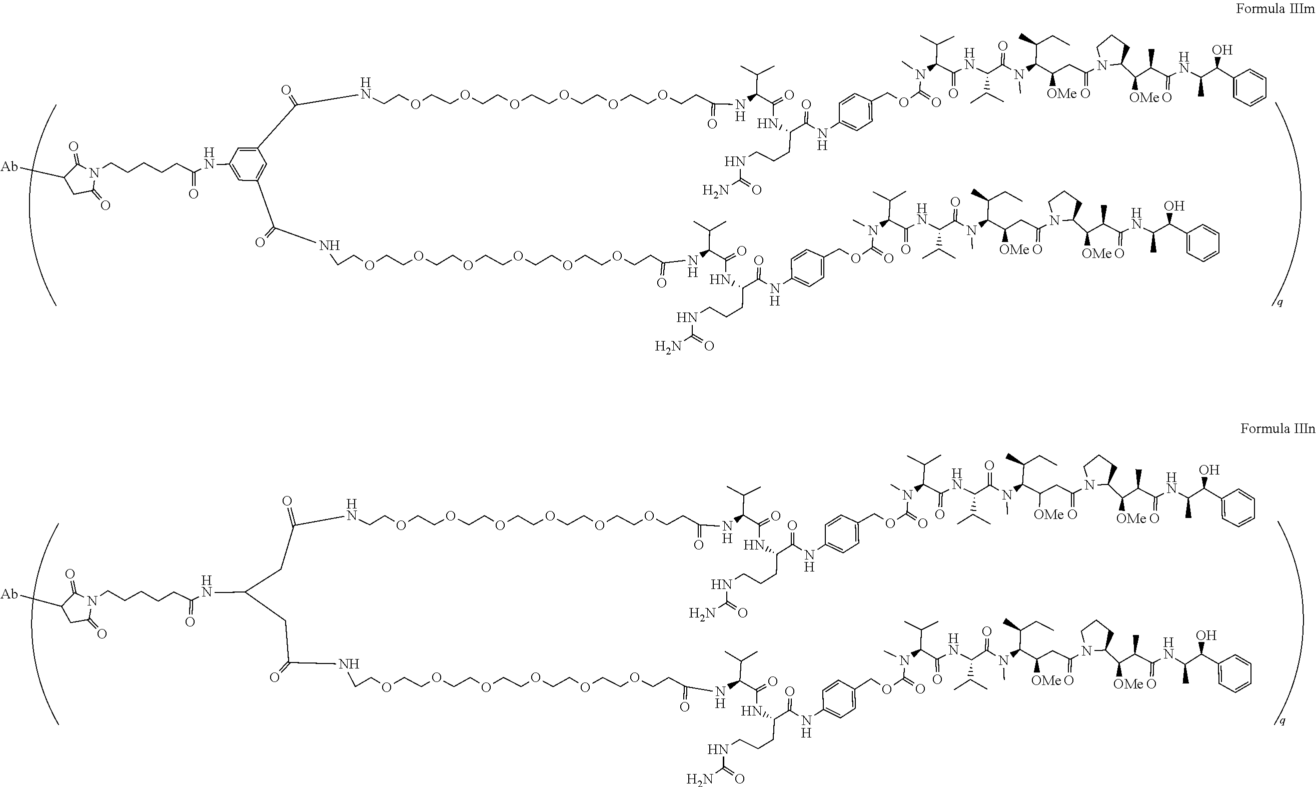

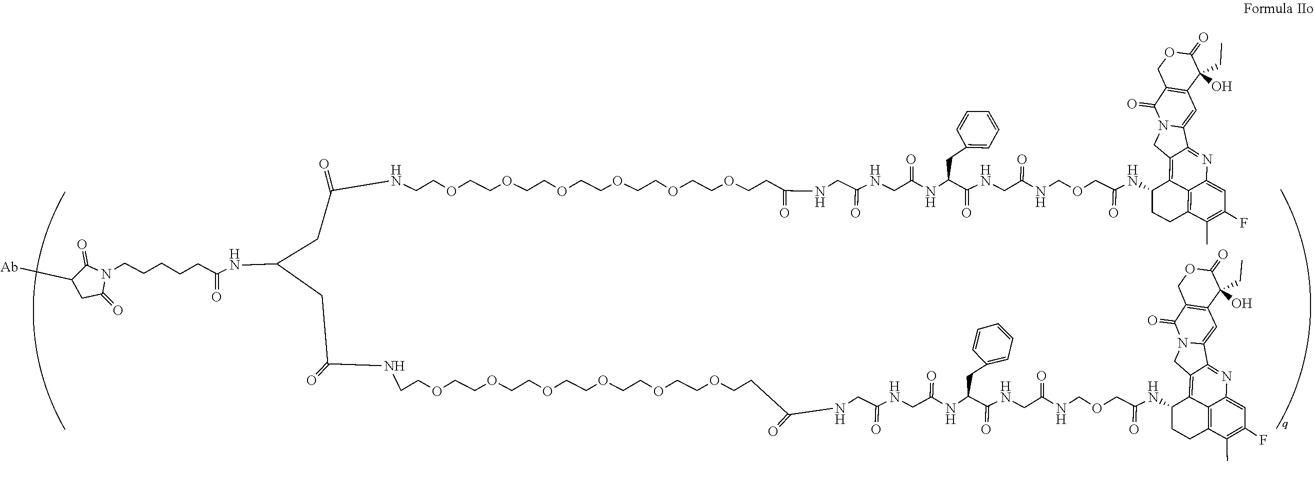

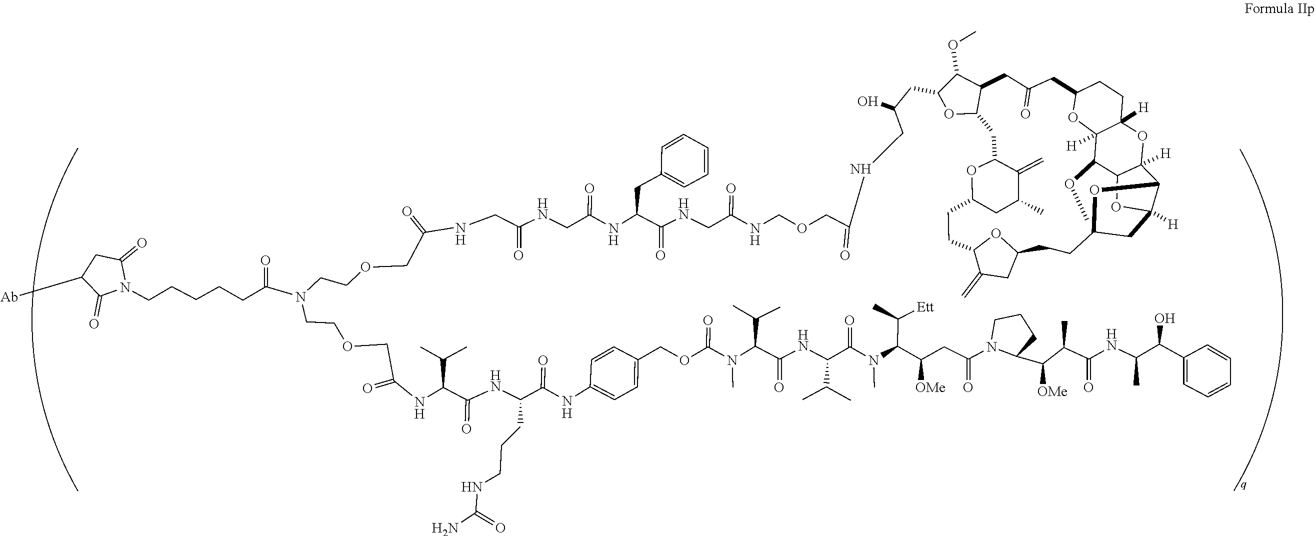

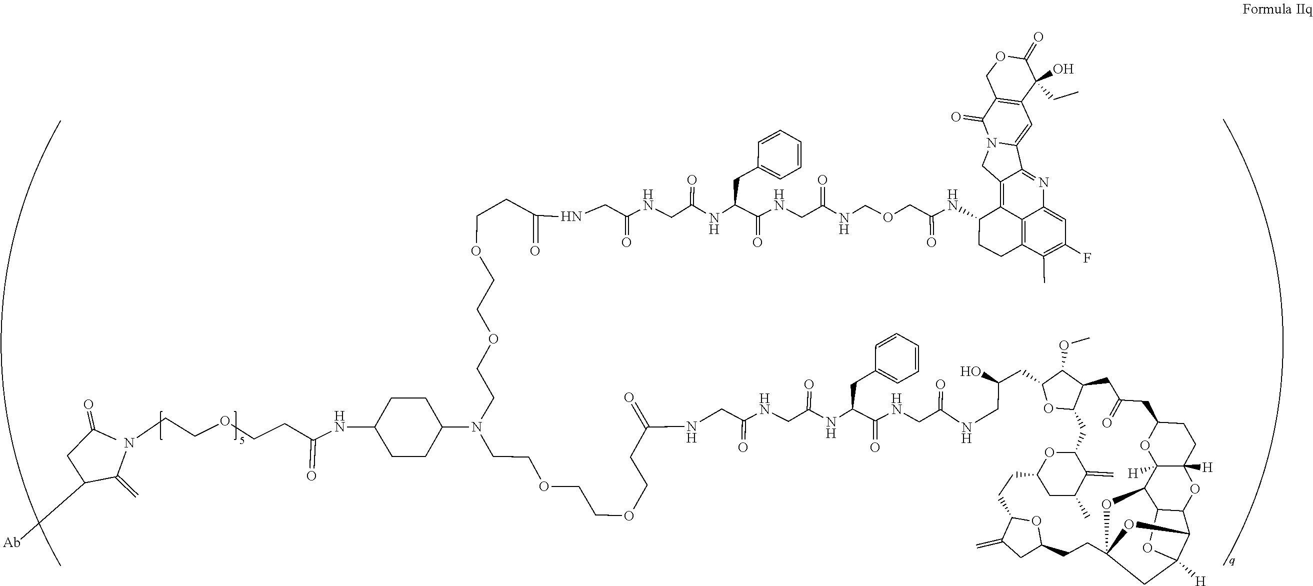

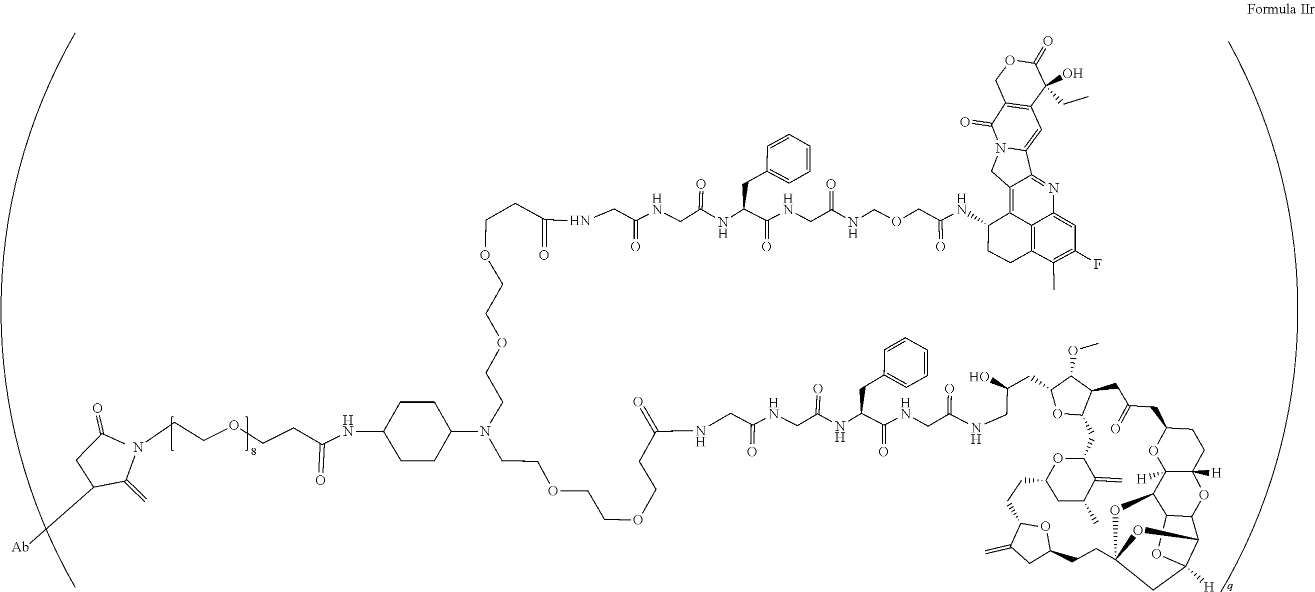

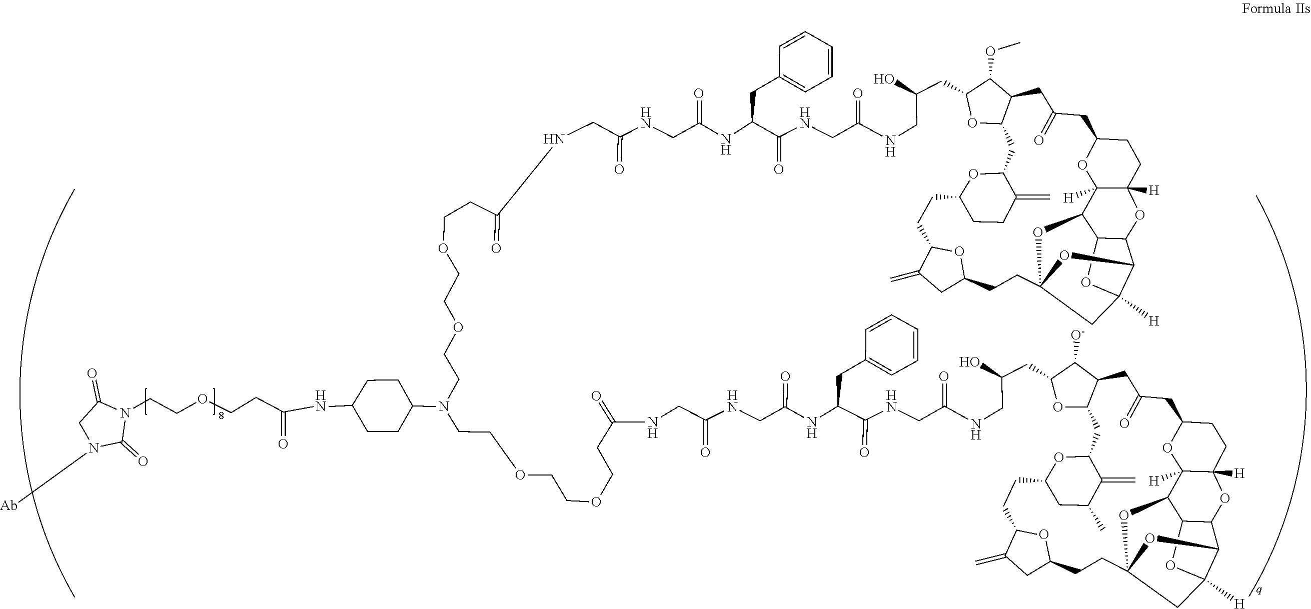

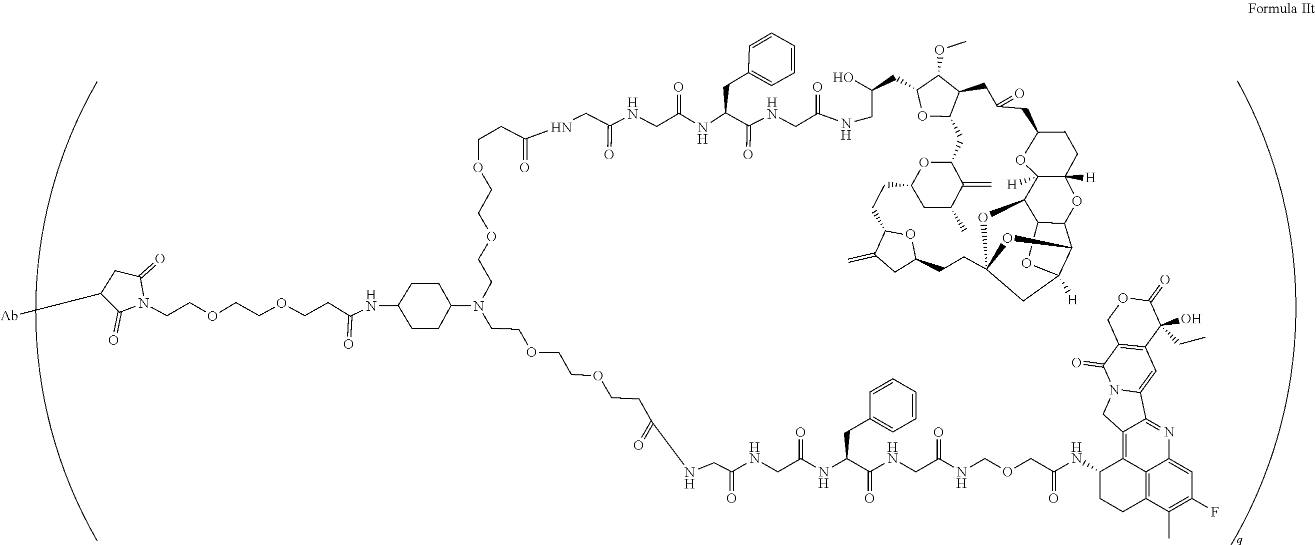

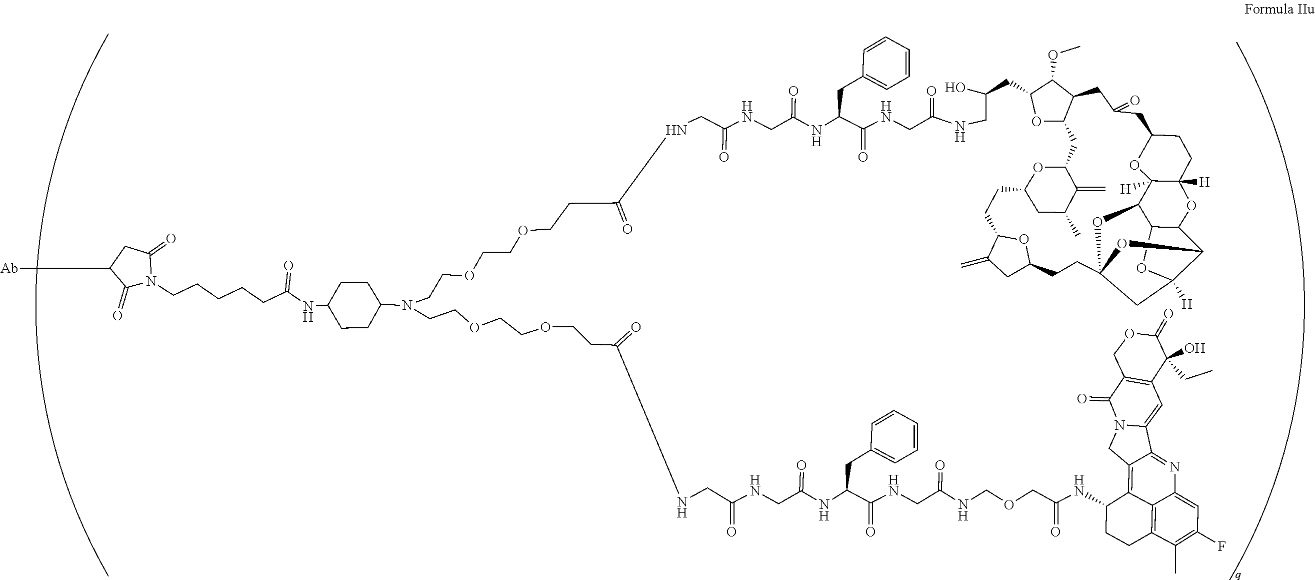

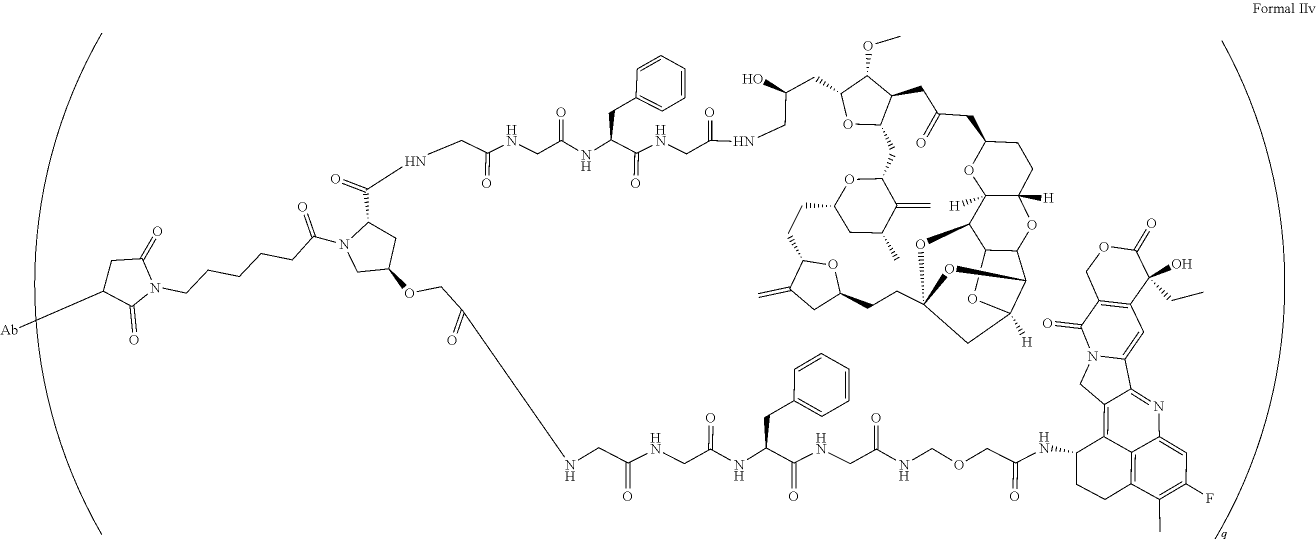

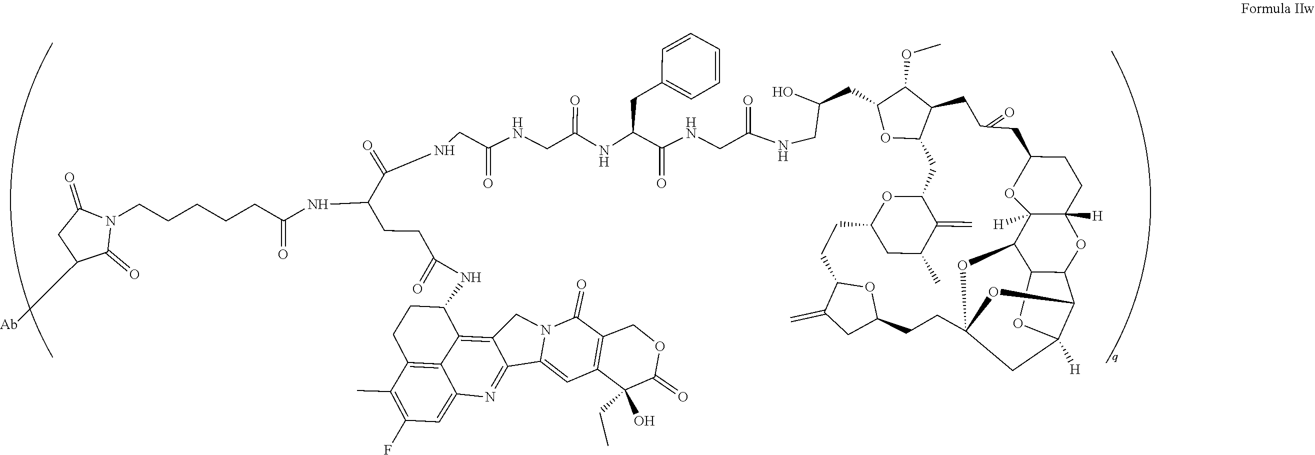

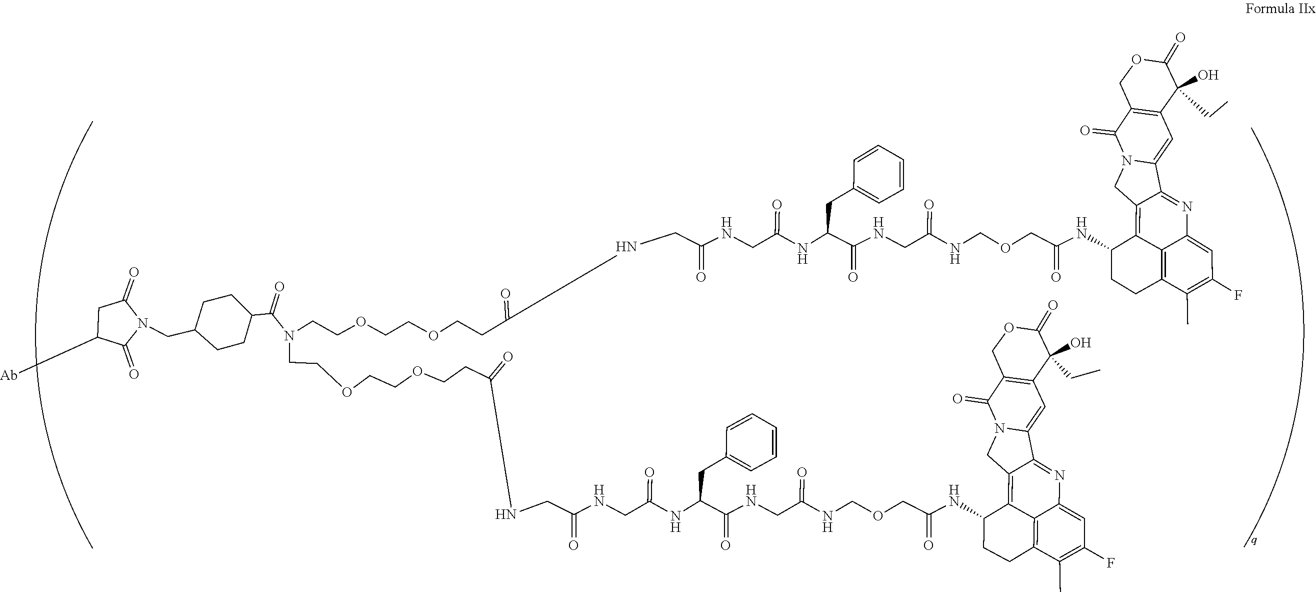

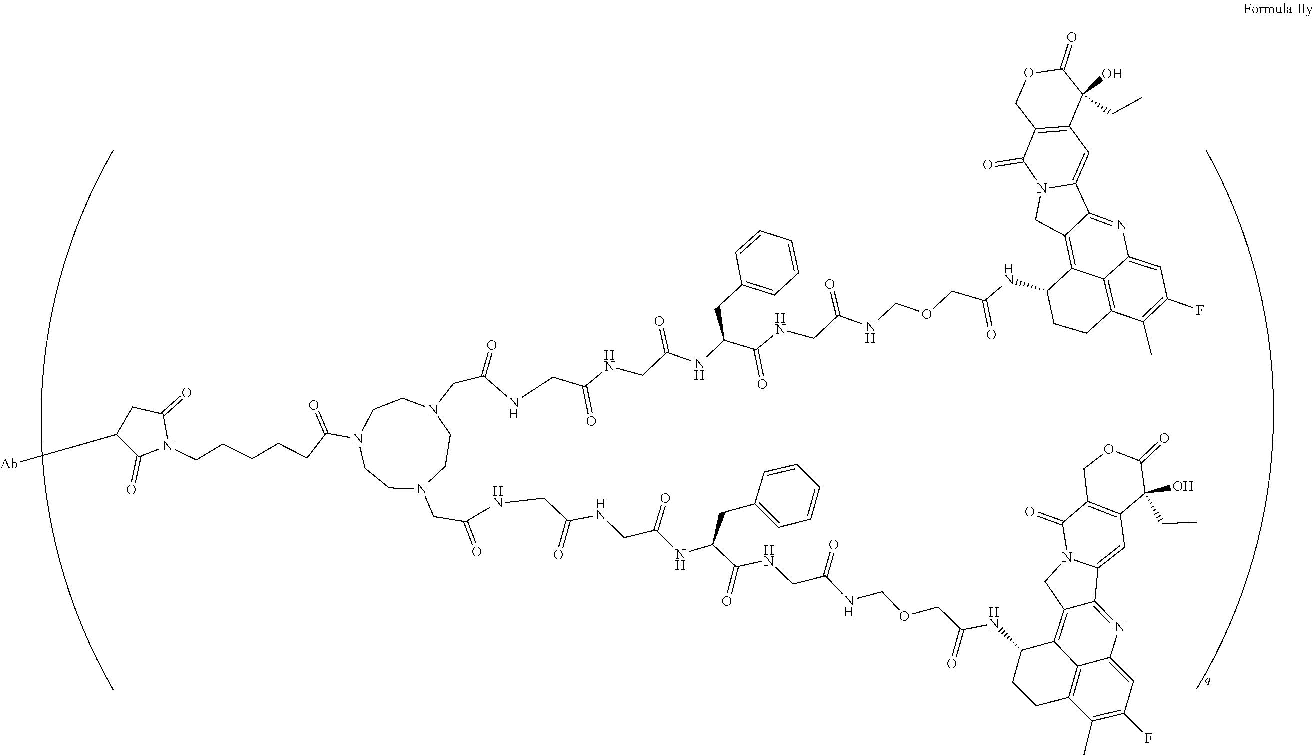

The present invention also provides a dual-drug targeting linker-drug conjugate, or a stereoisomer thereof, or an optical isomer thereof, or a deuterated compound thereof, characterized in that the dual-drug targeting linker-drug conjugate is obtained by connecting the targeting linker and the dual-drug link assembly units mentioned above; the targeting linker is a substance that can target and bind to the lesion site, and is preferably an antibody, an antibody fragment, a protein or an aptamer; the antibody is preferably an antibody targeting cell surface receptors and tumor-related antigens.

Further, the dual-drug targeting linker-drug conjugate is composed of two or more of dual-drug targeting linker-drug conjugates represented by formula XIa, with a DAR value of 3.5±1.0, preferably 3.5;

or, the dual-drug targeting linker-drug conjugate is composed of two or more of dual-drug targeting linker-drug conjugates represented by formula XIb, with a DAR value of 3.8±1.0, preferably 3.8;

or, the dual-drug targeting linker-drug conjugate is composed of two or more of dual-drug targeting linker-drug conjugates represented by formula XIc, with a DAR value of 3.9±1.0, preferably 3.9;

or, the dual-drug targeting linker-drug conjugate is composed of two or more of dual-drug targeting linker-drug conjugates represented by formula XId, with a DAR value of 4.1±1.0, preferably 4.1;

or, the dual-drug targeting linker-drug conjugate is composed of two or more of dual-drug targeting linker-drug conjugates represented by formula XIe, with a DAR value of 4.6±1.0, preferably 4.6;

or, the dual-drug targeting linker-drug conjugate is composed of two or more of dual-drug targeting linker-drug conjugates represented by formula XIf, with a DAR value of 3.7±1.0, preferably 3.7;

or, the dual-drug targeting linker-drug conjugate is composed of two or more of dual-drug targeting linker-drug conjugates represented by formula XIg, with a DAR value of 4.2±1.0, preferably 4.2;

or, the dual-drug targeting linker-drug conjugate is composed of two or more of dual-drug targeting linker-drug conjugates represented by formula XIh, with a DAR value of 4.1±1.0, preferably 4.1;

or, the dual-drug targeting linker-drug conjugate is composed of two or more of dual-drug targeting linker-drug conjugates represented by formula XIi, with a DAR value of 4.7±1.0, preferably 4.7;

or, the dual-drug targeting linker-drug conjugate is composed of two or more of dual-drug targeting linker-drug conjugates represented by formula XIj, with a DAR value of 2.8±1.0, preferably 2.8;

or, the dual-drug targeting linker-drug conjugate is composed of two or more of dual-drug targeting linker-drug conjugates represented by formula XIk, with a DAR value of 2.9±1.0, preferably 2.9;

or, the dual-drug targeting linker-drug conjugate is composed of two or more of dual-drug targeting linker-drug conjugates represented by formula XIl, with a DAR value of 4.5±1.0, preferably 4.5;

or, the dual-drug targeting linker-drug conjugate is composed of two or more of dual-drug targeting linker-drug conjugates represented by formula XIm, with a DAR value of 4.4±1.0, preferably 4.4;

or, the dual-drug targeting linker-drug conjugate is composed of two or more of dual-drug targeting linker-drug conjugates represented by formula XIn, with a DAR value of 3.7±1.0, preferably 3.7;

or, the dual-drug targeting linker-drug conjugate is composed of two or more of dual-drug targeting linker-drug conjugates represented by formula XIo, with a DAR value of 3.9±1.0, preferably 3.9;

or, the dual-drug targeting linker-drug conjugate is composed of two or more of dual-drug targeting linker-drug conjugates represented by formula XIp, with a DAR value of 3.5±1.0, preferably 3.5;

or, the dual-drug targeting linker-drug conjugate is composed of two or more of dual-drug targeting linker-drug conjugates represented by formula XIq, with a DAR value of 4.8±1.0, preferably 4.8;

or, the dual-drug targeting linker-drug conjugate is composed of two or more of dual-drug targeting linker-drug conjugates represented by formula XIr, with a DAR value of 4.9±1.0, preferably 4.9;

or, the dual-drug targeting linker-drug conjugate is composed of two or more of dual-drug targeting linker-drug conjugates represented by formula XIs, with a DAR value of 4.2±1.0, preferably 4.2;

or, the dual-drug targeting linker-drug conjugate is composed of two or more of dual-drug targeting linker-drug conjugates represented by formula XIt, with a DAR value of 5.3±1.0, preferably 5.3;

or, the dual-drug targeting linker-drug conjugate is composed of two or more of dual-drug targeting linker-drug conjugates represented by formula XIu, with a DAR value of 4.5±1.0, preferably 4.5;

or, the dual-drug targeting linker-drug conjugate is composed of two or more of dual-drug targeting linker-drug conjugates represented by formula XIv, with a DAR value of 4.9±1.0, preferably 4.9;

or, the dual-drug targeting linker-drug conjugate is composed of two or more of dual-drug targeting linker-drug conjugates represented by formula XIw, with a DAR value of 3.8±1.0, preferably 3.8;

or, the dual-drug targeting linker-drug conjugate is composed of two or more of dual-drug targeting linker-drug conjugates represented by formula XIx, with a DAR value of 4.9±1.0, preferably 4.9;

or, the dual-drug targeting linker-drug conjugate is composed of two or more of dual-drug targeting linker-drug conjugates represented by formula XIy, with a DAR value of 4.8±1.0, preferably 4.8.

“DAR value” represents the average number of dual-drug link assembly units conjugated with a targeting linker in a dual-drug targeting linker-drug conjugate, equivalent to the average of q values. The DAR value may not be an integer.

The present invention also provides the preparation method for the dual-drug targeting linker-drug conjugate molecule mentioned above or the dual-drug targeting linker-drug conjugate mentioned above, characterized in that the method comprises the following procedure: a targeting linker is conjugated with a dual-drug link assembly unit.

The present invention also provides a medicament for preventing and/or treating tumors, characterized in that it is a composition made from the dual-drug targeting linker-drug conjugate molecule mentioned above or the dual-drug targeting linker-drug conjugate mentioned above, as the active ingredient, in combination with pharmaceutically acceptable excipients.

The present invention also provides the use of the dual-drug targeting linker-drug conjugate molecule mentioned above or the dual-drug targeting linker-drug conjugate mentioned above in the manufacture of medicaments for the prevention and/or treatment of tumors.

Further, the tumor is HER2-positive.

Further, the tumors are selected from the group consisting of lung cancer, urethra cancer, large intestine cancer, prostate adenocarcinoma, ovarian cancer, pancreatic cancer, breast cancer, bladder cancer, gastric cancer, gastrointestinal stromal tumor, cervical cancer, esophageal cancer, squamous cell cancer, peritoneal cancer, liver cancer, colon cancer, rectal cancer, colorectal cancer, uterine cancer, salivary gland cancer, kidney cancer, vulva cancer, thyroid cancer, penile cancer, leukemia, malignant lymphoma, plasmacytoma, myeloma or sarcoma.

For the definition of terms used in the present invention: unless defined otherwise, the initial definition provided for the group or term herein applies to the group or term of the entire specification; for the terms that are not specifically defined herein, they should have the same meaning as commonly understood by one of ordinary skill in the art to which the disclosure belongs.

Tether group refers to the group that can be connected to a targeting linker.

Herein, “substitution” refers to the substitution of one, two, or more hydrogens in a molecule with other different atoms or molecules, including one, two, or more substitutions in the same or different atoms in the molecule.

“Aryl” refers to all carbon monocyclic or fused multicyclic (i.e. ring(s) sharing adjacent carbon atom pairs) groups with conjugated π electron systems, such as phenyl and naphthyl. The aryl ring can condense with other cyclic groups (including saturated and unsaturated rings), but cannot contain heteroatoms such as nitrogen, oxygen, or sulfur. At the same time, the point connecting the parent must be on the carbon of the ring with a conjugated π electron system. The aryl group can be either substituted or unsubstituted.

“Heteroaryl” refers to a heteroaromatic group containing one and more heteroatoms. The heteroatoms used herein include oxygen, sulfur, and nitrogen. For example, furanyl, thiophenyl, pyridyl, pyrazolyl, pyrrolyl, N-alkylpyrrolyl, pyrimidinyl, pyrazinyl, imidazolyl, tetrazolyl, etc. The heteroaryl ring can be condensed with aryl, heterocyclyl or cycloalkyl ring, wherein the ring connected to the parent structure is a heteroaryl ring. Heteroaryl can be optionally substituted or unsubstituted.

“Polycyclic alkyl” refers to a polycyclic cycloalkyl, in which two rings share two adjacent carbon atoms.

“Fused heterocyclyl” refers to a polycyclic heterocyclyl, in which two rings share two adjacent carbon or heteroatoms.

In the present invention, “q” represents an average value. For example, when 1≤q≤8, it means that the average value is a value of ≥1 and ≤8.

An “antibody” or “antibody unit” within its scope includes any part to which the antibody binds. This unit can bind, reactively correlate, or chelate with a receptor, antigen, or other receptor units contained in the targeting cell population. Antibodies can be any protein or protein molecule that can bind, chelate, or react with a moiety of the cell population to be treated or biologically modified.

The antibodies in the present invention can specifically bind to antigens. The designed antigens include tumor-associated antigens (TAA), cell surface receptor proteins and other cell surface molecules, cell survival regulators, cell proliferation regulators, molecules related to tissue growth or differentiation (such as those known or predicted to be functional), lymphokines, cytokines, factors involved in cell cycle regulation, molecules involved in angiogenesis, and molecules related to angiogenesis. Tumor-related antigens can be cluster differentiation antigens (such as CD proteins). The antigen that the antibody binds to in the present invention can be one or a subset of the aforementioned classifications, and other subsets contain other molecules/antigens with special properties.

The antibodies used in the dual-drug link assembly unit and the dual-drug targeting linker-drug conjugate of the present invention include but are not limited to antibodies targeting tumor-related antigens of cell surface receptors. Tumor-related antigens include but are not limited to the tumor-related antigens listed below, including names and gene library accession numbers. Antibodies target tumor-related antigens, including all amino acid sequence variants and homologies, with at least 70%, 80%, 85%, 90% or 95% homology with the confirmed sequences in the literature, or with biological properties and characteristics that are completely consistent with the tumor-related antigen sequences in the literature.

The tumor-related antigens include: BMPR1B (Genbank accession number: NM-001203), E16 (Genbank accession number: NM-003486), STEAP1 (Genbank accession number: NM-012449), 0772P (Genbank accession number: AF361486), MPF (Genbank accession number: NM-005823), Napi3b (Genbank accession number: NM-006424), Sema 5b (Genbank accession number: AB040878), PSCAhlg (Genbank accession number: AY358628), ETBR (Genbank accession number: AY275463), MSG783 (Genbank accession number: NM-017763), STEAP2 (Genbank accession number: AF455138), TrpM4 (Genbank accession number: NM-017636), CRIPTO (Genbank accession number: NP-003203 or NM-003212), CD21 (Genbank accession number: M26004), CD79B (Genbank accession number: NM-000626), FcRH2 (Genbank accession number: NM-030764), HER2 (Genbank accession number: M11730), NCA (Genbank accession number: M18728), MDP (Genbank accession number: BC017023), IL20Rα(Genbank accession number: AF184971), Brevican (Genbank accession number: AF229053), EpHB2R (Genbank accession number: NM-004442), GEDA (Genbank accession number: AY260763), BAFF—R (Genbank accession number: AF1164546), CD22 (Genbank accession number: AK026467), CD79a (Genbank accession number: NP-001774.1), CXCR5 (Genbank accession number: NP-001701.1), HLA-DOB (Genbank accession number: NP-002111.1), P2X5 (Genbank accession number: NP-002552.2), CD72 (Genbank accession number: NP-001773.1), LY64 (Genbank accession number: NP-005573.1), FcRH1(Genbank accession number: NP-443170.1), IRTA2 (Genbank accession number: NP-112571.1), and TENB2 (Genbank accession number: AF179274).

As used herein, “drug” or code name “D” generally refers to any compound with desired biological activity and reactive functional groups to prepare the conjugate compound described in the present invention. Further, the drug includes a cytotoxic compound used for cancer treatment, a biologically active protein or peptide, including but not limited to camptothecin derivatives such as SN-38, DXd, and DX-8951, as well as compounds that act on microtubule proteins such as Eribulin, MMAE, MMAF, maytasine, etc. (having the following structures).

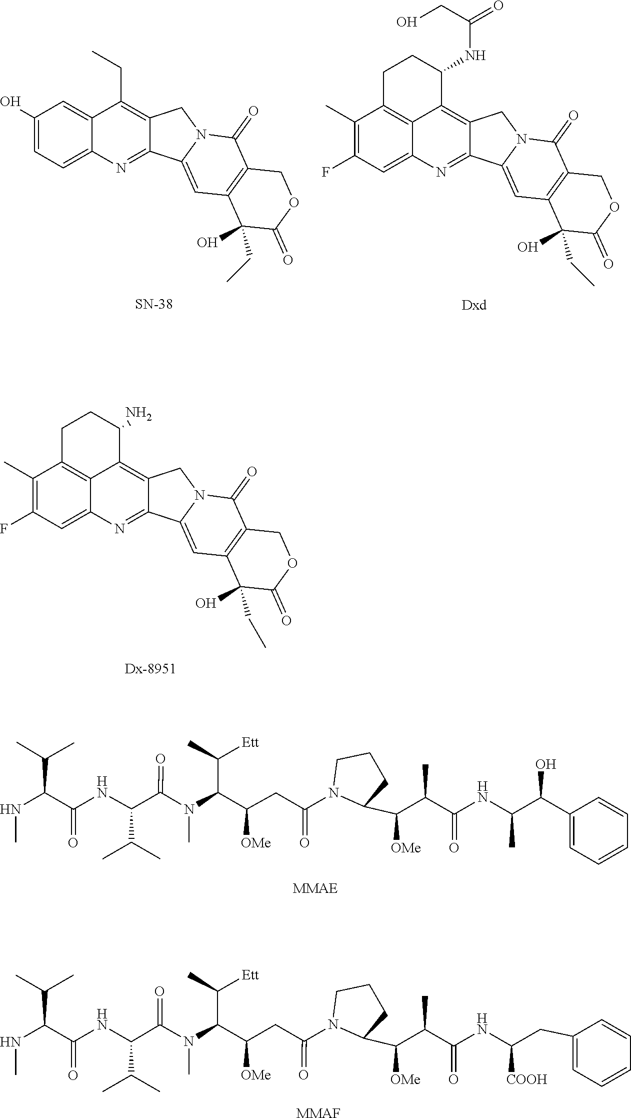

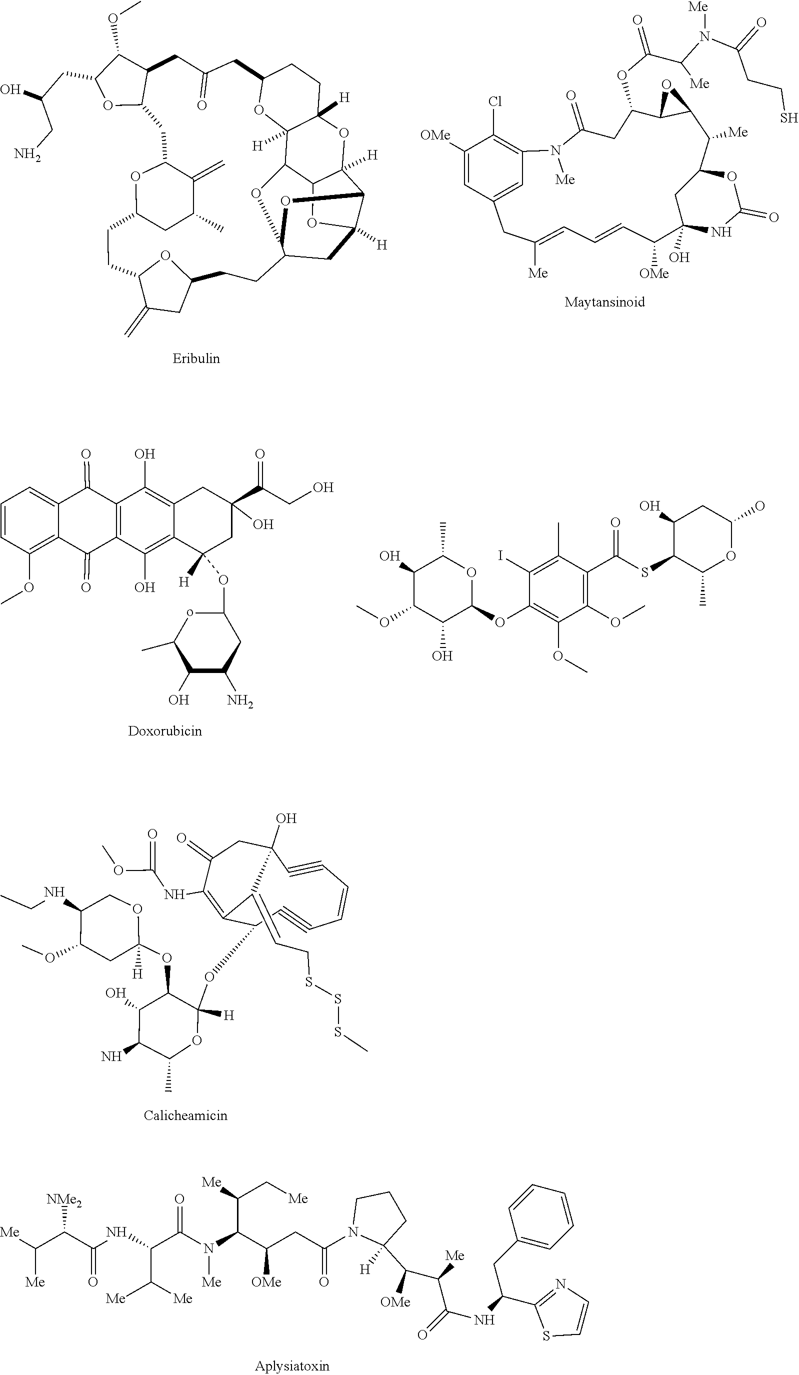

According to the intracellular drug release mechanism, the “linkers” or “antibody-drug conjugate linkers” mentioned herein can be divided into two categories: non-cleavable linkers and cleavable linkers.

For dual-drug antibody-drug conjugates containing non-cleavable linkers, the drug release mechanism is as follows: after the conjugates bind to the antigen and are internalized in the cells, the antibody is enzymatically hydrolyzed in the lysosome, releasing small molecule drugs, to which the linker and the antibody amino acid residues link together.

Cleavable linkers are broken and then the active drugs (small molecule drugs themselves) are released in the target cells. Cleavable linkers can be divided into two main categories: chemically unstable linkers and linkers which are unstable to enzyme.

Chemically unstable linkers can be selectively broken due to differences in plasma and cytoplasmic properties, including pH value, glutathione concentration, etc. The linkers which are unstable to the enzyme, such as peptide linkers, can better control drug release. Peptide linkers can be effectively cut off by proteases in the lysosome, such as cathepsin or fibrinolytic enzyme. This peptide binding is considered to be very stable in plasma, as inappropriate extracellular pH values and serum protease inhibitors often result in the protease not having activity outside the cells. Due to its high plasma stability and good intracellular cleavage selectivity and effectiveness, the linkers which are unstable to the enzyme are widely used as cleavable linkers for antibody-drug conjugates.

The term ‘self-stabilizing linker’ refers to the linker structure that connects anti-tumor compounds to antibodies in a dual-drug antibody-drug conjugate.

The present invention provides a dual-drug link assembly unit, which can be linked to a targeting linker to obtain a dual-drug targeting linker-drug conjugate, that can have targeting effects on tumor cells, reduce the toxic and side effects on normal cells, and at the same time, effectively overcome drug resistance and achieve a synergistic anti-tumor effect. Compared to the already listed ADC: DS-8201, the ADC provided in the present invention significantly improves the inhibitory effect on HER2 positive cell strains N87 and SK—BR—3d. The dual-drug link assembly unit and the dual-drug targeting linker-drug conjugate of the present invention have broad application prospects in the manufacture of medicaments for prevention and/or treatment of tumors.

Obviously, based on the above content of the present invention, according to the common technical knowledge and the conventional means in the field, without department from the above basic technical spirits, other various modifications, alternations, or changes can further be made.

With reference to the following specific examples of the embodiments, the above content of the present invention is further illustrated. But it should not be construed that the scope of the above subject matter of the present invention is limited to the following examples. The techniques realized based on the above content of the present invention are all within the scope of the present invention.

### EXAMPLES

The starting materials or instruments used in the present invention are conventional products that can be commercially available.

### Example 1. Preparation of the Dual-Drug Link Assembly Unit of Formula XIa According to the Present Invention

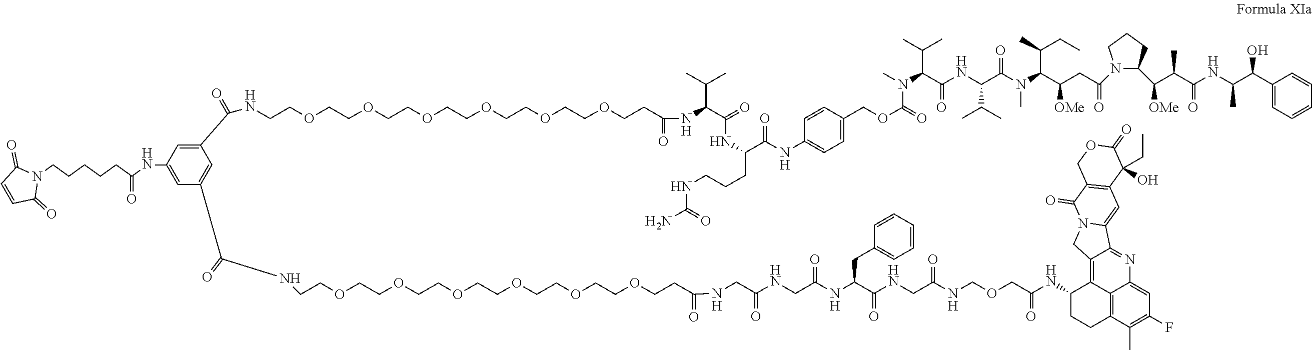

### Step 1: Preparation of Intermediate L-3

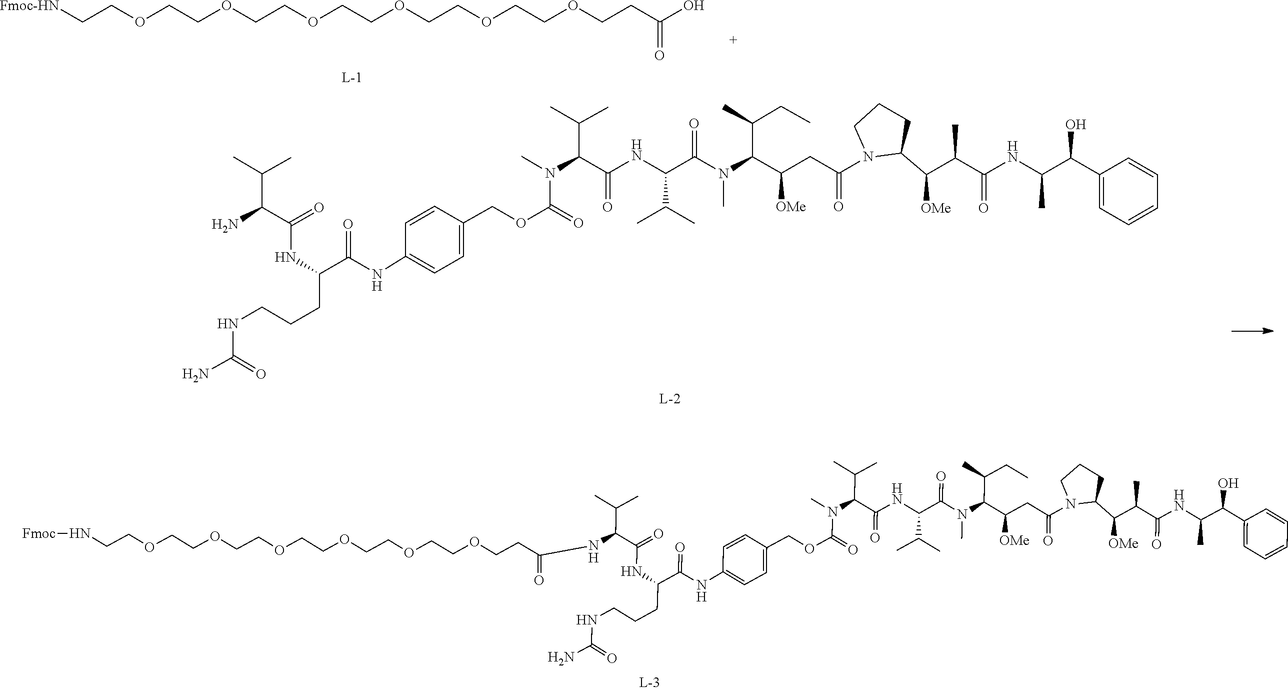

The preparation method of compound L-2 was the same as that described in the Chinese patent application with an application number 2020107518214.

To a 25 mL reaction flask, were added compound L-2 (179 mg, 0.15 mmol), compound L-1 (87 mg, 0.15 mmol), N,N,N′,N′-tetramethyl-O-(7-azabenzotriazol-1-yl)uronium hexafluorophosphate (69 mg, 0.18 mmol), N,N-diisopropylethylamine (29 mg, 0.227 mmol), and N,N-dimethylformamide (2 mL), and then the reaction was stirred and reacted at room temperature for 30 min. TLC indicated the completion of the reaction. N,N-dimethylformamide was removed by concentration under reduced pressure, and the residue was purified by TLC (developing agent:dichloromethane:methanol=10:1), to obtain the intermediate L-3 (132 mg), with a yield of 52.3%.

### Step 2: Preparation of Intermediate L-4

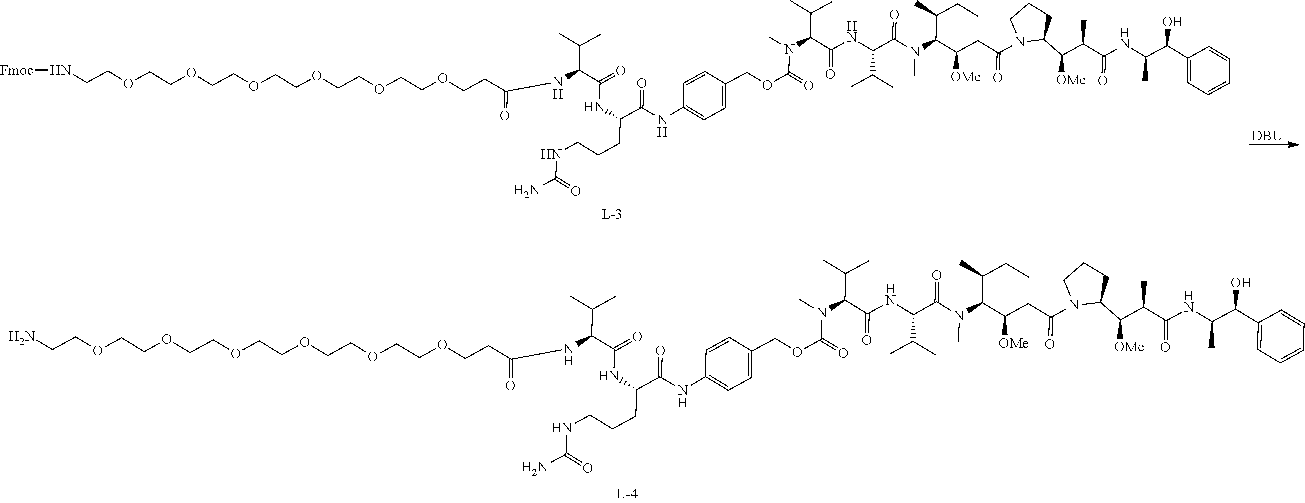

To a 25 mL reaction flask, were added compound L-3 (30 mg, 0.017 mmol), 1,8-diazabicyclo[5.4.0]undec-7-ene (2.7 mg, 0.017 mmol), and N,N-dimethylformamide (1 mL), and then the reaction was allowed to react under stirring at room temperature for 30 min. TLC indicated the completion of the reaction, and then the reaction was directly used in the next step.

### Step 3: Preparation of Intermediate L-6

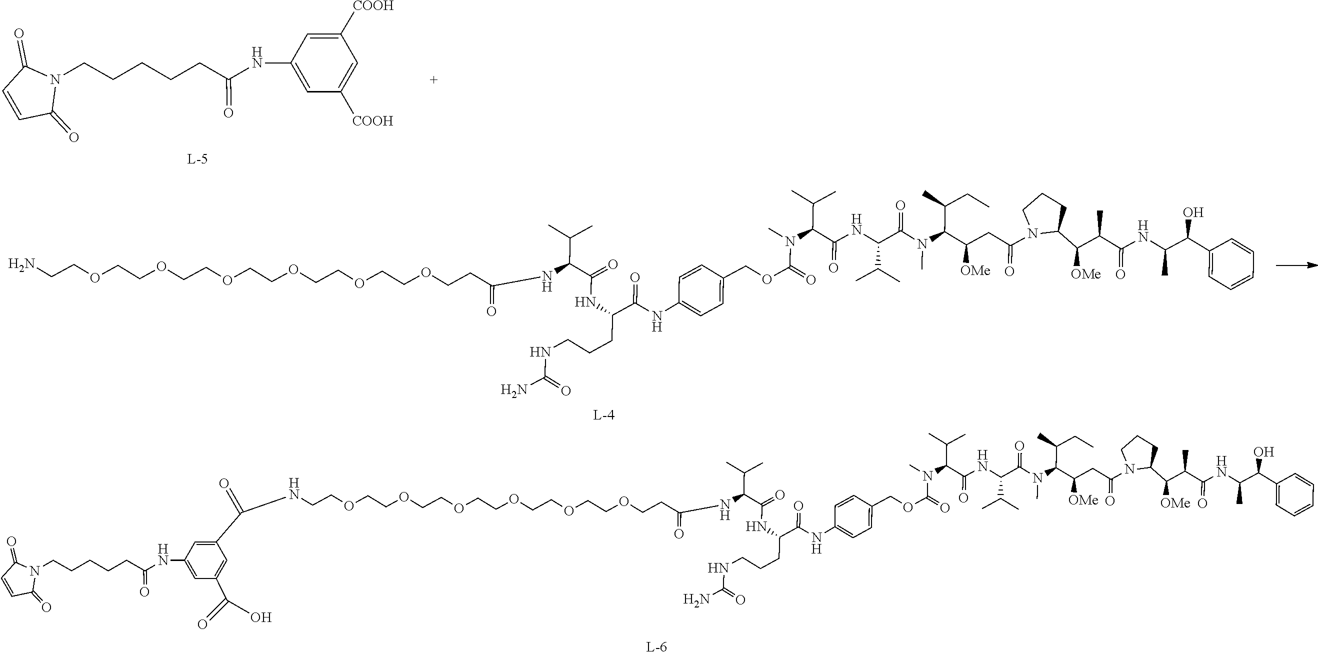

To a 25 mL reaction flask, were added intermediate L-4 (30 mg, 0.02 mmol), intermediate L-5 (26 mg, 0.07 mmol), N,N,N′,N′-tetramethyl-O-(7-azabenzotriazol-1-yl)uronium hexafluorophosphate (25 mg, 0.07 mmol), N,N-diisopropyl ethyl amine (9.2 mg, 0.07 mmol), and N,N-dimethylformamide (3 mL), and then the reaction was allowed to react at room temperature for 30 min. TLC indicated the disappearance of the starting material L-4, suggesting completion of the reaction. N,N-dimethylformamide was removed by concentration under reduced pressure, and the residue was purified by TLC (dichloromethane:methanol=10:1), to obtain the intermediate L-6 (17 mg), with a yield of 47%.

### Step 4: Preparation of Intermediate L-8

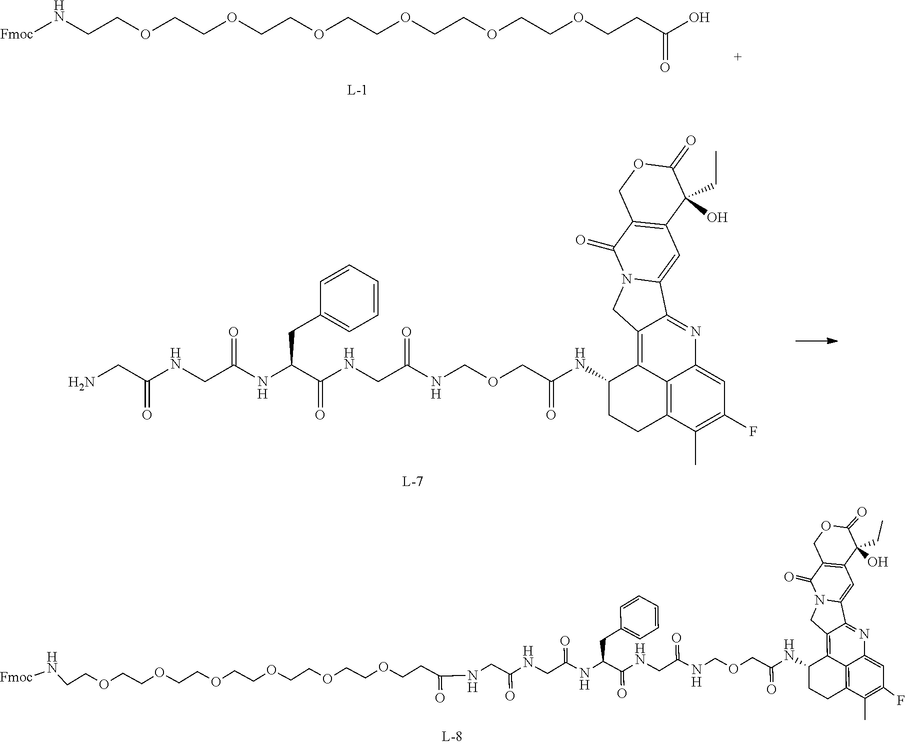

To a 25 mL reaction flask, were added intermediate L-7 (60 mg, 0.071 mmol), L-1 (26 mg, 0.07 mmol), N,N,N′,N′-tetramethyl-O-(7-azabenzotriazol-1-yl)uronium hexafluorophosphate (32.5 mg, 0.085 mmol), N,N-diisopropylethylamine (13.8 mg, 0.101 mmol), and N,N-dimethylformamide (1 mL), and then the reaction was allowed to react at room temperature under stirring for 0.5 h. TLC indicated the completion of the reaction. N,N-dimethylformamide was removed by concentration under reduced pressure, and the residue was purified by TLC (dichloromethane:methanol=10:1), to obtain the intermediate L-8 (58.4 mg), with a yield of 60%.

### Step 5: Preparation of Intermediate L-9

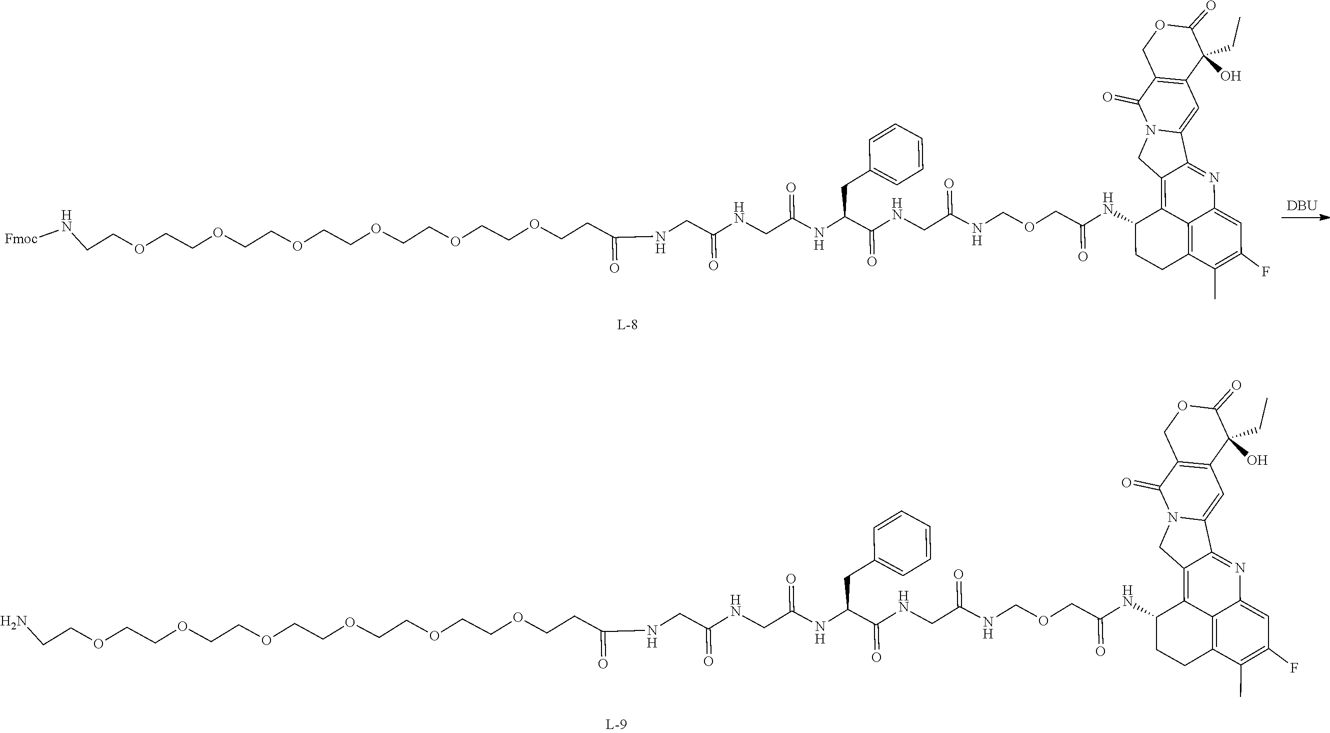

To a 10 mL reaction flask, were added L-8 (10 mg, 0.007 mmol), 1,8-diazabicyclo[5.4.0]undec-7-ene (2 mg, 0.010 mmol), and N,N-dimethylformamide (1.5 mL), and then the reaction was allowed to react under stirring at room temperature for 10 min. TLC indicated the disappearance of L-8, suggesting completion of the reaction, and then the reaction was directly used in the next step.

### Step 6: Preparation of Compound XIa

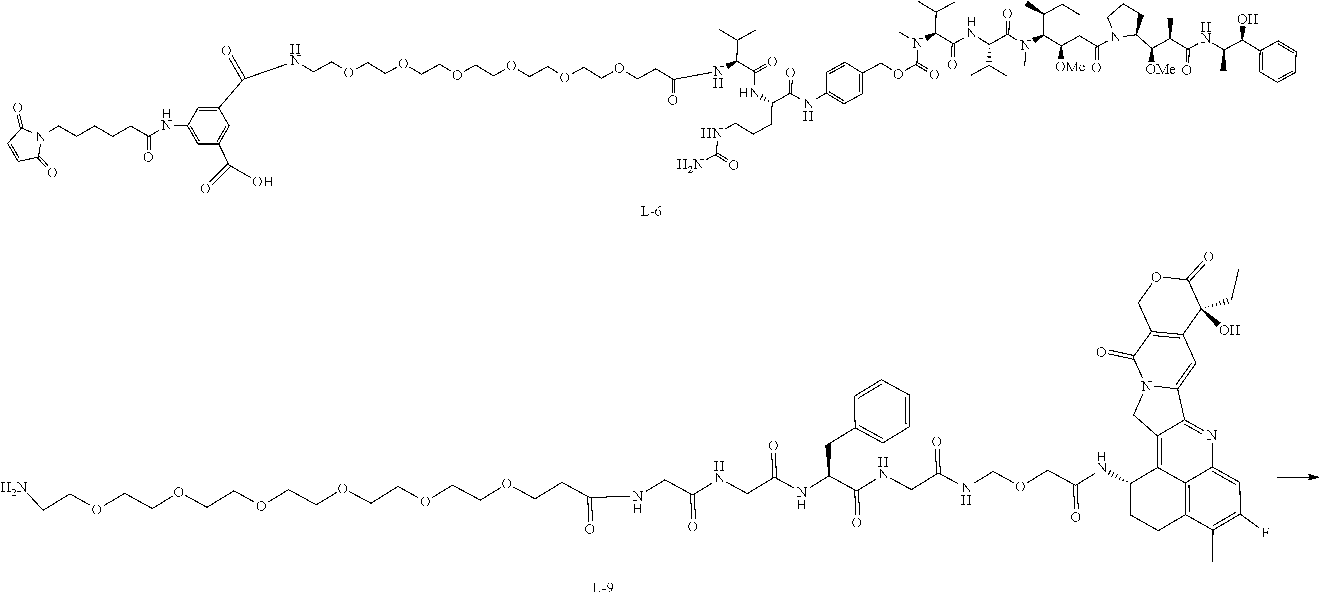 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCOCCOCCNC(=O)c2cc(NC(=O)CCCCCN3C(=O)C=CC3=O)cc(C(=O)O)c2)C(C)C)cc1)C(C)C ; CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCOCCOCCOCCOCCN)CC3]

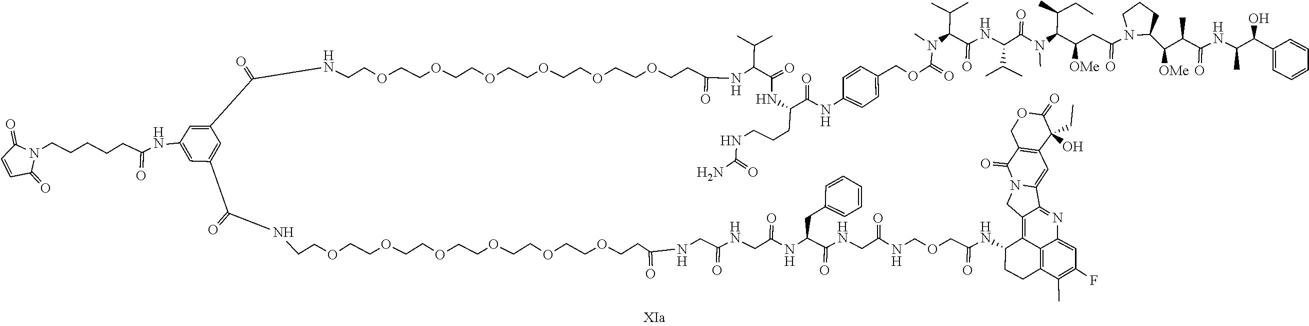 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)C(NC(=O)CCOCCOCCOCCOCCOCCOCCNC(=O)c2cc(NC(=O)CCCCCN3C(=O)C=CC3=O)cc(C(=O)NCCOCCOCCOCCOCCOCCOCCC(=O)NCC(=O)NCC(=O)N[C@@H](Cc3ccccc3)C(=O)NCC(=O)NCOCC(=O)N[C@H]3CCc4c(C)c(F)cc5nc6c(c3c45)Cn3c-6cc4c(c3=O)COC(=O)[C@]4(O)CC)c2)C(C)C)cc1)C(C)C]

To a 25 mL reaction flask, were added intermediate L-6 (10 mg, 0.005 mmol), L-9 (6 mg, 0.005 mmol), N,N-dimethylformamide (1.5 mL), N,N-diisopropylethylamine (1.2 mg, 0.007 mmol), and N,N,N′,N′-tetramethyl-O-(7-azabenzotriazol-1-yl)uronium hexafluorophosphate (2 mg, 0.006 mmol), and then the reaction was allowed to react in an ice water bath under stirring for 1 h. TLC indicated the disappearance of L-6, suggesting completion of the reaction. N,N-dimethylformamide was removed by concentration under reduced pressure, and the residue was purified by TLC (dichloromethane:methanol=10:1), to obtain compound XIa (11.7 mg), with a yield of 72%.

### Example 2. Preparation of the Dual-Drug Link Assembly Unit of Formula XIb According to the Present Invention

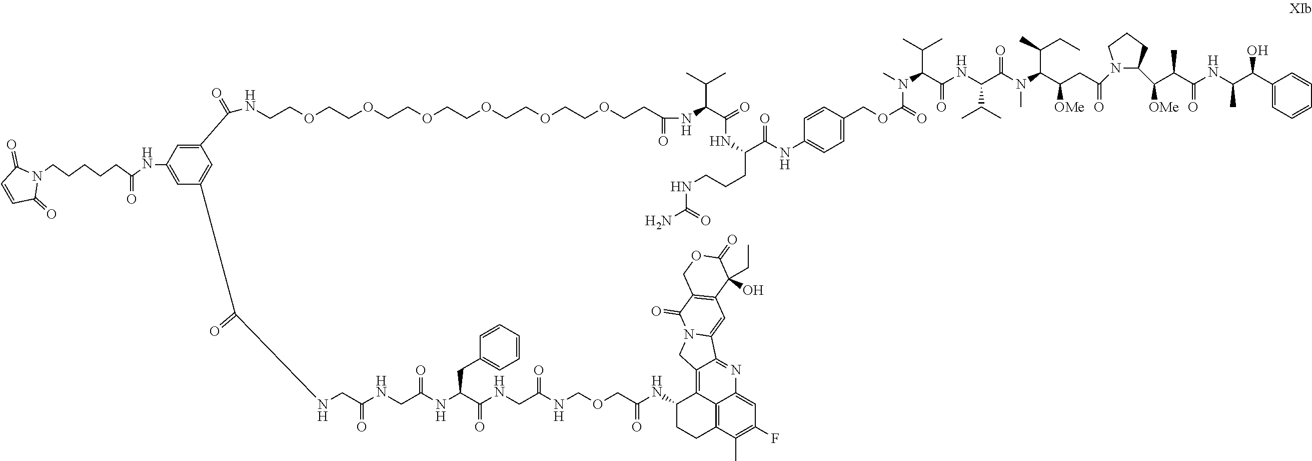

### Step 1: Preparation of Compound XIb

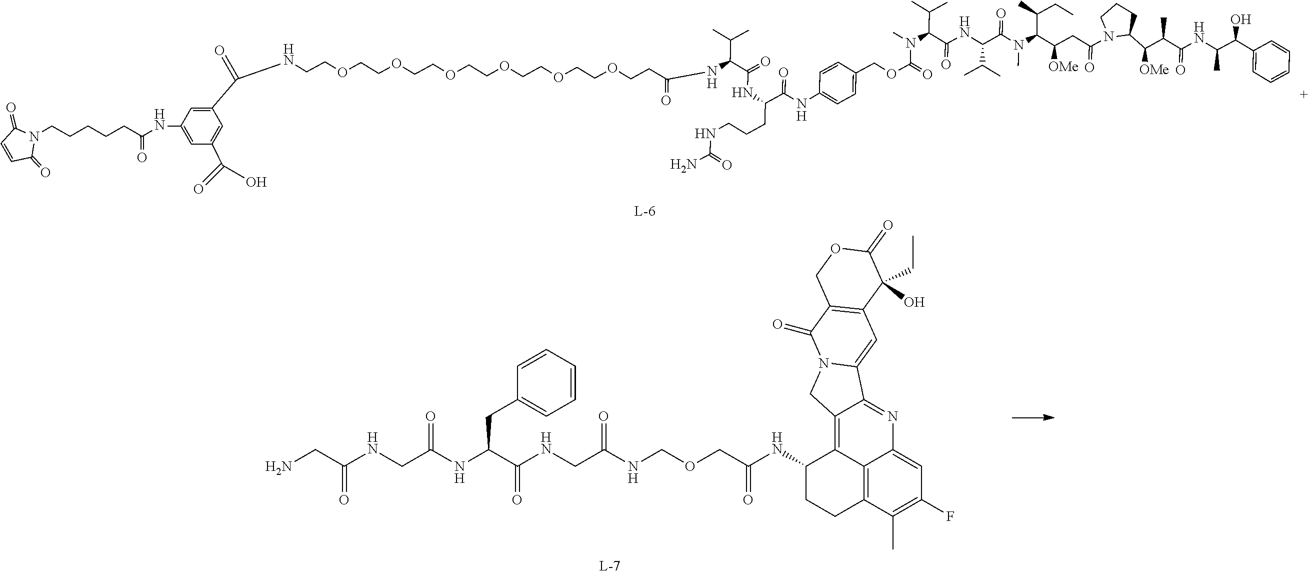 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCOCCOCCNC(=O)c2cc(NC(=O)CCCCCN3C(=O)C=CC3=O)cc(C(=O)O)c2)C(C)C)cc1)C(C)C ; CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CN)CC3]

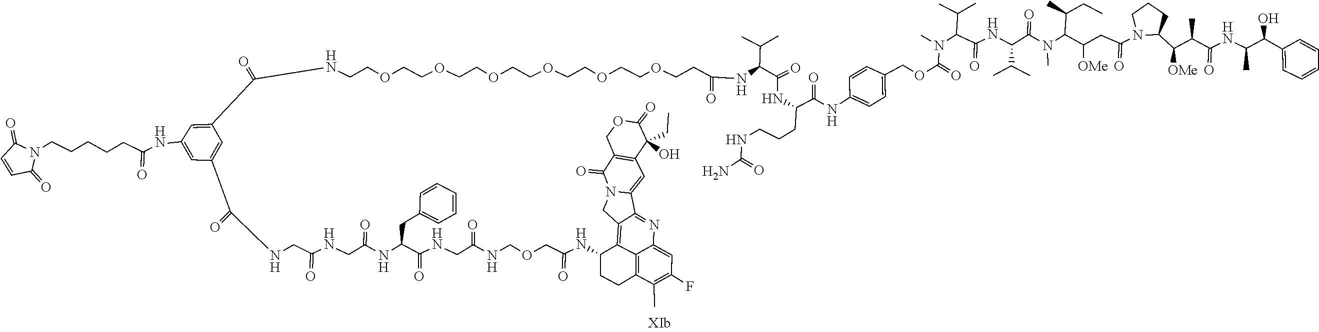 [SMILES: CC[C@H](C)C(C(CC(=O)N1CCC[C@H]1[C@H](OC)[C@@H](C)C(=O)N[C@H](C)[C@@H](O)c1ccccc1)OC)N(C)C(=O)[C@@H](NC(=O)C(C(C)C)N(C)C(=O)OCc1ccc(NC(=O)[C@H](CCCNC(N)=O)NC(=O)[C@@H](NC(=O)CCOCCOCCOCCOCCOCCOCCNC(=O)c2cc(NC(=O)CCCCCN3C(=O)C=CC3=O)cc(C(=O)NCC(=O)NCC(=O)N[C@@H](Cc3ccccc3)C(=O)NCC(=O)NCOCC(=O)N[C@H]3CCc4c(C)c(F)cc5nc6c(c3c45)Cn3c-6cc4c(c3=O)COC(=O)[C@]4(O)CC)c2)C(C)C)cc1)C(C)C]

To a 25 mL reaction flask, were added intermediate L-6 (10 mg, 0.005 mmol), intermediate L-7 (5.8 mg, 0.005 mmol), N,N,N′,N′-tetramethyl-O-(7-azabenzotriazol-1-yl)uronium hexafluorophosphate (2.5 mg, 0.006 mmol), N,N-diisopropylethylamine (1.2 mg, 0.007 mmol), and N,N-dimethylformamide (1 mL), and then the reaction was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIb (12 mg), with a yield of 96%.

### Example 3. Preparation of the Dual-Drug Link Assembly Unit of Formula XIc According to the Present Invention

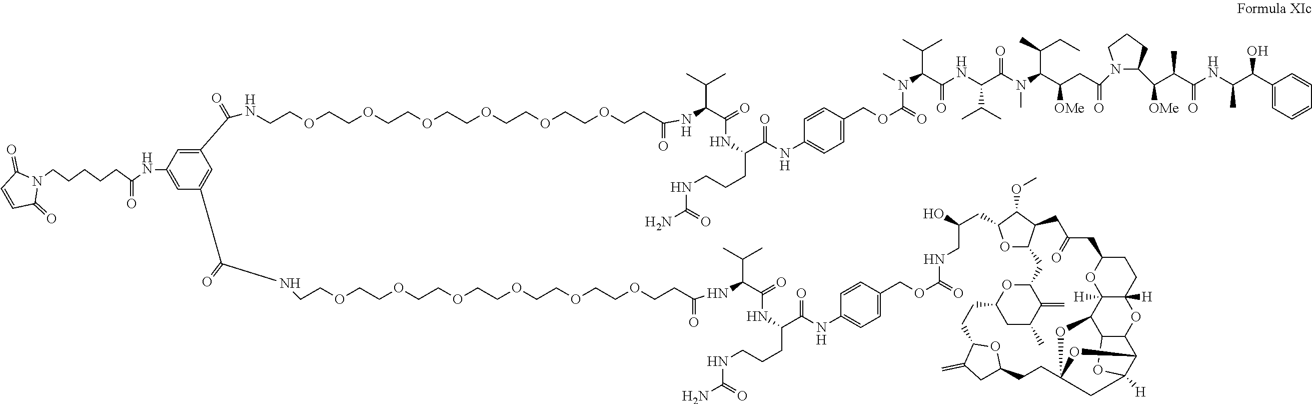

### Step 1: Preparation of Compound C-1

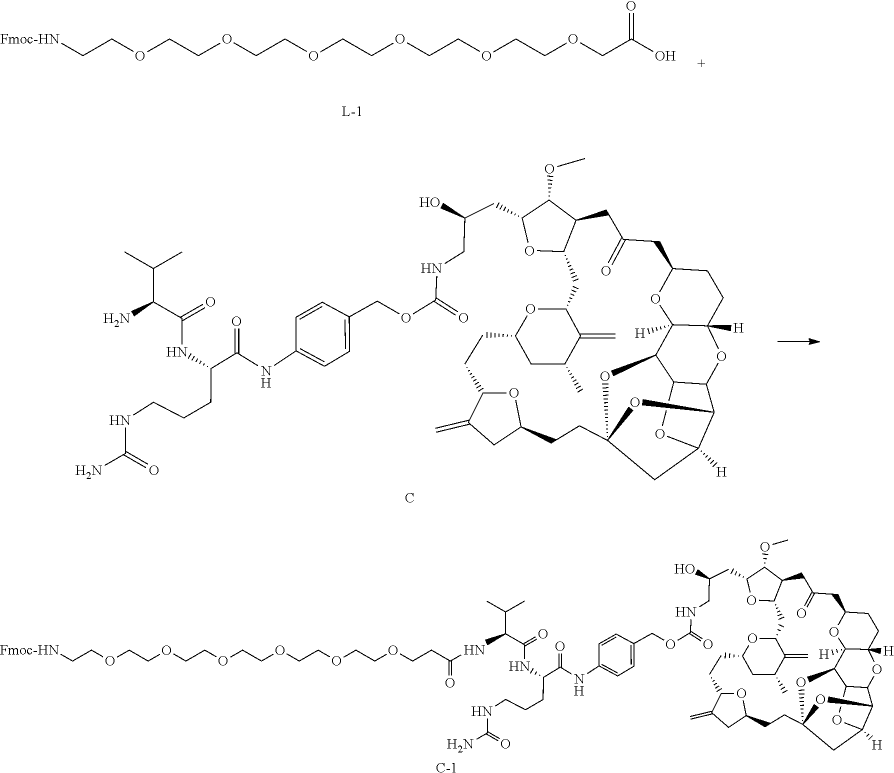

To a 25 mL reaction flask, were added compound L-1 (100 mg, 0.18 mmol), compound C (200 mg, 0.18 mmol), N,N,N′,N′-tetramethyl-O-(7-azabenzotriazol-1-yl)uronium hexafluorophosphate (101 mg, 0.27 mmol), N,N-diisopropylethylamine (45 mg, 0.35 mmol), and N,N-dimethylformamide (4 mL), and then the mixture was allowed to react at room temperature under stirring for 30 min. TLC indicated completion of the reaction. N,N-dimethylformamide was removed by concentration under reduced pressure, and the residue was purified by TLC (developing solvent:dichloromethane:methanol=10:1), to obtain intermediate C-1 (200 mg), with a yield of 67%.

### Step 2: Preparation of Compound C-2

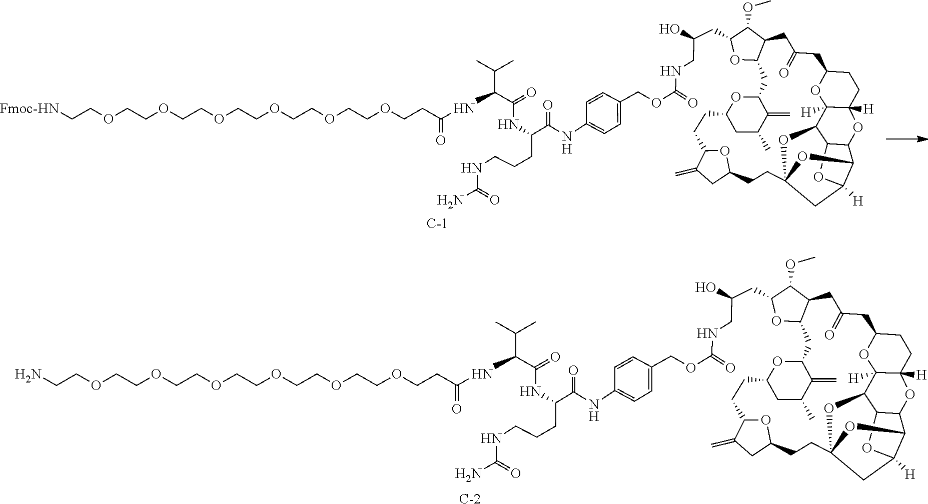

To a 25 mL reaction flask, were added intermediate C-1 (30 mg, 0.020 mmol), 1,8-diazabicyclo[5.4.0]undec-7-ene (4.5 mg, 0.030 mmol), and N,N-dimethylformamide (1 mL), and then the mixture was allowed to react under stirring at room temperature for 30 min TLC indicated completion of the reaction, which was directly used in the next step.

### Step 3: Preparation of Compound XIc

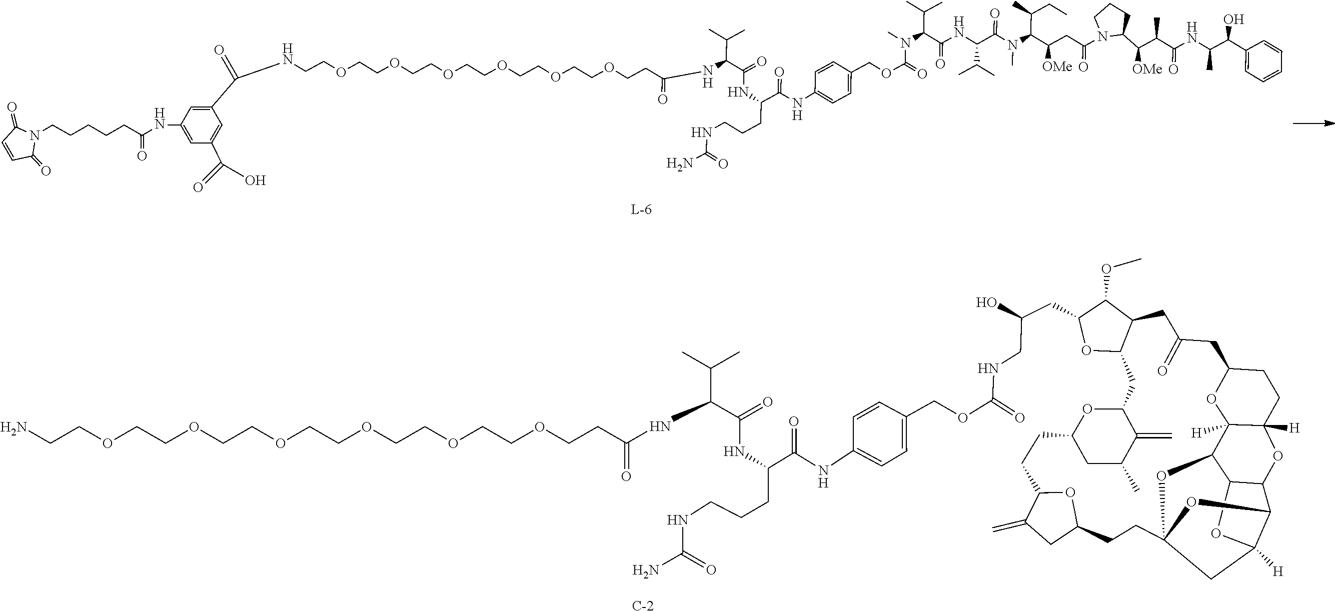

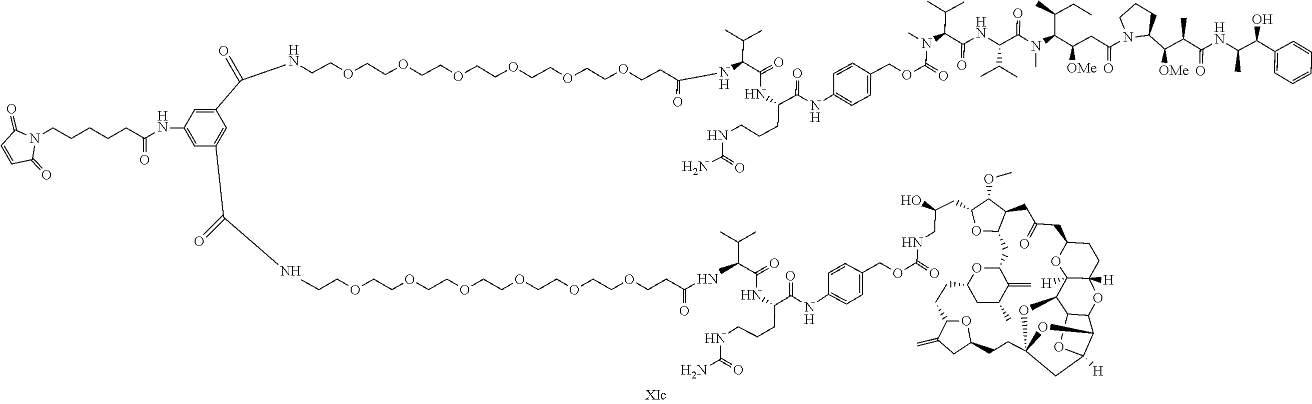

To a 25 mL reaction flask, were added intermediate L-6 (10 mg, 0.06 mmol), intermediate C-2 (8.2 mg, 0.006 mmol), N,N,N′,N′-tetramethyl-O-(7-azabenzotriazol-1-yl)uronium hexafluorophosphate (3.42 mg, 0.009 mmol), N,N-diisopropylethylamine (1.5 mg, 0.012 mmol), and N,N-dimethylformamide (1 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIc (14 mg), with a yield of 67%.

### Example 4. Preparation of the Dual-Drug Link Assembly Unit of Formula XId According to the Present Invention

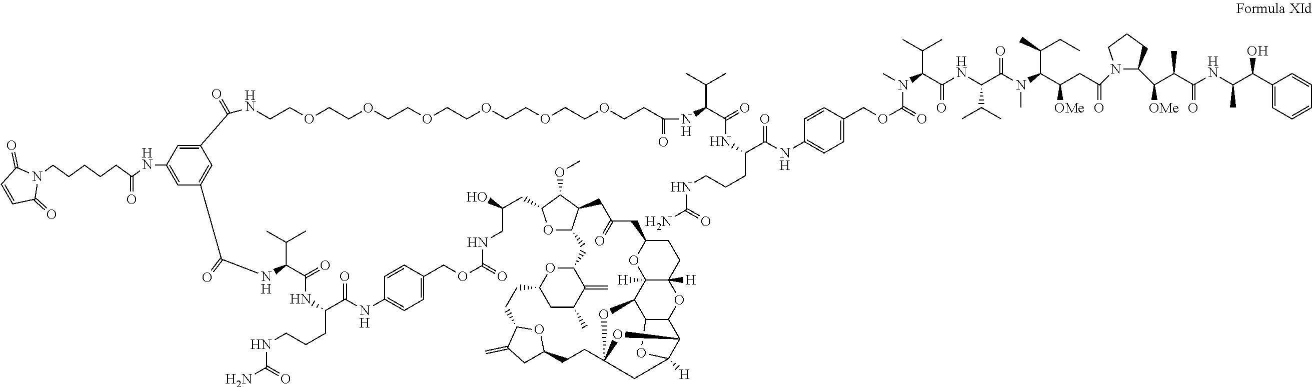

### Step 1: Preparation of Compound XId

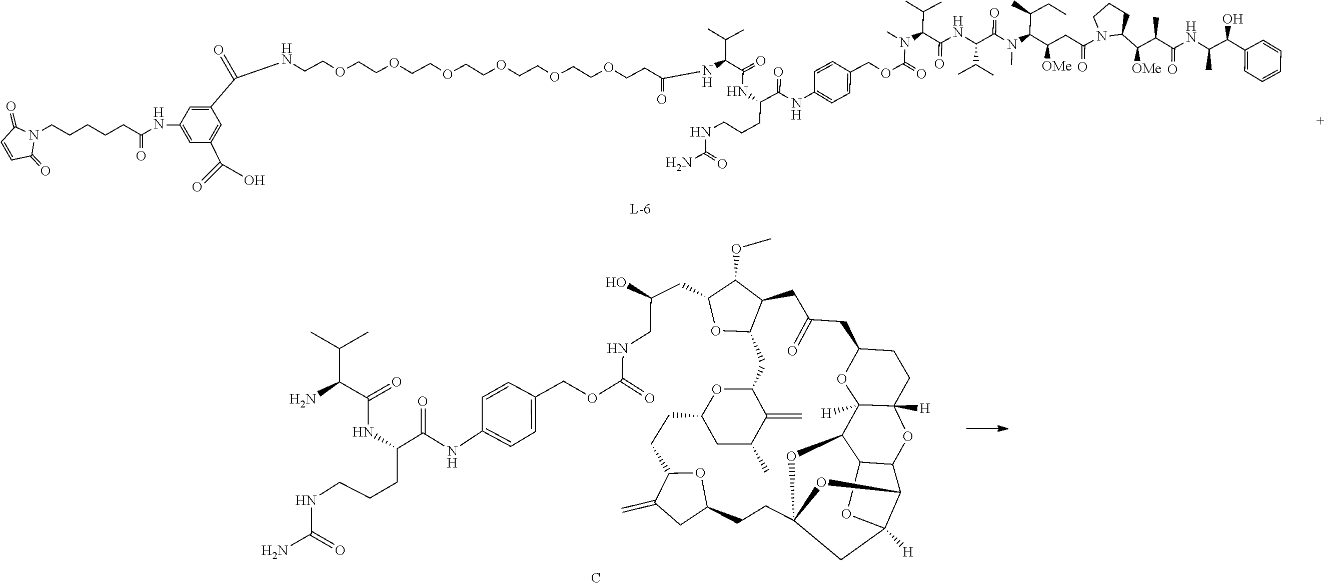

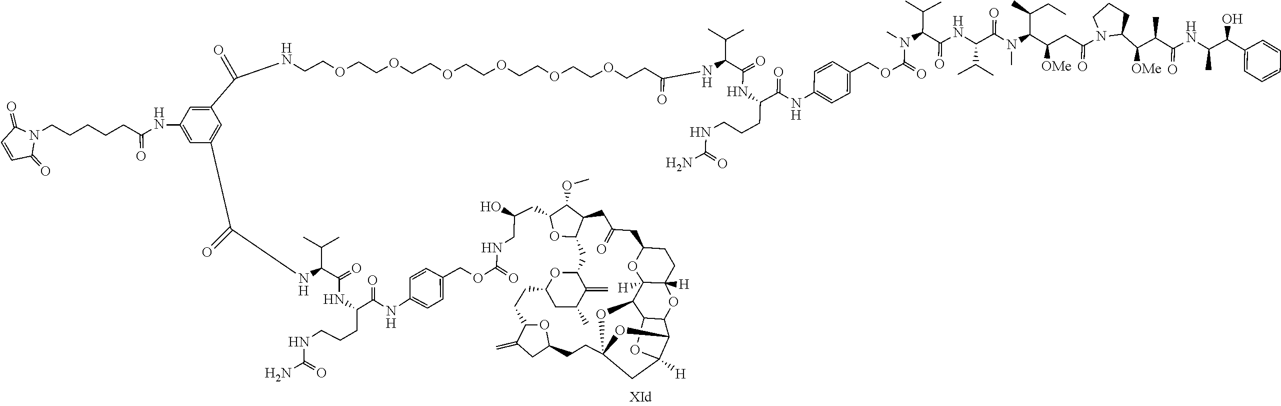

To a 25 mL reaction flask, were added intermediate L-6 (10 mg, 0.06 mmol), intermediate C (5 mg 006 mmol) N,N,N,′N′-tetramethyl-O-(7-azabenzotriazol-1-yl)uronium hexafluorophosphate (3.42 mg, 0.009 mmol), N,N-diisopropyl ethyl amine (1.5 mg, 0.012 mmol), and N,N-dimethylformamide (1 mL), and then the mixture was allowed to react at room temperature under stirring for 30 min TLC indicated completion of the reaction. N,N-dimethylformamide was removed by concentration under reduced pressure, and the residue was purified by TLC (dichloromethane:methanol=10:1), to obtain compound XId (8 mg), with a yield of 50%.

### Example 5. Preparation of the Dual-Drug Link Assembly Unit of Formula XIe According to the Present Invention

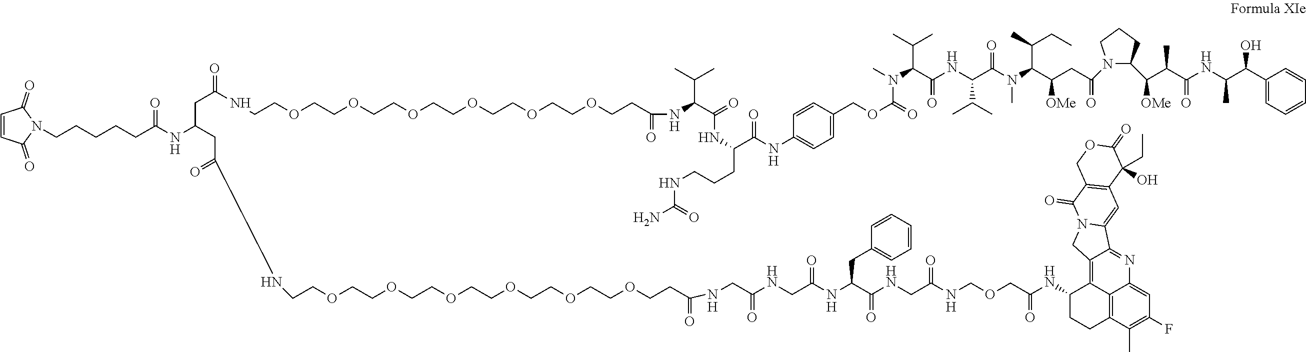

### Step 1: Preparation of Compound XIe

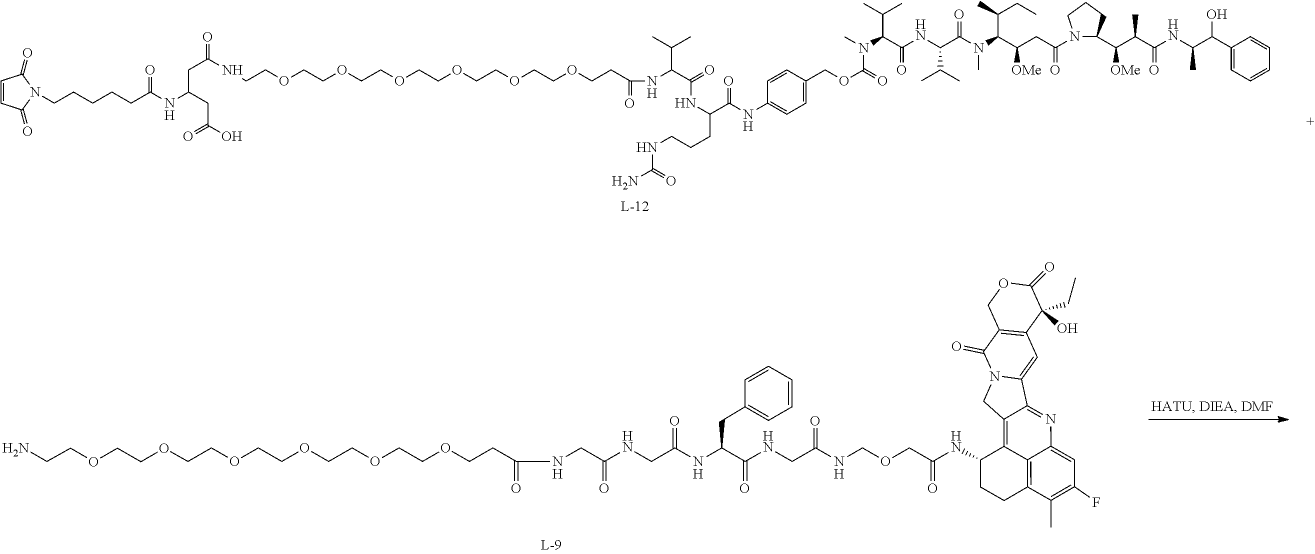 [SMILES: CC[C@H](C)[C@@H]([C@@H](CC(=O)N1CCC[C@H]1[C@H](OC)[C@@H](C)C(=O)N[C@H](C)C(O)c1ccccc1)OC)N(C)C(=O)[C@@H](NC(=O)[C@H](C(C)C)N(C)C(=O)OCc1ccc(NC(=O)C(CCCNC(N)=O)NC(=O)C(NC(=O)CCOCCOCCOCCOCCOCCOCCNC(=O)CC(CC(=O)O)NC(=O)CCCCCN2C(=O)C=CC2=O)C(C)C)cc1)C(C)C ; CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCOCCOCCOCCOCCN)CC3]

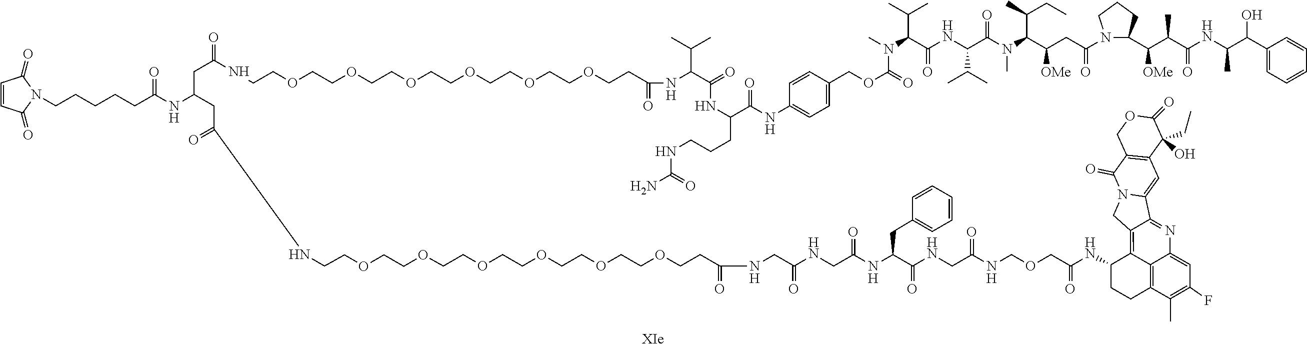 [SMILES: CC[C@H](C)[C@@H]([C@@H](CC(=O)N1CCC[C@H]1[C@H](OC)[C@@H](C)C(=O)N[C@H](C)C(O)c1ccccc1)OC)N(C)C(=O)[C@@H](NC(=O)[C@H](C(C)C)N(C)C(=O)OCc1ccc(NC(=O)C(CCCNC(N)=O)NC(=O)C(NC(=O)CCOCCOCCOCCOCCOCCOCCNC(=O)CC(CC(=O)NCCOCCOCCOCCOCCOCCOCCC(=O)NCC(=O)NCC(=O)N[C@@H](Cc2ccccc2)C(=O)NCC(=O)NCOCC(=O)N[C@H]2CCc3c(C)c(F)cc4nc5c(c2c34)Cn2c-5cc3c(c2=O)COC(=O)[C@]3(O)CC)NC(=O)CCCCCN2C(=O)C=CC2=O)C(C)C)cc1)C(C)C]

To a 25 mL reaction flask, were added intermediate L-12 (15 mg, 0.008 mmol), L-9 (10 mg, 0.008 mmol), N,N-dimethylformamide (1 mL), N,N-diisopropylethylamine (1.5 mg, 0.012 mmol), and N,N,N′,N′-tetramethyl-O-(7-azabenzotriazol-1-yl)uronium hexafluorophosphate (3.6 mg, 0.010 mmol), and then the reaction was allowed to react at room temperature under stirring for 20 min. LCMS indicated disappearane of L-12, suggesting completion of the reaction. The reaction was directly subjected to reversed-phase preparation, to obtain 12 mg of yellow solid, with a yield of 52%.

### Example 6. Preparation of the Dual-Drug Link Assembly Unit of Formula XIf According to the Present Invention

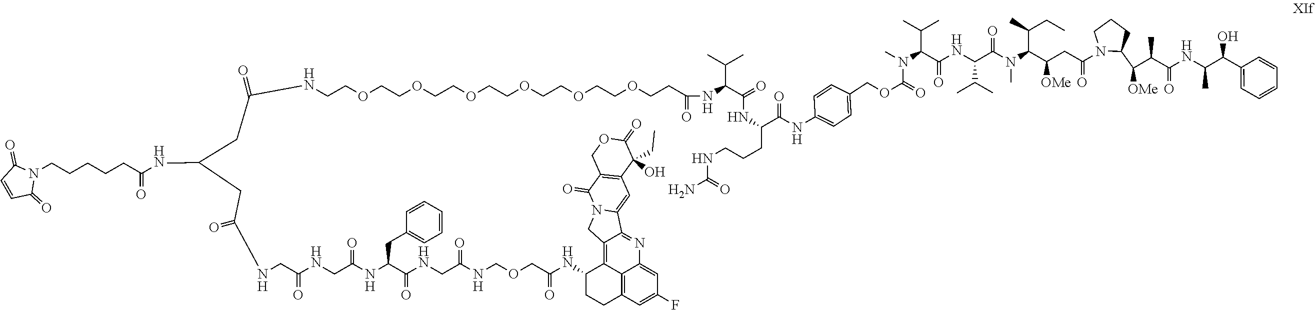

### Step 1: Preparation of Intermediate L-11

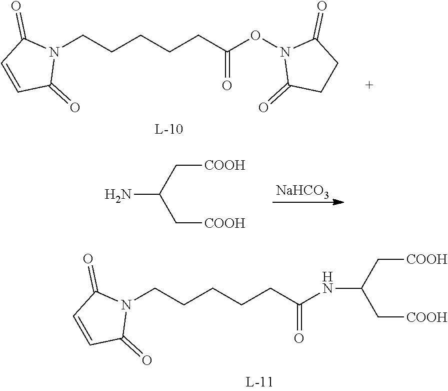

To a 25 mL reaction flask, were added L-10 (460 mg, 1.475 mmol), 3-aminopentanedioic acid (200 mg, 1.359 mmol), sodium bicarbonate (148 mg, 1.767 mmol), tetrahydrofuran (6 mL), and water (12 mL), and then the mixture was stirred and reacted at room temperature for 24 h, followed by filtration. The filtrate was extracted with 50 mL of ethyl acetate, and then the solution was separated. The water phase was adjusted to pH 2 with hydrochloric acid, extracted with 100 mL of ethyl acetate:methanol=40:1, dried over anhydrous sodium sulfate, and concentrated, to obtain compound L-11 (35 mg), with a yield of 7%.

¹H NMR (400 MHz, CDCl3) δ 12.01 (s, 1H), 11.90 (s, 1H), 8.14 (s, 1H), 7.83 (s, 1H), 7.74 (s, 1H), 4.24 (m, 1H), 4.00 (t, J=9.1 Hz, 2H), 2.42 (dd, J=12.8 Hz, 2H), 2.15 (t, J=9.8 Hz, 2H), 1.63 (m, 2H), 1.38-1.20 (m, 4H).

### Step 2: Preparation of Intermediate L-12

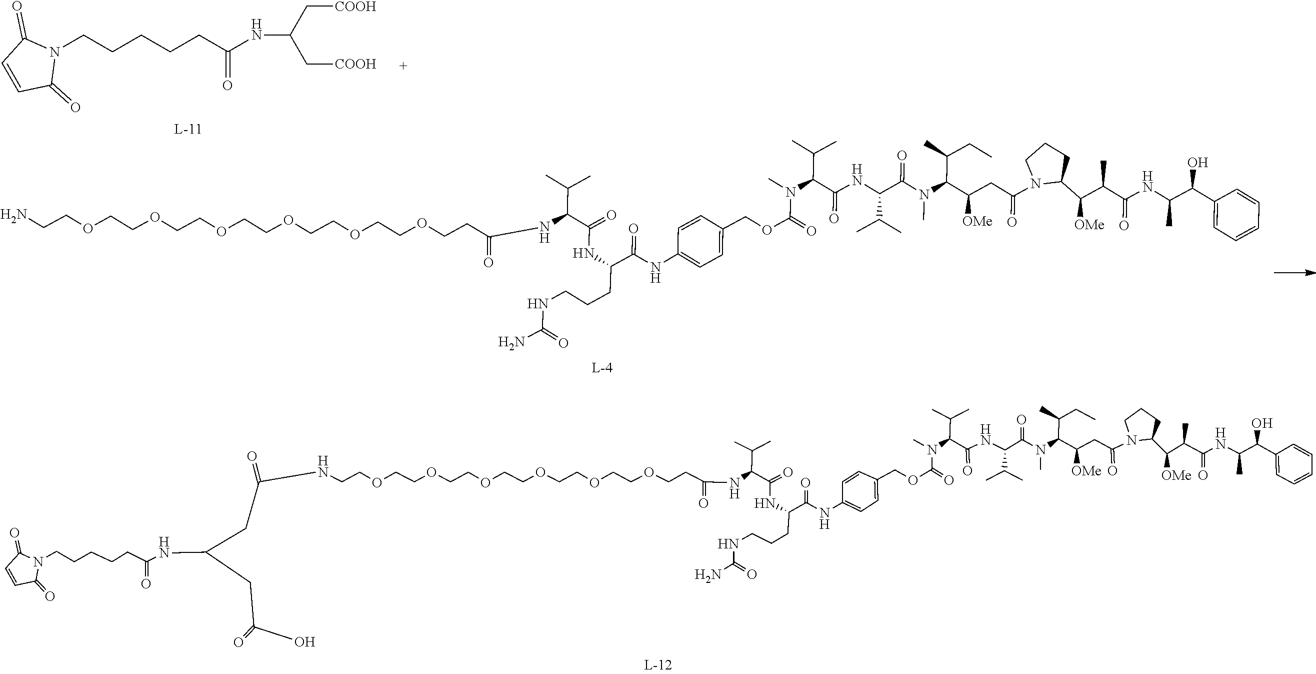

To a 25 mL reaction flask, were added L-11 (16 mg, 0.047 mmol), L-4 (20 mg, 0.014 mmol), N,N,N′,N′-tetramethyl-O-(7-azabenzotriazol-1-yl)uronium hexafluorophosphate (18 mg, 0.047 mmol), and N,N-diisopropylethylamine (6.1 mg, 0.047 mmol), and then the mixture was stirred and reacted at room temperature for 30 min. TLC indicated completion of the reaction, and then the reaction solution was concentrated under reduced pressure, to remove the solvent. The residue was purified by TLC (dichloromethane:methanol=10:1), to obtain compound L-12 (10 mg), with a yield of 40%.

### Step 3: Preparation of Compound XIf

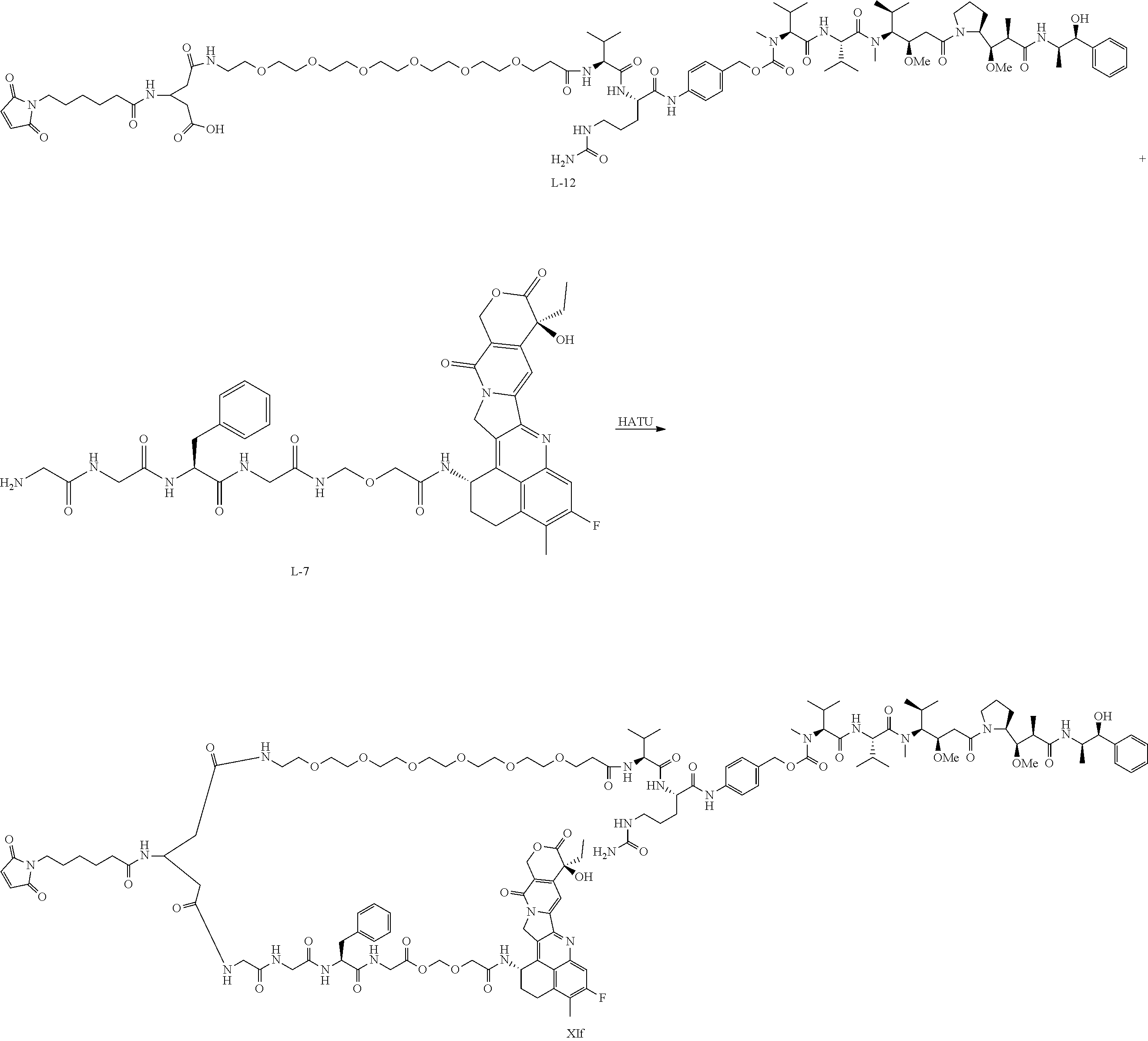

To a 25 mL reaction flask, were added compound L-12 (10 mg, 0.007 mmol), L-7 (5.8 mg, 0.007 mmol), N,N-dimethylformamide (1.5 mL), N,N-diisopropylethylamine (1.8 mg, 0.014 mmol), and N,N,N′,N′-tetramethyl-O-(7-azabenzotriazol-1-yl)uronium hexafluorophosphate (2.5 mg, 0.010 mmol), and then the mixture was stirred at room temperature for 20 min TLC indicated completion of the reaction. N,N-dimethylformamide was removed by concentration under reduced pressure, and the residue was purified by TLC (dichloromethane:methanol=8:1), to obtain compound XIf (6.8 mg).

### Example 7. Preparation of the Dual-Drug Link Assembly Unit of Formula XIg According to the Present Invention

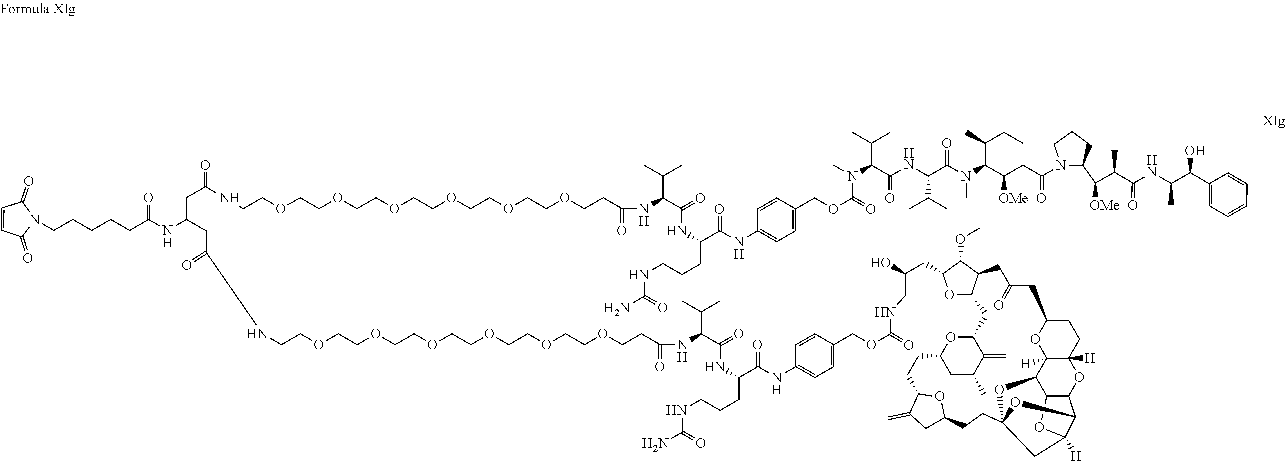

### Step 1: Preparation of Compound XIg

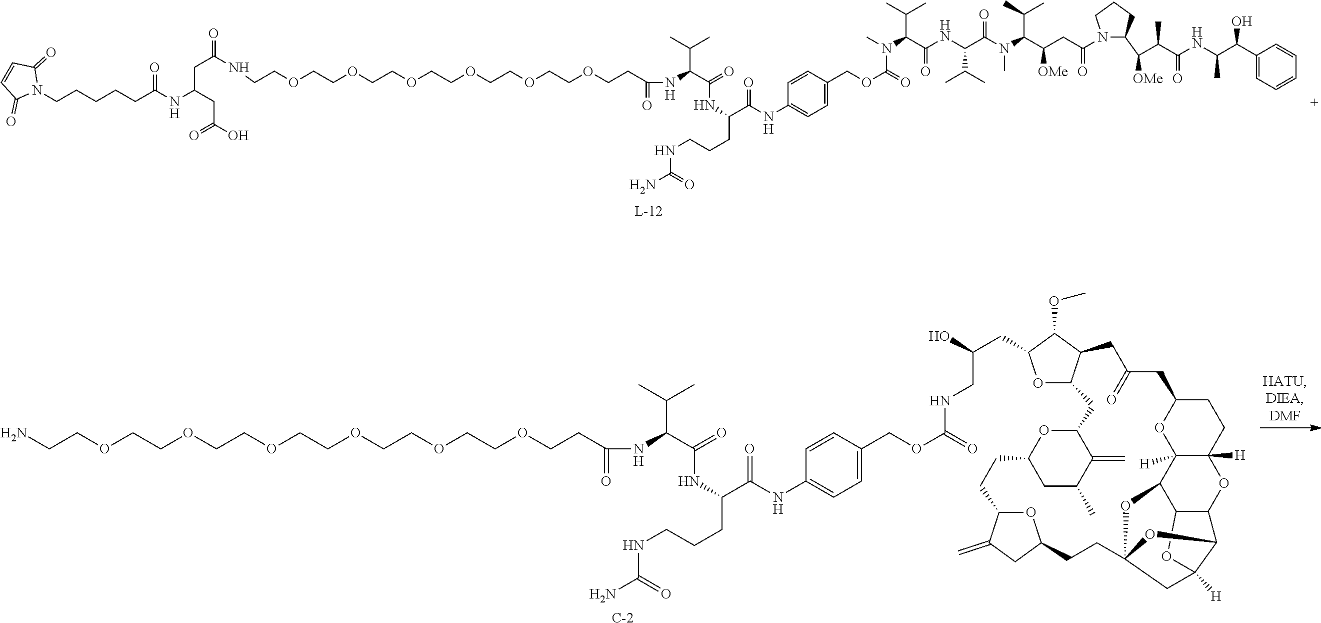

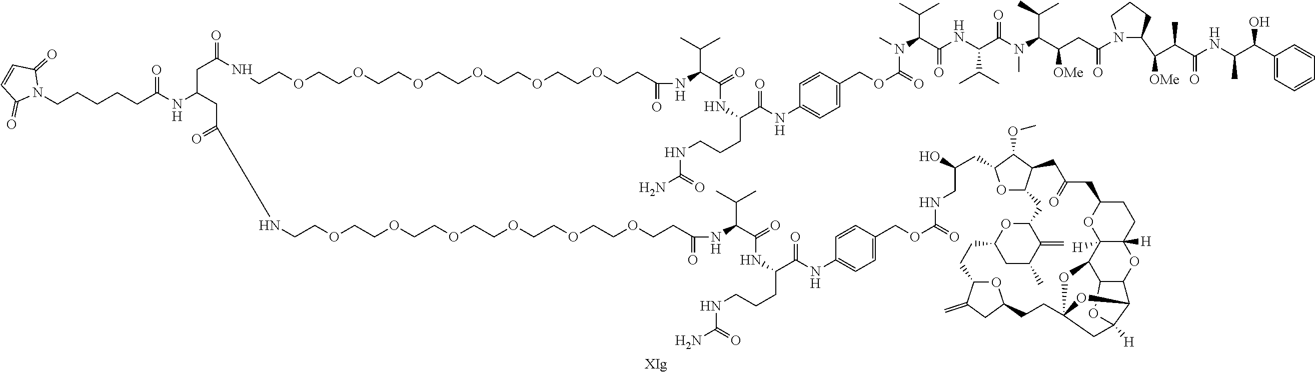

To a 25 mL reaction flask, were added intermediate L-12 (15 mg, 0.008 mmol), C-2 (12 mg, 0.008 mmol), N,N-dimethylformamide (1 mL), N,N-diisopropylethylamine (1.5 mg, 0.012 mmol), and N,N,N′,N′-tetramethyl-O-(7-azabenzotriazol-1-yl)uronium hexafluorophosphate (3.6 mg, 0.010 mmol), and then the reaction was allowed to react at room temperature under stirring for 20 min.

LCMS indicated disappearane of L-12, suggesting completion of the reaction. The reaction was directly subjected to reversed-phase preparation, to obtain 13 mg of white solid, with a yield of 50%.

### Example 8. Preparation of the Dual-Drug Link Assembly Unit of Formula XIh According to the Present Invention

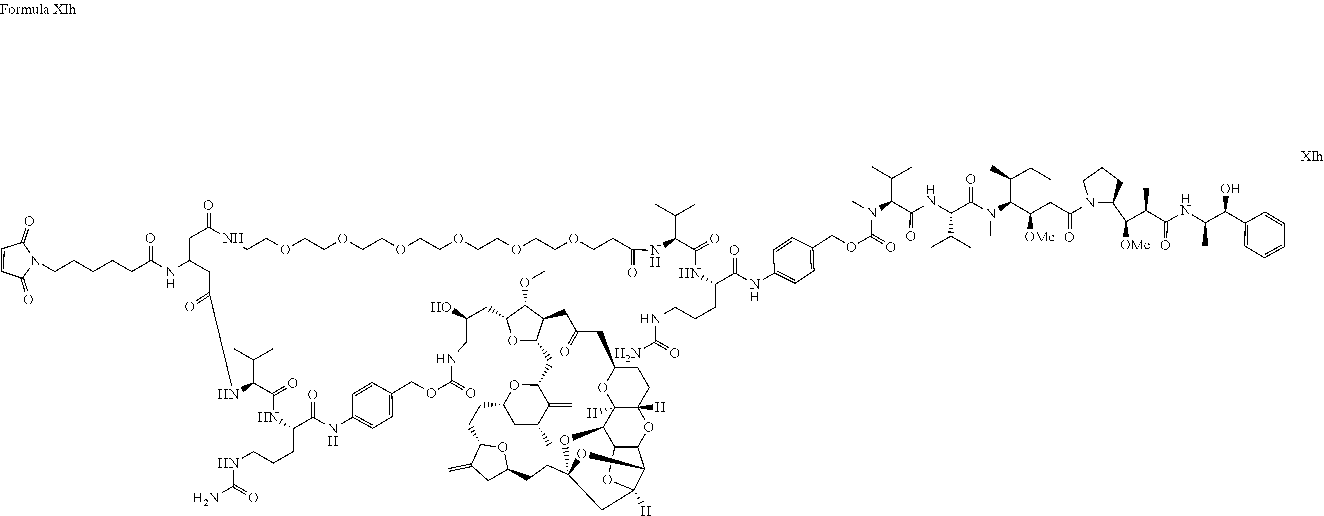

### Step 1: Preparation of Compound XIg

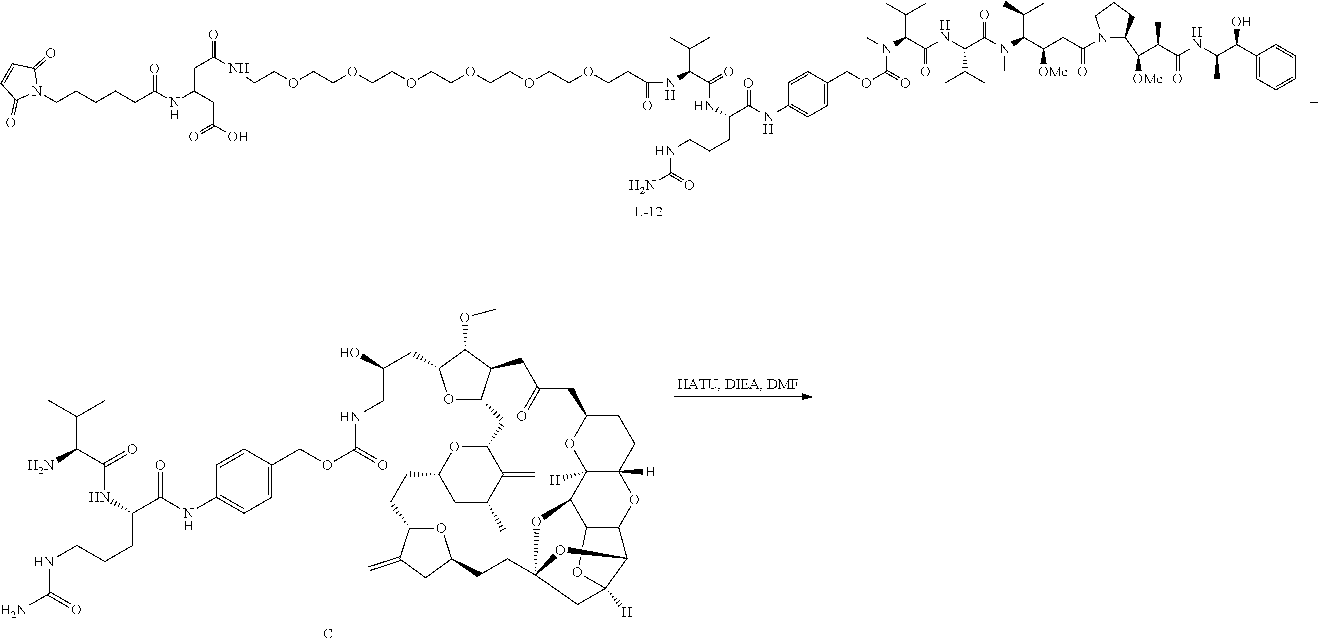

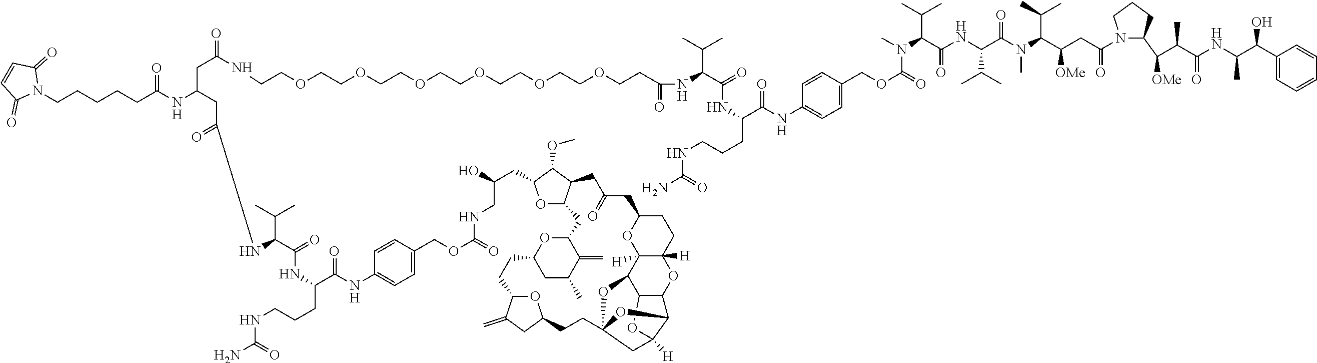

To a 25 mL reaction flask, were added intermediate L-12 (15 mg, 0.008 mmol), C (9 mg, 0.008 mmol), N,N-dimethylformamide (1 mL), N,N-diisopropylethylamine (1.5 mg, 0.012 mmol), and N,N,N′,N′-tetramethyl-O-(7-azabenzotriazol-1-yl)uronium hexafluorophosphate (3.6 mg, 0.010 mmol), and then the mixture was allowed to react at room temperature under stirring for 20 min.

LCMS indicated disappearane of L-12, suggesting completion of the reaction. The reaction was directly subjected to reversed-phase preparation, to obtain 10 mg of white solid, with a yield of 43%.

### Example 9. Preparation of the Dual-Drug Link Assembly Unit of Formula XIi According to the Present Invention

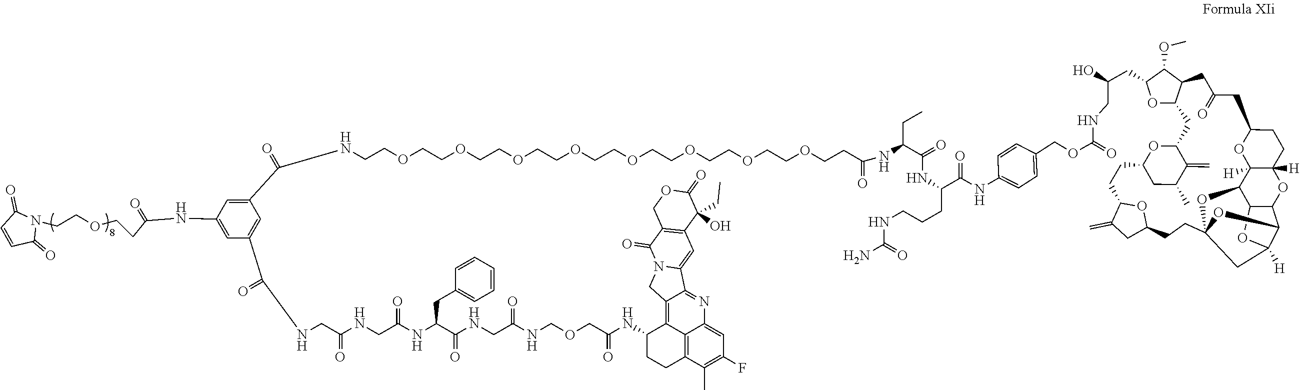

### Step 1: Preparation of Compound E-1

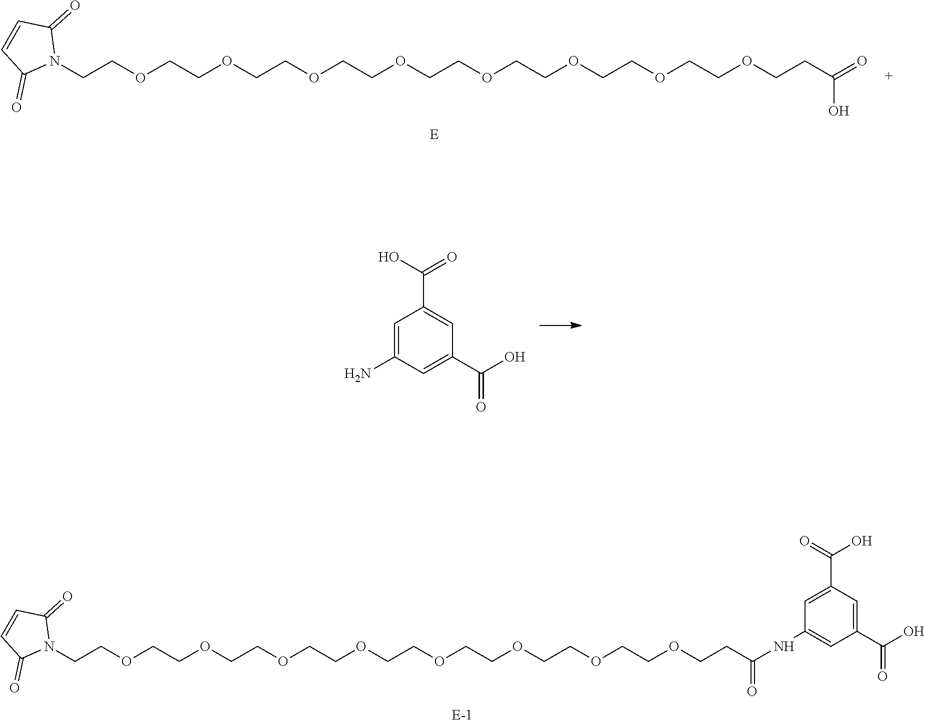 [SMILES: O=C(O)CCOCCOCCOCCOCCOCCOCCOCCOCCN1C(=O)C=CC1=O ; Nc1cc(C(=O)O)cc(C(=O)O)c1 ; O=C(CCOCCOCCOCCOCCOCCOCCOCCOCCN1C(=O)C=CC1=O)Nc1cc(C(=O)O)cc(C(=O)O)c1]

To a 25 mL reaction flask, were added intermediate E (100 mg, 0.19 mmol), N,N,N′,N′-tetramethyl-O-(7-azabenzotriazol-1-yl)uronium hexafluorophosphate (109 mg, 0.29 mmol), N,N-diisopropylethylamine (49 mg, 0.38 mmol), and N,N-dimethylformamide (4 mL), and then the mixture was allowed to react at room temperature under stirring for 5 min. After that, intermediate L-5 (34 mg, 0.19 mmol) was slowly added dropwise, and the reaction solution was reacted at room temperature for 30 min. TLC indicated completion of the reaction. N,N-dimethylformamide was removed by concentration under reduced pressure, and the residue was purified by TLC (dichloromethane:methanol=6:1), to obtain compound E-1 (34 mg), with a yield of 26%.

¹H NMR (400 MHz, CDCl₃) δ 12.74 (s, 1H), 12.56 (s, 1H), 10.10 (s, 1H), 8.51 (d, J=2.3 Hz, 1H), 8.42 (d, J=2.3 Hz, 1H), 8.16 (d, J=1.2 Hz, 1H), δ7.83 (s, 1H), 7.74 (s, 1H), 3.72 (m, 4H), 3.50-2.50 (m, 30H), 2.22 (dd, J=12.8 Hz, 2H).

### Step 2: Preparation of Compound E-2

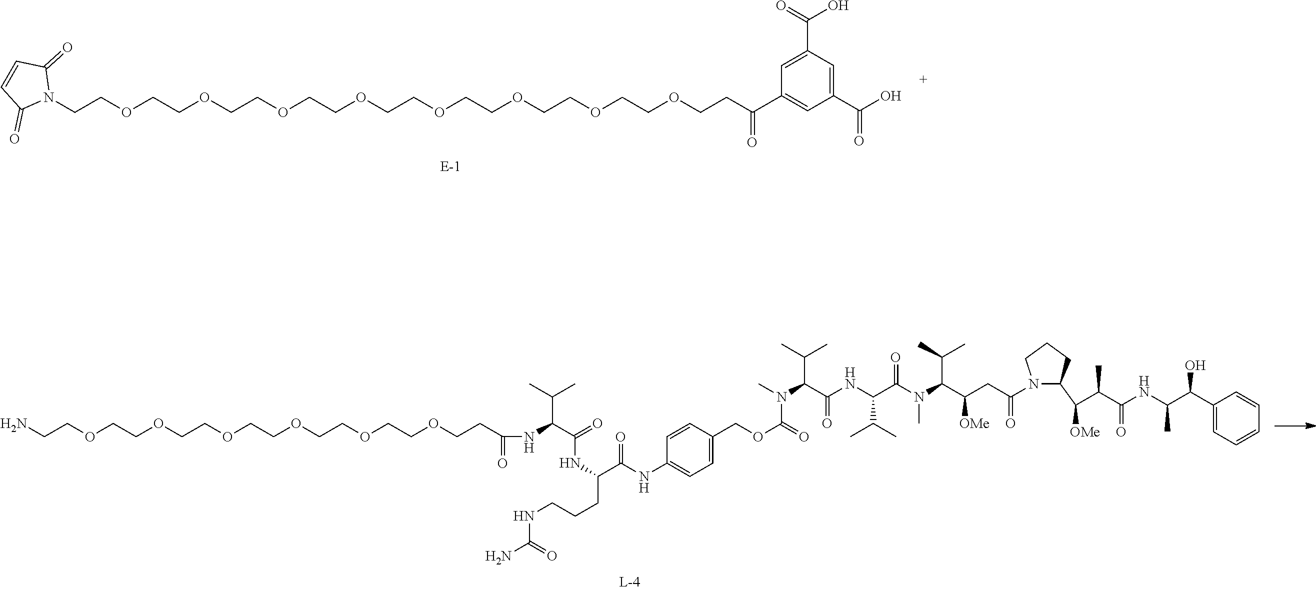

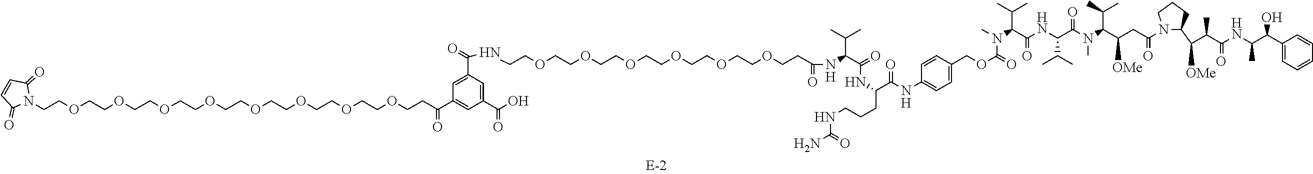

To a 25 mL reaction flask, were added intermediate E-1 (50 mg, 0.70 mmol), N,N,N′,N′-tetramethyl-O-(7-azabenzotriazol-1-yl)uronium hexafluorophosphate (54 mg, 0.14 mmol), N,N-diisopropylethylamine (18 mg, 0.14 mmol), and N,N-dimethylformamide (4 mL), and then the mixture was allowed to react at room temperature under stirring for 5 min. After that, the solution of intermediate L-4 (58 mg, 0.04 mmol) in N,N-dimethylformamide (1 mL) was slowly added dropwise, and the reaction solution was reacted at room temperature for 30 min. TLC indicated completion of the reaction. N,N-dimethylformamide was removed by concentration under reduced pressure, and the residue was purified by TLC (dichloromethane:methanol=8:1), to obtain compound E-2 (32 mg), with a yield of 38%.

### Step 3: Preparation of Compound XIi

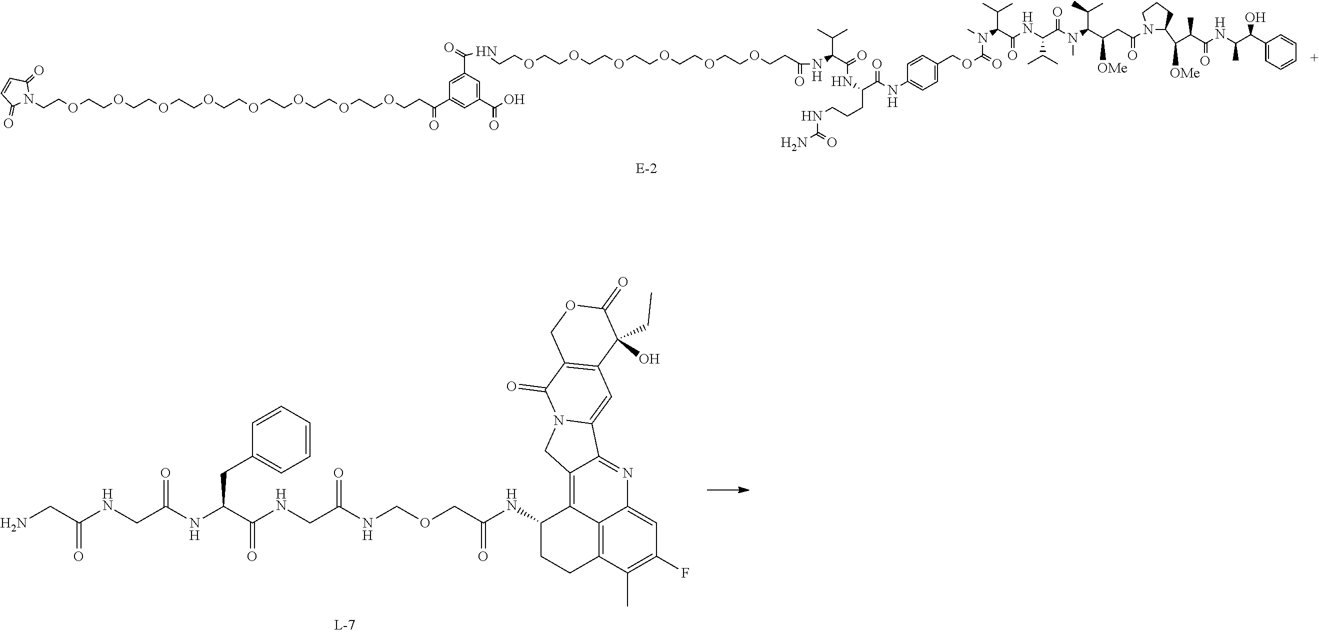

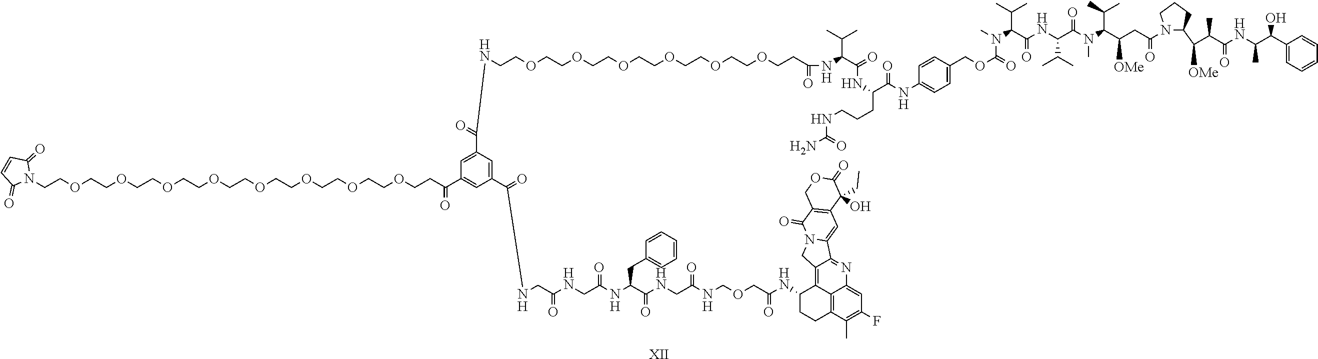

To a 25 mL reaction flask, were added intermediate E-2 (10 mg, 0.005 mmol), intermediate L-7 (3.9 mg, 0.005 mmol), N,N,N′,N′-tetramethyl-O-(7-azabenzotriazol-1-yl)uronium hexafluorophosphate (2.8 mg, 0.007 mmol), N,N-diisopropyl ethyl amine (1.2 mg, 0.010 mmol), and N,N-dimethylformamide (1 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIi (8 mg), with a yield of 57%.

### Example 10. Preparation of the Dual-Drug Link Assembly Unit of Formula XIj According to the Present Invention

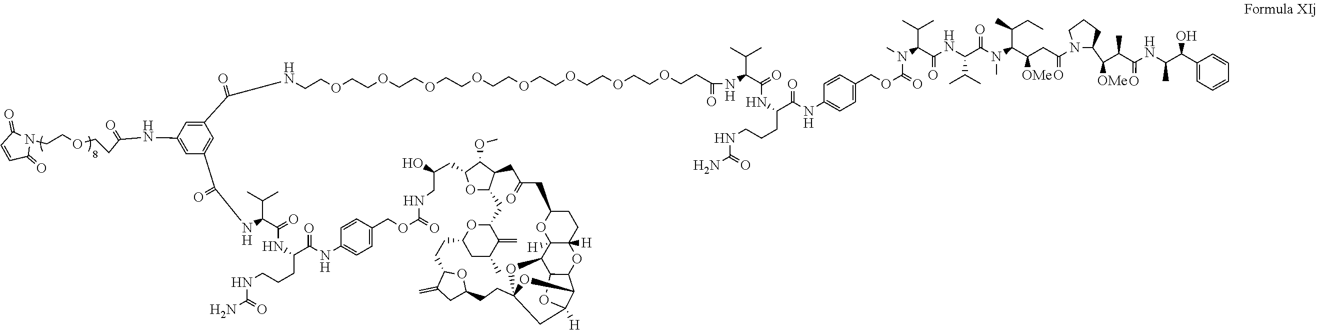

### Step 1: Preparation of Compound XIj

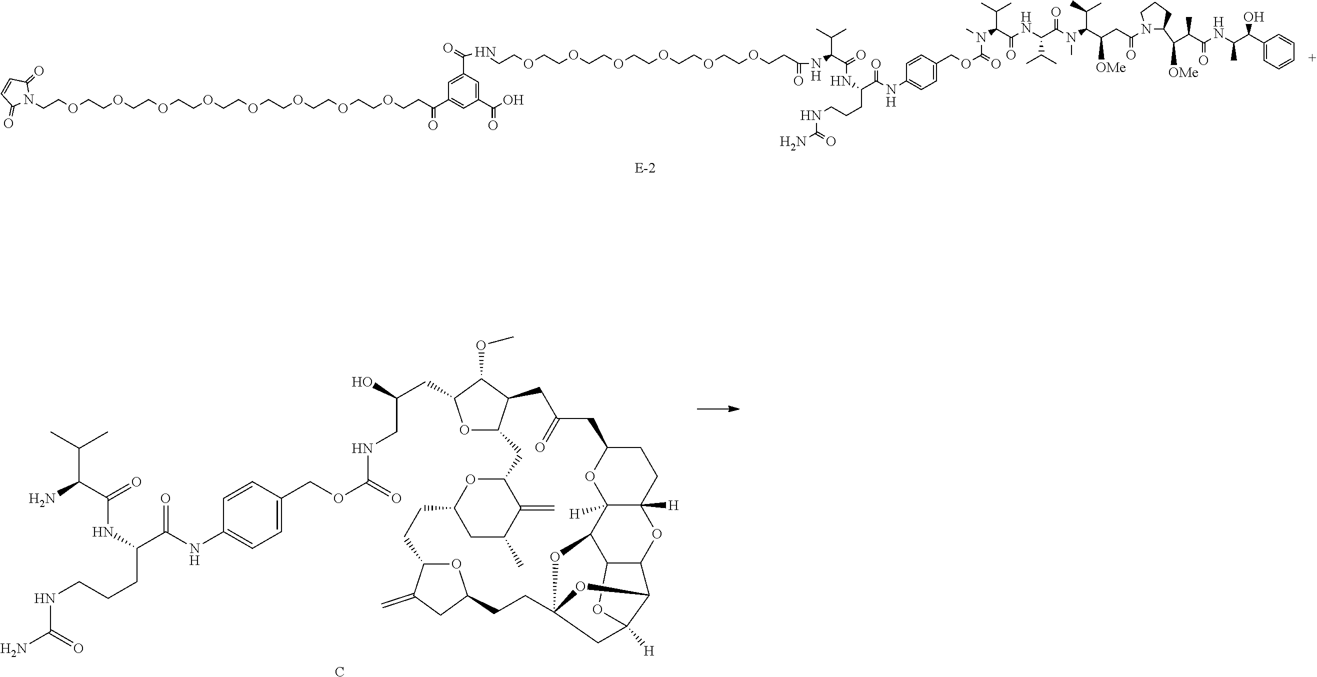

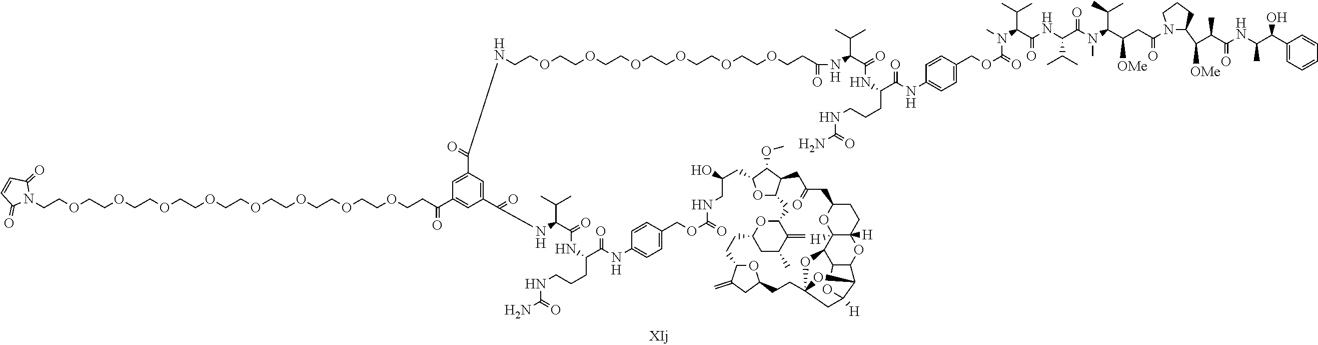

To a 25 mL reaction flask, were added intermediate E-2 (10 mg, 0.005 mmol), intermediate C (5.3 mg, 0.005 mmol), N,N,N′,N′-tetramethyl-O-(7-azabenzotriazol-1-yl)uronium hexafluorophosphate (2.8 mg, 0.007 mmol), N,N-diisopropylethylamine (1.2 mg, 0.010 mmol), and N,N-dimethylformamide (1 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIj (7.8 mg), with a yield of 56%.

### Example 11. Preparation of the Dual-Drug Link Assembly Unit of Formula XIk According to the Present Invention

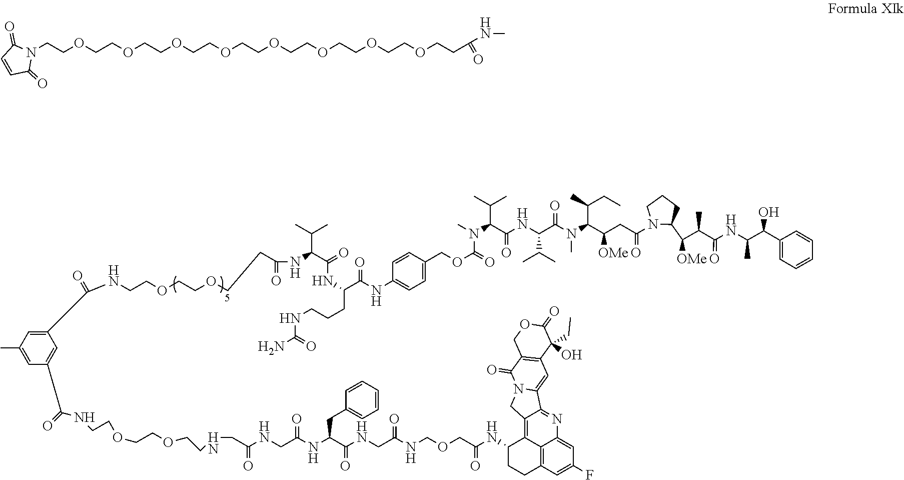

### Step 1: Preparation of Compound B-1

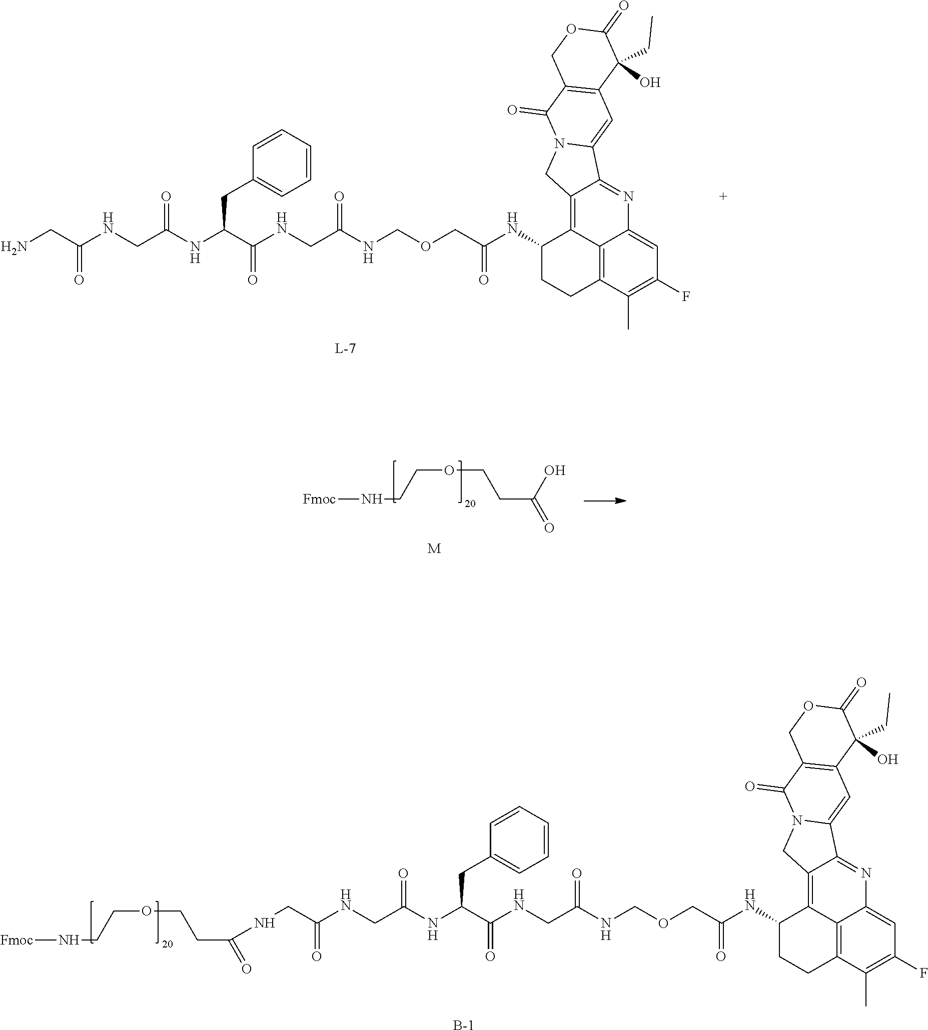

To a 25 mL reaction flask, were added compound L-7 (100 mg, 0.12 mmol), intermediate M (143 mg, 0.12 mmol), N,N,N′,N′-tetramethyl-O-(7-azabenzotriazol-1-yl)uronium hexafluorophosphate (68 mg, 0.18 mmol), N,N-diisopropylethylamine (30 mg, 0.24 mmol), and N,N-dimethylformamide (4 mL), and then the mixture was allowed to react at room temperature under stirring for 30 min TLC indicated completion of the reaction. N,N-dimethylformamide was removed by concentration under reduced pressure, and the residue was purified by TLC (developing solvent:dichloromethane:methanol=10:1), to obtain compound C-1 (80 mg), with a yield of 34%.

### Step 2: Preparation of Compound B-2

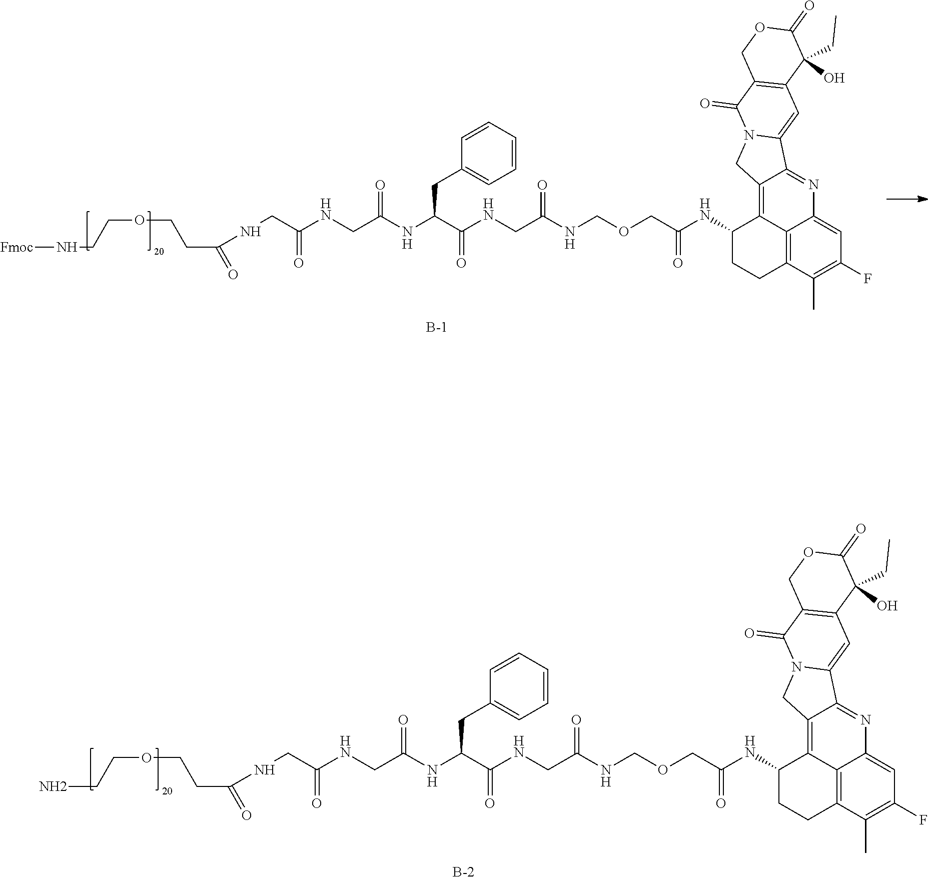

To a 25 mL reaction flask, were added intermediate B-2 (80 mg, 0.04 mmol), 1,8-diazabicyclo[5.4.0]undec-7-ene (9.1 mg, 0.06 mmol), and N,N-dimethylformamide (1 mL), and then the reaction was allowed to react under stirring at room temperature for 30 min. TLC indicated completion of the reaction, which was directly used in the next step.

### Step 3: Preparation of Compound XIk

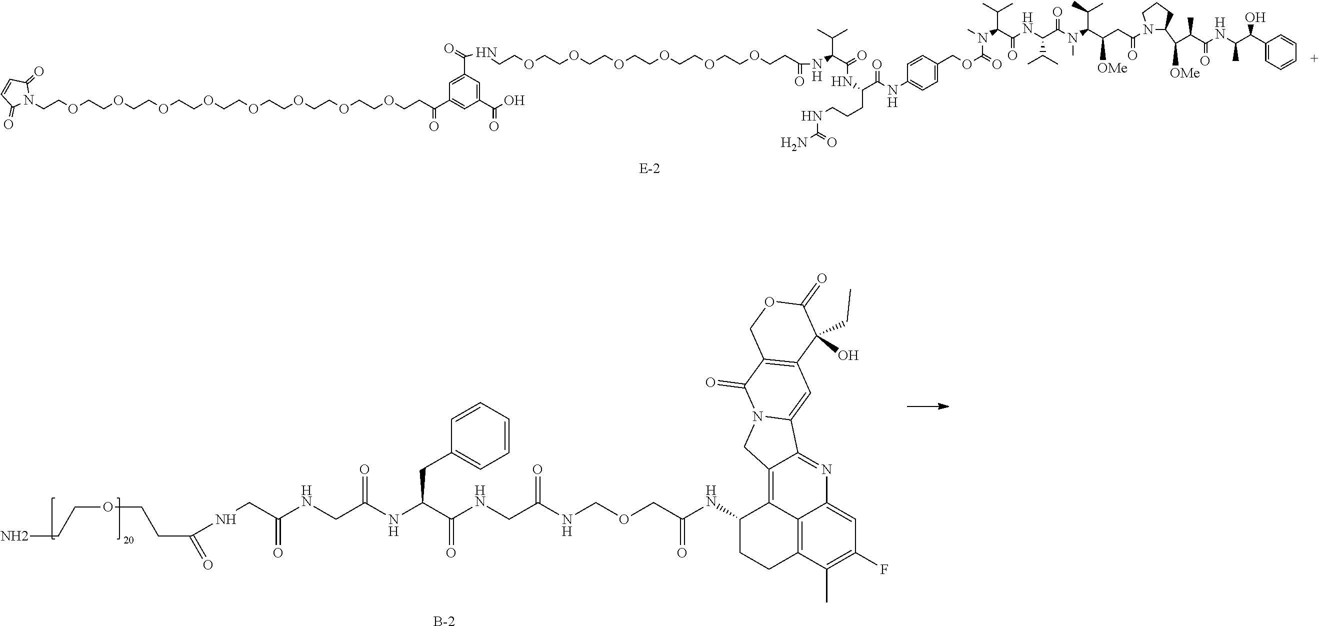

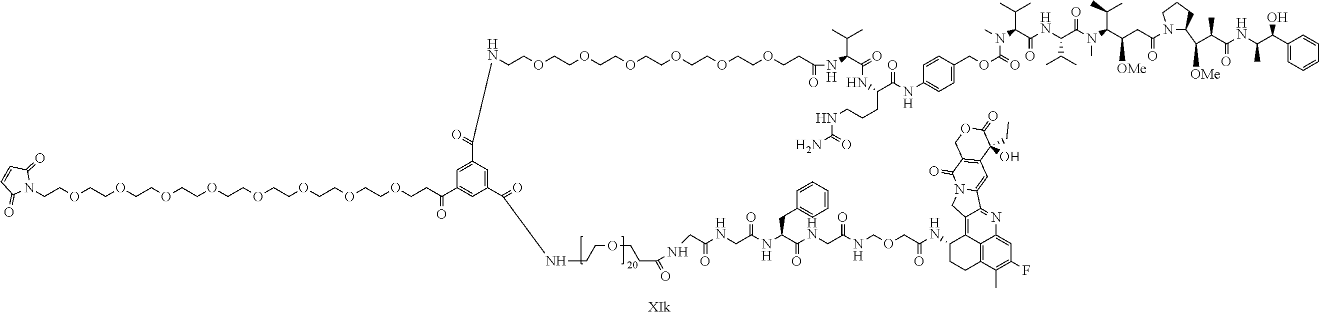

To a 25 mL reaction flask, were added compound E-2 (10 mg, 0.005 mmol), intermediate B-2 (10 mg, 0.005 mmol), N,N,N′,N′-tetramethyl-O-(7-azabenzotriazol-1-yl)uronium hexafluorophosphate (2.8 mg, 0.007 mmol), N,N-diisopropylethylamine (1.2 mg, 0.010 mmol), and N,N-dimethylformamide (1 mL), and then the mixture was allowed to react at room temperature under stirring for 30 min TLC indicated completion of the reaction. N,N-dimethylformamide was removed by concentration under reduced pressure, and the residue was purified by TLC (dichloromethane:methanol=10:1), to obtain compound XIK (13 mg), with a yield of 65%0.

### Example 12. Preparation of the Dual-Drug Link Assembly Unit of Formula XlI According to the Present Invention

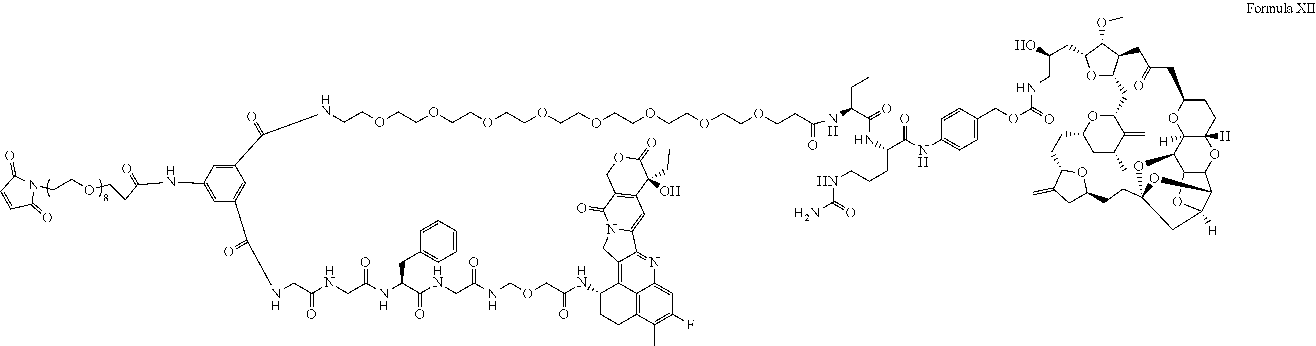

Step 1: Preparation of compound C-3

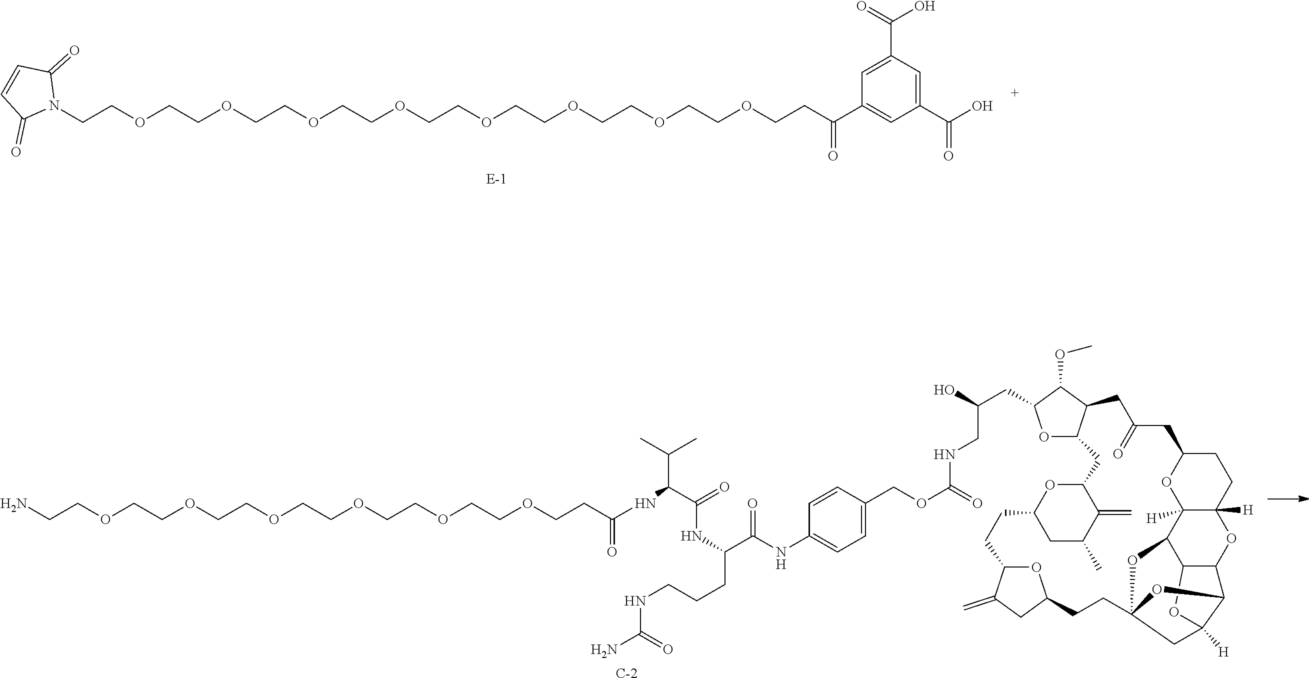

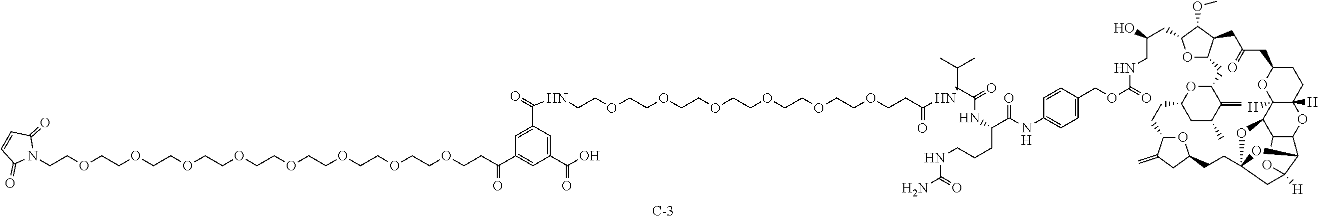

To a 25 mL reaction flask, were added intermediate E-1 (50 mg, 0.70 mmol), N,N,N′,N′-tetramethyl-O-(7-azabenzotriazol-1-yl)uronium hexafluorophosphate (54 mg, 0.14 mmol), N,N-diisopropylethylamine (18 mg, 0.14 mmol), and N,N-dimethylformamide (4 mL), and then the mixture was allowed to react at room temperature under stirring for 5 min. After that, the solution of intermediate C-2 (58 mg, 0.04 mmol) in N,N-dimethylformamide (1 mL) was slowly added dropwise, and the reaction solution was reacted at room temperature for 30 min. TLC indicated completion of the reaction. N,N-dimethylformamide was removed by concentration under reduced pressure, and the residue was purified by TLC (dichloromethane:methanol=8:1), to obtain compound C-3 (33 mg), with a yield of 38%.

### Step 2: Preparation of Compound XII

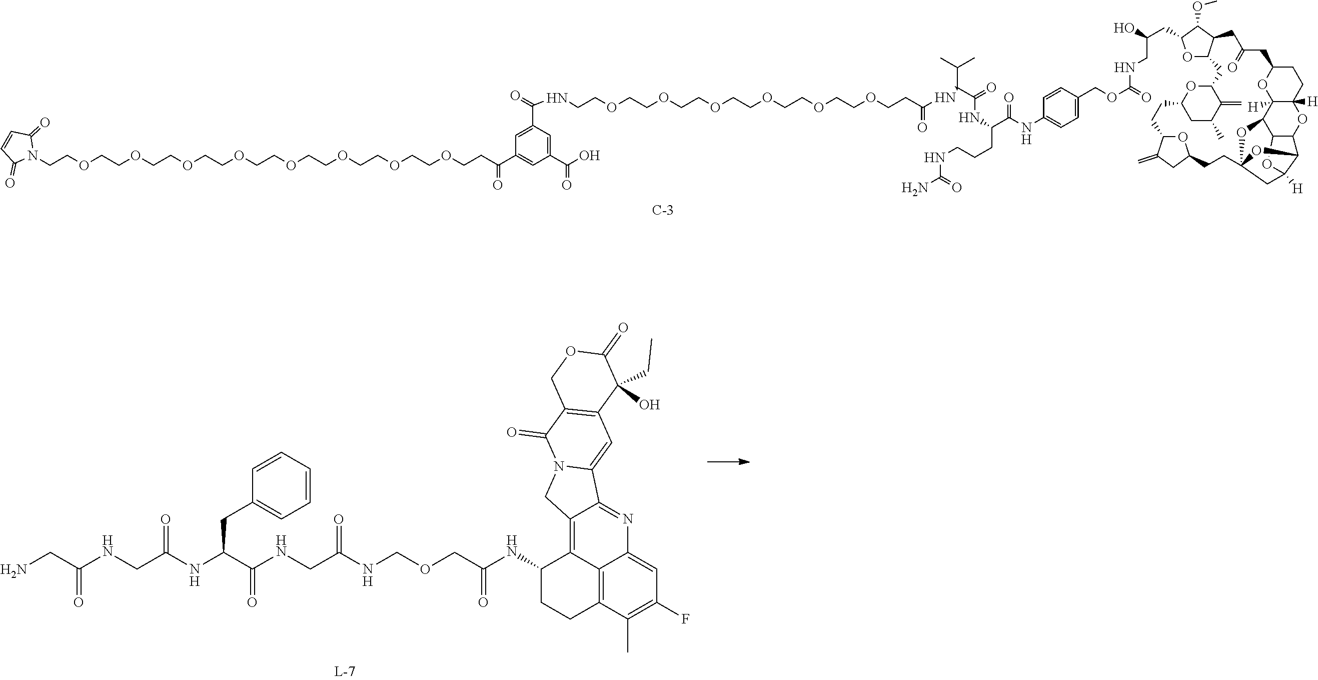

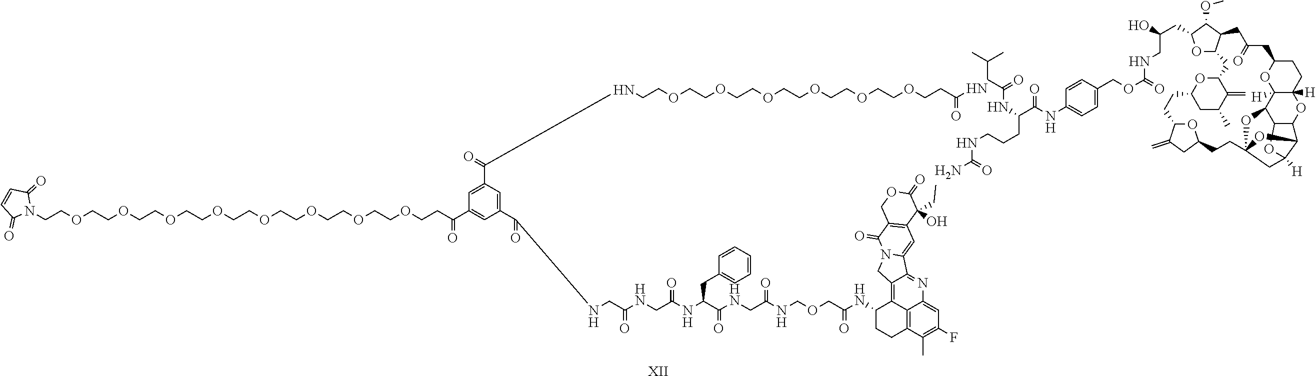

To a 25 mL reaction flask, were added compound C-3 (10 mg, 0.005 mmol), intermediate L-7 (3.9 mg, 0.005 mmol), N,N,N′,N′-tetramethyl-O-(7-azabenzotriazol-1-yl)uronium hexafluorophosphate (2.8 mg, 0.007 mmol), N,N-diisopropylethylamine (1.2 mg, 0.010 mmol), and N,N-dimethylformamide (1 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XII (7.3 mg), with a yield of 55%.

### Example 13. Preparation of the Dual-Drug Link Assembly Unit of Formula XIm According to the Present Invention

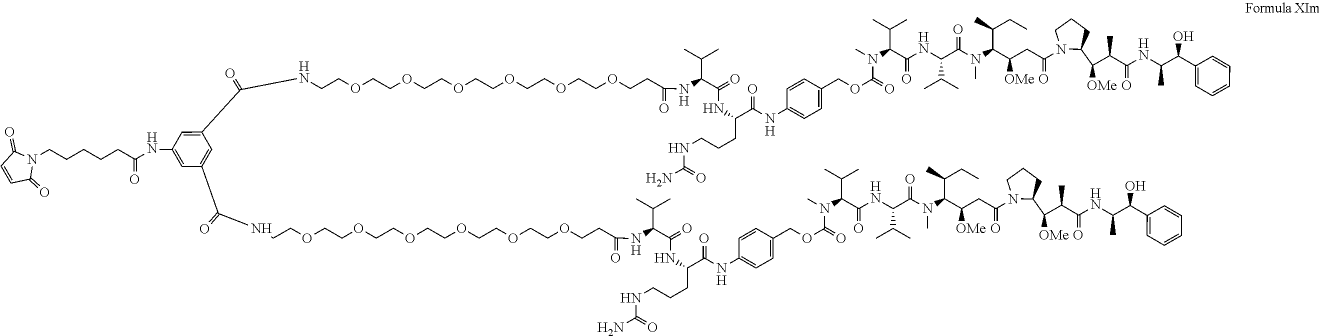

### Step 1: Preparation of Compound XIm

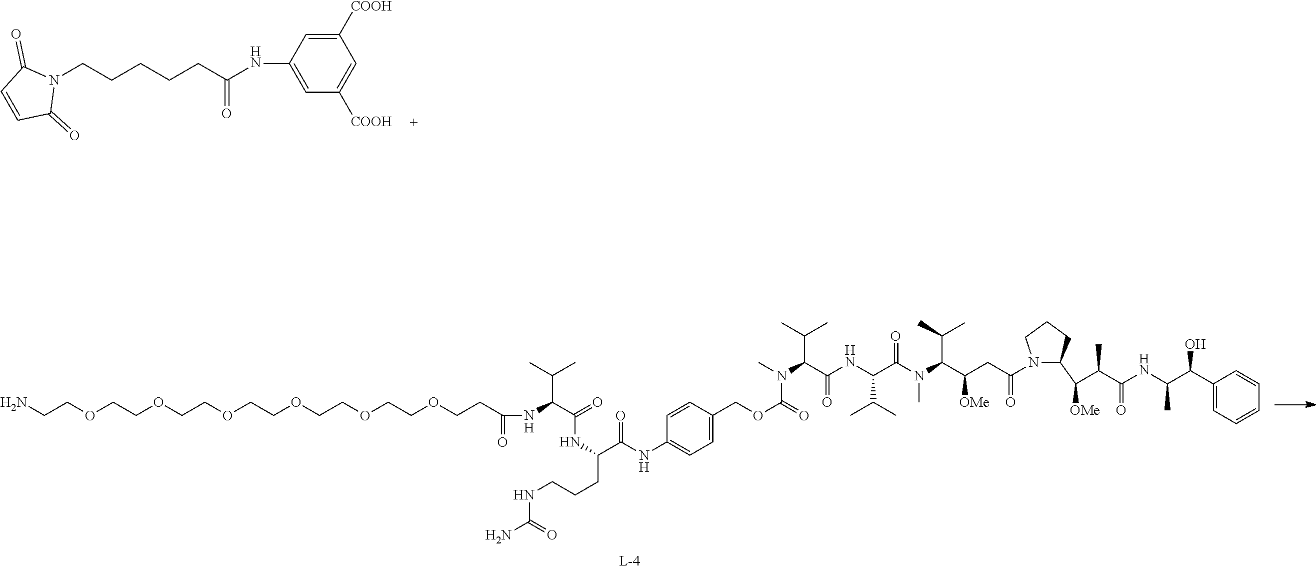

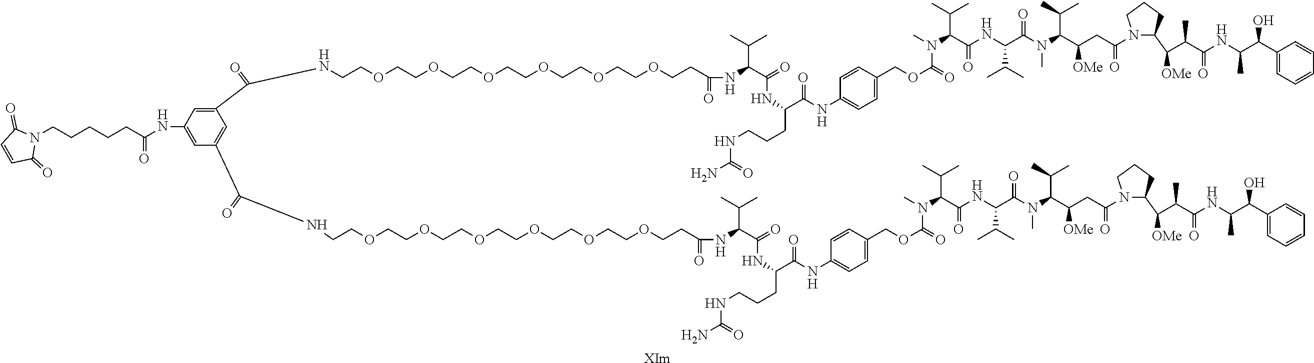

To a 25 mL reaction flask, were added intermediate L-4 (15 mg, 0.010 mmol), intermediate L-5 (1 mg, 0.005 mmol), N,N,N′,N′-tetramethyl-O-(7-azabenzotriazol-1-yl)uronium hexafluorophosphate (3.8 mg, 0.010 mmol), N,N-diisopropylethylamine (2.0 mg, 0.015 mmol), and N,N-dimethylformamide (3 mL), and then the mixture was allowed to react at room temperature under stirring for 30 min TLC indicated disappearance of L-4, suggesting completion of the reaction. N,N-dimethylformamide was removed by concentration under reduced pressure, and the residue was purified by TLC (dichloromethane:methanol=10:1), to obtain product XIm (5 mg), with a yield of 31%.

### Example 14. Preparation of the Dual-Drug Link Assembly Unit of Formula XIn According to the Present Invention

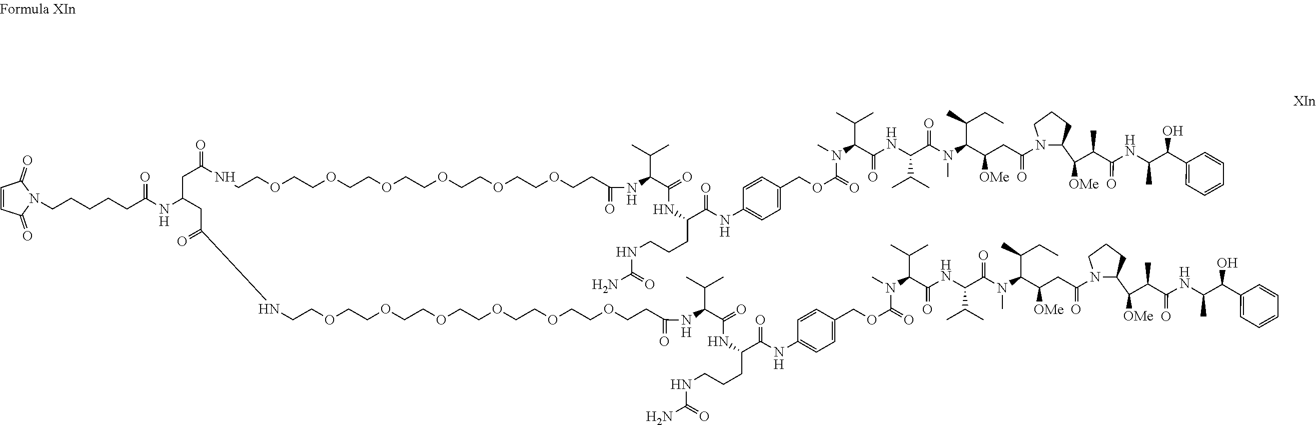

### Step 1: Preparation of Compound XIn

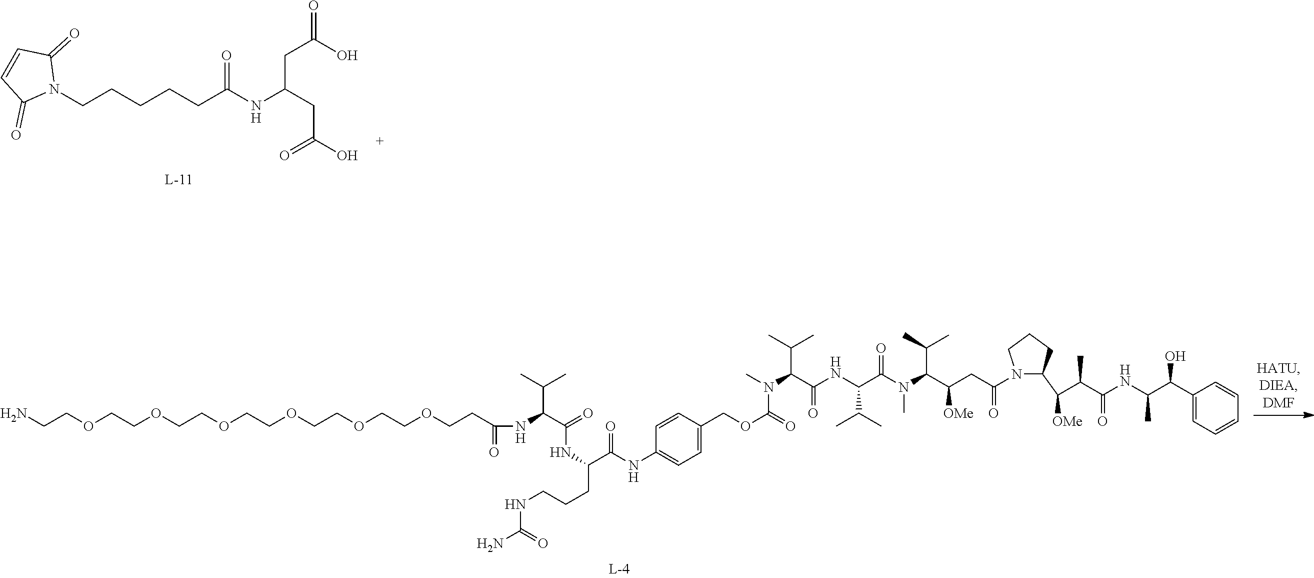

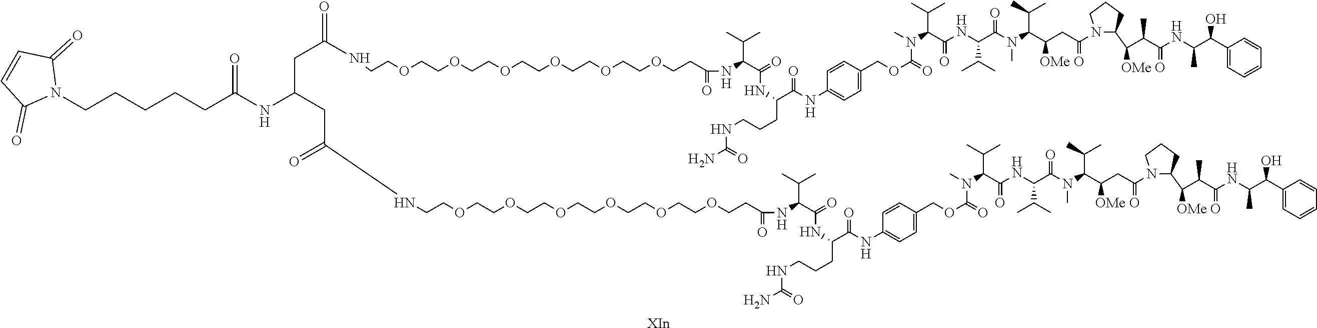

To a 25 mL reaction flask, were added intermediate L-11 (2 mg, 0.004 mmol), intermediate L-4 (12 mg, 0.008 mmol), N,N-dimethylformamide (1 mL), N,N-diisopropylethylamine (1.5 mg, 0.012 mmol), and N,N,N′,N′-tetramethyl-O-(7-azabenzotriazol-1-yl)uronium hexafluorophosphate (3.6 mg, 0.010 mmol), and then the mixture was allowed to react at room temperature under stirring for 20 min. LCMS indicated disappearance of L-12, suggesting completion of the reaction. The reaction product was directly purified by reversed-phase preparation, to obtain 8 mg of white solid, with a yield of 61%.

### Example 15. Preparation of the Dual-Drug Link Assembly Unit of Formula XIo According to the Present Invention

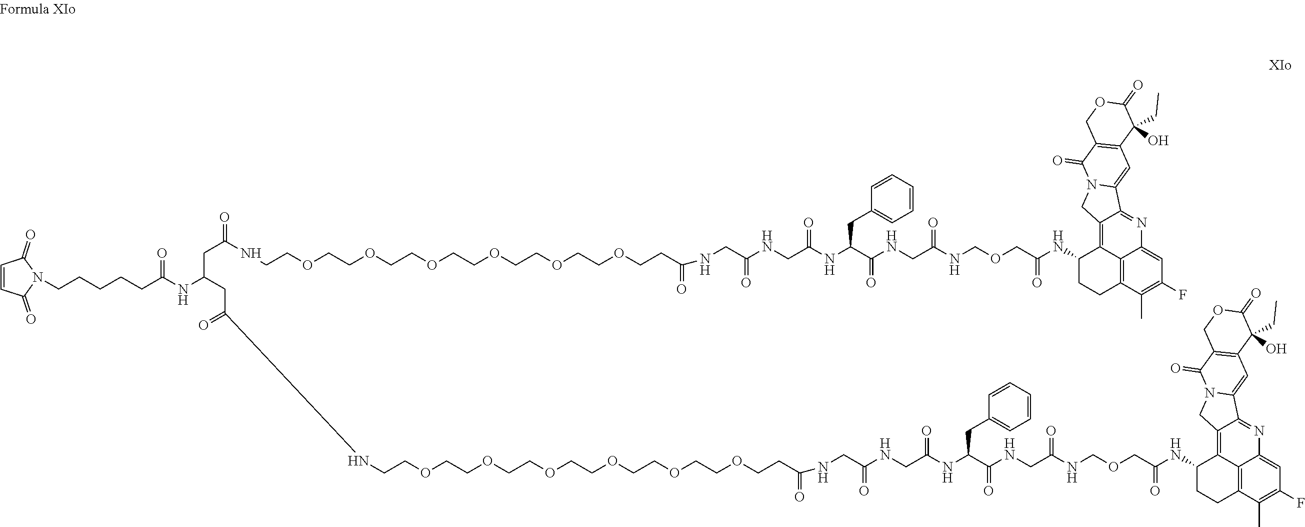

### Step 1: Preparation of Compound XIo

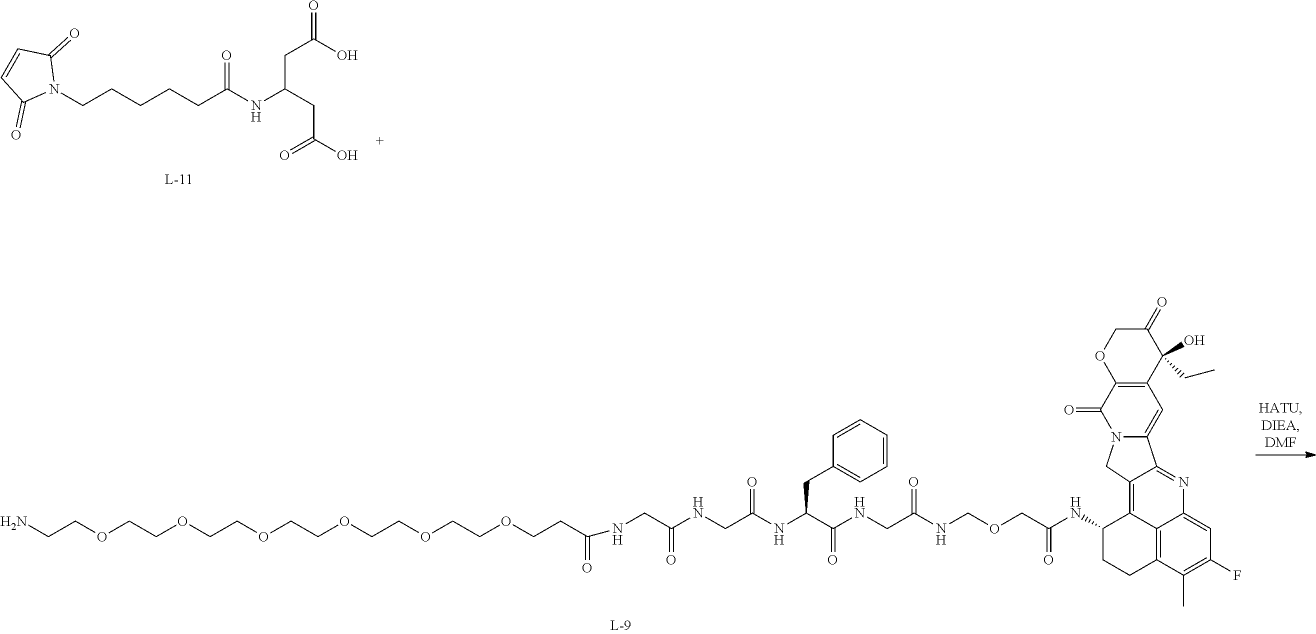 [SMILES: O=C(O)CC(CC(=O)O)NC(=O)CCCCCN1C(=O)C=CC1=O ; CC[C@@]1(O)C(=O)COc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCOCCOCCOCCOCCN)CC3]

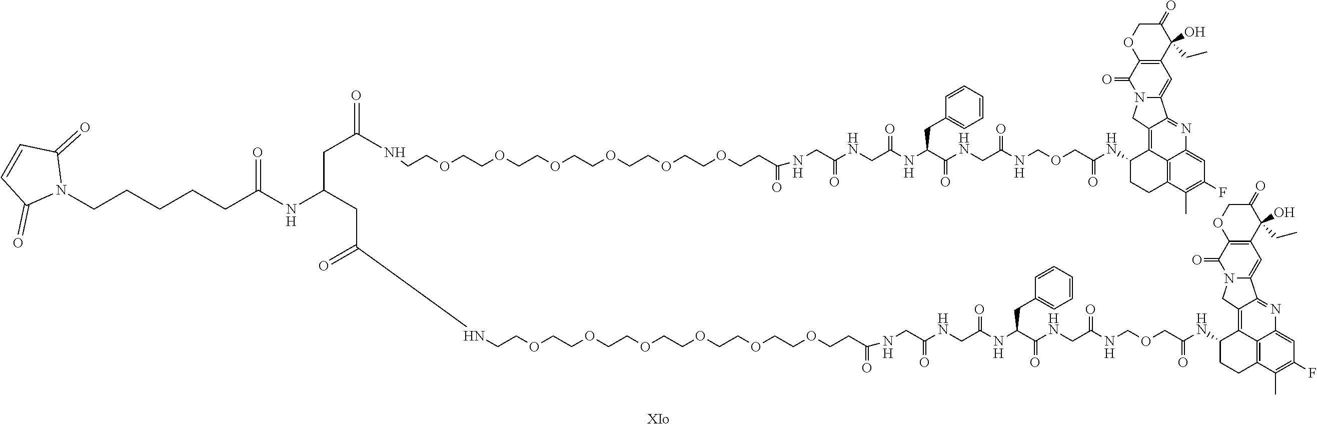 [SMILES: CC[C@@]1(O)C(=O)COc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCOCCOCCOCCOCCNC(=O)CC(CC(=O)NCCOCCOCCOCCOCCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)OCC(=O)[C@]2(O)CC)NC(=O)CCCCCN1C(=O)C=CC1=O)CC3]

To a 25 mL reaction flask, were added intermediate L-11 (2 mg, 0.004 mmol), intermediate L-9 (9.5 mg, 0.008 mmol), N,N-dimethylformamide (1 mL), N,N-diisopropylethylamine (1.5 mg, 0.012 mmol), and N,N,N′,N′-tetramethyl-O-(7-azabenzotriazol-1-yl)uronium hexafluorophosphate (3.6 mg, 0.010 mmol), and then the mixture was allowed to react at room temperature under stirring for 20 min. LCMS indicated disappearance of L-12, suggesting completion of the reaction. The reaction product was directly purified by reversed-phase preparation, to obtain 6 mg of yellow solid, with a yield of 56%.

### Example 16. Preparation of the Dual-Drug Link Assembly Unit of Formula XIp According to the Present Invention

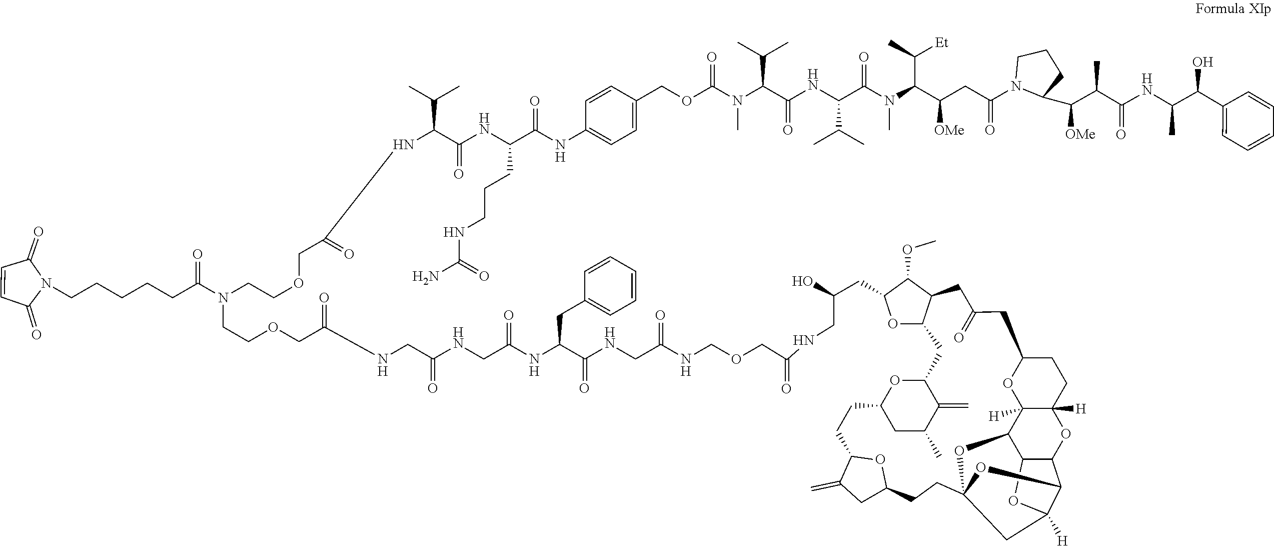

### Step 1: Preparation of Intermediate D-1

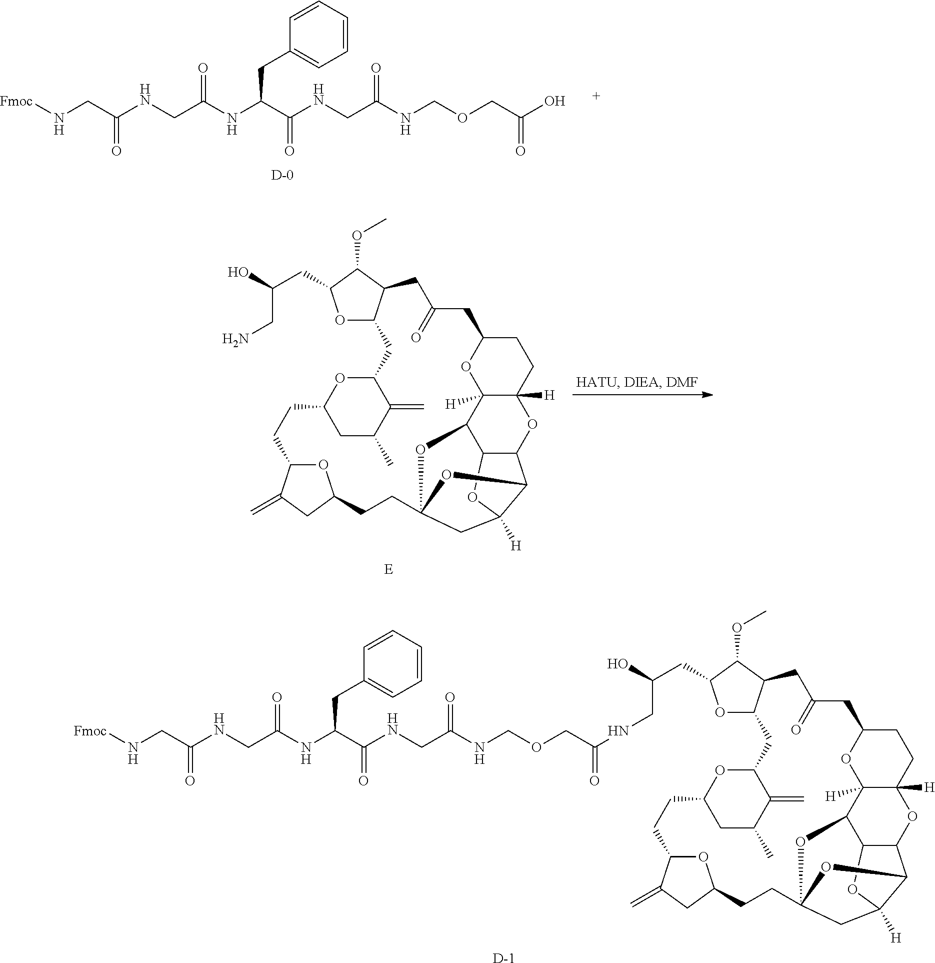

To a 25 mL reaction flask, were added intermediate D-0 (50 mg, 0.077 mmol), E (59 mg, 0.081 mmol), N,N-dimethylformamide (4 mL), N,N-diisopropylethylamine (1.2 mg, 0.115 mmol), and N,N,N′,N′-tetramethyl-O-(7-azabenzotriazol-1-yl)uronium hexafluorophosphate (35 mg, 0.092 mmol), and then the mixture was allowed to react at room temperature under stirring for 1 h. TLC indicated disappearance of D-0, suggesting completion of the reaction. N,N-dimethylformamide was removed by concentration under reduced pressure, and the residue was purified by TLC (dichloromethane:methanol=10:1), to obtain compound D-1 (65 mg), with a yield of 62%.

### Step 2: Preparation of Intermediate D-2

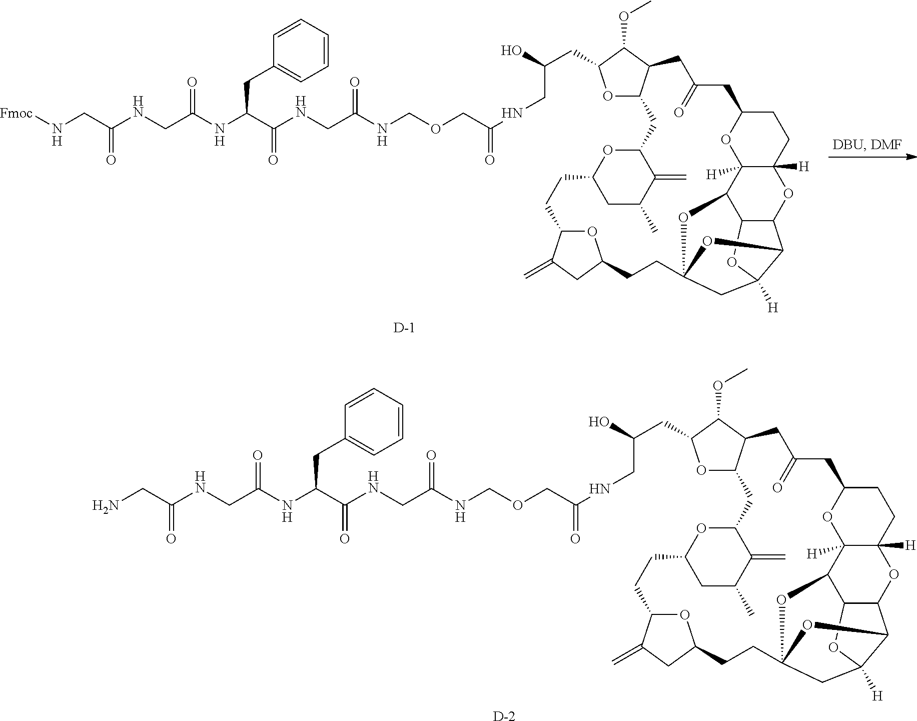

To a 10 mL reaction flask, were added D-1 (65 mg, 0.048 mmol), 1,8-diazabicyclo[5.4.0]undec-7-ene (11 mg, 0.072 mmol), and N,N-dimethylformamide (2 mL), and then the mixture was allowed to react under stirring at room temperature for 10 min. TLC indicated disappearance of D-1, and thus the reaction was completed. The reaction product was subjected to reversed-phase preparation, to obtain 35 mg of white solid, with a yield of 65%.

### Step 3: Preparation of Intermediate D-3

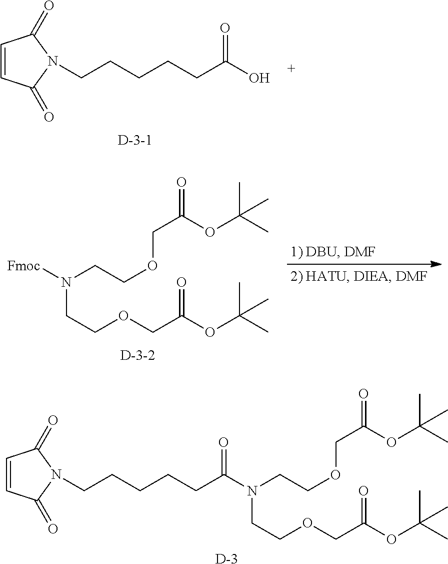

To a 10 mL reaction flask, were added D-3-2 (100 mg, 0.180 mmol), 1,8-diazabicyclo[5.4.0]undec-7-ene (42 mg, 0.270 mmol), and N,N-dimethylformamide (2 mL), and then the reaction was allowed to react under stirring at room temperature for 10 min. TLC indicated disappearance of D-3-2, and thus the reaction was completed. The resultant reaction solution was added dropwise to the reaction flask containing D-3-1 (32 mg, 0.150 mmol), N,N,N′,N′-tetramethyl-O-(7-azabenzotriazol-1-yl)uronium hexafluorophosphate (85 mg, 0.225 mmol), N,N-diisopropylethylamine (30 mg, 0.300 mmol), and N,N-dimethylformamide (2 mL). The mixture was stirred and reacted at room temperature for 30 min. LCMS indicated disappearance of starting materials, to which was added water (5 mL), and then the resultant solution was extracted with ethyl acetate (15 mL×3). The organic phase was dried with anhydrous sodium sulfate, filtered, and rotatory evaporated to dry. To the residue, was added a small amount of methanol, and after reversed-phase preparation, 50 mg of oily liquid was obtained, with a yield of 63%.

¹H NMR (400 MHz, CDCl₃) δ 7.86 (s, 1H), 7.81 (s, 1H), 4.09 (m, 2H), 3.70-3.50 (m, 12H), 2.42 (m, 4H), 2.11-1.62 (m, 8H), 1.37 (s, 18H).

### Step 4: Preparation of Intermediate D-4

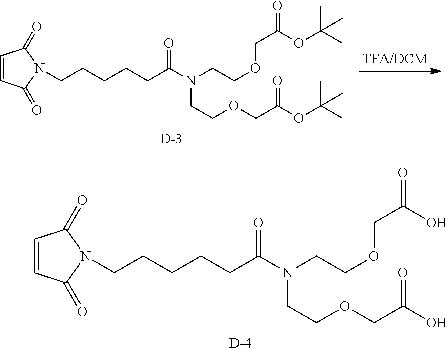

To a 10 mL reaction flask, were added D-3 (50 mg, 0.095 mmol), trifluoroacetic acid (1 mL), and dichloromethane (1 mL), and then the reaction mixture was stirred and reacted at room temperature for 4 h. LCMS showed the complete reaction of the reaction material. Dichloromethane was removed by rotatory evaporation, and then a small amount of methanol was added to the residue. After reversed-phase preparation, 30 mg of oily liquid was obtained, with a yield of 77%.

¹H NMR (400 MHz, CDCl₃) δ12.09 (s, 1H), 11.91 (s, 1H), δ7.86 (s, 1H), 7.81 (s, 1H), 4.12 (m, 2H), 3.65-3.47 (m, 12H), 2.45 (m, 4H), 2.08-1.67 (m, 8H).

### Step 5: Preparation of Intermediate D-5

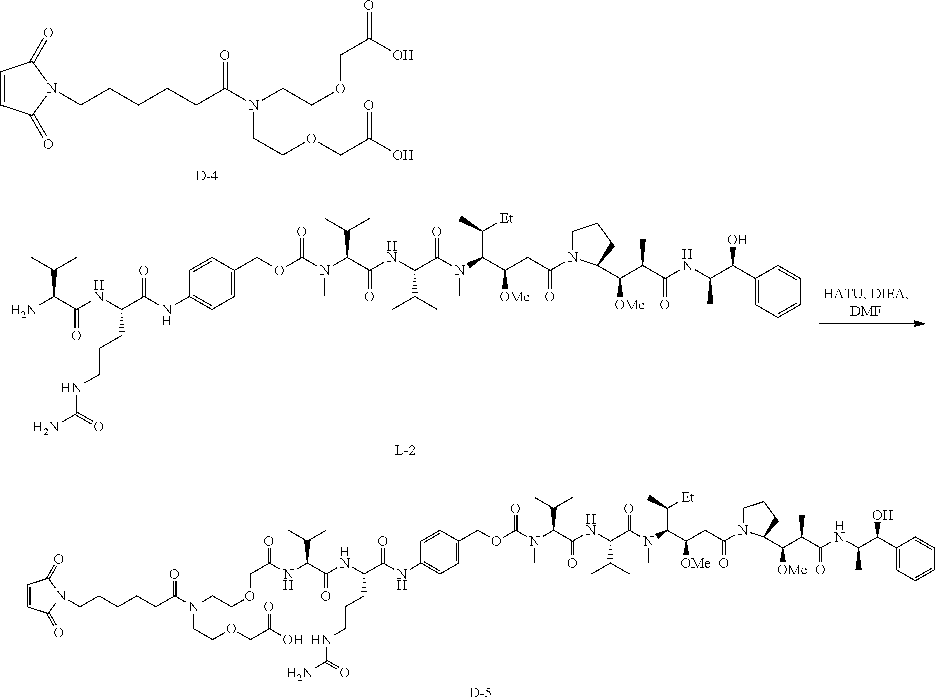

To a 25 mL reaction flask, were added intermediate D-4 (30 mg, 0.072 mmol), L-2 (27 mg, 0.024 mmol), N,N-dimethylformamide (3 mL), N,N-diisopropylethylamine (9 mg, 0.072 mmol), and N,N,N′,N′-tetramethyl-O-(7-azabenzotriazol-1-yl)uronium hexafluorophosphate (27 mg, 0.072 mmol), and then the mixture was allowed to react at room temperature under stirring for 20 min. TLC indicated disappearance of L-2, suggesting completion of the reaction. N,N-dimethylformamide was removed by concentration under reduced pressure, and then a small amount of methanol was added to the residue. After reversed-phase preparation, 23 mg of white solid was obtained, with a yield of 64%.

### Step 6: Preparation of Compound XIp

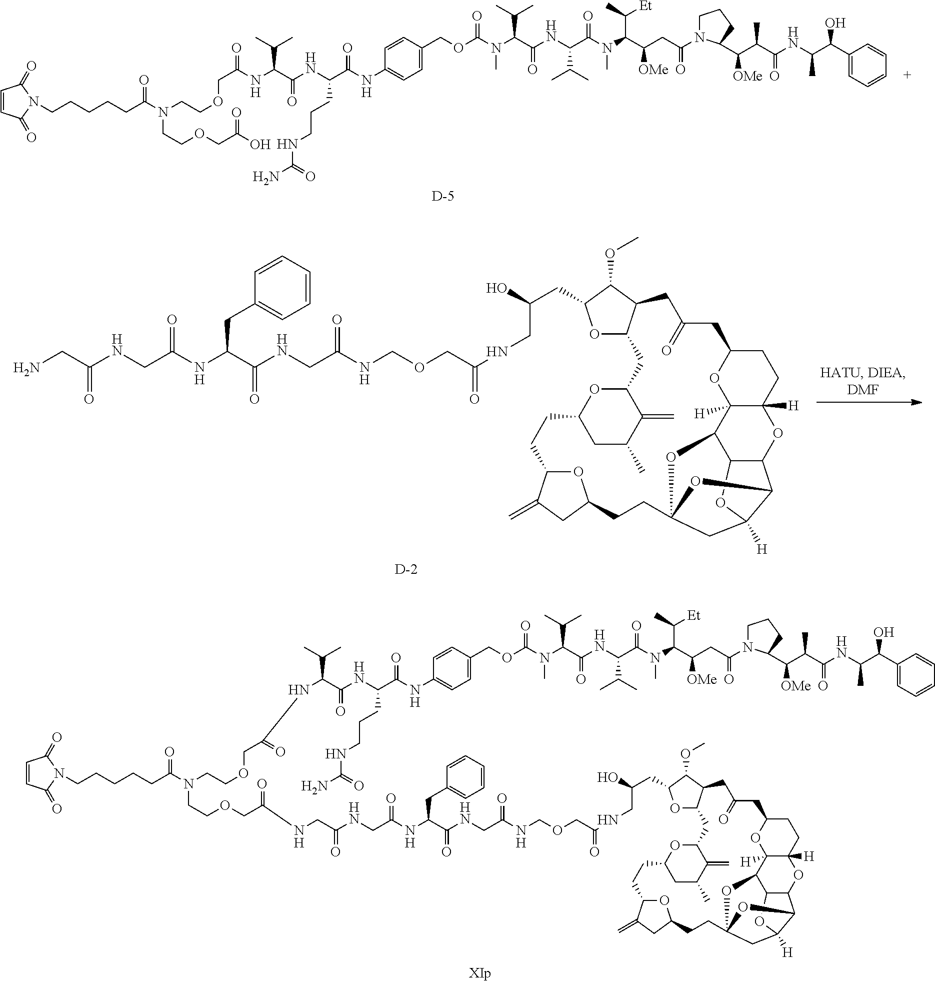

To a 25 mL reaction flask, were added intermediate D-5 (23 mg, 0.015 mmol), D-2 (17 mg, 0.015 mmol), N,N-dimethylformamide (2 mL), N,N-diisopropylethylamine (3 mg, 0.022 mmol), and N,N,N′,N′-tetramethyl-O-(7-azabenzotriazol-1-yl)uronium hexafluorophosphate (8 mg, 0.022 mmol), and then the mixture was allowed to react at room temperature under stirring for 20 min TL C indicated disappearance of D-5, suggesting completion of the reaction. The reaction product was directly purified by reversed-phase preparation, to provide 15 mg of white solid, with a yield of 3800.

### Example 17. Preparation of Intermediate GGFGE

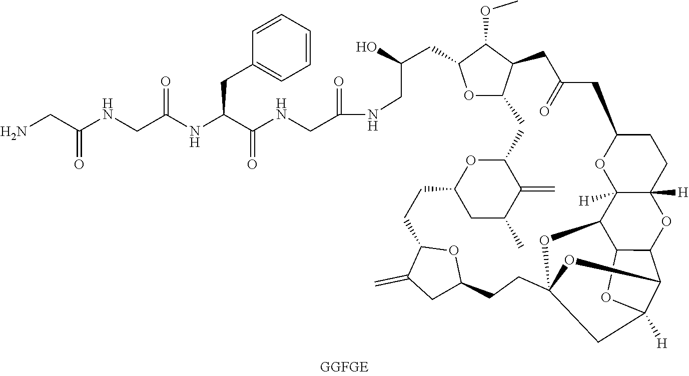 [SMILES: C=C1C[C@H](CC[C@@]23C[C@H]4OC5C(O[C@H]6CC[C@H](CC(=O)C[C@@H]7[C@@H](OC)[C@@H](C[C@H](O)CNC(=O)CNC(=O)[C@H](Cc8ccccc8)NC(=O)CNC(=O)CN)O[C@H]7C)O[C@@H]6[C@@H]5O2)[C@H]4O3)O[C@H]1CC[C@H]1C[C@@H](C)C(=C)[C@@H](C)O1]

### Step 1: Preparation of Compound D2

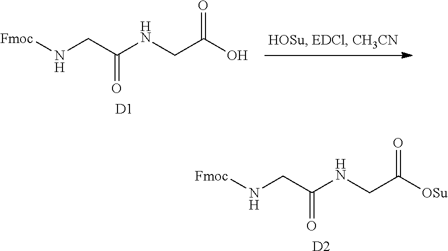

To a 500 mL reaction flask, were added intermediate D1 (20 g, 56.49 mmol) and acetonitrile (200 mL), and after the solution became clear, 1-(3-dimethylaminopropyl)-3-ethylcarbodiimide hydrochloride (11 g, 56.49 mmol) and N-hydroxysuccinimide (7 g, 56.49 mmol) were added. The reaction solution was stirred and reacted at room temperature for 12 h. TLC indicated completion of the reaction. The reaction solution was filtered, and the solid was dried, to obtain compound D2 (22 g), with a yield of 90%.

### Step 2: Preparation of Compound D3

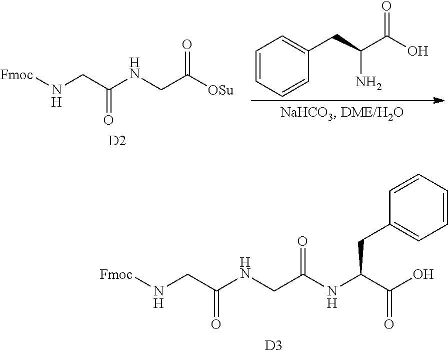

To a 500 mL reaction flask, were added intermediate L-phenylalanine (8 g, 48.48 mmol), sodium bicarbonate (8 g, 96.96 mmol), and water (200 mL), and after the solution became clear, compound D2 (22 g, 48.48 mmol) was dissolved in ethylene glycol dimethyl ether (50 mL) and then slowly added to the above reaction solution. The resultant solution was stirred and reacted at room temperature for 12 h. TLC indicated completion of the reaction. Ethylene glycol dimethyl ether was removed by concentration under reduced pressure. The residue was added dropwise to 0.5 M of hydrochloric acid aqueous solution. Lots of solid was precipitated, which was filtered and dried, to obtain compound D3 (15 g), with a yield of 62%.

¹H NMR (400 MHz, CDCl₃) δ12.51 (s, 1H), 9.04 (s, 1H), 8.31 (s, 1H), 7.95 (s, 1H), 7.90 (d, J=8.0 Hz, 2H), 7.56 (d, J=7.8 Hz, 2H), 7.38-7.28 (m, 4H), 7.19-7.14 (m, 5H), 4.85 (t, J=8.2 Hz, 1H), 4.71 (d, J=8.2 Hz, 2H), 4.39 (t, J=8.4 Hz, 1H), 4.10-3.83 (m, 4H), 3.12 (d, J=9.6 Hz, 1H), 2.85 (d, J=9.6 Hz, 1H).

### Step 3: Preparation of Compound D4

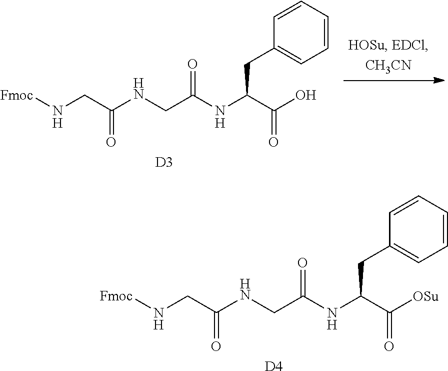

To a 500 mL reaction flask, were added intermediate D3 (15 g, 29.9 mmol), acetonitrile (200 mL), 1-(3-dimethylaminopropyl)-3-ethylcarbodiimide hydrochloride (6 g, 29.9 mmol), and N-hydroxysuccinimide (4 g, 29.9 mmol), and then the mixture was stirred and reacted at room temperature for 12 h. TLC indicated completion of the reaction. The reaction solution was filtered, and the solid was dried, to provide compound D4 (13 g), with a yield of 72%.

### Step 4: Preparation of Compound D5

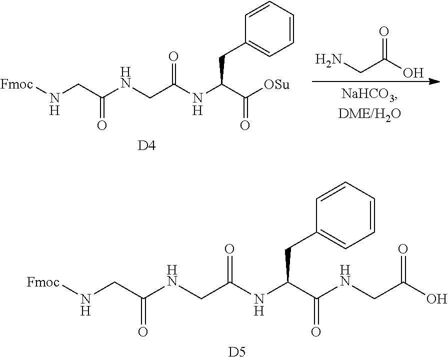

To a 500 mL reaction flask, were added intermediate glycine (3 g, 40.0 mmol), sodium bicarbonate (7 g, 80.0 mmol), and water (150 mL), and after the solution became clear, compound D4 (13 g, 40.0 mmol) was dissolved in ethylene glycol dimethyl ether (40 mL) and then slowly added to the above reaction solution. The resultant solution was stirred and reacted at room temperature for 12 h. TLC indicated completion of the reaction. The solvent was removed by concentration under reduced pressure. The residue was added dropwise to 0.5 M of hydrochloric acid aqueous solution. Lots of solid was precipitated, which was filtered and dried, to obtain compound D5 (15 g), with a yield of 71%.

¹H NMR (400 MHz, CDCl₃) δ13.01 (s, 1H), 9.01 (s, 1H), 8.27 (s, 1H), 7.98 (s, 1H), 7.89 (d, J=8.0 Hz, 2H), 7.54 (d, J=7.8 Hz, 2H), 7.34-7.23 (m, 4H), 7.19-7.14 (m, 5H), 4.75 (t, J=8.2 Hz, 1H), 4.61 (d, J=8.2 Hz, 2H), 4.30 (t, J=8.4 Hz, 1H), 4.04-3.83 (m, 6H), 3.10 (d, J=9.6 Hz, 1H), 2.75 (d, J=9.6 Hz, 1H).

### Step 5: Preparation of Compound D6

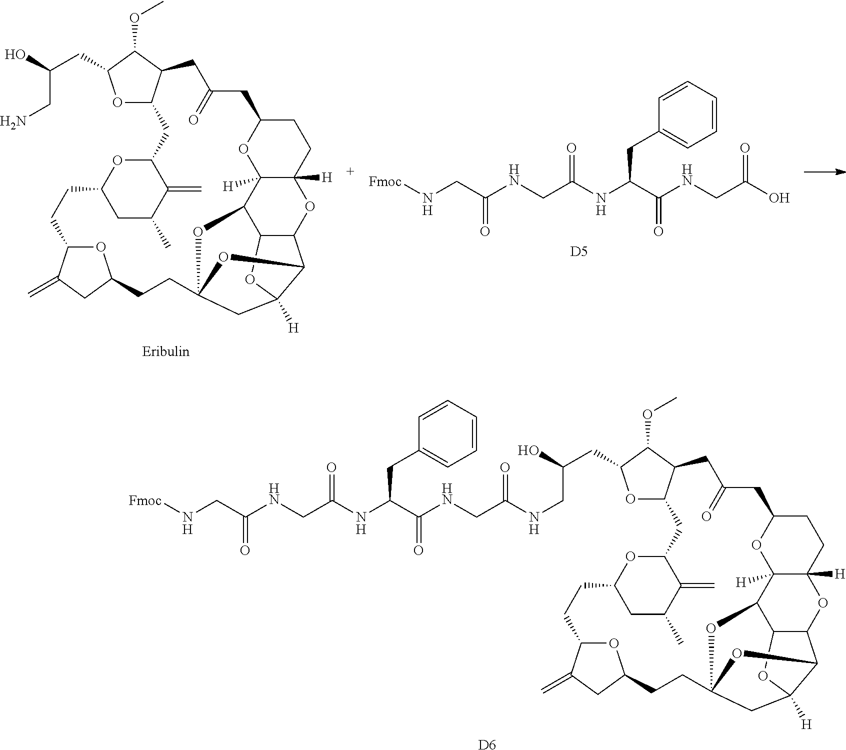

To a 25 mL reaction flask, were added intermediate D5 (200 mg, 0.36 mmol), N,N,N′,N′-tetramethyl-O-(7-azabenzotriazol-1-yl)uronium hexafluorophosphate (163 mg, 0.43 mmol), N,N-diisopropylethylamine (66.9 mg, 0.54 mmol), and N,N-dimethylformamide (5 mL), and then the mixture was allowed to react at room temperature under stirring for 5 min, followed by addition of eribulin (263.1 mg, 0.36 mmol). TLC indicated completion of the reaction. N,N-dimethylformamide was removed by concentration under reduced pressure, and the residue was purified by TLC (dichloromethane:methanol=10:1), to obtain compound D6 (300 mg), with a yield of 50%.

### Step 6: Preparation of Compound GGFGE

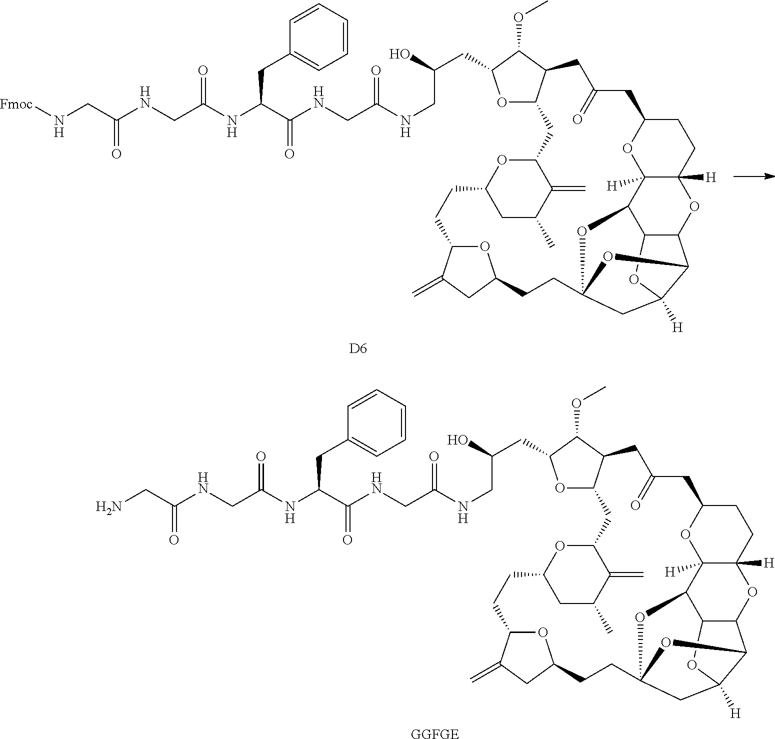

To a 25 mL reaction flask, were added intermediate D6 (20 mg, 0.016 mmol), 1,8-diazabicyclo[5.4.0]undec-7-ene (5 mg, 0.032 mmol), and N,N-dimethylformamide (3 mL), and then the mixture was stirred and reacted at room temperature for 30 min. TLC indicated completion of the reaction. N,N-dimethylformamide was removed by concentration under reduced pressure, and the residue was purified by TLC (dichloromethane:methanol=10:1), to obtain compound GGFGE (10 mg), with a yield of 62%.

### Example 18. Preparation of the Dual-Drug Link Assembly Unit of Formula XIq According to the Present Invention

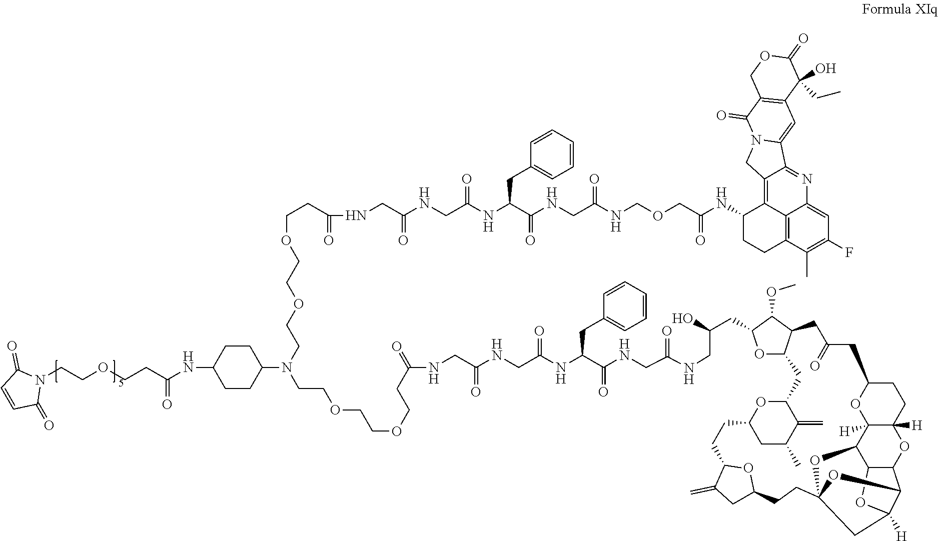

### Step 1: Preparation of Compound M-1

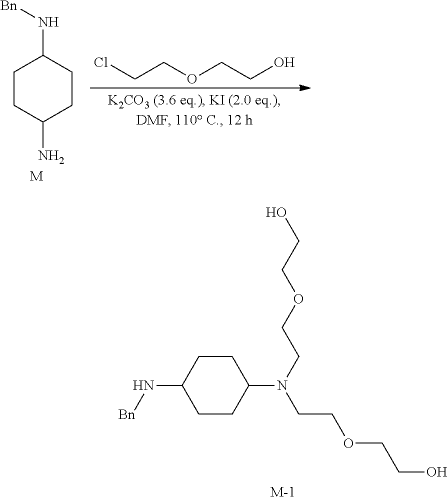

To a 25 mL reaction flask, were successively added compound M (1.00 g, 4.9 mmol) and the solvent N,N-dimethylformamide (15 mL), and after the solution became clear, potassium carbonate (2.43 g, 17.6 mmol), potassium iodide (1.63 g, 9.8 mmol), and 2-chloroethoxy-2-ethoxydiethanol (1.63 g, 9.8 mmol) were sequentially added, and then the resultant solution was stirred and reacted at 110° C. for 12 h. TLC indicated completion of the reaction. After filtration, the excess N,N-dimethylformamide was removed by concentration under reduced pressure, and the residue was purified by HPLC, to obtain intermediate M-1 (1.5 g), with a yield of 83%.

¹H NMR (400 MHz, CDCl₃) δ 7.32-7.29 (m, 5H), 4.51 (s, 2H), 4.43 (s, 1H), 3.72-3.69 (m, 4H), 3.55-3.50 (m, 8H), 2.65-2.50 (m, 6H), 1.59-1.34 (m, 8H).

### Step 2: Preparation of Compound M-2

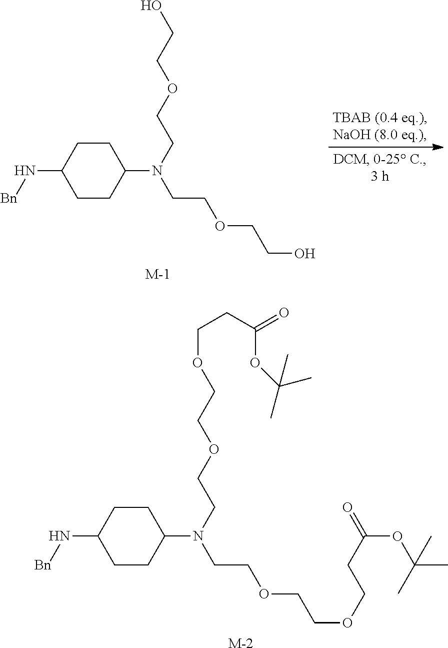 [SMILES: OCCOCCN(CCOCCO)C1CCC(NCc2ccccc2)CC1 ; "M-1" ; CC(C)(C)OC(=O)CCOCCOCCN(CCOCCOCCC(=O)OC(C)(C)C)C1CCC(NCc2ccccc2)CC1 ; "M-2"]

To a 25 mL reaction flask, were added intermediate M-1 (1.50 g, 3.95 mmol), tetrabutylammonium bromide (2.10 g, 1.58 mmol), and dichloromethane (10 mL), and then the mixture was allowed to react at 0° C. for 30 min. NaOH (1.26 g, 31.60 mmol) was dissolved in water (1.2 mL), and after becoming clear, the NaOH aqueous solution was slowly added to the above reaction solution dropwise. The resultant solution was allowed to further react at 0° C. for 10 min, and then the ice bath was removed, followed by reaction at room temperature for 3 h. LC-MS indicated the reaction was completed. The reaction solution was adjusted to weak acidity with dilute hydrochloric acid at pH=3, and extracted with dichloromethane (10 mL*3). The organic phase was combined, and washed with saturated NaCl aqueous solution. The organic phase was dried with anhydrous sodium sulfate and filtered. The filtrate was evaporated under reduced pressure to remove excess dichloromethane, and then 1.7 g of target product was obtained, with a yield of 75%, which can be directly used in the next step.

### Step 3: Preparation of Compound M-3

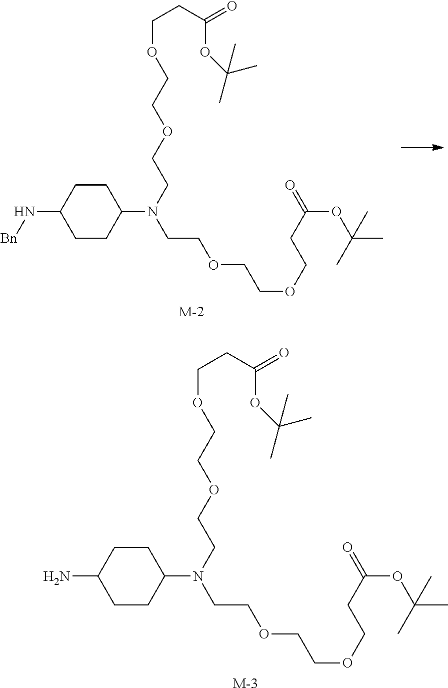 [SMILES: CC(C)(C)OC(=O)CCOCCOCCN(CCOCCOCCC(=O)OC(C)(C)C)C1CCC(NCc2ccccc2)CC1 ; "M-2" ; CC(C)(C)OC(=O)CCOCCOCCN(CCOCCOCCC(=O)OC(C)(C)C)C1CCC(N)CC1 ; "M-3"]

To a 25 mL reaction flask, were added intermediate M-2 (1 g, 1.57 mmol), methanol (10 mL), and catalytic amount of Pd/C, and then under hydrogen atmosphere, the reaction solution was reacted under stirring at room temperature for 2 h. LC-MS showed that the reaction was completed. Pd/C were filtered out, and then the excess solvent was removed by concentration under reduced pressure. The residue was purified by TLC (dichloromethane:methanol=10:1), to provide compound M-3 (900 mg), with a yield of 75%.

¹H NMR (400 MHz, CDCl₃) δ 3.72-3.69 (m, 4H), 3.55-3.50 (m, 12H), 2.65-2.42 (m, 10H), 1.59-1.34 (m, 8H), 1.49 (s, 18H).

### Step 4: Preparation of Compound M-4

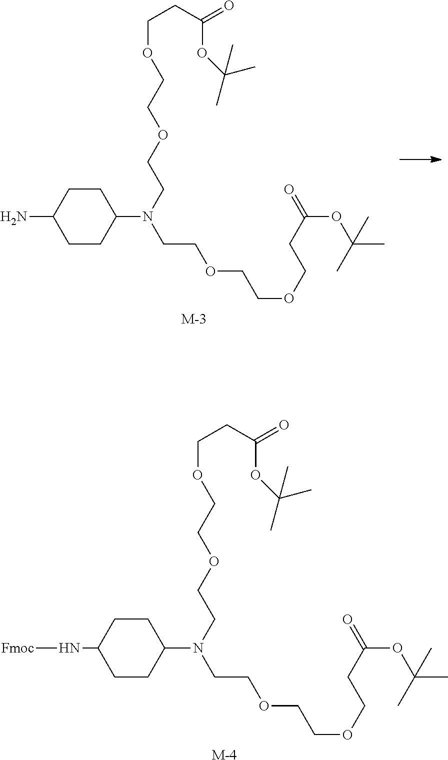

To a 25 mL reaction flask, were added intermediate M-3 (900 mg, 1.57 mmol), THE (10 mL) and water (2.5 mL), and after the reaction solution became clear, sodium bicarbonate (210 mg, 2.50 mmol) and 9-fluorenylmethyl-N-succinimidyl carbonate (530 mg, 1.57 mmol) were successively added. The resultant solution was stirred and reacted at room temperature for 2 h. LC-MS indicated completion of the reaction, and then the excess solvent was removed by concentration under reduced pressure. The residue was purified by TLC (dichloromethane:methanol=10:1), to provide compound M-4 (1 g), with a yield of 74%.

¹H NMR (400 MHz, CDCl₃) δ 7.90 (d, J=8.9 Hz, 2H), 7.55 (d, J=9.2 Hz, 2H), 7.38-7.28 (m, 4H), 4.70 (d, J=11.2 Hz, 2H), 4.46 (t, J=11.2 Hz, 1H), 3.71-3.67 (m, 4H), 3.53-3.48 (m, 12H), 2.55-2.41 (m, 10H), 1.51-1.24 (m, 8H), 1.41 (s, 18H).

### Step 5: Preparation of Compound M-5

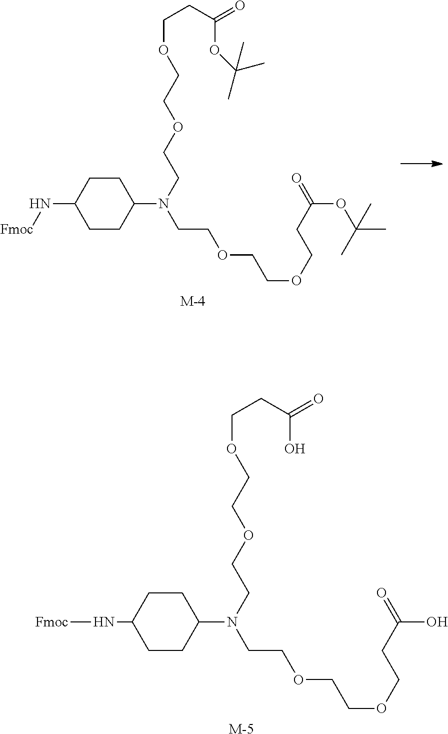

To a 25 mL reaction flask, were added intermediate M-4 (1 g, 1.54 mmol), dichloromethane (5 mL) and trifluoroacetic acid (5 mL), and then the mixture was stirred and reacted at room temperature for 2 h. LC-MS indicated completion of the reaction, and then the excess solvent was removed by concentration under reduced pressure. The residue was purified by TLC (dichloromethane:methanol=10:1), to provide compound M-5 (600 mg), with a yield of 75%.

### Step 6: Preparation of Compound M-6

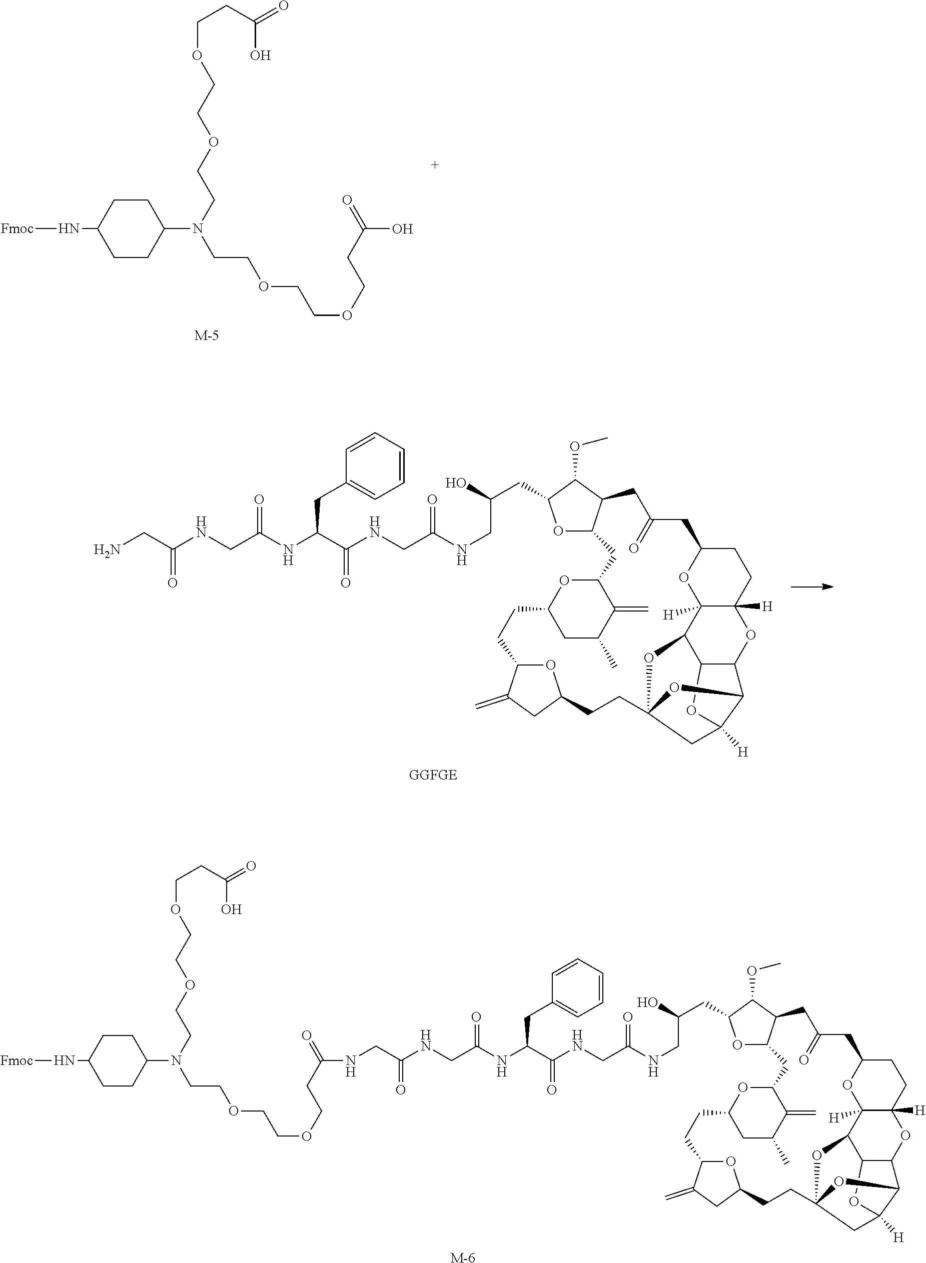

To a 25 mL reaction flask, were added intermediate GGFGE (50 mg, 0.05 mmol), intermediate M-5 (50 mg, 0.10 mmol), N,N,N′,N′-tetramethyl-O-(7-azabenzotriazol-1-yl)uronium hexafluorophosphate (38 mg, 0.10 mmol), N,N-diisopropylethylamine (12.9 mg, 0.10 mmol), and N,N-dimethylformamide (4 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound M-6 (50 mg), with a yield of 33%.

### Step 7: Preparation of Compound M-7

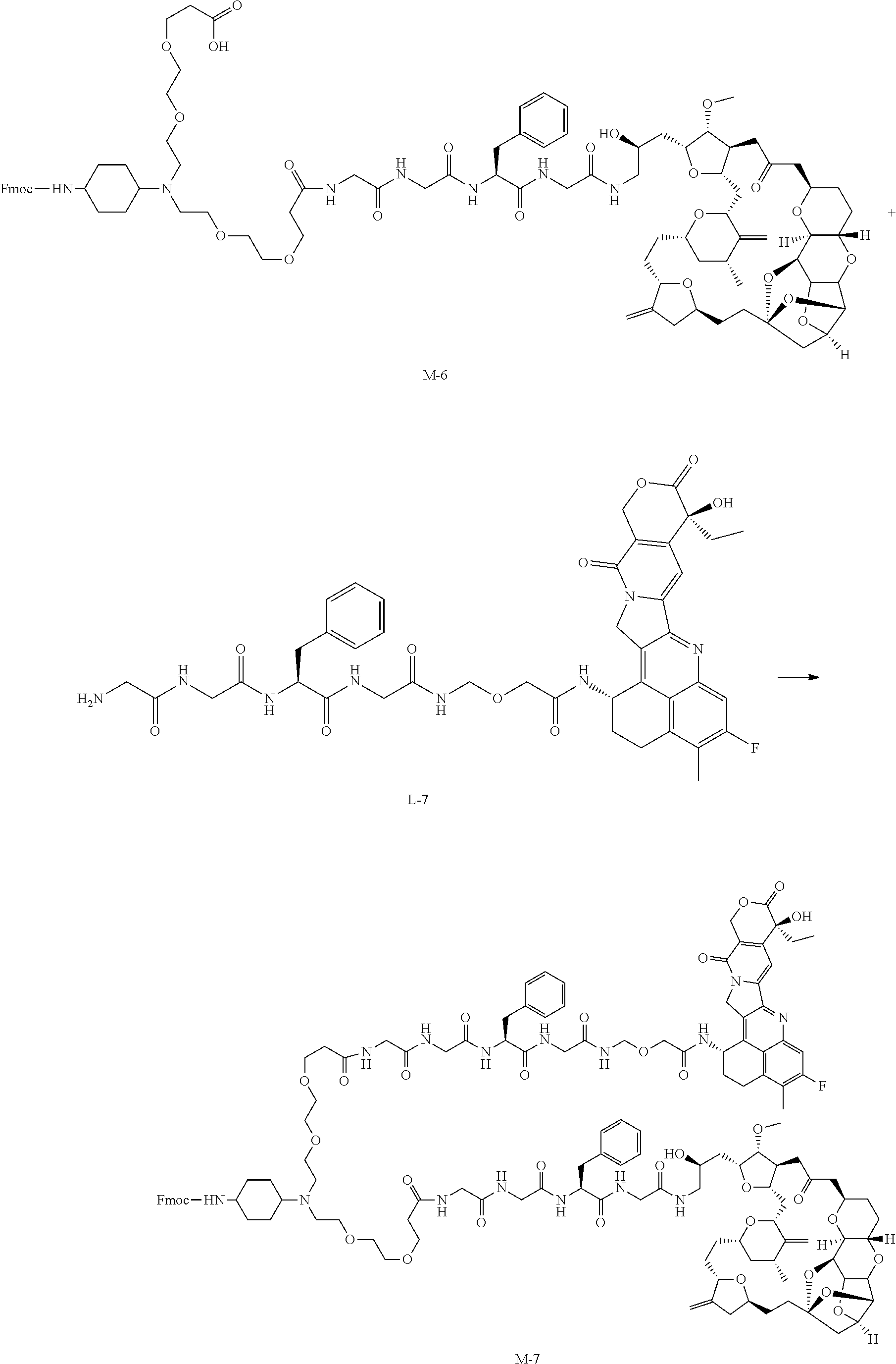

To a 25 mL reaction flask, were added intermediate M-6 (30 mg, 0.02 mmol), intermediate L-7 (16 mg, 0.02 mmol), N,N,N′,N′-tetramethyl-O-(7-azabenzotriazol-1-yl)uronium hexafluorophosphate (7.6 mg, 0.02 mmol), N,N-diisopropylethylamine (2.58 mg, 0.02 mmol), and N,N-dimethylformamide (3 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound M-7 (20 mg), with a yield of 44%.

### Step 8: Preparation of Compound M-8

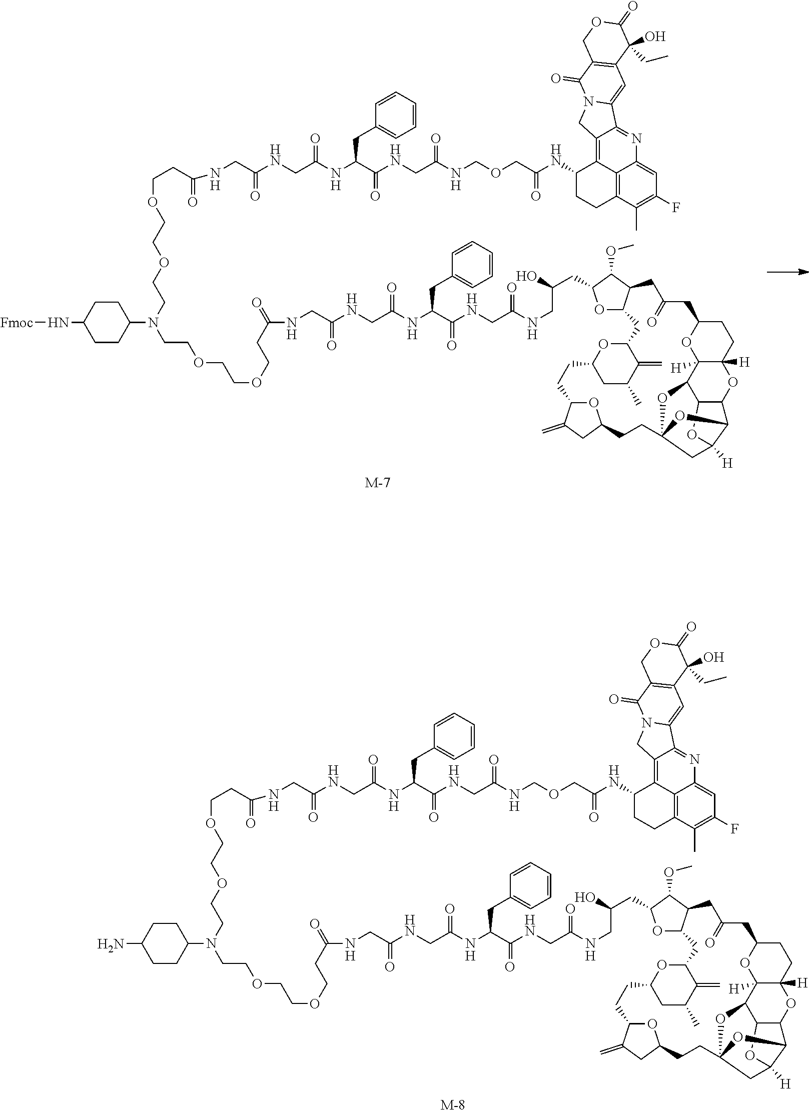

To a 25 mL reaction flask, were added intermediate M-7 (20 mg, 0.008 mmol), 1,8-diazabicyclo[5.4.0]undec-7-ene (2.45 mg, 0.016 mmol), and N,N-dimethylformamide (3 mL), and then the mixture was stirred and reacted at room temperature for 30 min. TLC indicated completion of the reaction. N,N-dimethylformamide was removed by concentration under reduced pressure, and the residue was purified by TLC (dichloromethane:methanol=10:1), to obtain compound M-8 (15 mg), with a yield of 75%.

### Step 9: Preparation of Compound XIq

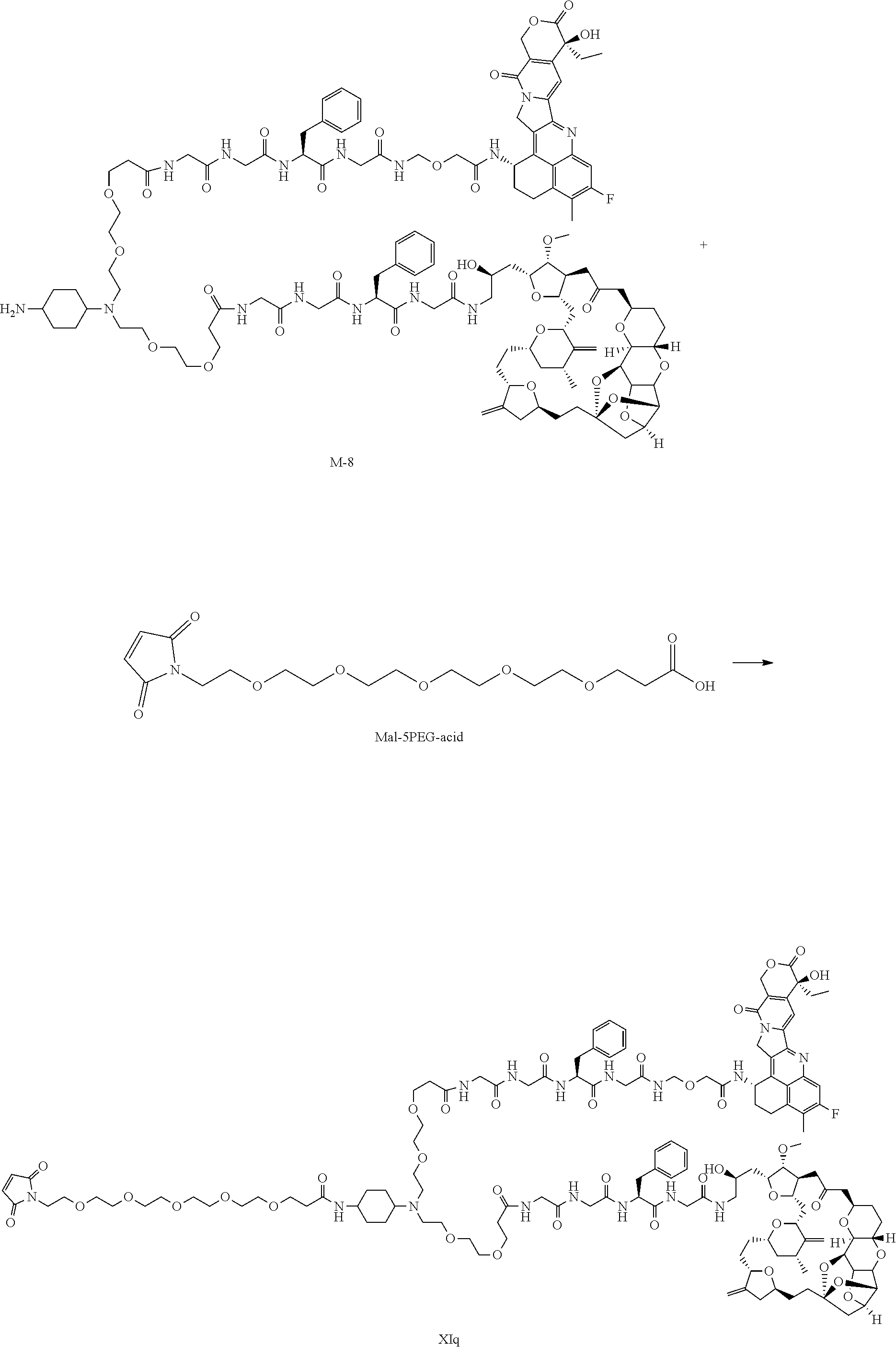

To a 25 mL reaction flask, were added intermediate M-8 (10 mg, 0.004 mmol), intermediate Mal-5PEG-acid (1.60 mg, 0.004 mmol), N,N,N′,N′-tetramethyl-O-(7-azabenzotriazol-1-yl)uronium hexafluorophosphate (1.52 mg, 0.004 mmol), N,N-diisopropylethylamine (0.52 mg, 0.004 mmol), and N,N-dimethylformamide (3 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIq (7 mg), with a yield of 44%.

### Example 19. Preparation of the Dual-Drug Link Assembly Unit of Formula XIr According to the Present Invention

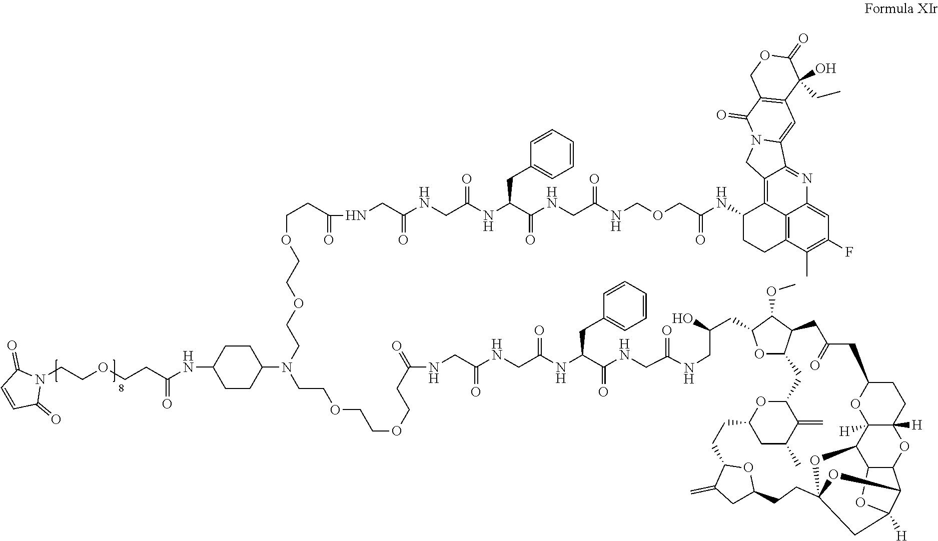

### Step 1: Preparation of Compound XIr

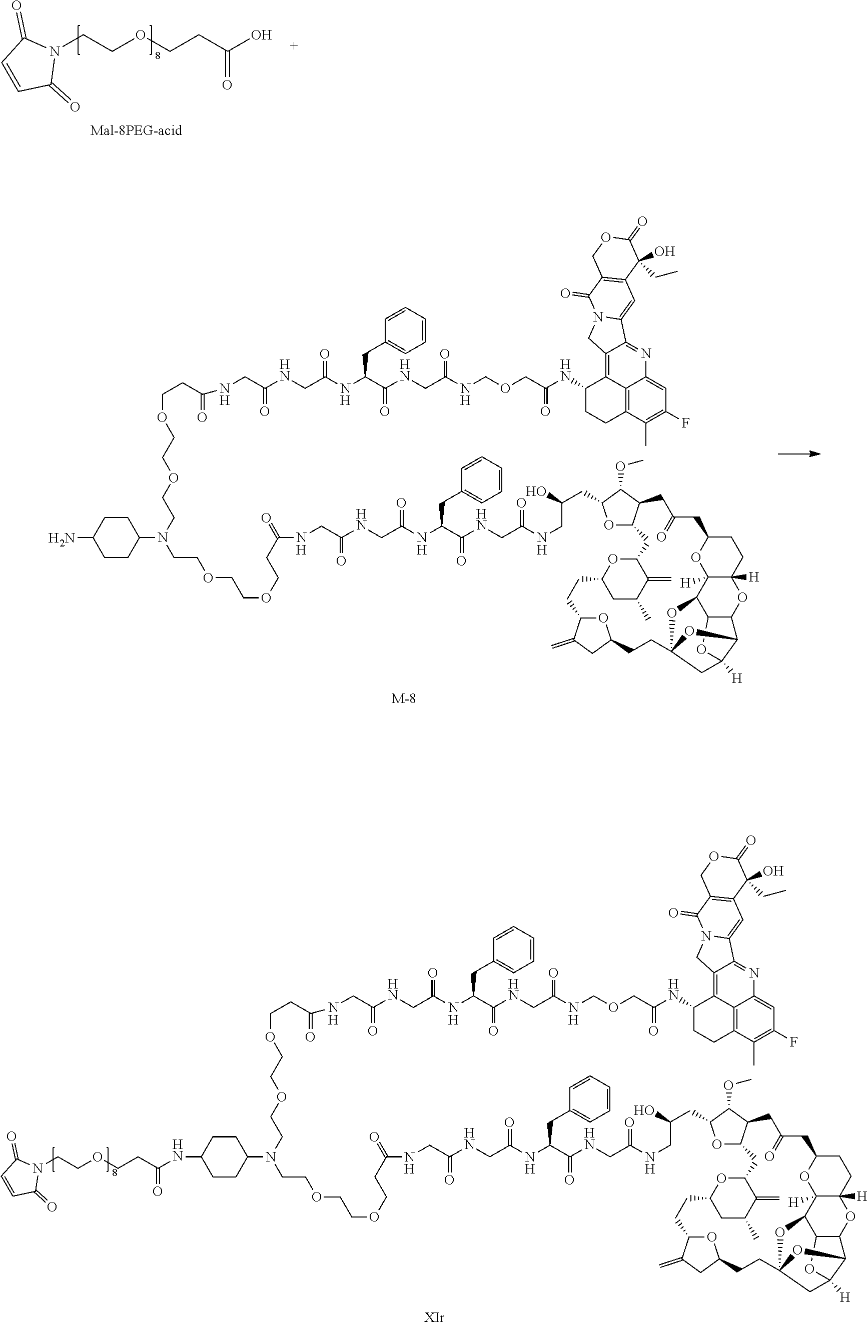

To a 25 mL reaction flask, were added intermediate M-8 (10 mg, 0.004 mmol), intermediate Mal-8PEG-acid (1.70 mg, 0.004 mmol), N,N,N′,N′-tetramethyl-O-(7-azabenzotriazol-1-yl)uronium hexafluorophosphate (1.52 mg, 0.004 mmol), N,N-diisopropylethylamine (0.52 mg, 0.004 mmol), and N,N-dimethylformamide (3 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIr (6.5 mg), with a yield of 45%.

### Example 20. Preparation of the Dual-Drug Link Assembly Unit of Formula XIs According to the Present Invention

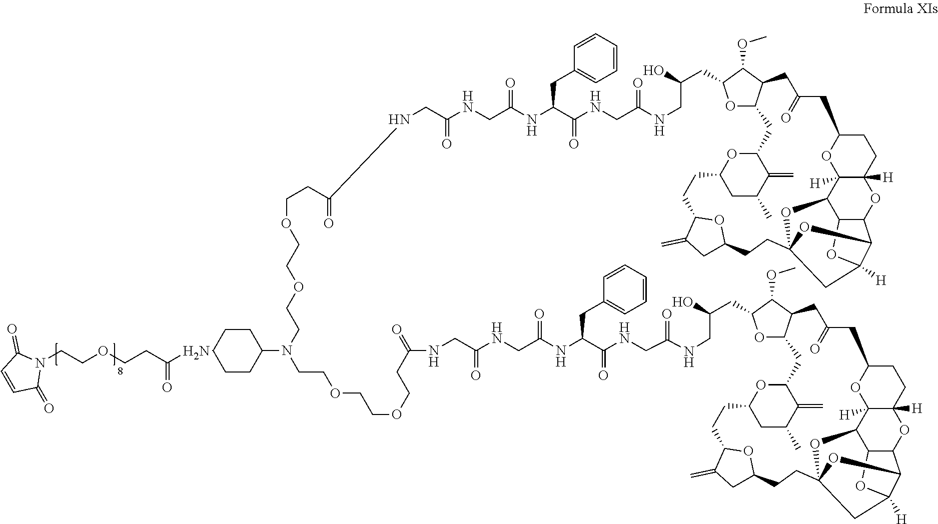

### Step 1: Preparation of Compound M-9

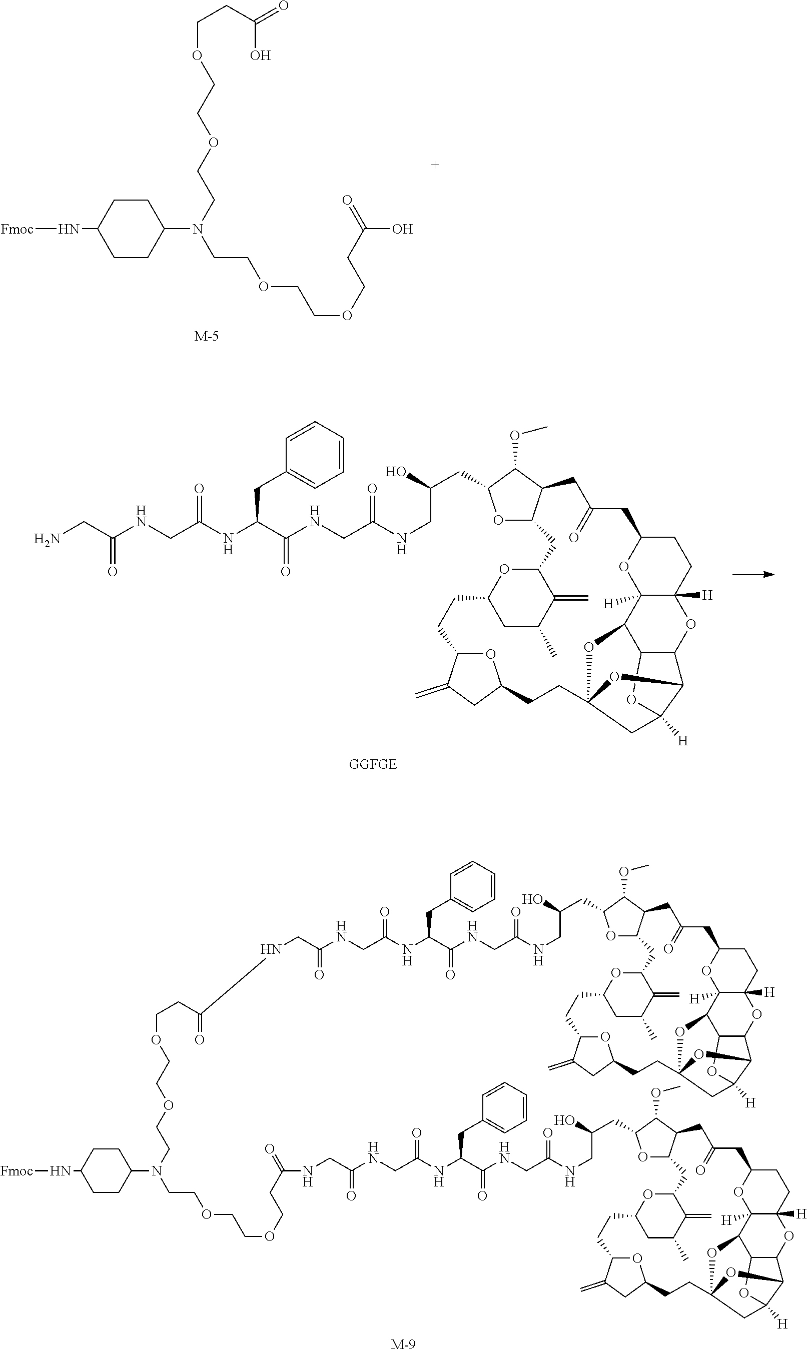

To a 25 mL reaction flask, were added intermediate GGFGE (50 mg, 0.05 mmol), intermediate M-5 (13 mg, 0.03 mmol), N,N,N′,N′-tetramethyl-O-(7-azabenzotriazol-1-yl)uronium hexafluorophosphate (38 mg, 0.10 mmol), N,N-diisopropylethylamine (13 mg, 0.10 mmol), and N,N-dimethylformamide (4 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound M-9 (40 mg), with a yield of 81%.

### Step 2: Preparation of Compound M-10

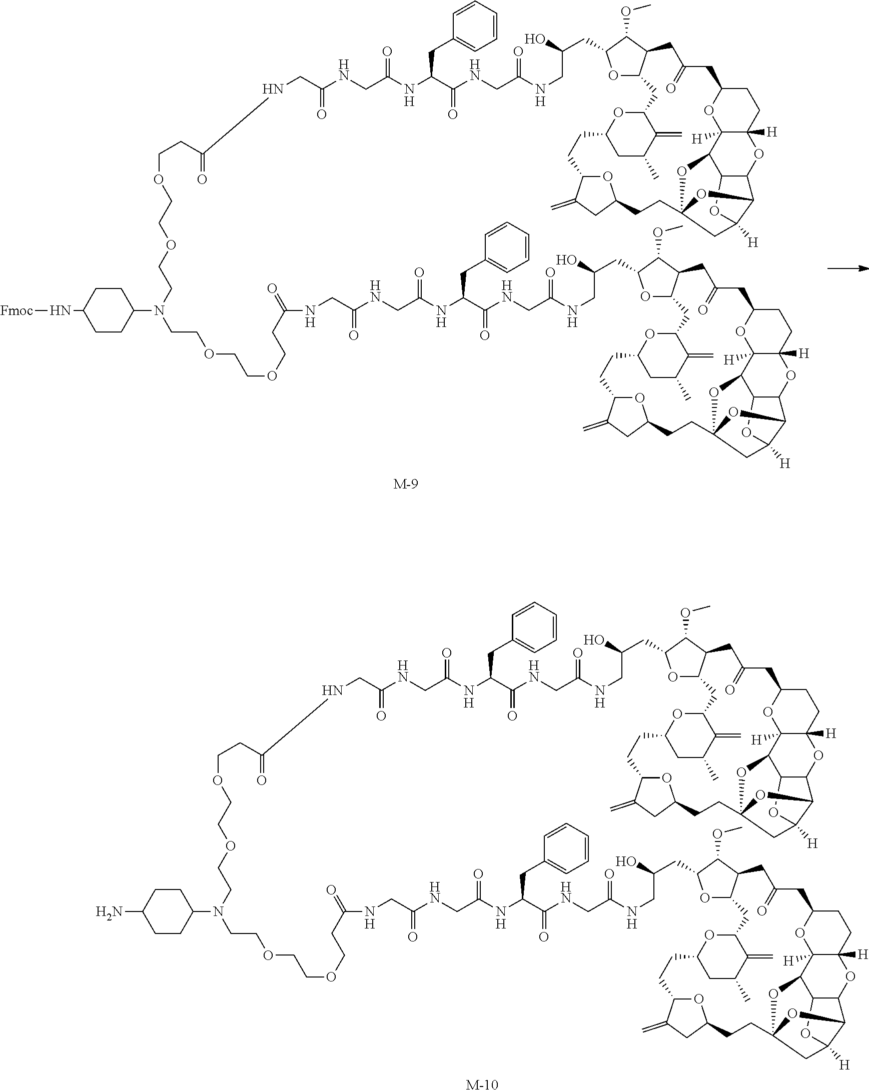

To a 25 mL reaction flask, were added intermediate M-9 (20 mg, 0.007 mmol), 1,8-diazabicyclo[5.4.0]undec-7-ene (2.35 mg, 0.014 mmol), and N,N-dimethylformamide (3 mL), and then the mixture was stirred and reacted at room temperature for 30 min. TLC indicated completion of the reaction. N,N-dimethylformamide was removed by concentration under reduced pressure, and the residue was purified by TLC (dichloromethane:methanol=10:1), to obtain compound M-10 (15 mg), with a yield of 83%.

### Step 3: Preparation of Compound XIs

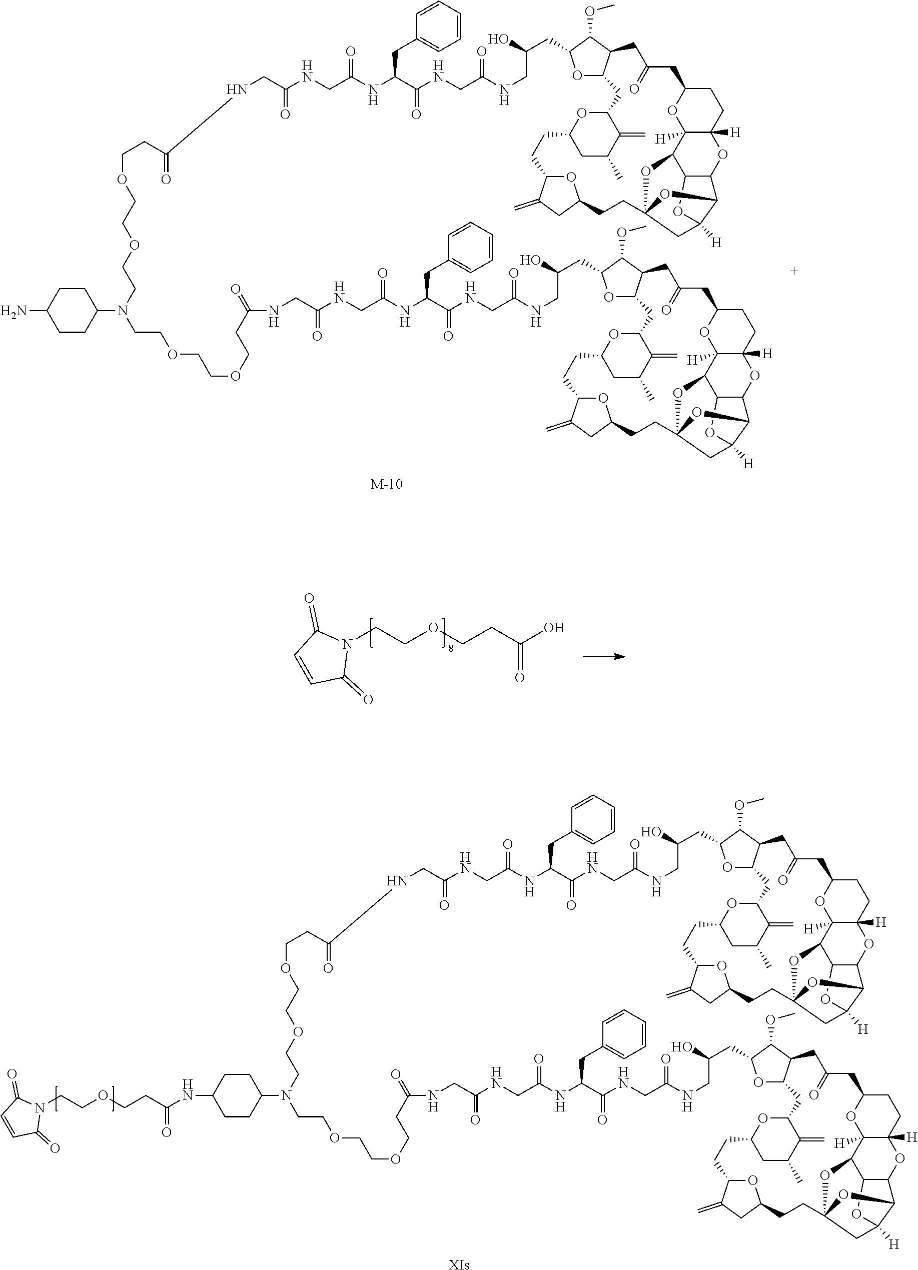

To a 25 mL reaction flask, were added intermediate M-10 (10 mg, 0.004 mmol), intermediate Mal-8PEG-acid (2 mg, 0.004 mmol), N,N,N′,N′-tetramethyl-O-(7-azabenzotriazol-1-yl)uronium hexafluorophosphate (2 mg, 0.004 mmol), N,N-diisopropylethylamine (1 mg, 0.004 mmol), and N,N-dimethylformamide (3 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIs (6.5 mg), with a yield of 45%.

### Example 21. Preparation of the Dual-Drug Link Assembly Unit of Formula XIt According to the Present Invention

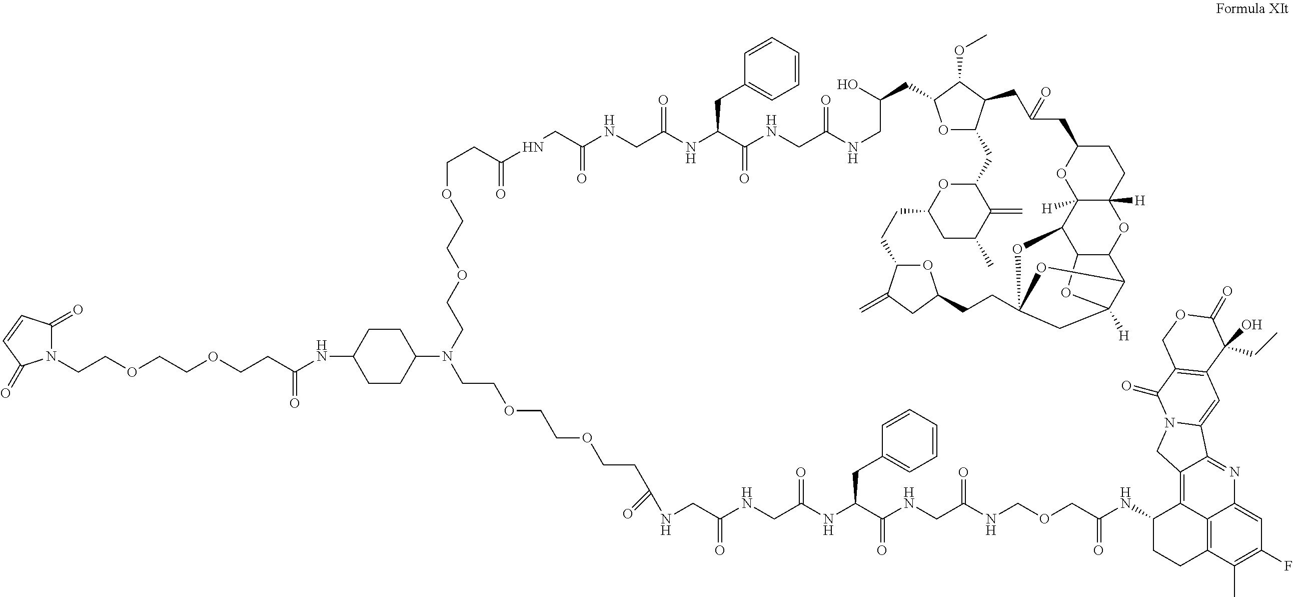

### Step 1: Preparation of Compound XIt

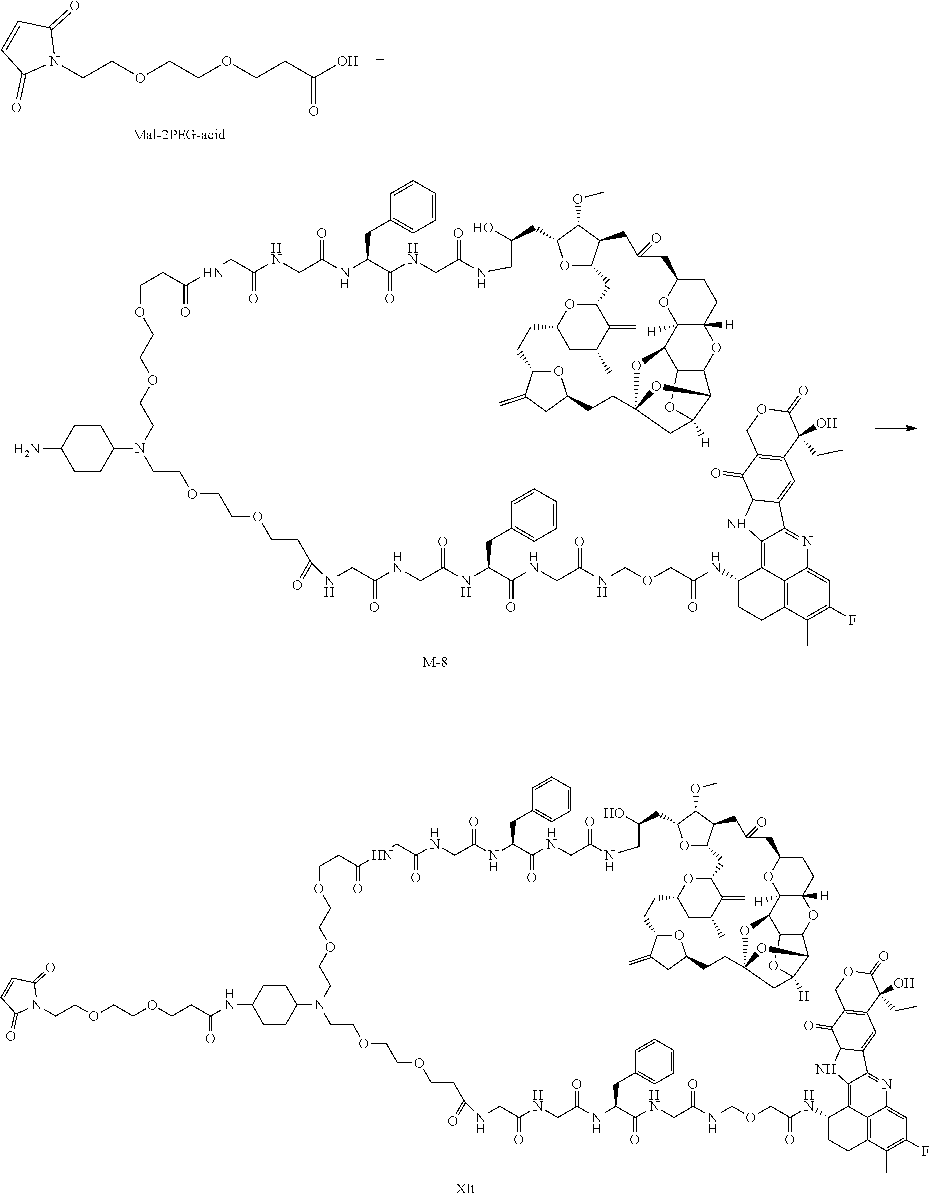

To a 25 mL reaction flask, were added intermediate M-8 (10 mg, 0.004 mmol), intermediate Mal-2PEG-acid (1.34 mg, 0.004 mmol), N,N,N′,N′-tetramethyl-O-(7-azabenzotriazol-1-yl)uronium hexafluorophosphate (1.52 mg, 0.004 mmol), N,N-diisopropylethylamine (0.52 mg, 0.004 mmol), and N,N-dimethylformamide (3 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIt (5.9 mg), with a yield of 53%.

### Example 22. Preparation of the Dual-Drug Link Assembly Unit of Formula XIu According to the Present Invention

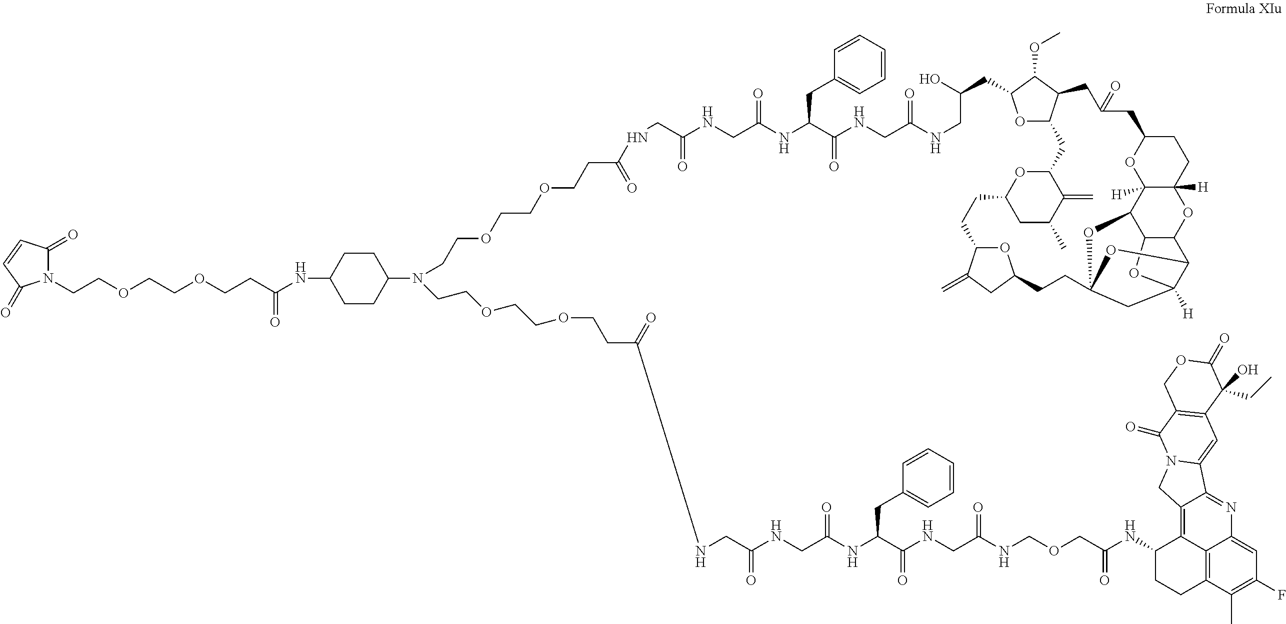

### Step 1: Preparation of Compound XIu

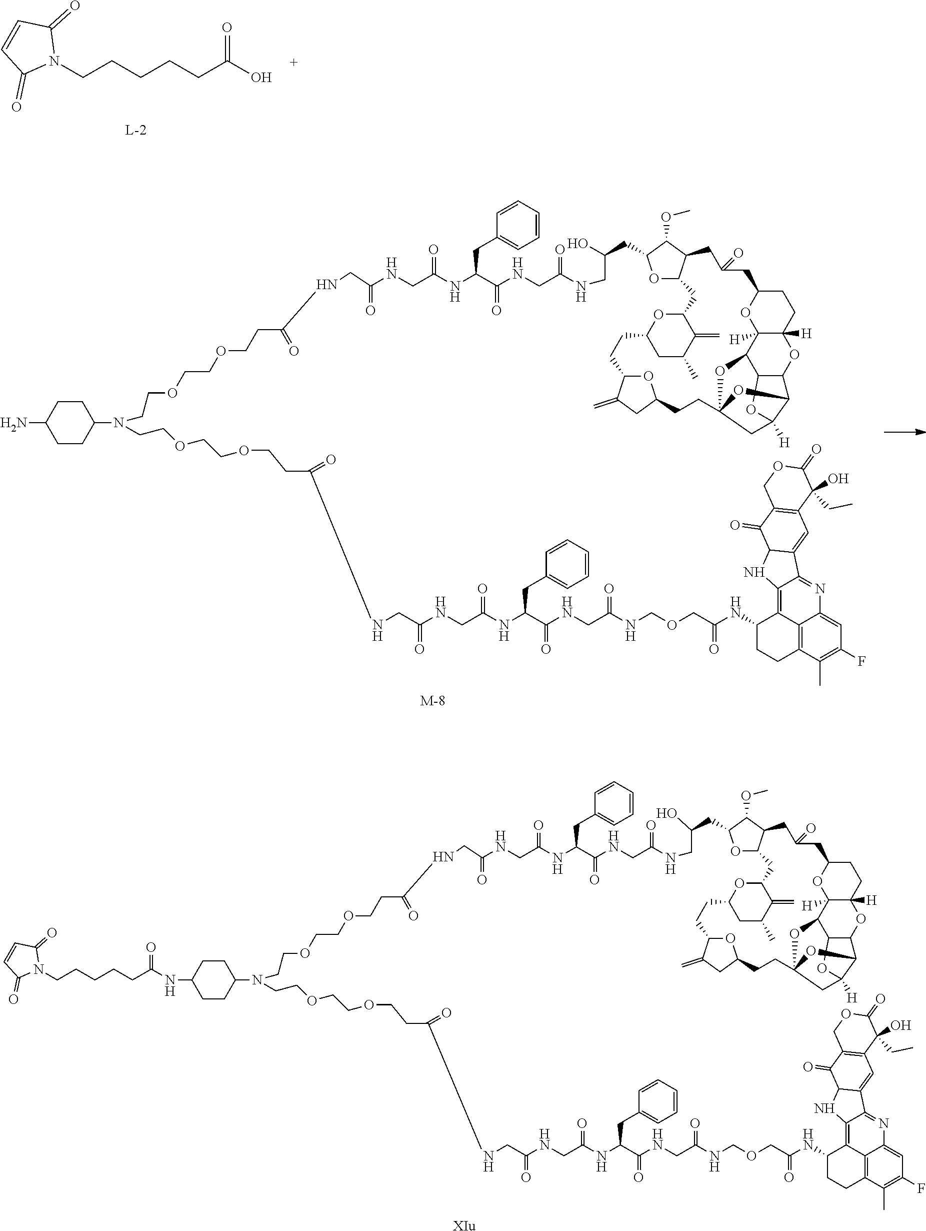

To a 25 mL reaction flask, were added intermediate M-8 (10 mg, 0.004 mmol), intermediate L-2 (1.34 mg, 0.004 mmol), N,N,N′,N′-tetramethyl-O-(7-azabenzotriazol-1-yl)uronium hexafluorophosphate (1.52 mg, 0.004 mmol), N,N-diisopropylethylamine (0.52 mg, 0.004 mmol), and N,N-dimethylformamide (3 mL), and then the mixture was allowed to react at room temperature under stirring for 30 min. TLC indicated completion of the reaction. N,N-dimethylformamide was removed by concentration under reduced pressure, and the residue was purified by TLC (dichloromethane:methanol=10:1), to obtain compound XIu (5.6 mg), with a yield of 52%.

### Example 23. Preparation of the Dual-Drug Link Assembly Unit of Formula XIv According to the Present Invention

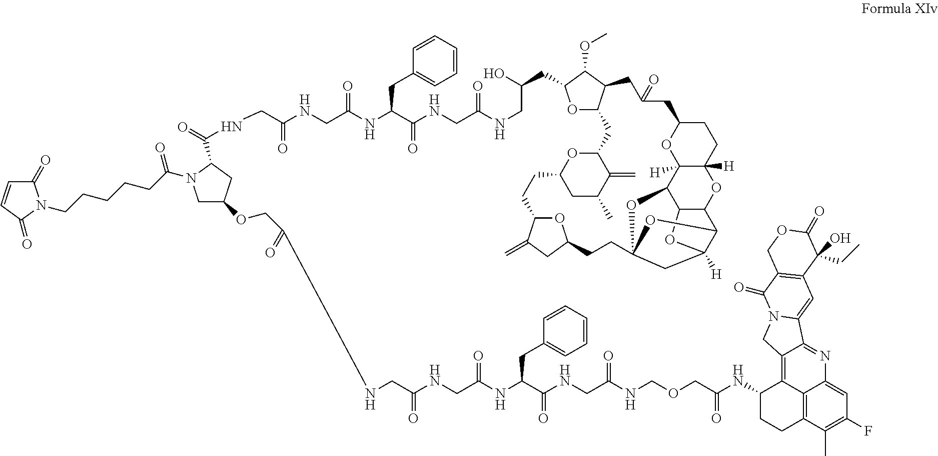

### Step 1: Preparation of Intermediate N-1

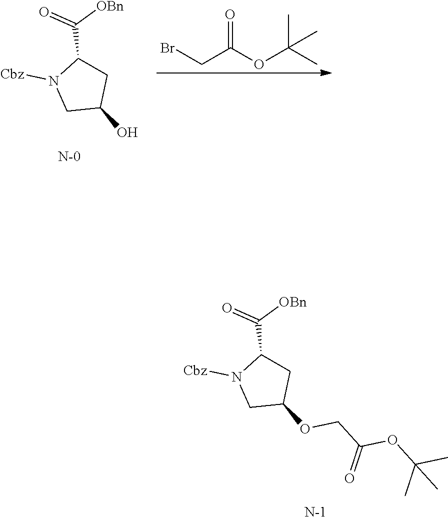

To a 25 mL reaction flask, were added intermediate N-0 (2.0 g, 5.63 mmol) and N,N-dimethylformamide (10 mL), and then under nitrogen protection, the reaction solution was cooled in an ice water bath for 15 min, to which was added sodium hydride (0.4 g, 8.44 mmol) in batches. The mixture was stirred for 5 min, and then tert-butyl bromoacetate (1.6 g, 8.44 mmol) was added. The reaction solution was slowly warmed to room temperature, stirred and reacted for 1 h. TLC showed that N-0 disappeared, indicating completion of the reaction. 15 mL of water was added to the reaction solution, and then the resultant solution was extracted with ethyl acetate (30 mL*3), dried over anhydrous sodium sulfate, filtered, and rotatory evaporated. To the residue, was added a small amount of methanol, and after reversed-phase preparation, 1.7 g of oily liquid was obtained, with a yield of 50%.

¹H NMR (400 MHz, CDCl3) δ 7.55-7.33 (m, 10H), 5.34 (s, 2H), 5.02 (s, 2H), 4.33 (s, 2H), 4.28 (t, J=11.2 Hz, 1H), 3.71-3.46 (m, 3H), 2.40-2.25 (m, 2H), 1.42 (s, 9H).

### Step 2: Preparation of Intermediate N-2

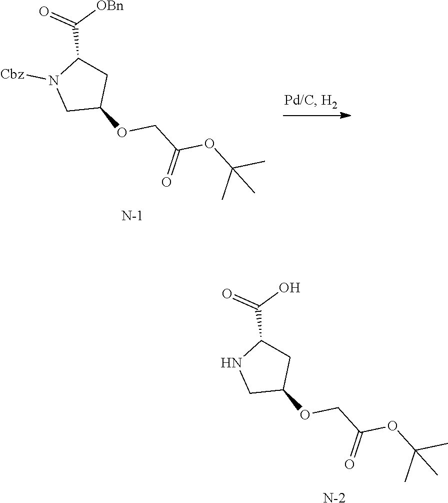

To a 25 mL reaction flask, were added N-1 (1.7 g, 3.62 mmol), Pd/C (0.2 g, 10%), and methanol (20 mL), and then the mixture was stirred and reacted at room temperature for 2 h in a hydrogen atmosphere. LCMS indicated that N-1 disappeared and the reaction was completed. The reaction solution was filtered, and rotatory evaporated, to obtain 800 mg of crude product, which was directly used in the next step.

### Step 3: Preparation of Intermediate N-3

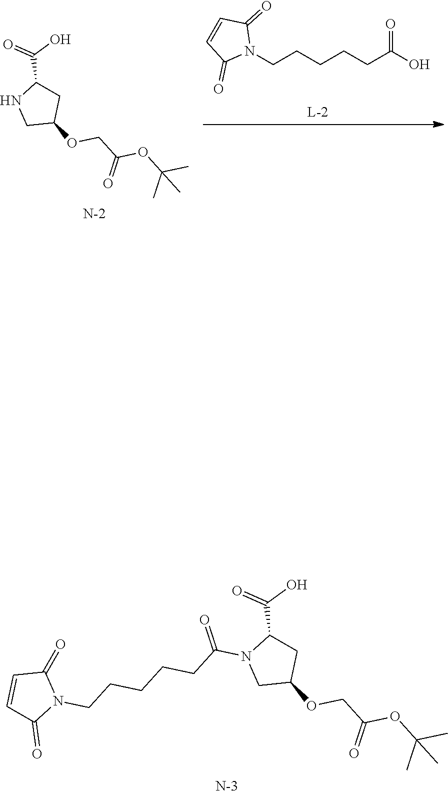

To a 25 mL reaction flask, were added L-2 (413 mg, 1.96 mmol), N,N,N′,N′-tetramethyl-O-(7-azabenzotriazol-1-yl)uronium hexafluorophosphate (744 mg, 1.96 mmol), DIEA (315 mg, 2.45 mmol), and N,N-dimethylformamide (3 mL), and then the mixture was allowed to react at room temperature under stirring for 10 min. Then, N-2 (400 mg, 1.63 mmol) was added dropwise, and the reaction solution was further stirred for 30 min. LCMS indicated the complete reaction of starting material. 15 mL of water was added to the reaction solution, and then the resultant solution was extracted with ethyl acetate (30 mL*3), dried over anhydrous sodium sulfate, filtered, and rotatory evaporated. To the residue, was added a small amount of methanol, and after reversed-phase preparation, 500 mg of oily liquid was obtained, with a yield of 70%.

¹H NMR (400 MHz, CDCl₃) δ 12.22 (s, 1H), 7.90 (s, 1H), 7.85 (s, 1H), 4.33 (m, 3H), 4.02 (t, J=11.2 Hz, 2H), 3.71-3.46 (m, 3H), 2.46 (m, 1H), 2.21 (m, 1H), 2.02 (t, J=9.8 Hz, 2H), 1.61-1.24 (m, 6H), 1.40 (s, 9H).

### Step 4: Preparation of Intermediate N-4

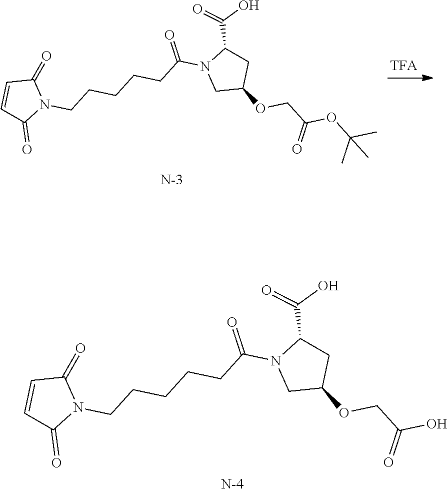

To a 10 mL reaction flask, were added N-3 (500 mg, 1.14 mmol), trifluoroacetic acid (5 mL), and dichloromethane (5 mL), and the mixture was stirred and reacted at room temperature for 4 h. LCMS indicated the complete reaction of the raw materials, and then the excess solvents were removed by rotatory evaporation under reduced pressure. To the residue, was added a small amount of methanol, and after reversed-phase preparation, 300 mg of oily liquid was obtained, with a yield of 78%.

¹H NMR (400 MHz, CDCl₃) δ 12.80 (s, 1H), 12.25 (s, 1H), 7.89 (s, 1H), 7.80 (s, 1H), 4.31 (m, 3H), 4.00 (t, J=11.2 Hz, 2H), 3.77-3.46 (m, 3H), 2.45 (m, 1H), 2.20 (m, 1H), 2.05 (t, J=9.8 Hz, 2H), 1.63 (m, 2H), 1.53 (m, 2H), 1.32 (m, 2H).

### Step 5: Preparation of Intermediate N-5

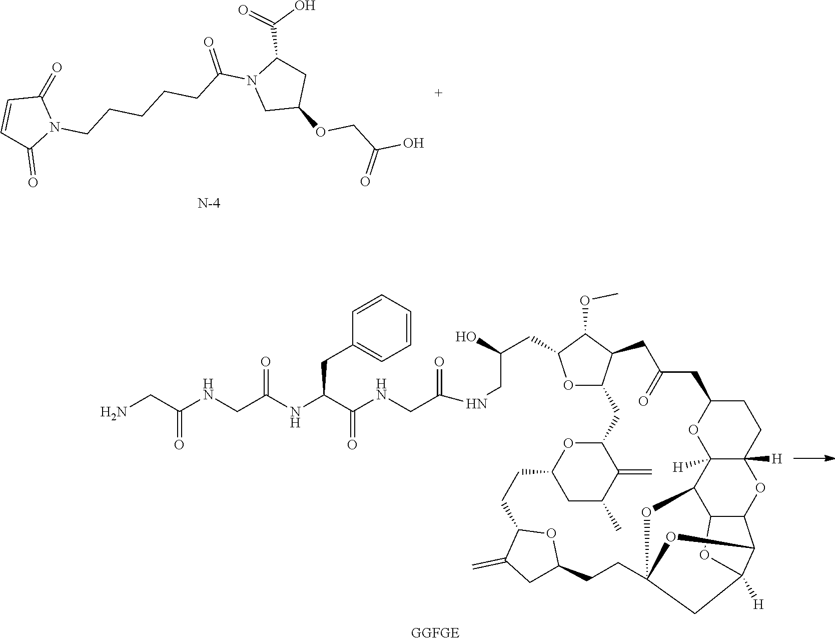

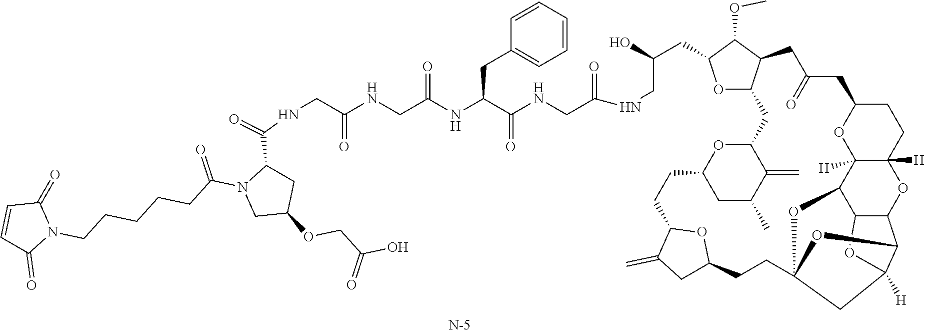

To a 25 mL reaction flask, were added intermediate N-4 (40 mg, 0.105 mmol), N,N-dimethylformamide (3 mL), N,N-diisopropylethylamine (13 mg, 0.105 mmol), and N,N,N′,N′-tetramethyl-O-(7-azabenzotriazol-1-yl)uronium hexafluorophosphate (40 mg, 0.105 mmol), and then the mixture was stirred and reacted at room temperature for 10 min. Then, GGFGE (27 mg, 0.026 mmol, dissolved in 1 mL of N,N-dimethylformamide) was added dropwise, and the resultant solution was allowed to further react under stirring for 30 min. LCMS indicated disappearance of GGFGE and completion of the reaction. After reversed-phase preparation, 20 mg of white solid was obtained, with a yield of 54%.

### Step 6: Preparation of Compound XIv

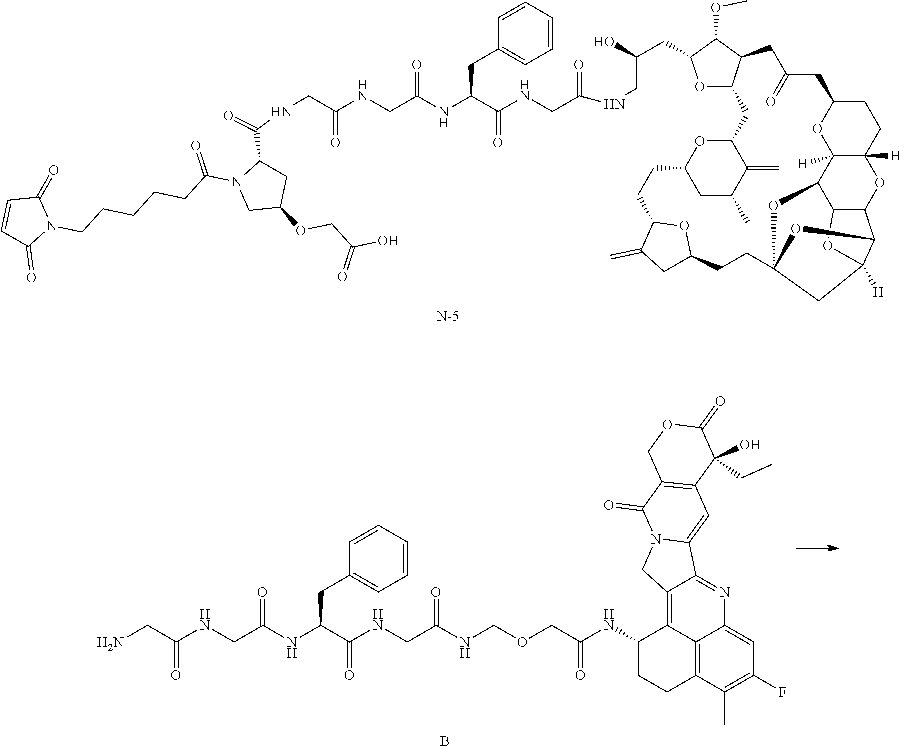

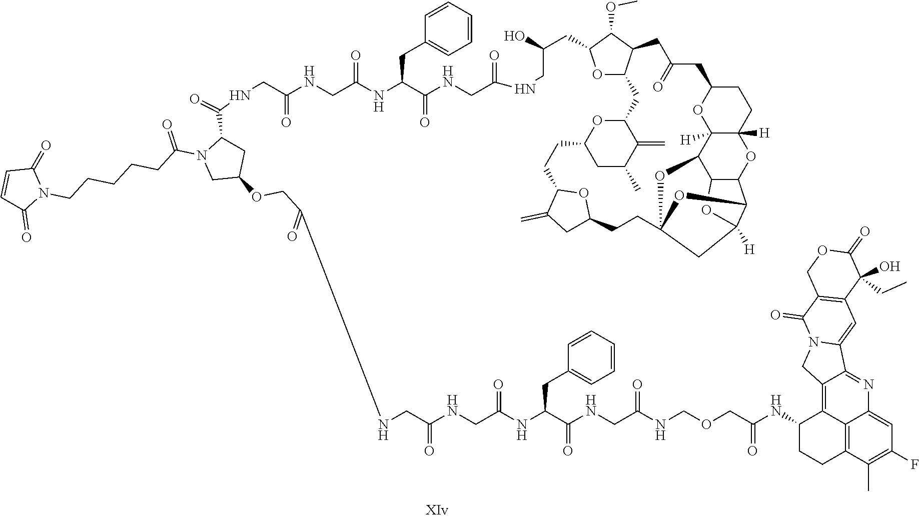 [SMILES: C=C1C[C@@H]2CC[C@@]34C[C@H]5OC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)[C@@H]9C[C@@H](OCC(=O)NCC(=O)NCC(=O)N[C@@H](Cc%10ccccc%10)C(=O)NCC(=O)NCOCC(=O)N[C@H]%10CCc%11c(C)c(F)cc%12nc%13c(c%10c%11%12)Cn%10c-%13cc%11c(c%10=O)COC(=O)[C@]%11(O)CC)CN9C(=O)CCCCCN9C(=O)C=CC9=O)O[C@H]8C[C@H]8O[C@@H](CC[C@@H]1O2)C[C@@H](C)C8=C)O[C@@H]7[C@@H]6O3)[C@H]5O4]

To a 25 mL reaction flask, were added intermediate N-5 (20 mg, 0.014 mmol), B (12 mg, 0.014 mmol), N,N-dimethylformamide (2 mL), N,N-diisopropylethylamine (3 mg, 0.021 mmol), and N,N,N′,N′-tetramethyl-O-(7-azabenzotriazol-1-yl)uronium hexafluorophosphate (6 mg, 0.017 mmol), and then the mixture was stirred and reacted at room temperature for 20 min. LCMS indicated disappearance of N-5 and completion of the reaction. After reversed-phase preparation, 15 mg of yellow solid was obtained, with a yield of 48%.

### Example 24. Preparation of the Dual-Drug Link Assembly Unit of Formula XIw According to the Present Invention

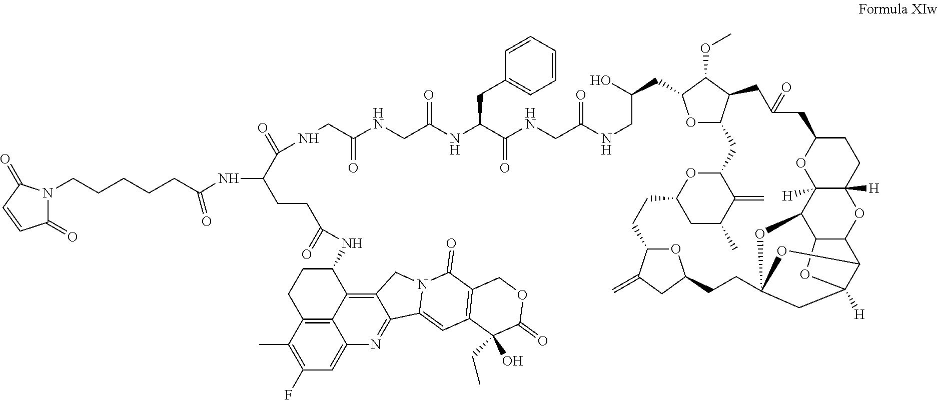

### Step 1: Preparation of Compound N-7

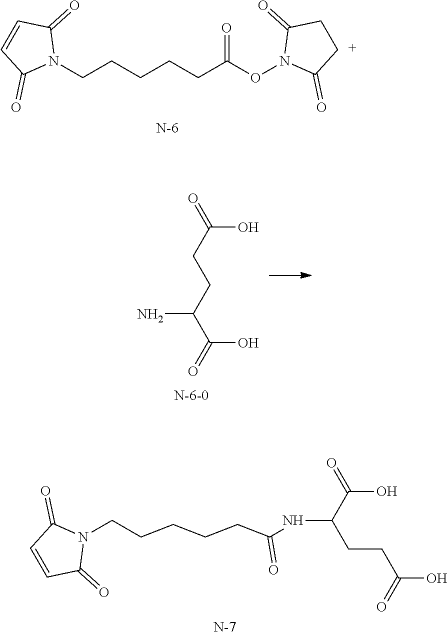

To a 25 mL reaction flask, were added intermediate N-6 (1.0 g, 3.25 mmol), N-6-0 (0.6 g, 3.89 mmol), sodium bicarbonate (1.1 g, 13.00 mmol), ethylene glycol dimethyl ether (15 mL), and water (15 mL), and then the mixture was stirred and reacted at room temperature for 20 h. LCMS indicated most of the product was the molecular weight of the target compound. After concentrating half of the solvent, reversed-phase preparation provided 110 mg of colorless liquid, with a yield of 10%.

¹H NMR (400 MHz, CDCl₃) δ 12.66 (s, 1H), 12.01 (s, 1H), 7.90 (s, 1H), 7.76 (s, 1H), 4.55 (t, J=9.8 Hz, 1H), 4.05 (t, J=11.2 Hz, 2H), 2.33 (t, J=9.2 Hz, 2H), 2.05 (m, 4H), 1.65 (m, 2H), 1.32-1.28 (m, 4H).

### Step 2: Preparation of Compound N-8

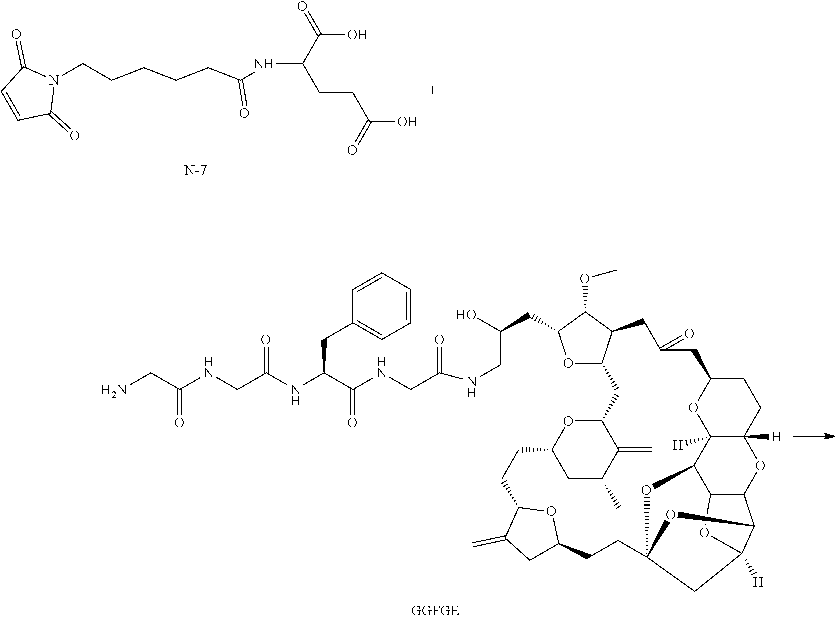

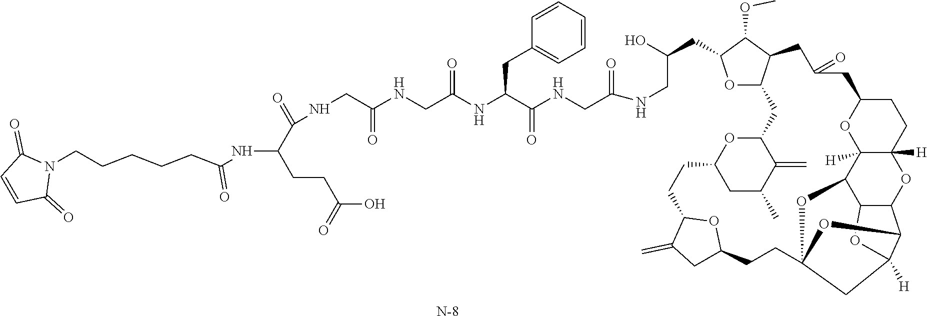

To a 25 mL reaction flask, were added intermediate N-7 (26 mg, 0.076 mmol), N,N-dimethylformamide (2 mL), N,N-diisopropylethylamine (10 mg, 0.076 mmol), and N,N,N′,N′-tetramethyl-O-(7-azabenzotriazol-1-yl)uronium hexafluorophosphate (29 mg, 0.076 mmol), and then the mixture was stirred and reacted at room temperature for 10 min. Then, GGFGE (20 mg, 0.019 mmol, dissolved in 1 mL of N,N-dimethylformamide) was added dropwise, and the resultant solution was allowed to further react under stirring for 30 min. LCMS indicated disappearance of GGFGE and completion of the reaction. After reversed-phase preparation, 18 mg of white solid was obtained, with a yield of 69%.

### Step 3: Preparation of Compound XIw

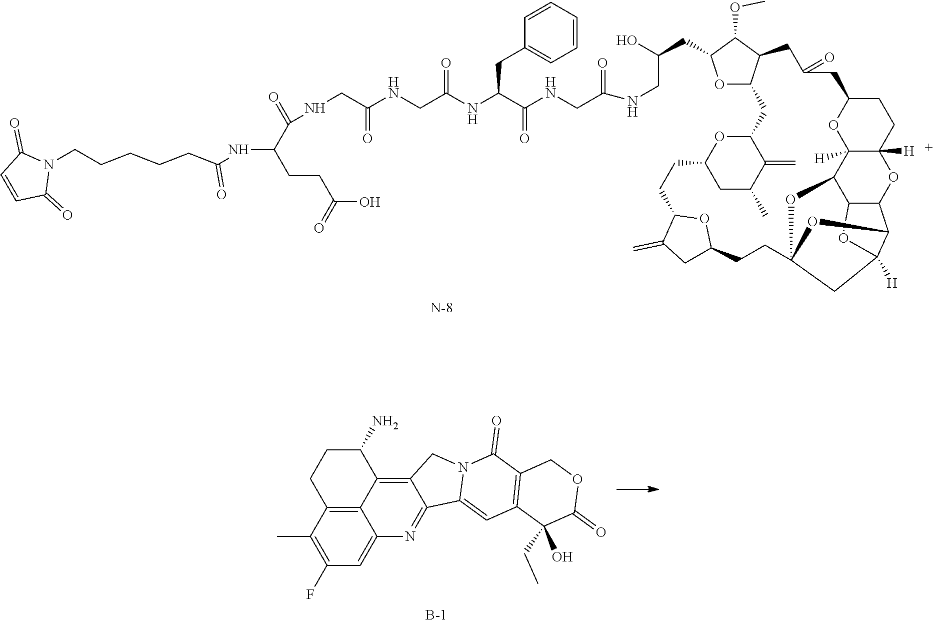

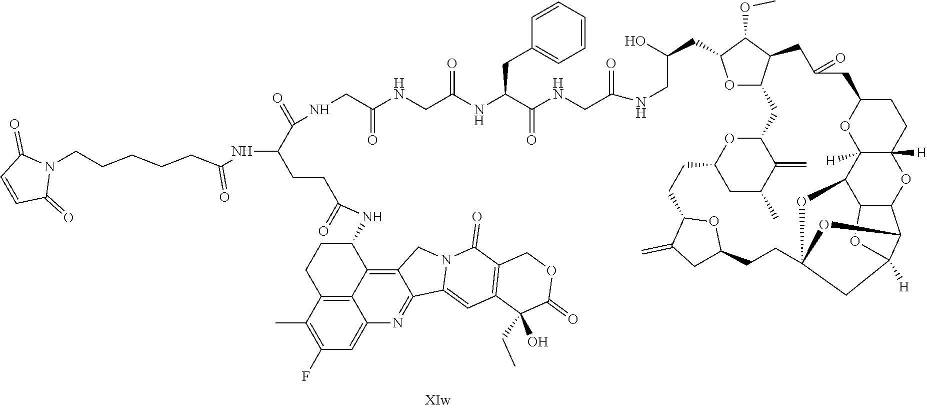 [SMILES: C=C1C[C@@H]2CC[C@@]34C[C@H]5OC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)C(CCC(=O)N[C@H]9CCc%10c(C)c(F)cc%11nc%12c(c9c%10%11)Cn9c-%12cc%10c(c9=O)COC(=O)[C@]%10(O)CC)NC(=O)CCCCCN9C(=O)C=CC9=O)O[C@H]8C[C@H]8O[C@@H](CC[C@@H]1O2)C[C@@H](C)C8=C)O[C@@H]7[C@@H]6O3)[C@H]5O4]

To a 25 mL reaction flask, were added intermediate N-8 (18 mg, 0.013 mmol), B-1 (6 mg, 0.013 mmol), N,N-dimethylformamide (2 mL), N,N-diisopropylethylamine (3 mg, 0.021 mmol), and N,N,N′,N′-tetramethyl-O-(7-azabenzotriazol-1-yl)uronium hexafluorophosphate (6 mg, 0.015 mmol), and then the mixture was stirred and reacted at room temperature for 20 min. LCMS indicated disappearance of N-8 and completion of the reaction. After reversed-phase preparation, 14 mg of yellow solid was obtained, with a yield of 60%.

### Example 25. Preparation of the Dual-Drug Link Assembly Unit of Formula XIx According to the Present Invention

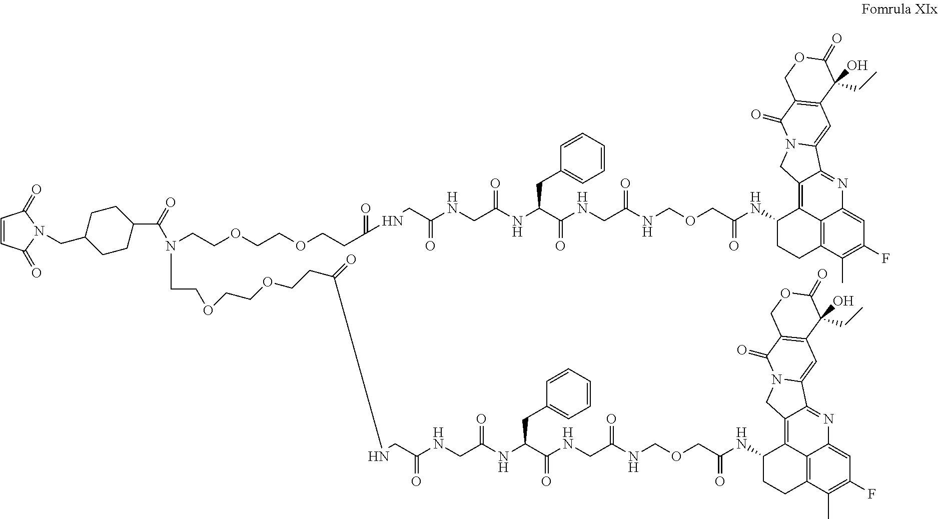

### Step 1: Preparation of Compound N-10

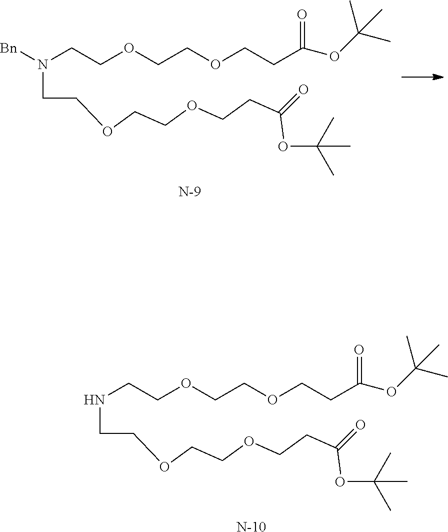

To a 25 mL reaction flask, were added intermediate N-10 (200 mg, 0.37 mmol), Pd/C (20 mg, 10%), and methanol (5 mL), and then in H₂ atmosphere, the reaction solution was reacted under stirring at room temperature for 2 h. LC-MS showed that N-10 disappeared and the reaction was completed. The reaction solution was directly filtered and concentrated, to provide 150 mg of crude oily liquid, with a yield of 90%.

### Step 2: Preparation of Compound N-11

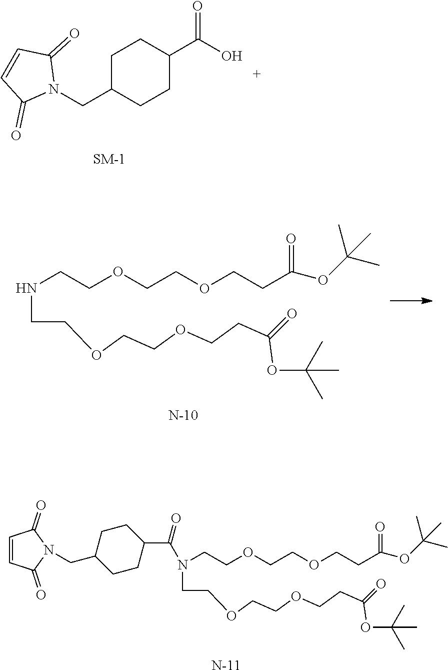

To a 25 mL reaction flask, were added intermediate N-10 (150 mg, 0.33 mmol), SM-1 (79 mg, 0.33 mmol), N,N-dimethylformamide (5 mL), N,N-diisopropylethylamine (65 mg, 0.50 mmol), and N,N,N′,N′-tetramethyl-O-(7-azabenzotriazol-1-yl)uronium hexafluorophosphate (152 mg, 0.40 mmol), and then the mixture was stirred and reacted at room temperature for 10 min. LCMS indicated most of the product was the molecular weight of the target compound, suggesting completion of the reaction. The reaction solution was directly subjected to reversed-phase preparation, to provide 110 mg of colorless liquid, with a yield of 49%.

¹H NMR (400 MHz, CDCl₃) δ 7.76 (s, 1H), 7.55 (s, 1H), 3.73-3.67 (m, 8H), 3.52-3.48 (m, 8H), 3.37 (m, 4H), 3.40 (d, J=8.2 Hz, 2H), 2.42-2.38 (m, 5H), 2.06 (m, 1H), 1.73-1.70 (m, 4H), 1.42 (s, 18H), 1.38 (m, 4H).

### Step 3: Preparation of Compound N-12

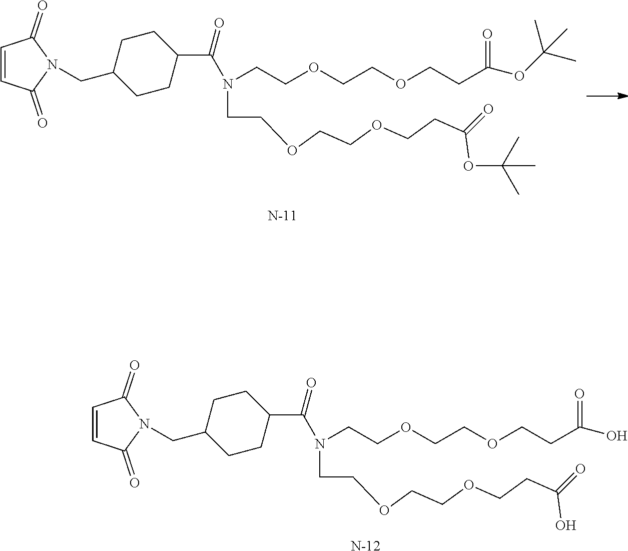

To a 10 mL reaction flask, were added N-11 (110 mg, 0.16 mmol), trifluoroacetic acid (3 mL), and dichloromethane (3 mL), and then the reaction mixture was stirred and reacted at room temperature for 4 h. LCMS showed the complete reaction of the reaction material. The excess solvent was removed by rotatory evaporation under reduced pressure, and then a small amount of methanol was added to the residue. After reversed-phase preparation, 60 mg of oily liquid was obtained, with a yield of 67%.

¹H NMR (400 MHz, CDCl₃) δ 12.10 (s, 1H), 11.93 (s, 1H), 7.86 (s, 1H), 7.65 (s, 1H), 3.61-3.52 (m, 16H), 3.40-3.37 (m, 6H), 2.40-2.38 (m, 5H), 2.03 (m, 1H), 1.72-1.70 (m, 4H), 1.36 (m, 4H).

### Step 4: Preparation of Compound XIx

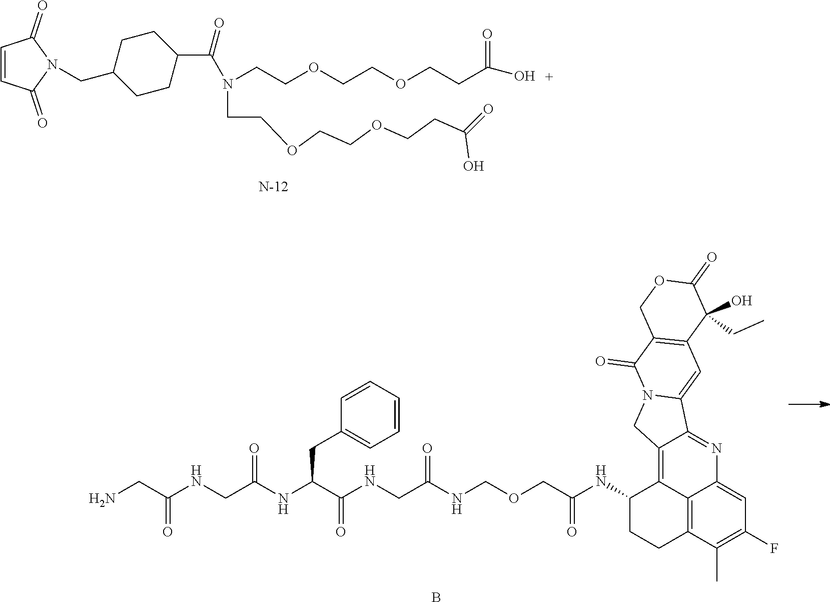

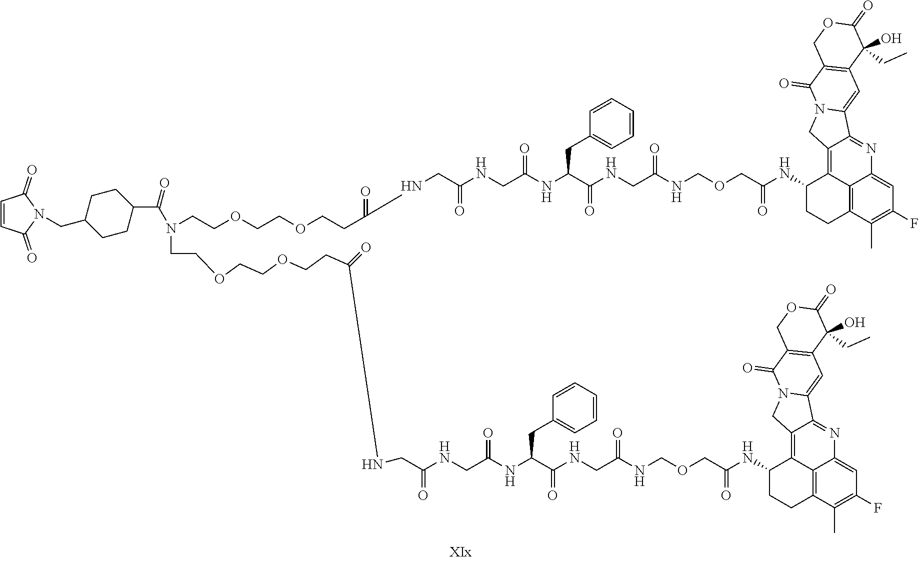 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCN(CCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)C(=O)C1CCC(CN2C(=O)C=CC2=O)CC1)CC3]

To a 25 mL reaction flask, were added intermediate N-12 (5 mg, 0.008 mmol), B (15 mg, 0.018 mmol), N,N-dimethylformamide (2 mL), N,N-diisopropylethylamine (3 mg, 0.021 mmol), and N,N,N′,N′-tetramethyl-O-(7-azabenzotriazol-1-yl)uronium hexafluorophosphate (7 mg, 0.018 mmol), and then the mixture was stirred and reacted at room temperature for 20 min. LCMS indicated disappearance of N-12 and completion of the reaction. After reversed-phase preparation, 10 mg of yellow solid was obtained, with a yield of 55%.

### Example 26. Preparation of the Dual-Drug Link Assembly Unit of Formula XIy According to the Present Invention

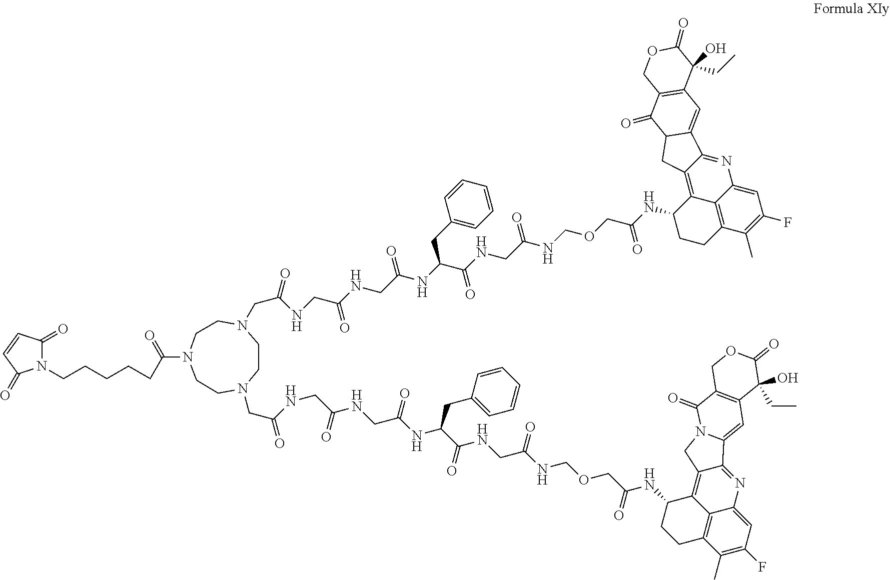

### Step 1: Preparation of Compound N-13

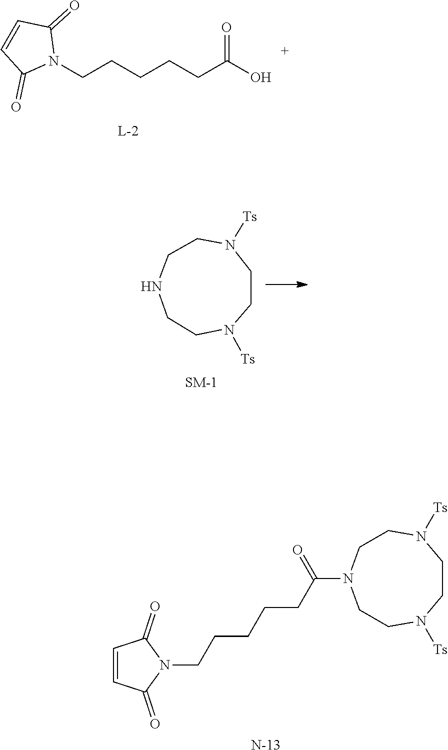

To a 25 mL reaction flask, were added intermediate L-2 (97 mg, 046 mmol), SM-1 (200 mg, 0.46 mmol), N,N-dimethylformamide (5 mL), N,N-diisopropylethylamine (89 mg, 0.68 mmol), and N,N,N′,N′-tetramethyl-O-(7-azabenzotriazol-1-yl)uronium hexafluorophosphate (209 mg, 0.55 mmol), and then the mixture was stirred and reacted at room temperature for 20 min. LCMS indicated disappearance of L-2 and completion of the reaction. After reversed-phase preparation, 200 mg of white solid was obtained, with a yield of 69%.

¹H NMR (400 MHz, CDCl₃) δ 7.86 (s, 1H), 7.75 (s, 1H), 7.65-7.56 (m, 4H), 7.51-7.45 (m, 4H), 4.00 (t, J=11.2 Hz, 2H), 3.42-3.40 (m, 4H), 3.32-3.28 (m, 8H), 2.45 (s, 3H), 2.41 (s, 3H), 2.27 (t, J=9.8 Hz, 2H), 1.63-1.53 (m, 4H), 1.32 (m, 2H).

### Step 2: Preparation of Compound N-14

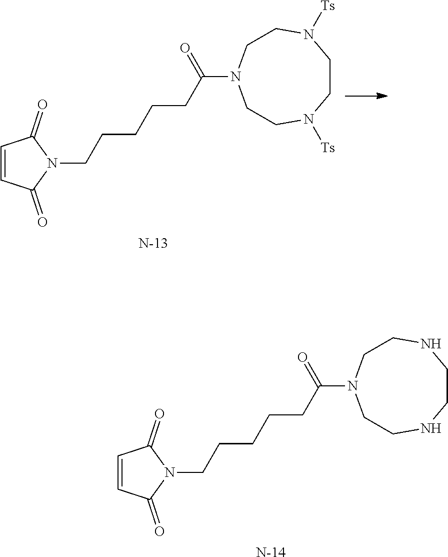

Intermediate N-13 (200 mg, 0.32 mmol), phenol (149 mg, 1.59 mmol), and 33% HBr/acetic acid (5 mL) were added to a 25 mL reaction flask, and then the mixture was stirred at room temperature for 20 h. LCMS showed that N-13 disappeared and the reaction was completed. The reaction solution was directly added into methyl tert-butyl ether (30 mL) dropwise, to precipitate the solid, which was filtered to provide 140 mg of brown solid, with a yield of 92%.

### Step 3: Preparation of Compound N-15

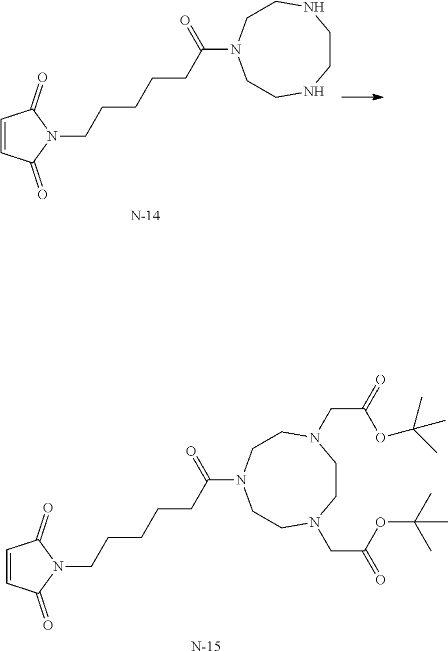

To a 25 mL reaction flask, were added intermediate N-14 (140 mg, 0.29 mmol), tert-butyl bromoacetate (130 mg, 0.67 mmol), potassium carbonate (160 mg, 1.16 mmol), and N,N-dimethylformamide (5 mL), and then the mixture was allowed to react at room temperature under stirring for 20 h. LCMS indicated disappearance of N-14 and completion of the reaction. The reaction solution was filtered and concentrated, and after reversed-phase preparation, 120 mg of oily liquid was obtained, with a yield of 75%.

¹H NMR (400 MHz, CDCl₃) δ 7.84 (s, 1H), 7.73 (s, 1H), 4.05 (t, J=10.2 Hz, 2H), 3.32-3.30 (m, 8H), 2.62 (m, 4H), 2.46 (m, 4H), 2.29 (t, J=10.1 Hz, 2H), 1.64-1.52 (m, 4H), 1.42 (s, 18H), 1.31 (m, 2H).

### Step 4: Preparation of Compound N-16

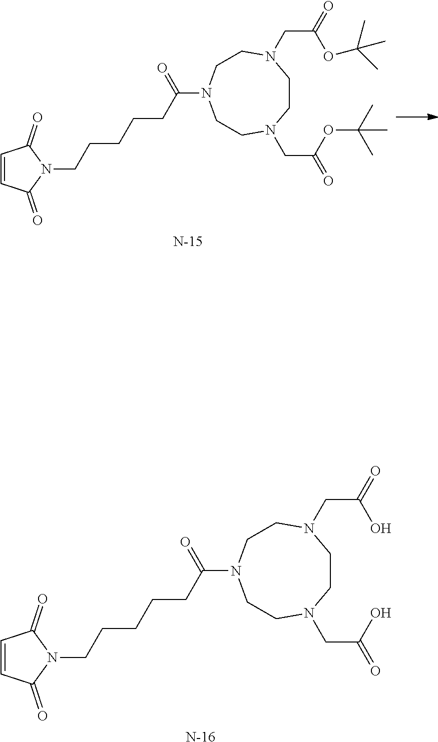

To a 10 mL reaction flask, were added N-15 (120 mg, 0.22 mmol), trifluoroacetic acid (3 mL), and dichloromethane (3 mL), and then the reaction mixture was stirred and reacted at room temperature for 4 h. LCMS showed the complete reaction of the reaction material. The excess solvent was removed by rotatory evaporation under reduced pressure, and then a small amount of methanol was added to the residue. After reversed-phase preparation, 60 mg of oily liquid was obtained, with a yield of 63%.

### Step 5: Preparation of Compound XIy

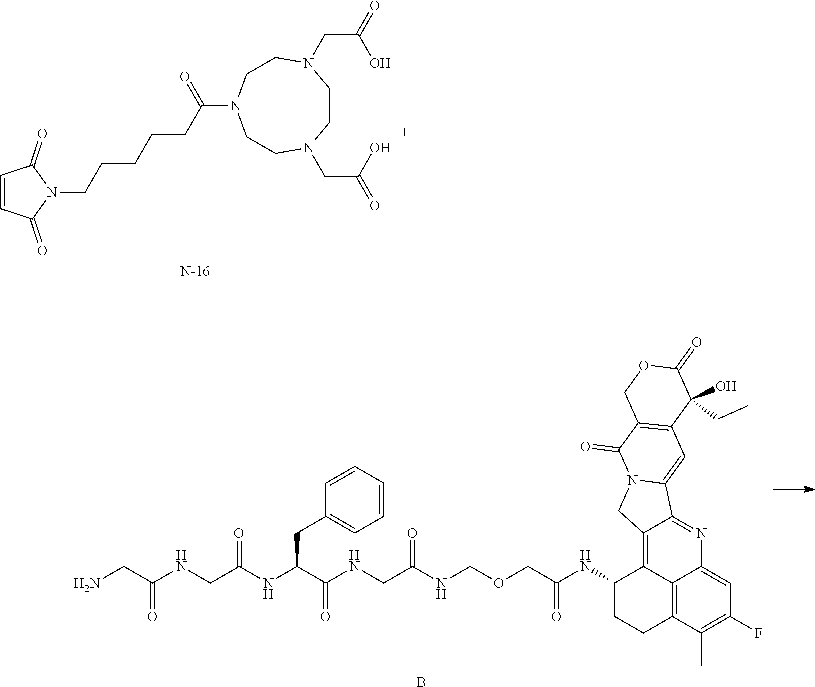

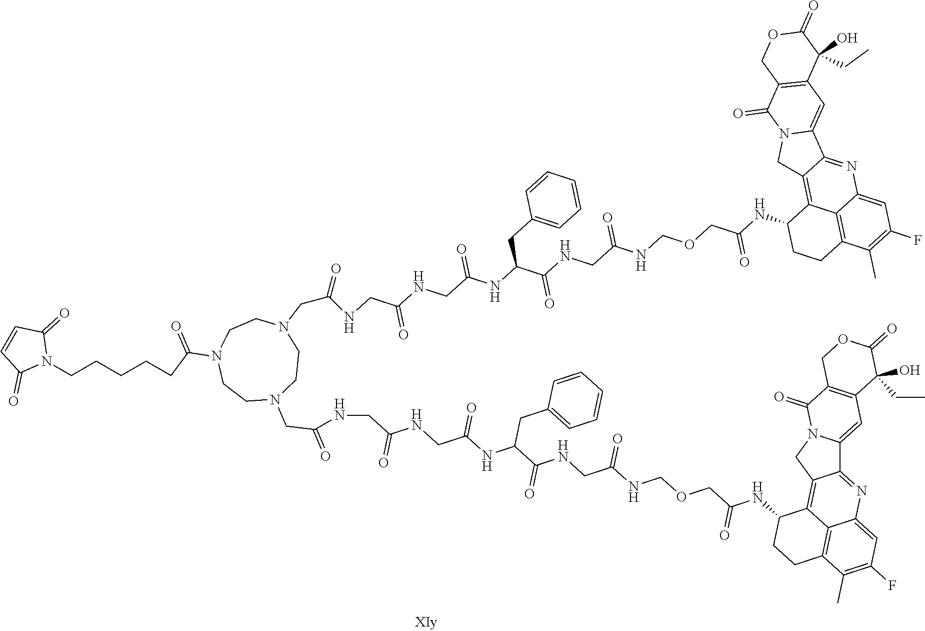 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)C(Cc1ccccc1)NC(=O)CNC(=O)CNC(=O)CN1CCN(CC(=O)NCC(=O)NCC(=O)N[C@@H](Cc2ccccc2)C(=O)NCC(=O)NCOCC(=O)N[C@H]2CCc4c(C)c(F)cc5nc6c(c2c45)Cn2c-6cc4c(c2=O)COC(=O)[C@]4(O)CC)CCN(C(=O)CCCCCN2C(=O)C=CC2=O)CC1)CC3]

To a 25 mL reaction flask, were added intermediate N-16 (4 mg, 0.008 mmol), B (15 mg, 0.018 mmol), N,N-dimethylformamide (2 mL), N,N-diisopropylethylamine (3 mg, 0.021 mmol), and N,N,N′,N′-tetramethyl-O-(7-azabenzotriazol-1-yl)uronium hexafluorophosphate (7 mg, 0.018 mmol), and then the mixture was stirred and reacted at room temperature for 20 min. LCMS indicated disappearance of N-16 and completion of the reaction. After reversed-phase preparation, 10 mg of yellow solid was obtained, with a yield of 58%.

### Example 27. Preparation of Antibody-Drug Coujugate (ADC)

### 1. Preparation

Step (1) Reduction of antibodies: The antibody medium was replaced with pH 7.2 PBS/EDTA buffer, to prepare an antibody solution with a concentration of 4 mg/mL. The solution (0.5 mL) was transferred into a 1.5 mL polypropylene test tube, to which was addd TCEPPBS solution (8 μl: 3 equivalents relative to one molecule of antibody), and incubated at 25° C. for 1 h to reduce part of the disulfide bonds in antibody.

Step (2) Conjugating of antibody and drug linker: At 25° C., DMSO (43 μL) and the solution of the dual-drug link assembly unit obtained in Examples 1 to 26 (6.7 μL: 5 equivalents relative to one molecule of antibody) in DMSO were added to the above solution, mixed well with a tube mixer, and stirred at room temperature for 120 min, to connect the drug linker to the antibody. Then, 100 mM of NAC aqueous solution (2 μL: 18.4 equivalents relative to one molecule of antibody) was added, and stirred at room temperature for 20 min, to terminate the reaction of the drug linker. Thereby, each ADC: Xia-XIy was obtained.

### 2. Characterization

Exclusion chromatography was used to measure the aggregation degree of ADC, LCMS was used to test the DAR value of ADC, and HIC was used to detect the content of naked antibodies in ADC.

TABLE 1
Characterization results of antibody-drug conjugates XIa~Xly.

| Name of antibody- | | | |
|---|---|---|---|
| drug conjugates | DAR | Degree of aggregation | Naked antibody |
| XIa | 3.5 | 5.7 | 6.3 |
| XIb | 3.8 | 5.9 | 1.8 |
| XIc | 3.9 | 10.2 | 0.2 |
| XId | 4.1 | 8.7 | 2.8 |
| XIe | 4.6 | 8.9 | 8.9 |
| XIf | 3.7 | 4.1 | 13 |
| XIg | 4.2 | 16.1 | 5.9 |
| XIh | 4.1 | 8.4 | 7.8 |
| XIi | 4.7 | 6.9 | 3.2 |
| XIj | 2.8 | 9.4 | 25 |
| XIk | 2.9 | 6.2 | 19 |
| XIl | 4.5 | 1.9 | 5.6 |
| XIm | 4.4 | 1.3 | 7.3 |
| XIn | 3.7 | 9.9 | 8.2 |
| XIo | 3.9 | 2.8 | 3.6 |
| XIp | 3.5 | 8.9 | 7.1 |
| XIq | 4.8 | 10.2 | 45 |
| XIr | 4.9 | 4.9 | 2.1 |
| XIs | 4.2 | 1.1 | 3.9 |
| XIt | 5.3 | 18 | 1.7 |
| XIu | 4.5 | 25 | 6.3 |
| XIv | 4.9 | 17.4 | 8.1 |
| XIw | 3.8 | 9.9 | 5.6 |
| XIx | 4.9 | 16 | 3.2 |
| XIy | 4.8 | 1.7 | 3.8 |

The beneficial effects of the present invention were demonstrated with reference to the experimental example.

### Experimental Example 1. Cell Activity Testing

### 1. Experimental Method

Cells: N87 cells, SK—BR—3 cells, MDA-MB-468 cells.

Procedures: cells in logarithmic growth phase were collected and counted by a cell counter, then resuspended in the culture medium. The cell suspension was adjusted to a suitable concentration, and then inoculated into a 96-well plate at 100 μL/well. Cells were cultured in a 37° C., 100% relative humidity, 5% CO₂ incubator for 24 h. On the second day, the tested ADC was diluted to corresponding concentrations with culture medium, and then added into the cells in the 96-well plate at 25 μL/well. The cells were cultured in a 37° C., 100% relative humidity, 5% CO₂ incubator for 14 h. CCK-8 was added at 10 μL/well, followed by incubating in a 37° C. incubator for 2-4 h. After gently shaking, the absorbance was detected at a wavelength of 450 nm on the SpectraMax i3X Reader, using the absorbance at 650 nm as a reference (i.e. 450 nm absorbance-650 nm absorbance), to obtain the inhibition rate, which was further used to calculate the IC₅₀ value.

A known ADC DS-8201 on the market was used as the positive control.

### 2. Experimental Results

TABLE 2
Cytotoxicity test results.

| IC₅₀ (ng/ml) | N87 | SK-BR-3 | MDA-MB-468 |
|---|---|---|---|
| DS-8201 | 113 | 30 | >1000 |
| XIa | 15.8 | 3.3 | >1000 |
| XIb | 12.1 | 1.7 | >1000 |
| XIc | 17.5 | 5.8 | >1000 |
| XId | 21.3 | 8.7 | >1000 |
| XIe | 20.5 | 8.9 | >1000 |
| XIf | 11.1 | 1.8 | >1000 |
| XIg | 13.9 | 3.1 | >1000 |
| XIh | 11.1 | 2.1 | >1000 |
| XIi | 25 | 11.3 | >1000 |
| XIj | 18.9 | 9 | >1000 |
| XIk | 17.1 | 6.2 | >1000 |
| XIl | 9.8 | 1.1 | >1000 |
| XIm | 12.6 | 1.3 | >1000 |
| XIn | 16.5 | 5.5 | >1000 |
| XIo | 12.3 | 2.3 | >1000 |
| XIp | 18.7 | 6.8 | >1000 |
| XIq | 37 | 21 | >1000 |
| XIr | 19.4 | 8.6 | >1000 |
| XIs | 8.7 | 1.1 | >1000 |
| XIt | 17.7 | 9.5 | >1000 |
| XIu | 13.3 | 6.9 | >1000 |
| XIV | 24.3 | 13.2 | >1000 |
| XIw | 13.5 | 6.5 | >1000 |
| XIx | 20.1 | 11.8 | >1000 |
| Xly | 11.1 | 1.9 | >1000 |

These data demonstrated that the ADC of the present invention had good anti-proliferation activity on HER2-positive cell lines N87 and SK—BR—3, but no activity on HER2 negative cell lines MDA-MB-468, indicating that the ADC of the present invention had good anti-tumor effect and antigen specificity.

In addition, the ADC provided in the present invention was more effective on HER2 positive cell lines N87 and SK—BR—3 than the already marketed ADC DS-8201.

In summary, the present invention provided a dual-drug link assembly unit, which could be linked to a targeting linker to obtain a dual-drug targeting linker-drug conjugate, that could specifically kill tumor cells expressing targeted antigen and reduce the toxicity and side effects on normal tissues. Probably, they may also effectively overcome drug resistance and achieve a synergistic anti-tumor effect. Compared to the already listed ADC DS-8201, the ADC provided in the present invention significantly improved the anti-proliferation activity on HER2 positive cell strains N87 and SK—BR—3. The dual-drug link assembly unit and the dual-drug targeting linker-drug conjugate of the present invention had broad application prospects in the manufacture of medicaments for prevention and/or treatment of tumors.

1. A dual-drug link assembly unit represented by formula III, or a stereoisomer thereof, or an optical isomer thereof, or a deuterated compound thereof: 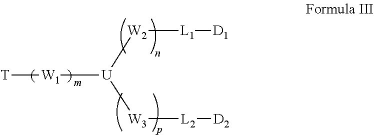
wherein, T is a tether group which can be connected to the targeting linker; the targeting linker is a substance that can target and bind to the lesion site;
U is a Y-shaped linker moiety, with a structure of 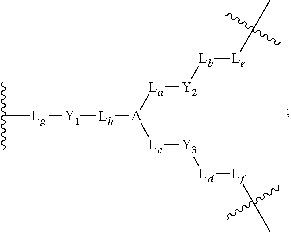
wherein, Y₁, Y₂, and Y₃ are each independently selected from the group consisting of CONH, NHCO, CO, NH, COO, OCO, O, S, 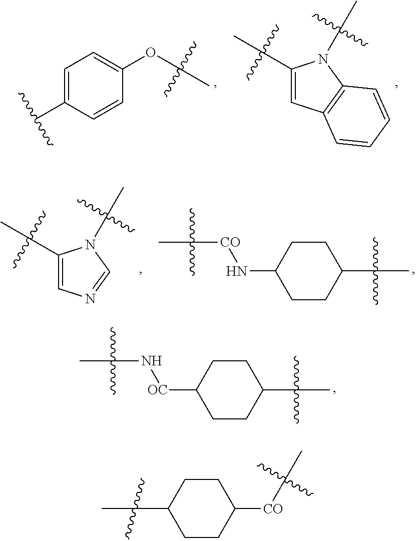
or absence; Lₐ, L_{b}, L_{c}, L_{d}, Lₑ, L_{f}, L_{g}, Lₕ are each independently selected from the group consisting of 0-8 methylenes; A is selected from N, as well as the following substituted or unsubstituted groups: aryl, heteroaryl, chain alkyl, fused cycloalkyl, fused heterocycloalkyl, saturated cycloalkyl or saturated heterocycloalkyl, and the substituent is each independently selected from the group consisting of halogen, cyano, hydroxyl, C₁₋₆ alkyl or C₁₋₆ alkoxy;
m, n, and p are each independently selected from an integer of 0 to 30, as well as n and p are not both 0;
W₁, W₂, and W₃ are each independently selected from the group consisting of methylene, 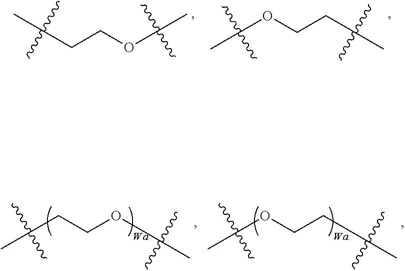
alkenylene, alkynylene, 3-8 membered aryl, 3-8 membered heteroaryl; Wa is selected from an integer of 2 to 8;
L₁ and L₂ are cleavable or non-cleavable linking groups;
D₁ and D₂ are the first and second drug structural units, respectively, with the same or different structures.

2. The dual-drug link assembly unit according to claim 1, or a stereoisomer thereof, or an optical isomer thereof, or a deuterated compound thereof, characterized in that said T can react and connect with thiol or amino groups in the targeting linker.

3. The dual-drug link assembly unit according to claim 1, or a stereoisomer thereof, or an optical isomer thereof, or a deuterated compound thereof, characterized in that the structure of said 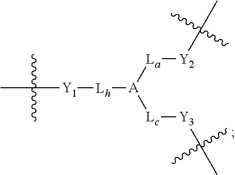
is selected from the group consisting of:

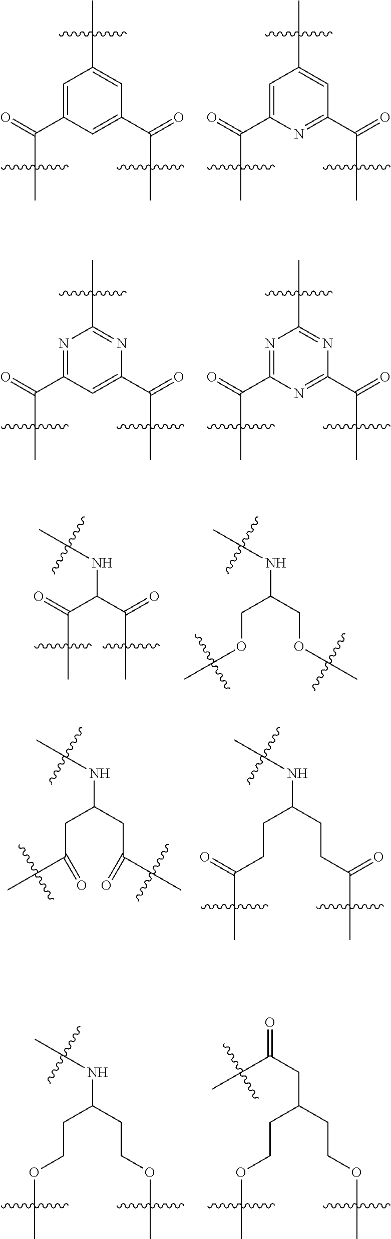

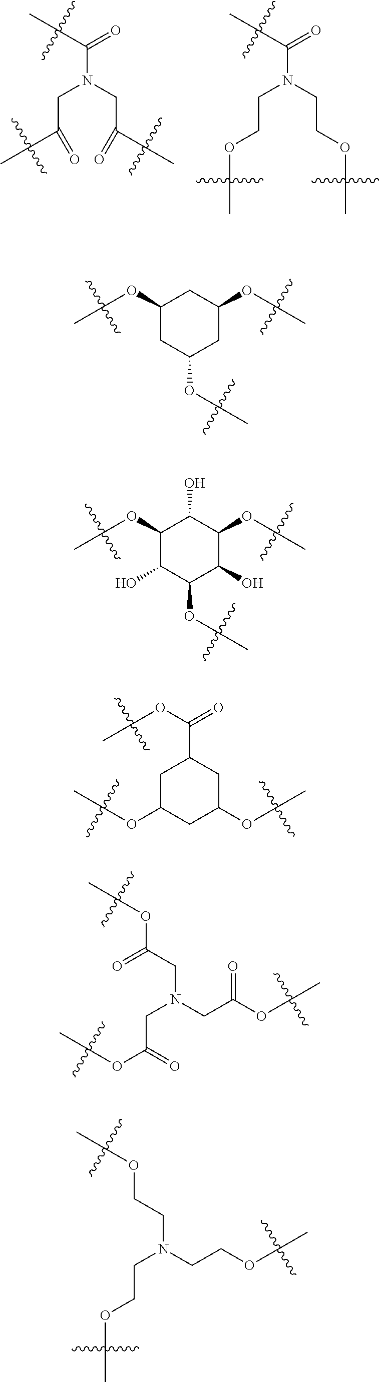

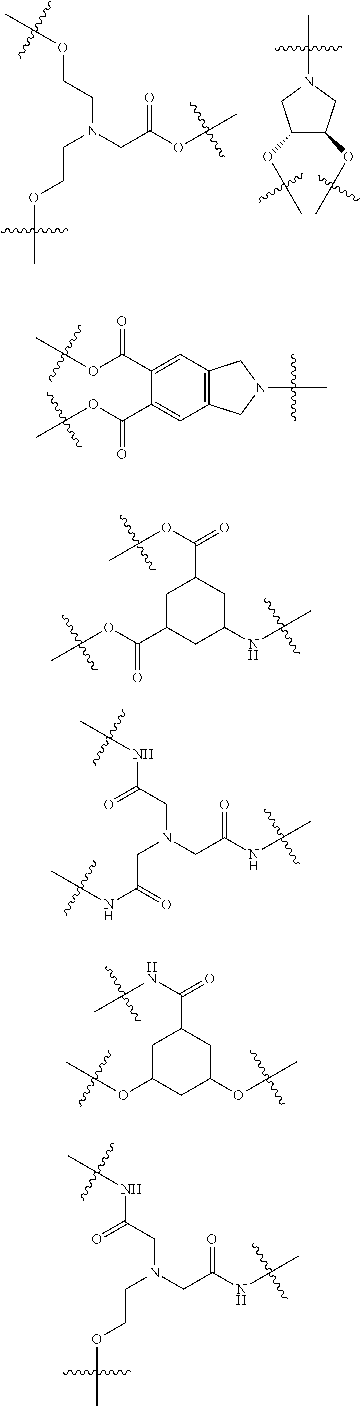

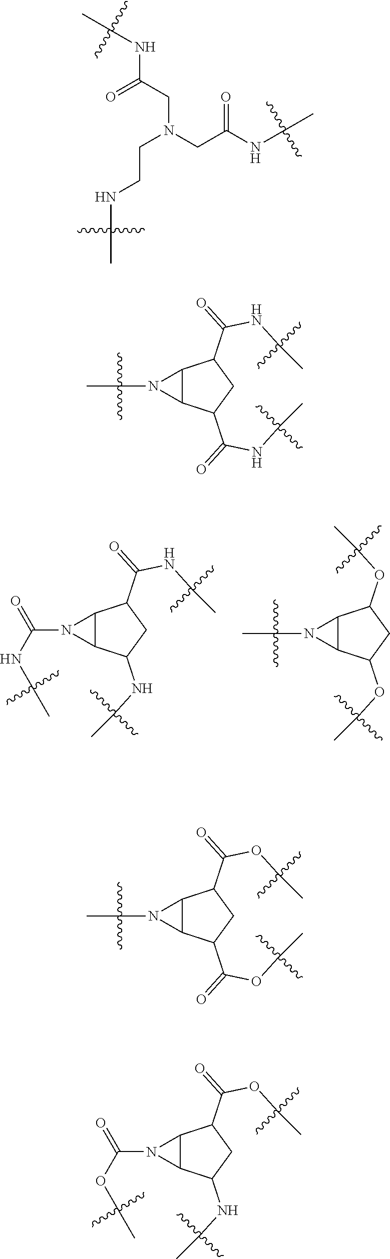 [SMILES: *NCCN(CC(=O)N*)CC(=O)N* ; *NC(=O)C1CC(C(=O)N*)C2C1N2* ; *NC(=O)C1CC(N*)C2C1N2C(=O)N* ; *OC1CC(O*)C2C1N2* ; *OC(=O)C1CC(C(=O)O*)C2C1N2* ; *NC1CC(C(=O)O*)C2C1N2C(=O)O*]

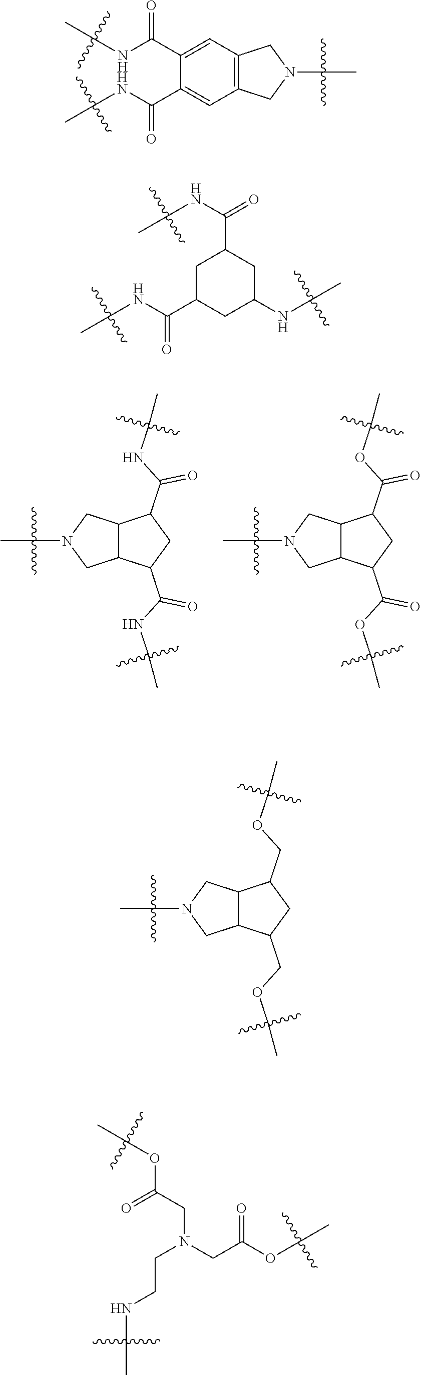 [SMILES: *NC(=O)c1cc2c(cc1C(=O)N*)CN(*)C2 ; *NC(=O)C1CC(N*)CC(C(=O)N*)C1 ; *NC(=O)C1CC(C(=O)N*)C2CN(*)CC12 ; *OC(=O)C1CC(C(=O)O*)C2CN(*)CC12 ; *OCC1CC(CO*)C2CN(*)CC12 ; *NCCN(CC(=O)O*)CC(=O)O*]

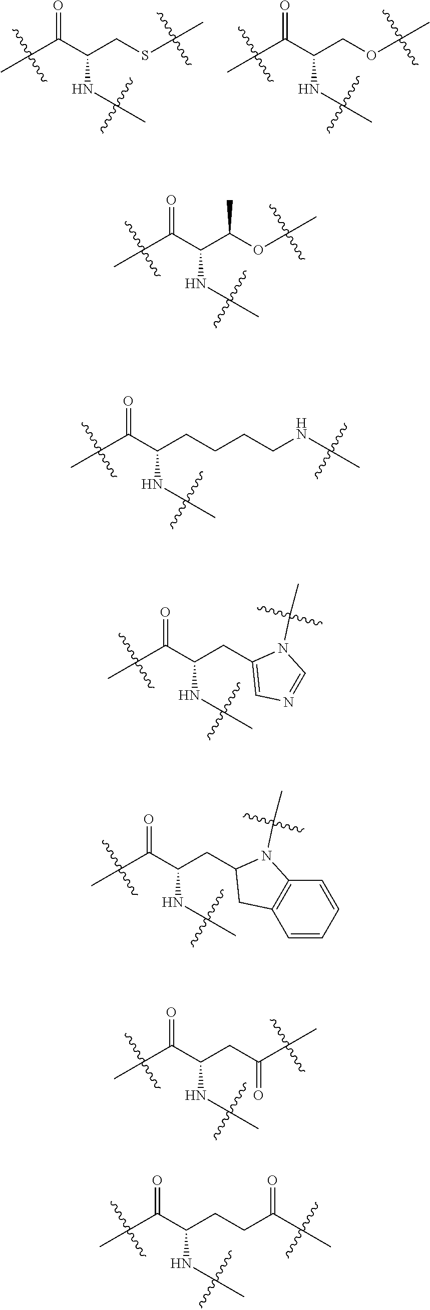

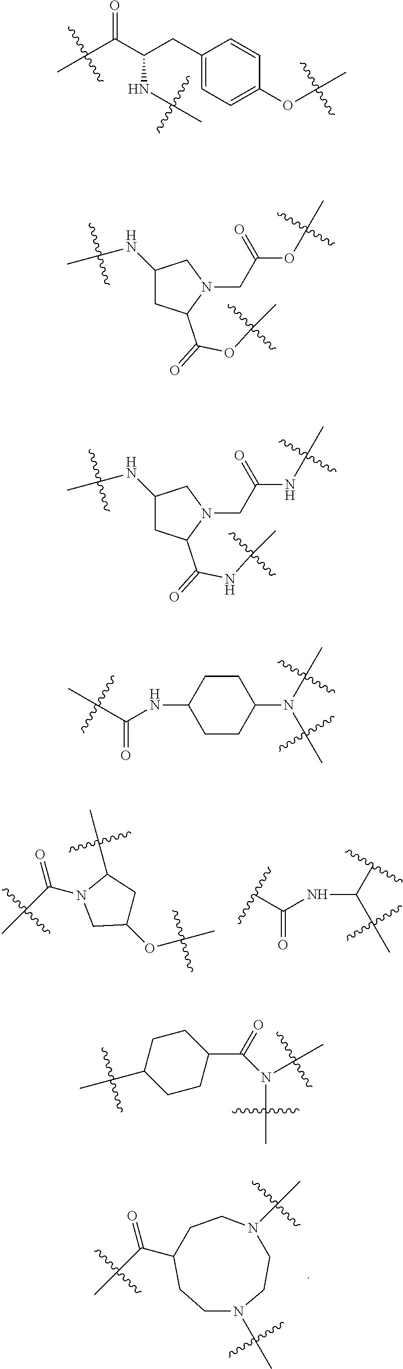

4. The dual-drug link assembly unit according to claim 1, or a stereoisomer thereof, or an optical isomer thereof, or a deuterated compound thereof, characterized in that the structure of said dual-drug link assembly unit is as represented by formula IV: 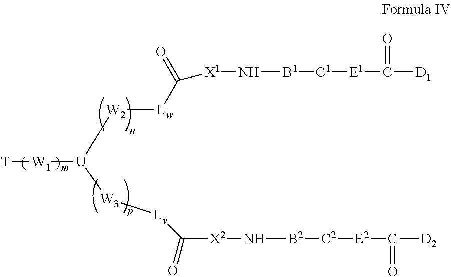
wherein, the structure of U is 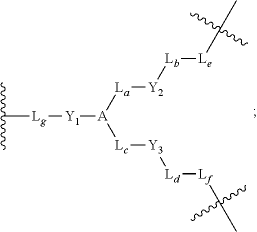
in which Y₁, Y₂, and Y₃ are each independently selected from the group consisting of CONH, CO, NH, O, 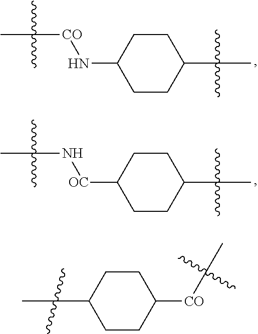
or absence; Lₐ, L_{b}, L_{c}, L_{d}, Lₑ, L_{f}, L_{g}, L_{w}, and Lᵥ, are each independently selected from the group consisting of 0-4 methylenes; A is selected from N, as well as the following substituted or unsubstituted groups: aryl, heteroaryl, chain alkyl, saturated cycloalkyl or saturated heterocycloalkyl, and the substituent is each independently selected from halogen, cyano, hydroxyl, C₁₋₆ alkyl or C₁₋₆ alkoxy;
m, n, and p are each independently selected from an integer of 0 to 30, as well as n and p are not both 0;
W₁, W₂, and W₃ are each independently selected from the group consisting of methylene, 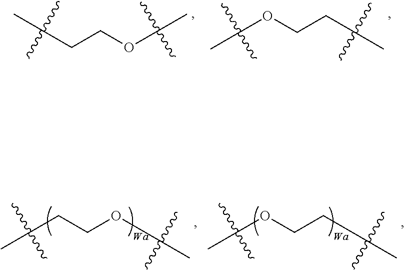
alkenylene, alkynylene, 3-8 membered aryl, 3-8 membered heteroaryl; Wa is selected from an integer of 2 to 4;
X¹ and X² are each independently selected from the group consisting of 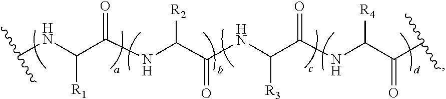
wherein, a, b, c, and d are each independently selected from 0 or 1; R₁, R₂, R₃, and R₄ are each independently selected from the group consisting of H, C₁₋₅ alkyl, substituted or unsubstituted benzyl, and -L₇NHCONH₂; L₇ is 0-3 methylenes;
B¹, B², C¹, C², E¹, and E² are each independently selected from the group consisting of the following substituted or unsubstituted groups: 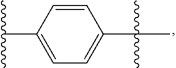 [SMILES: *c1ccc(*)cc1]
L₈NHL₃, L₄OL₅ or absence; the substituent is each independently selected from 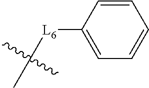
and C_{1˜5} alkyl; wherein L₈, L₃, L₄, L₅, and L₆ are each independently selected from 0˜2 methylenes;
D₁ and D₂ are independently selected from cytotoxic medicaments, medicaments for treating autoimmune diseases, or anti-inflammatory medicaments;
T is as defined in claim 1.

5. The dual-drug link assembly unit according to claim 4, or a stereoisomer thereof, or an optical isomer thereof, or a deuterated compound thereof, characterized in that the structure of said dual-drug link assembly unit is as represented by formula V: 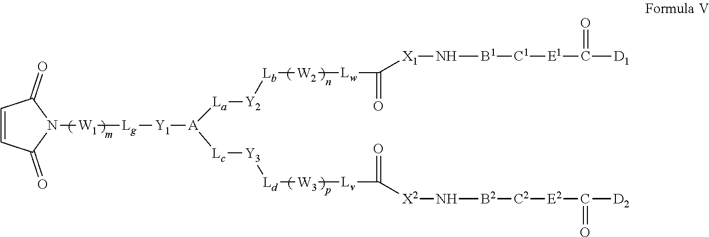
wherein, Y₁, Y₂, and Y₃ are each independently selected from the group consisting of CONH, CO, NH, O, 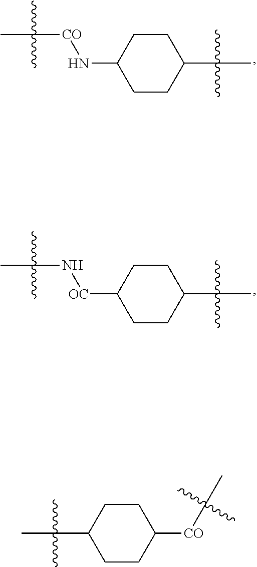
or absence; Lₐ, L_{b}, L_{c}, L_{d}, Lₑ, L_{f}, L_{g}, L_{w}, and Lᵥ, are each independently selected from 0-4 methylenes; A is selected from the group consisting of N, substituted or unsubstituted phenyl, and substituted or unsubstituted 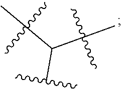 [SMILES: *C(*)*]
and the substituent is each independently selected from halogen, cyano, hydroxyl, C₁₋₆ alkyl or C₁₋₆ alkoxy;
m, n, and p are each independently selected from an integer of 0 to 30, as well as n and p are not both 0;
W₁, W₂, and W₃ are each independently selected from the group consisting of methylene, 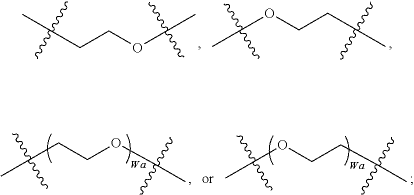
Wa is selected from an integer of 2 to 3; and at least one of W₂ and W₃ is 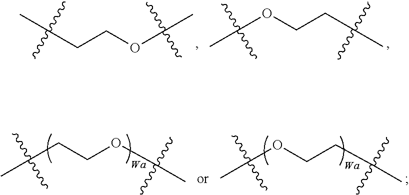
X¹, X², B¹, B², C¹, C², E¹, E², D₁, and D₂ are as defined in claim 4.

6. The dual-drug link assembly unit according to claim 5, or a stereoisomer thereof, or an optical isomer thereof, or a deuterated compound thereof, characterized in that the structure of said dual-drug link assembly unit is as represented by formula VI-1, VI-2, VI-3 or VI-4: 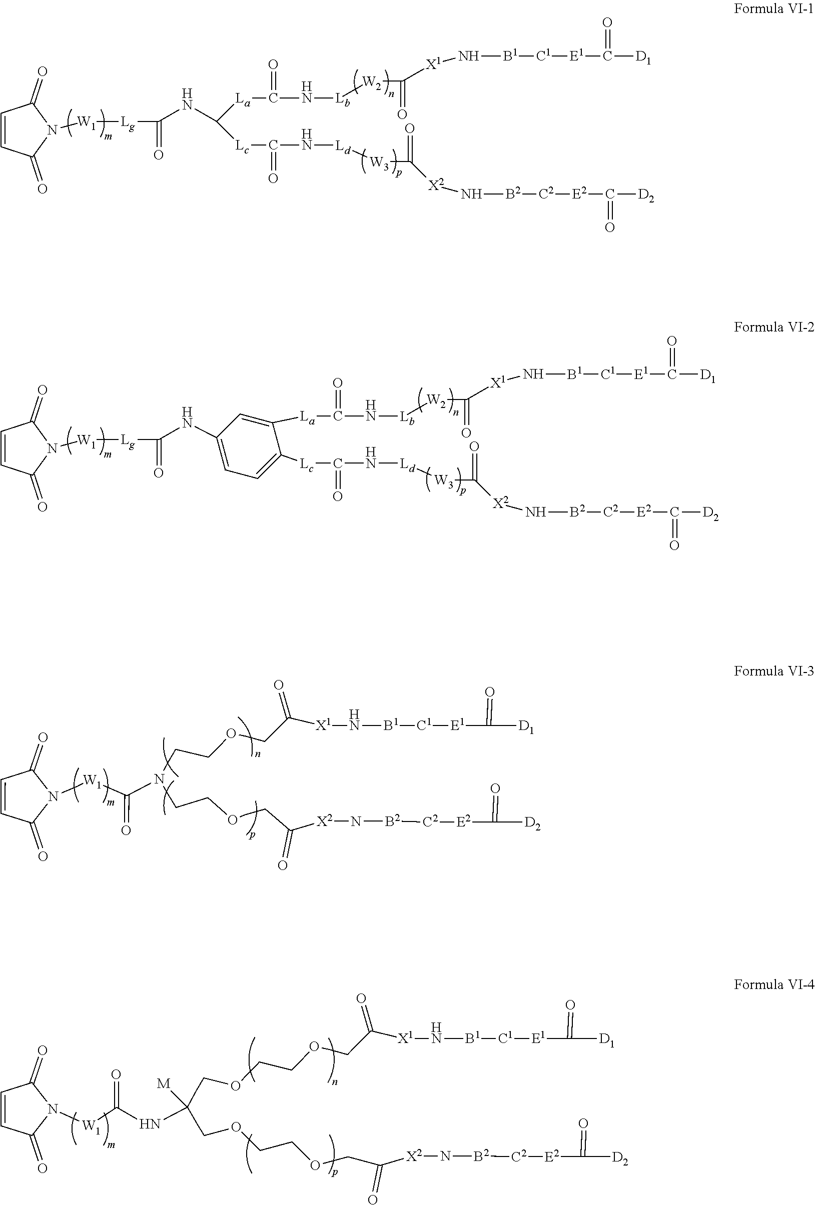
wherein, m, n, and p are each independently selected from an integer of 0 to 30;
W₁, W₂, and W₃ are each independently selected from the group consisting of methylene, 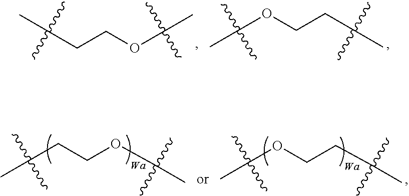
Wa is selected from an integer of 2 to 3; and at least one of W₂ and W₃ is 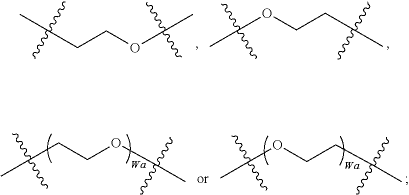
M is selected from the group consisting of halogen, cyano, hydroxyl, C₁₋₆ alkyl or C₁₋₆ alkoxy;
Lₐ, L_{b}, L_{c}, L_{d}, and L_{g} are each independently selected from 0-2 methylenes;
X¹, X², B¹, B², C¹, C², E¹, E², D₁, and D₂ are as defined in claim 5.

7. The dual-drug link assembly unit according to claim 6, or a stereoisomer thereof, or an optical isomer thereof, or a deuterated compound thereof, characterized in that the structure of said dual-drug link assembly unit is selected from one of the following structures: 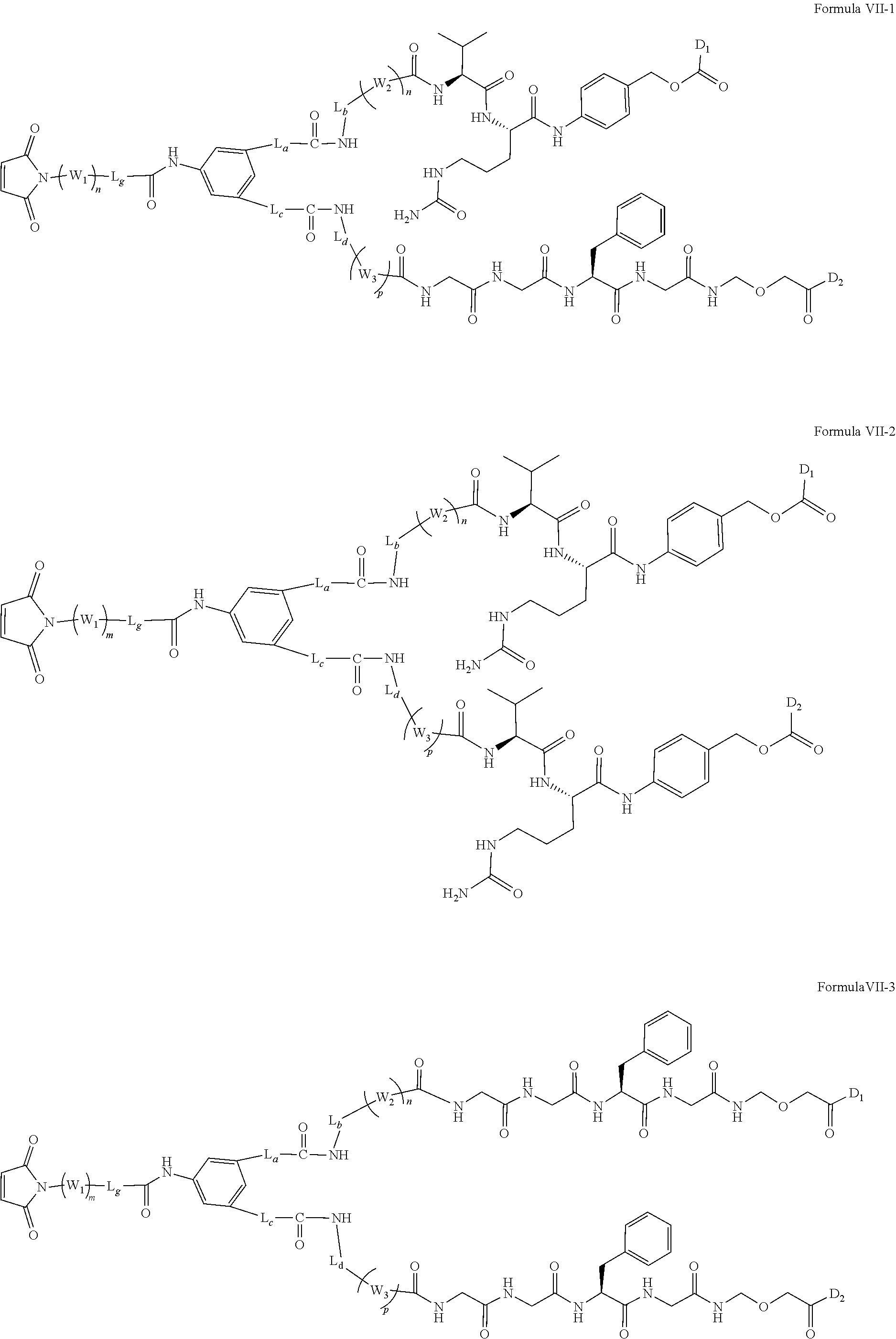 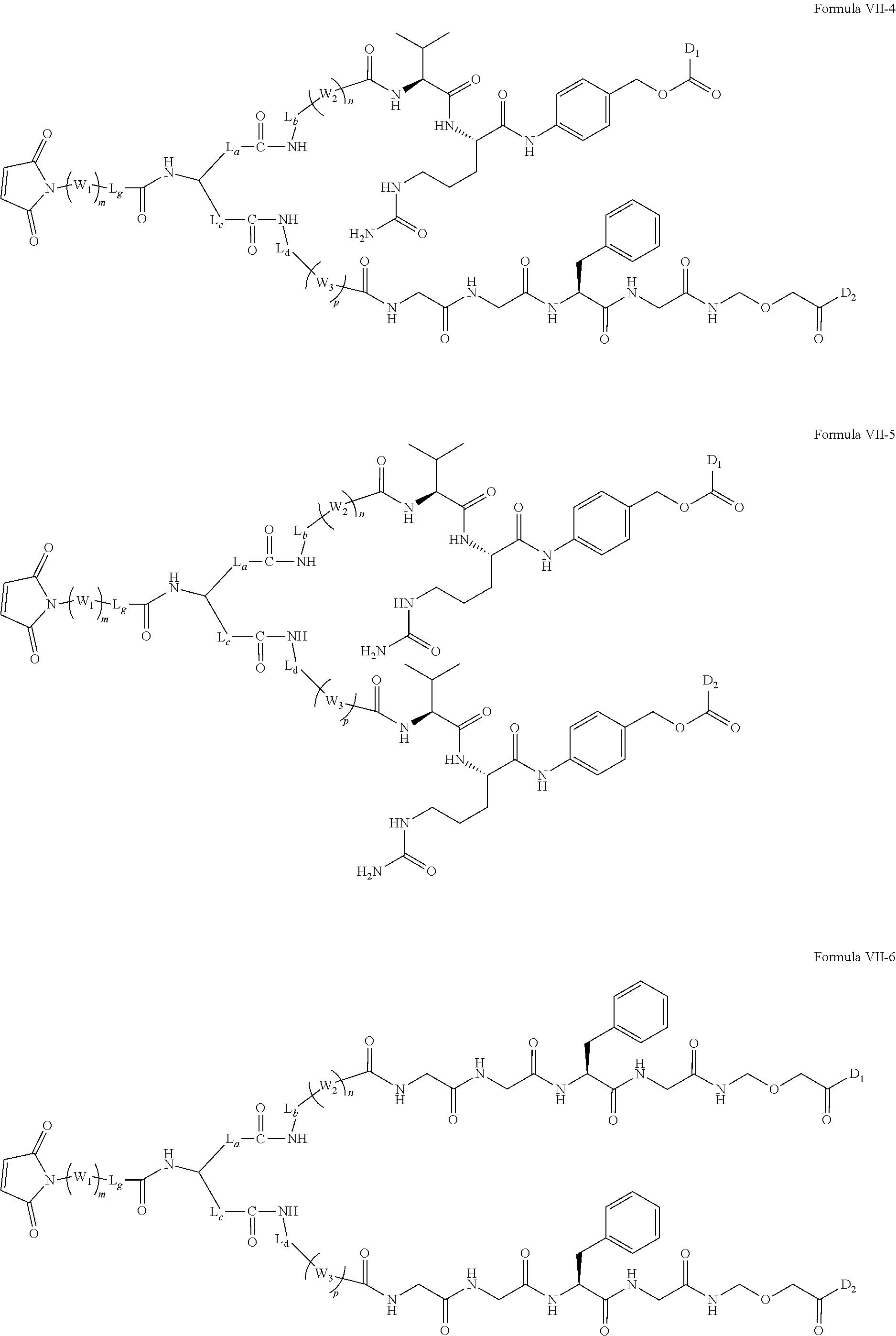 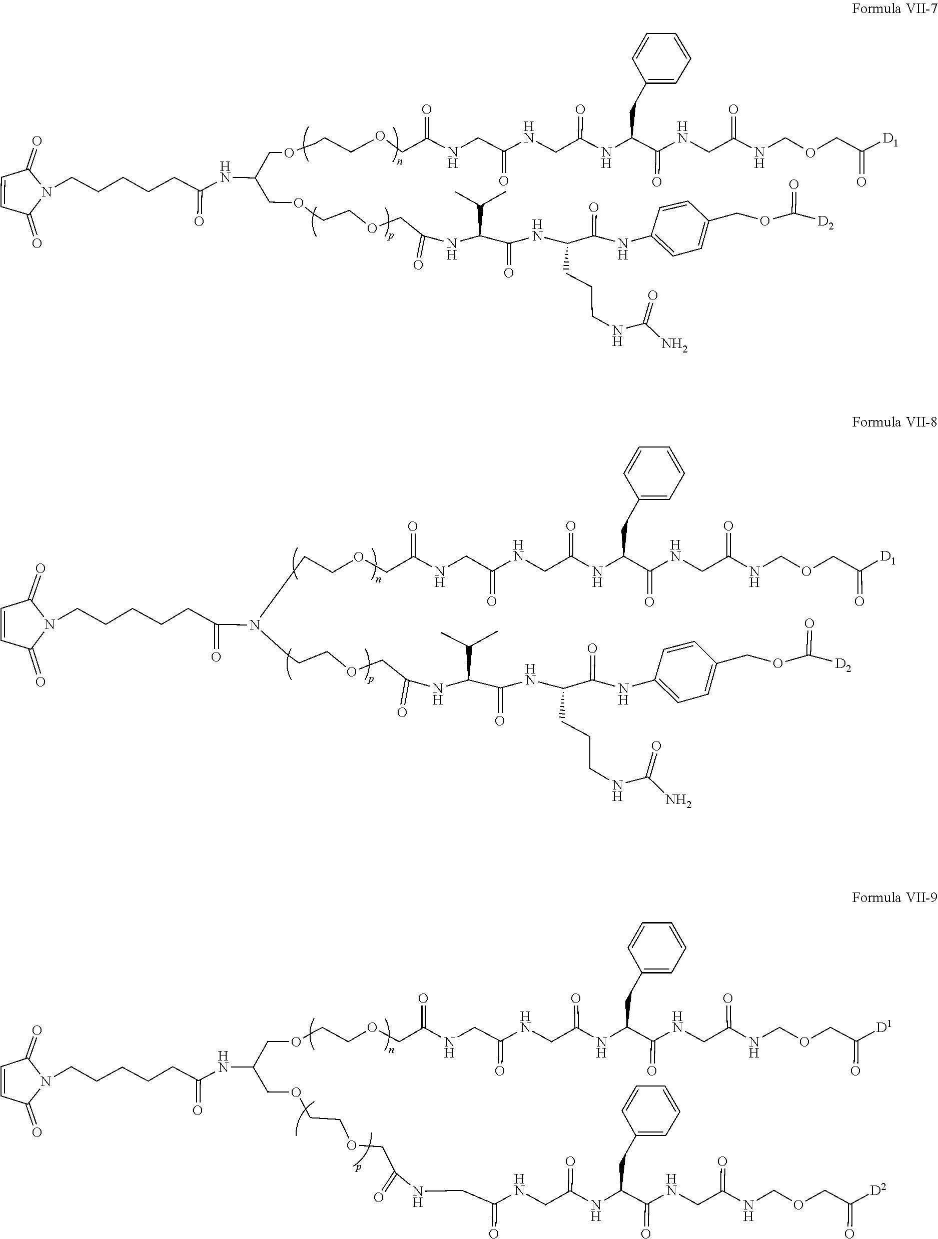 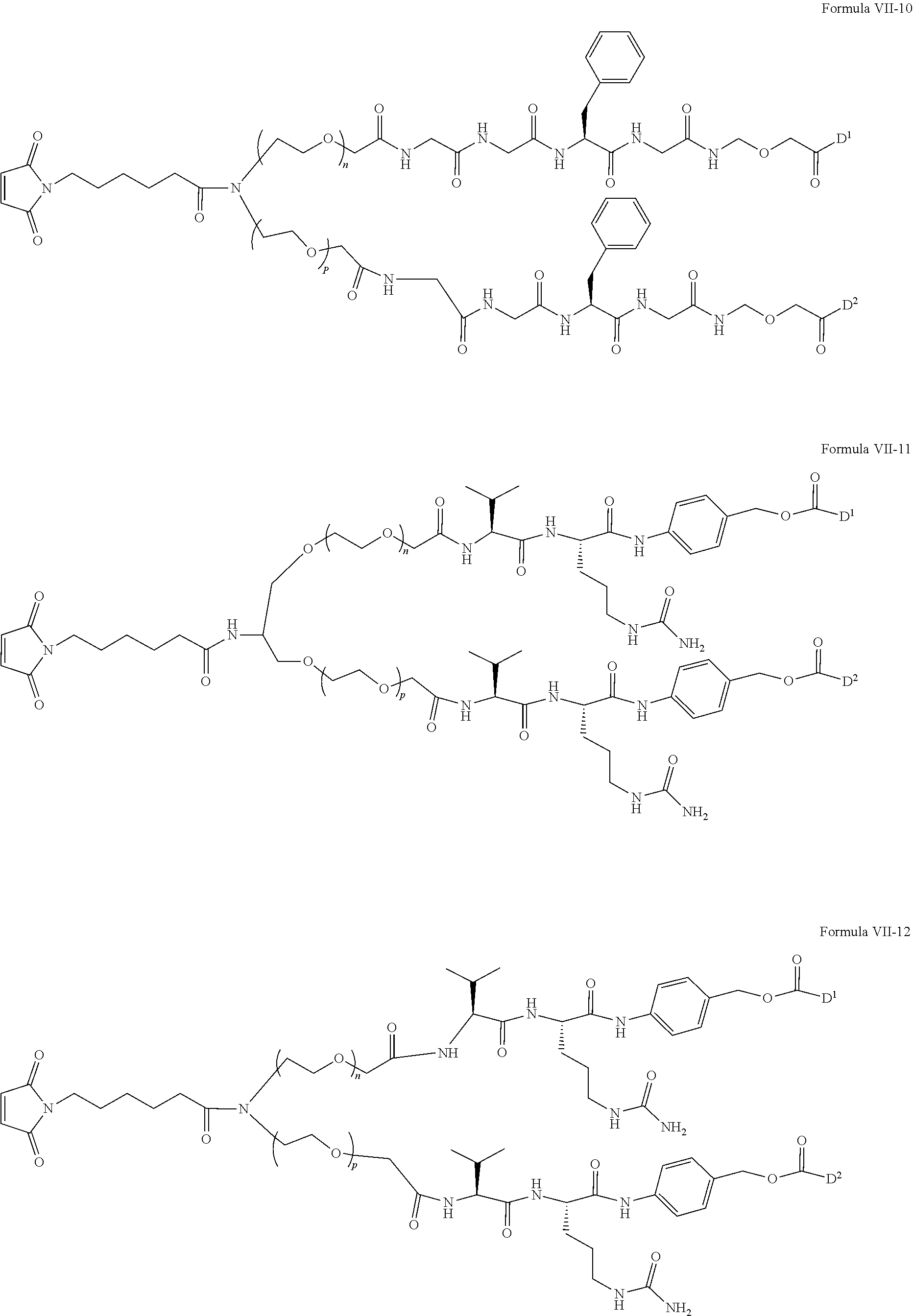
wherein, m is selected from an integer of 0-8;
n and p are each independently selected from an integer of 0-20, as well as n and p are not both 0;
W₁, W₂, and W₃ are each independently selected from the group consisting of methylene, 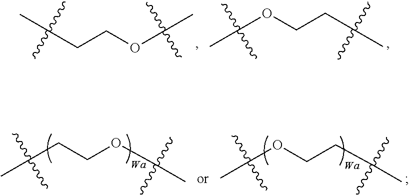
Wa is selected from an integer of 2-3; and at least one of W₂ and W₃ 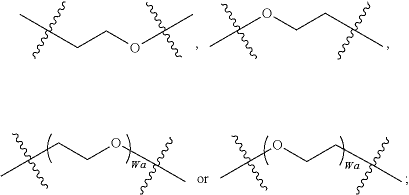
Lₐ, L_{b}, L_{c}, L_{d}, and L_{g} are each independently selected from the group consisting of absence, methylene or ethylene;
D₁ and D₂ are as defined in claim 6.

8. The dual-drug link assembly unit according to claim 7, or a stereoisomer thereof, or an optical isomer thereof, or a deuterated compound thereof, characterized in that the structure of said dual-drug link assembly unit is one of the following structures: 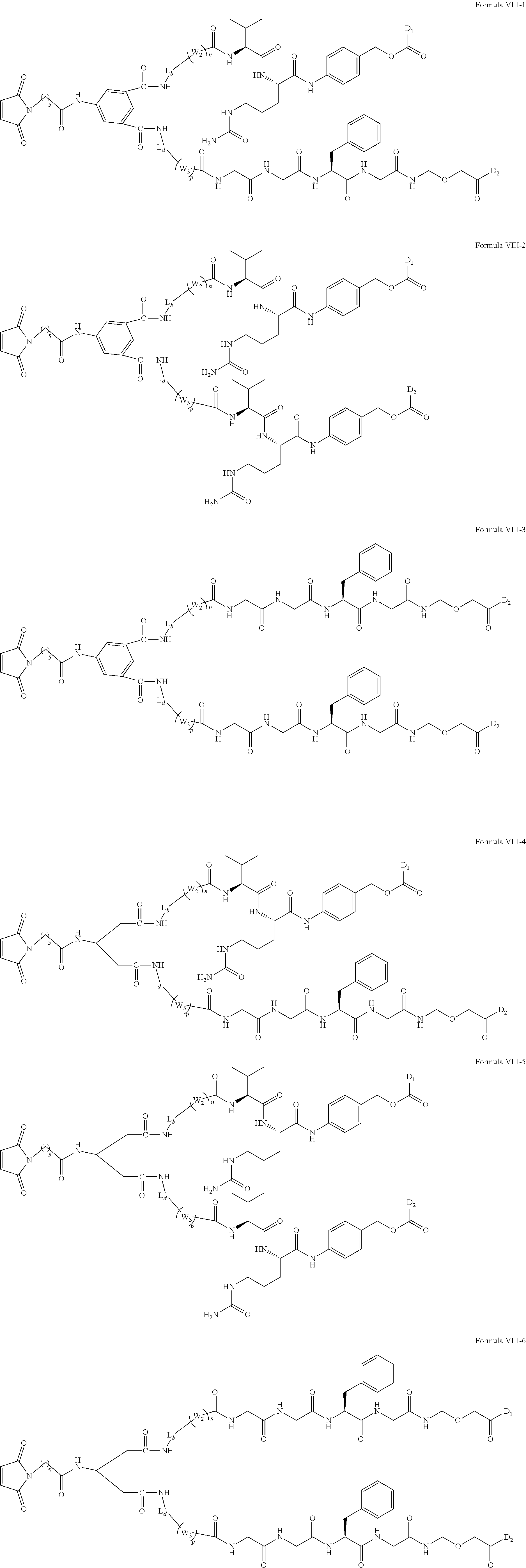 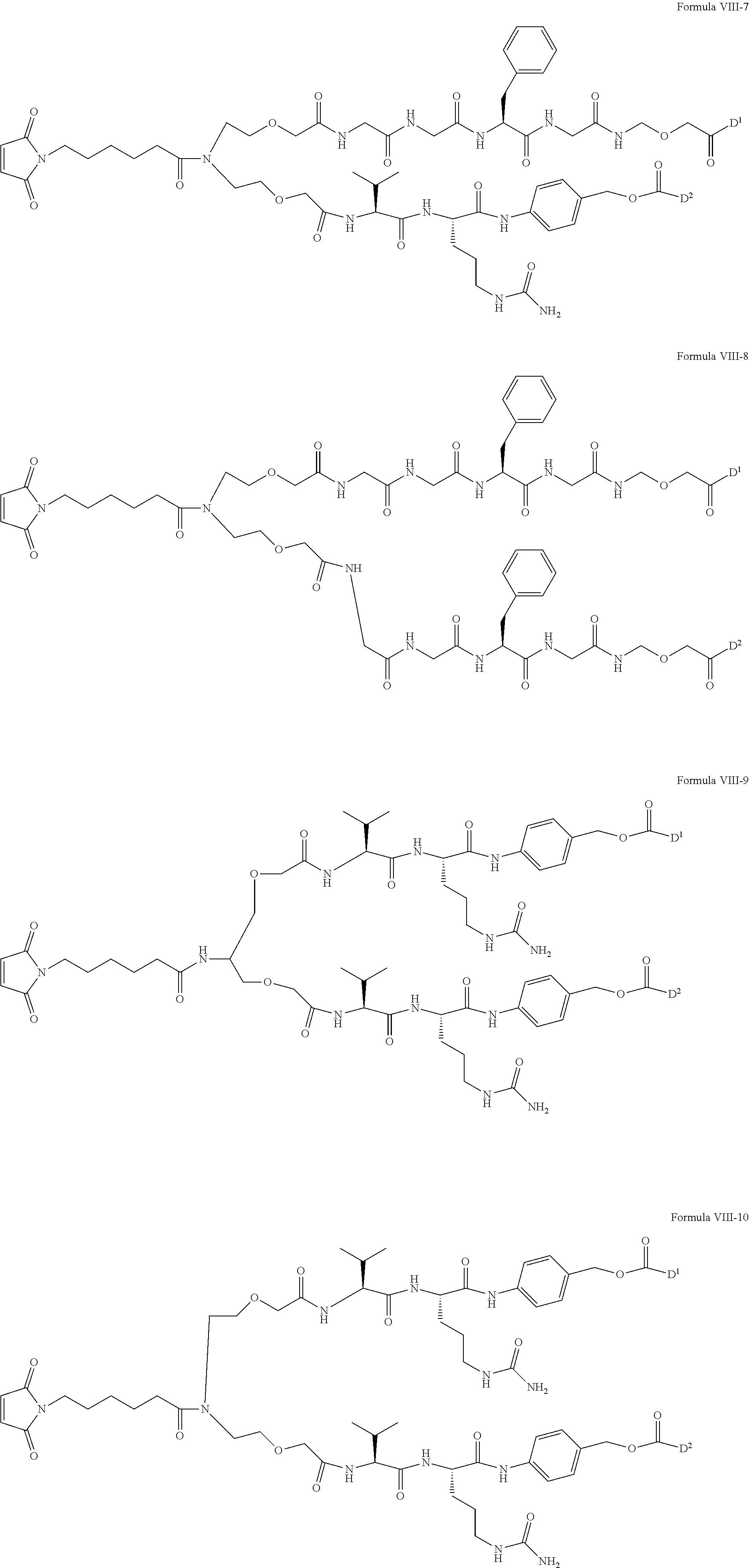
wherein, n and p are each independently selected from an integer of 0-20, and n and p are not both 0; W₂ and W₃ are each independently selected from the group consisting of methylene, 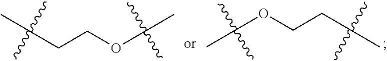 [SMILES: *CCO* ; *CCO*]
and at least one of W₂ and W₃ is 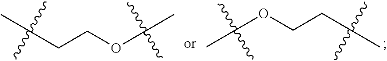 [SMILES: *CCO* ; *CCO*]
L_{b} and L_{d} are each independently selected from absence or ethylene;
D₁ and D₂ are as defined in claim 7.

9. The dual-drug link assembly unit according to claim 1, or a stereoisomer thereof, or an optical isomer thereof, or a deuterated compound thereof, characterized in that said D₁ and D₂ are each independently selected from the drug unit targeting TOP isomerase or the drug unit targeting microtubule proteins; the drug units targeting TOP isomerase are preferably SN-38, DXd, DX-8951 or derivatives thereof, and/or, and the drug units targeting microtubule proteins are preferably Eribulin, MMAE, MMAF, maytansine or derivatives thereof.

10. The dual-drug link assembly unit according to claim 1, or a stereoisomer thereof, or an optical isomer thereof, or a deuterated compound thereof, characterized in that the structure of said dual-drug link assembly unit is one of the following structures:

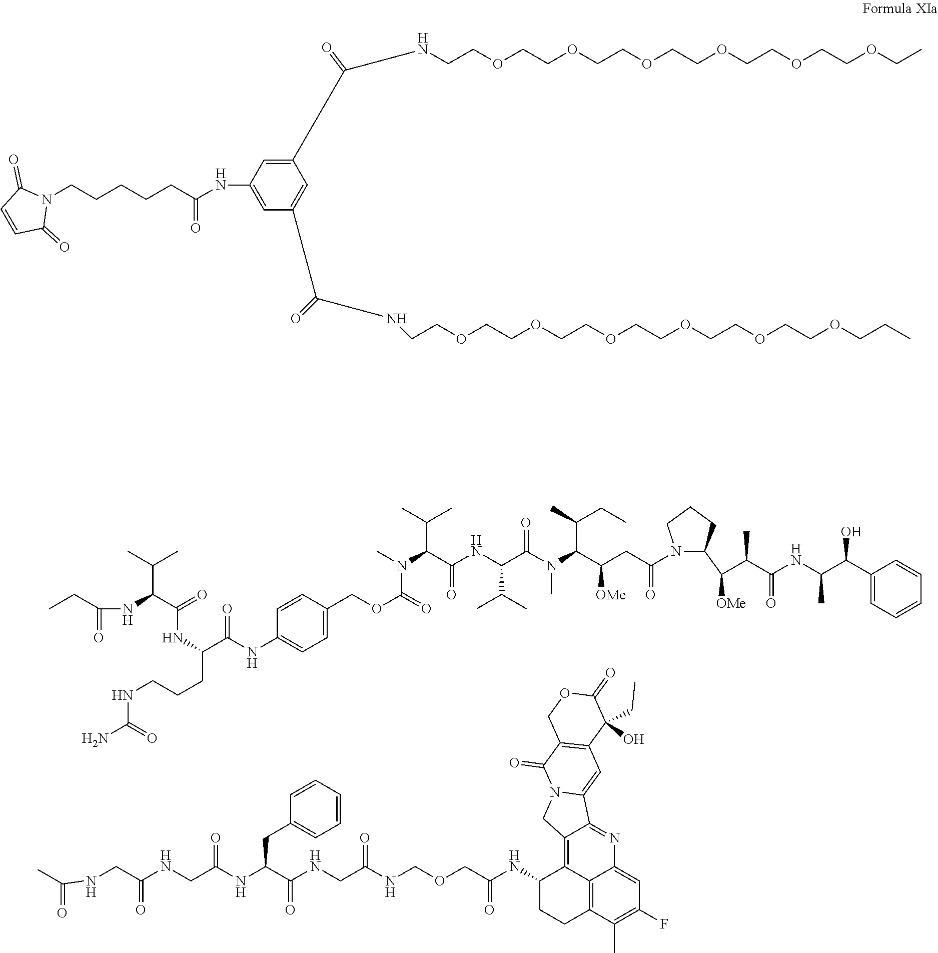

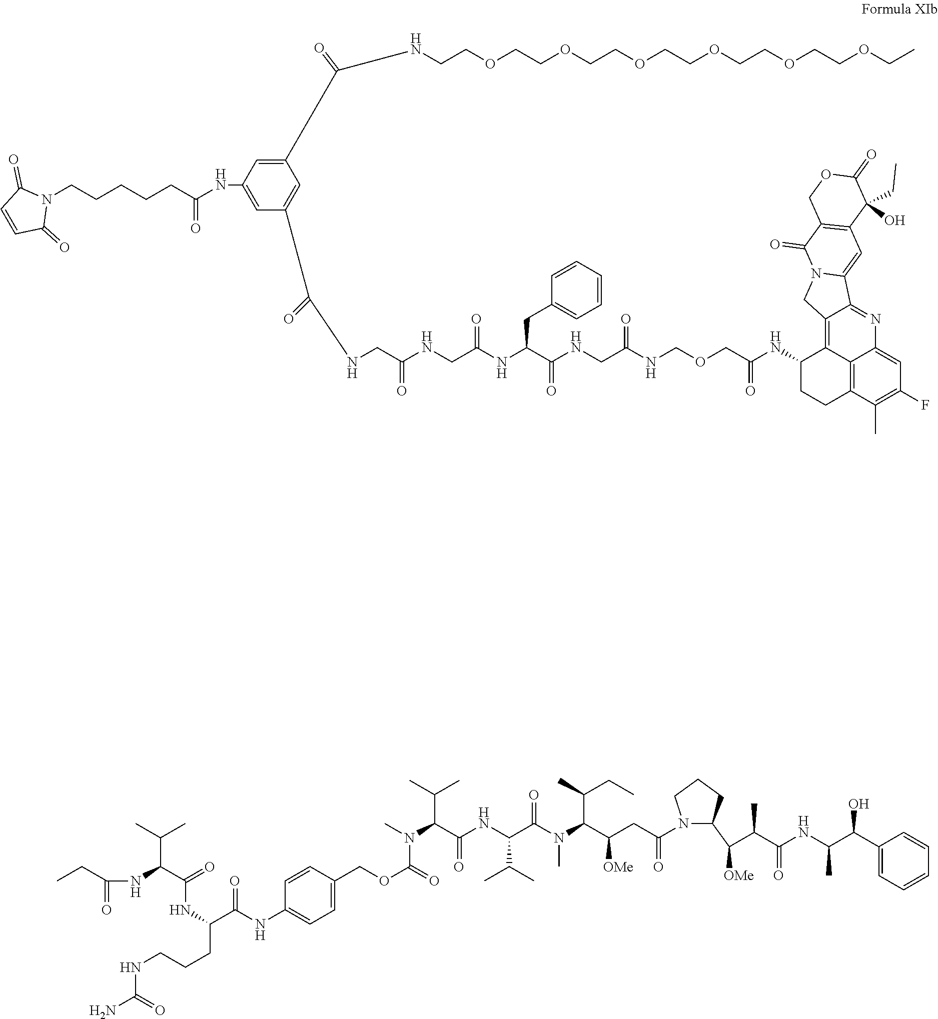 [SMILES: CCOCCOCCOCCOCCOCCOCCNC(=O)c1cc(NC(=O)CCCCCN2C(=O)C=CC2=O)cc(C(=O)NCC(=O)NCC(=O)N[C@@H](Cc2ccccc2)C(=O)NCC(=O)NCOCC(=O)N[C@H]2CCc3c(C)c(F)cc4nc5c(c2c34)Cn2c-5cc3c(c2=O)COC(=O)[C@]3(O)CC)c1 ; CCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C]

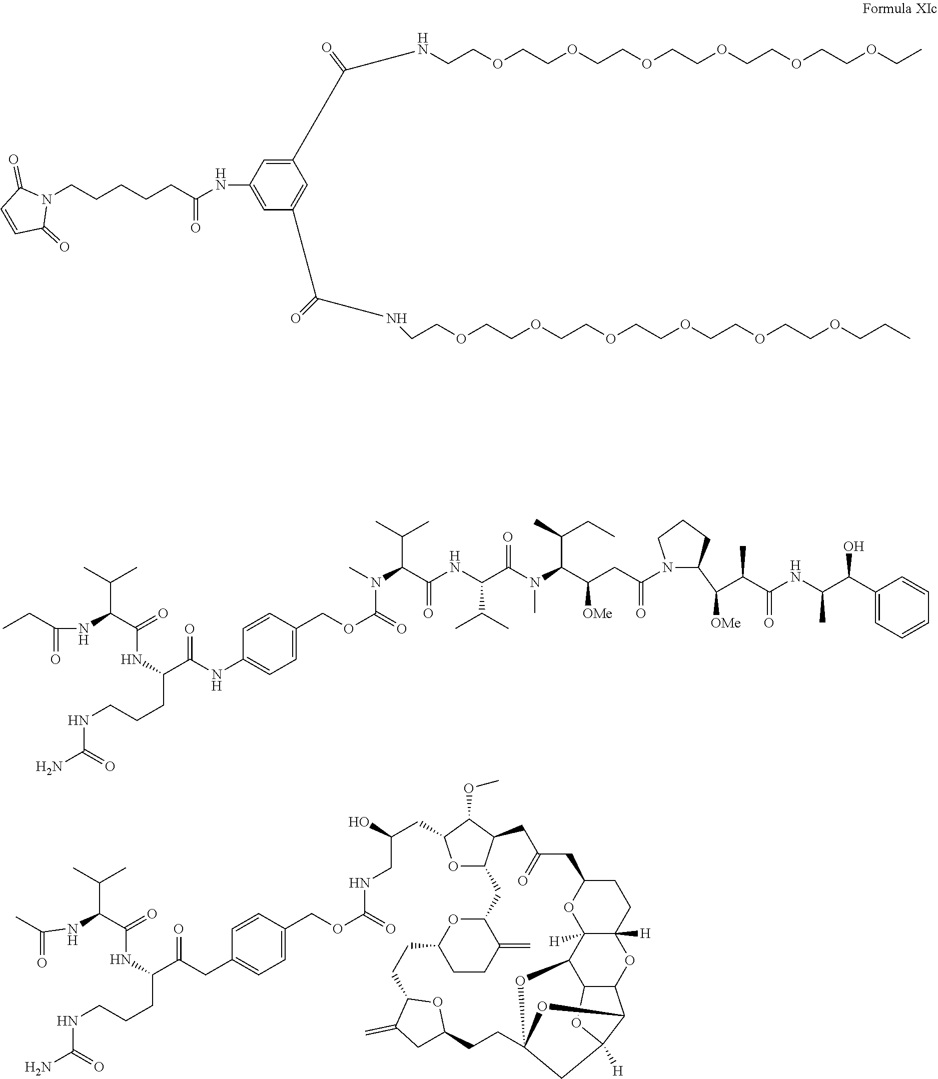

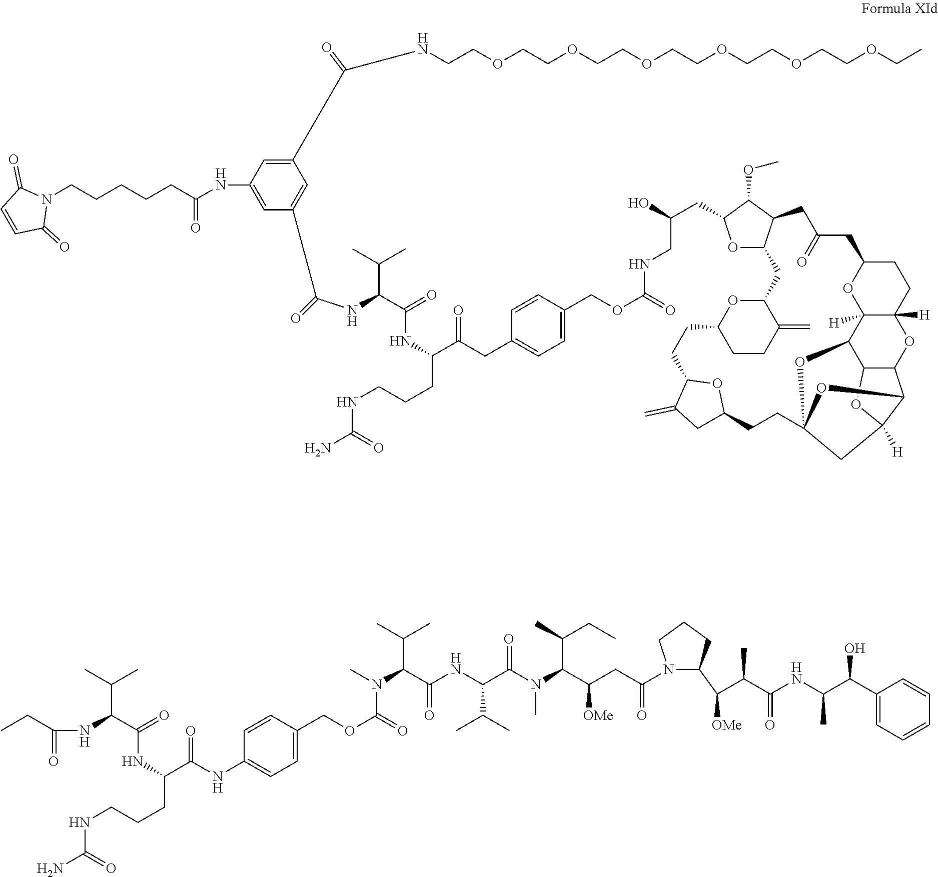

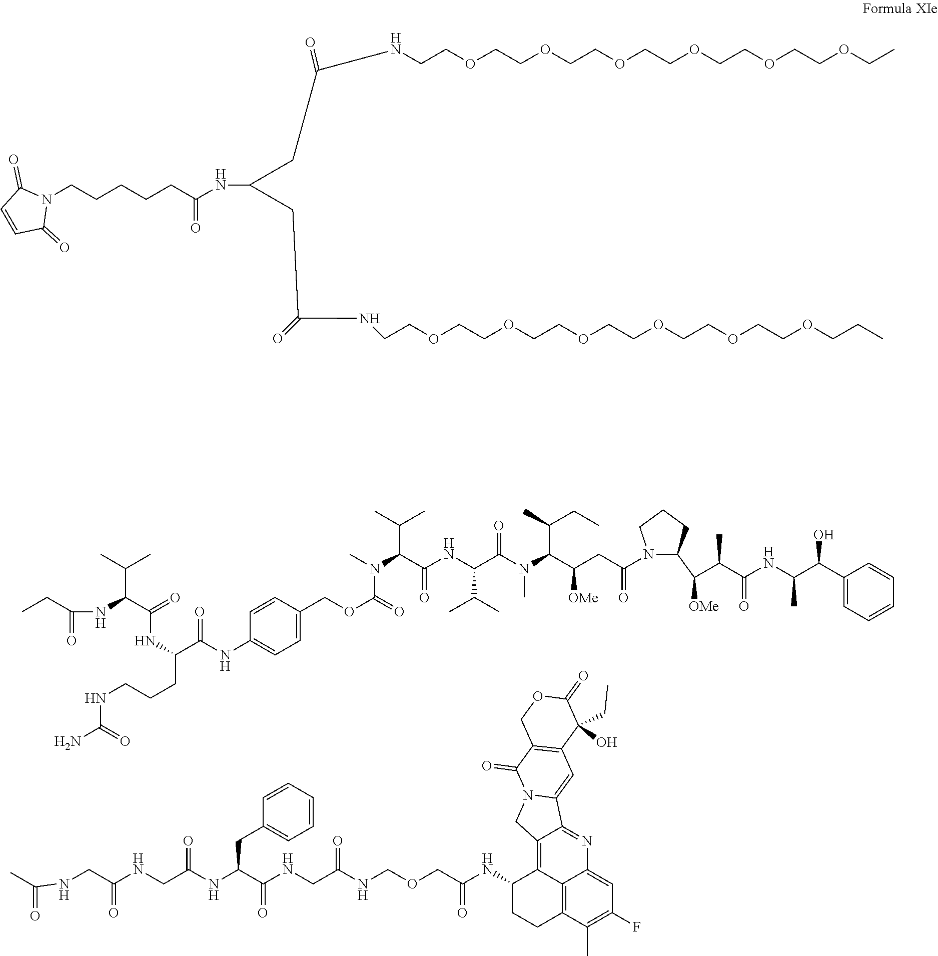

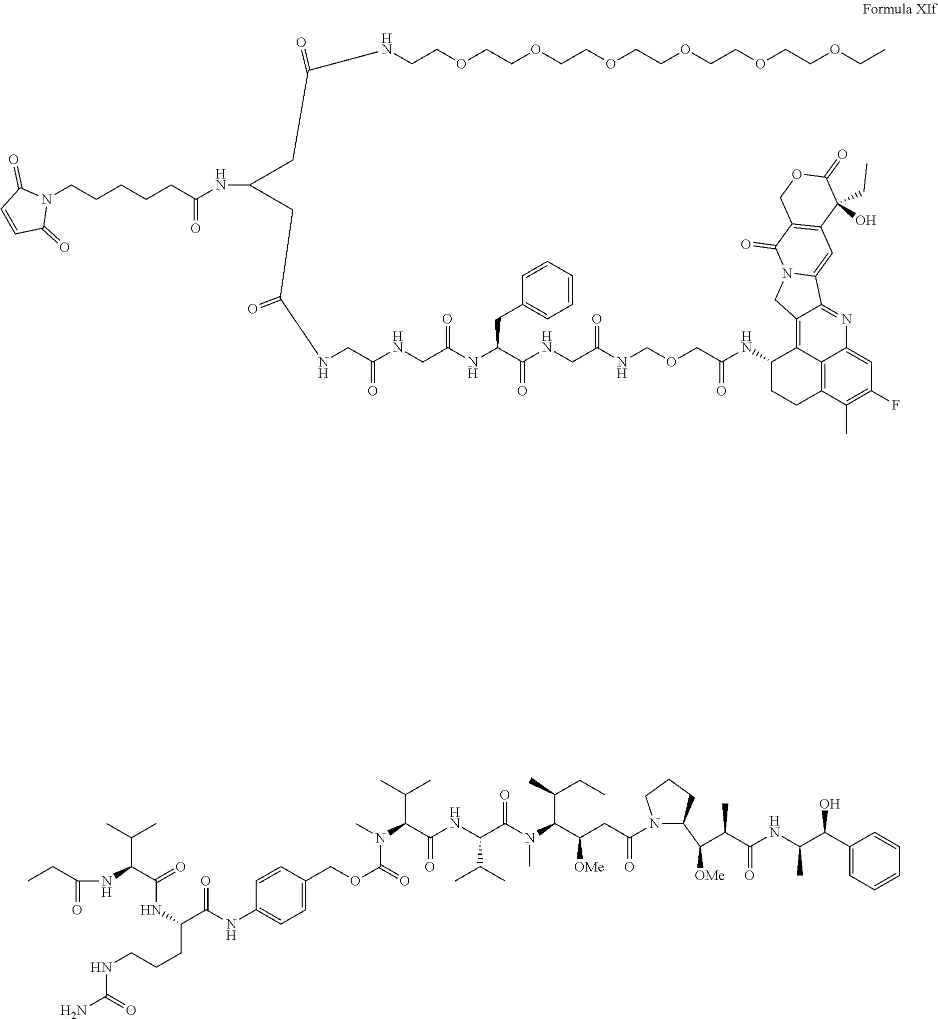 [SMILES: CCOCCOCCOCCOCCOCCOCCNC(=O)CC(CC(=O)NCC(=O)NCC(=O)N[C@@H](Cc1ccccc1)C(=O)NCC(=O)NCOCC(=O)N[C@H]1CCc2c(C)c(F)cc3nc4c(c1c23)Cn1c-4cc2c(c1=O)COC(=O)[C@]2(O)CC)NC(=O)CCCCCN1C(=O)C=CC1=O ; CCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C]

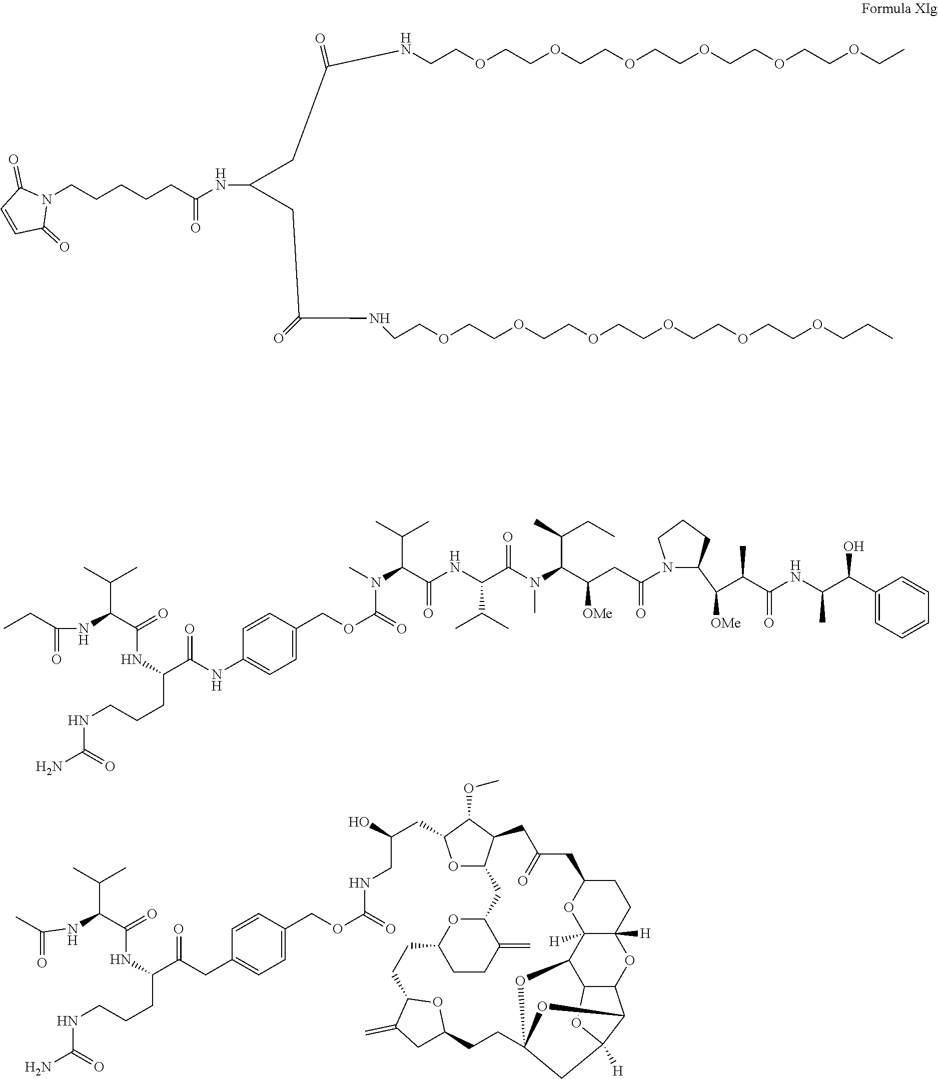

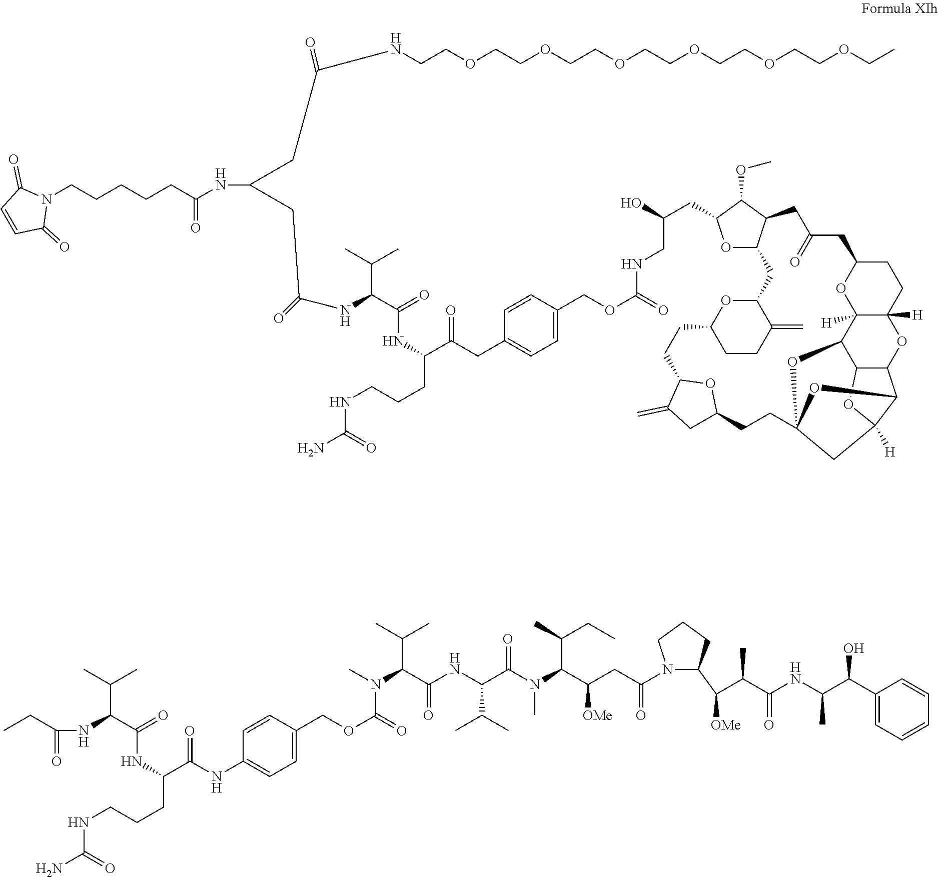 [SMILES: C=C1C[C@@H]2CC[C@@]34C[C@H]5OC6C(O[C@H]7CC[C@H](CC(=O)C[C@@H]8[C@@H](OC)[C@@H](C[C@H](O)CNC(=O)OCc9ccc(CC(=O)[C@H](CCCNC(N)=O)NC(=O)[C@@H](NC(=O)CC(CC(=O)NCCOCCOCCOCCOCCOCCOCC)NC(=O)CCCCCN%10C(=O)C=CC%10=O)C(C)C)cc9)O[C@H]8C[C@H]8O[C@H](CCC8=C)CC[C@@H]1O2)O[C@@H]7[C@@H]6O3)[C@H]5O4 ; CCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C]

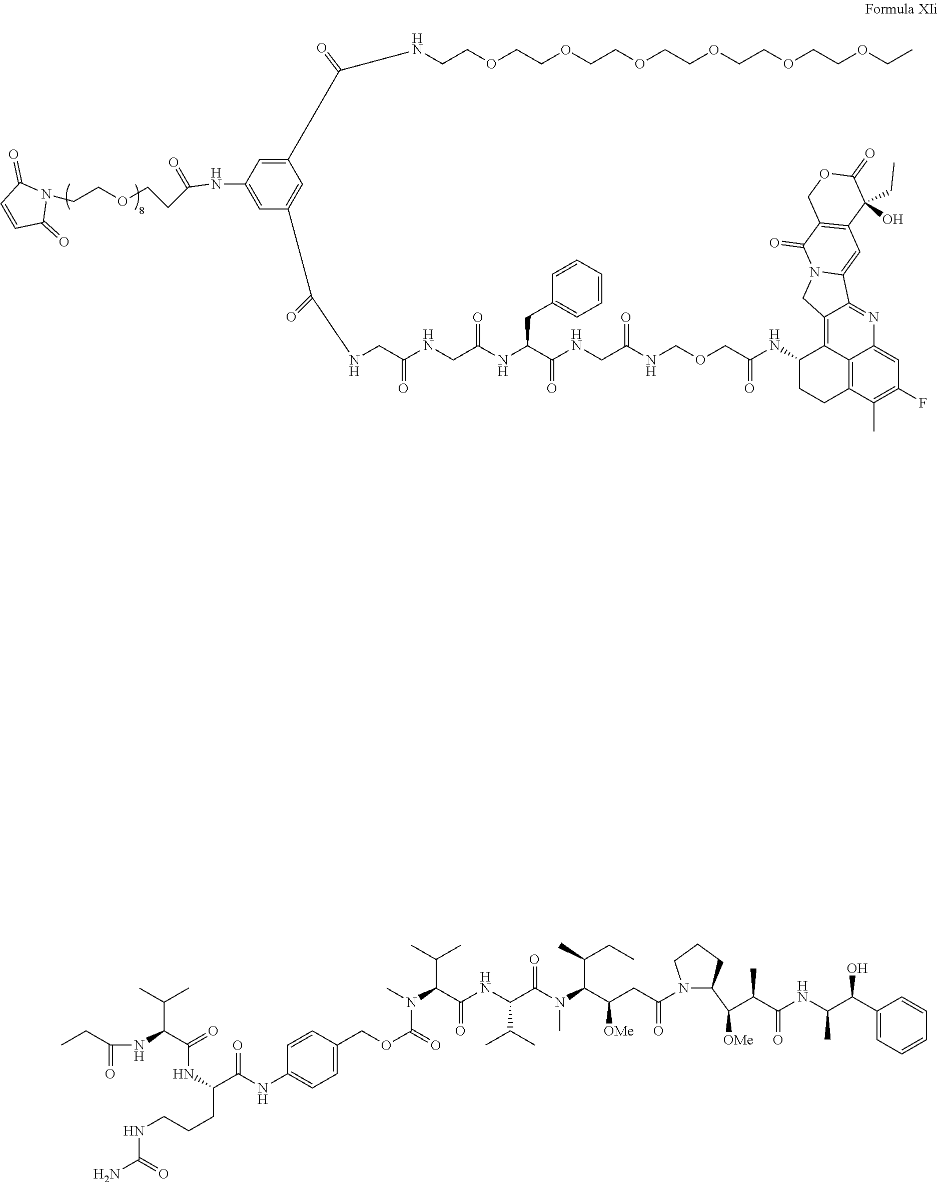

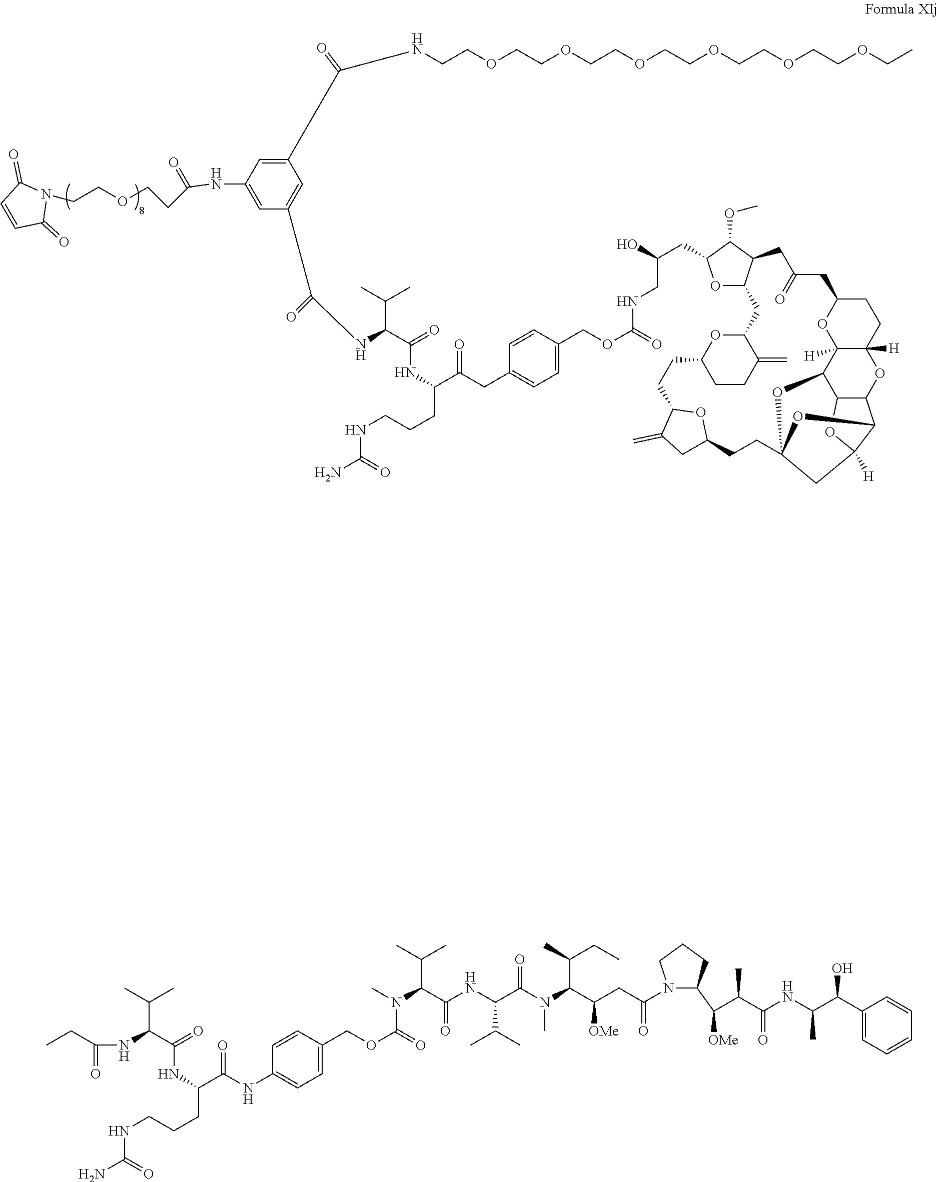

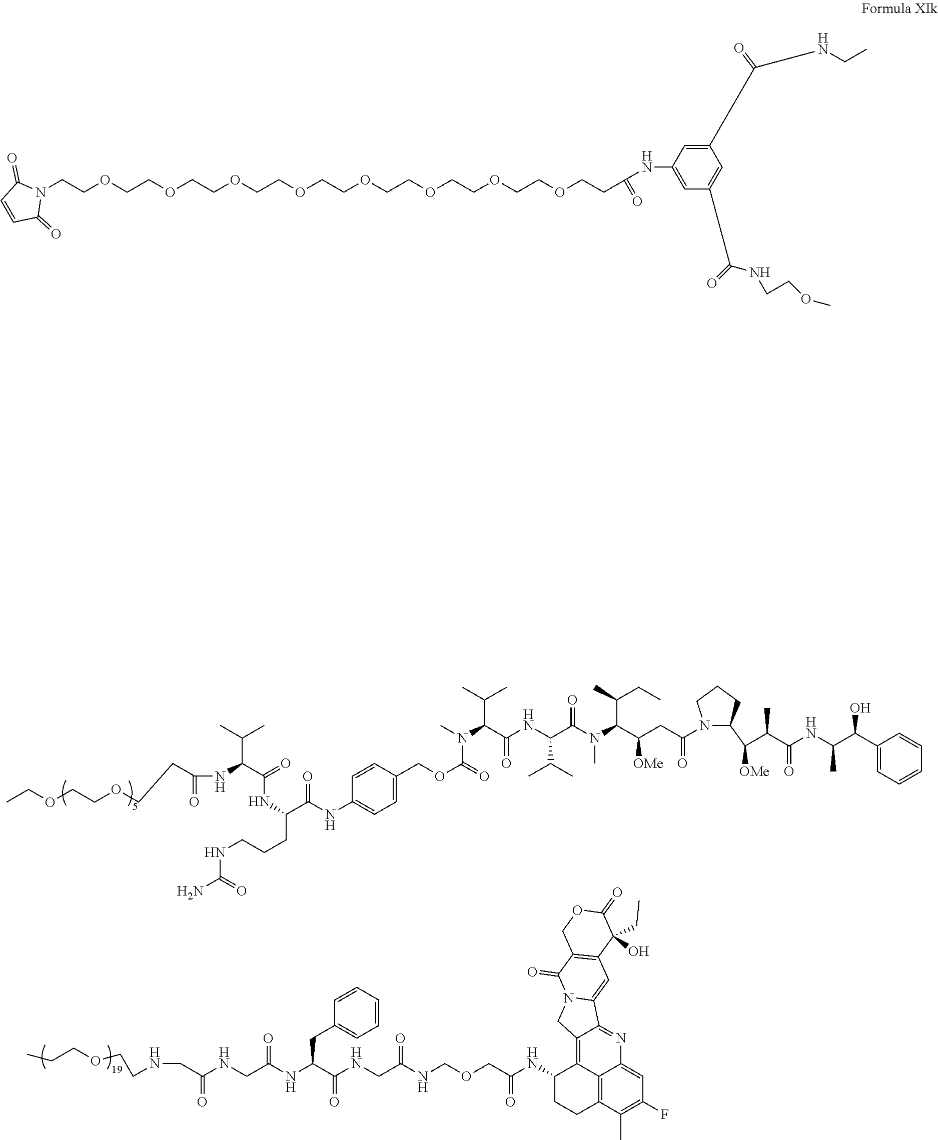

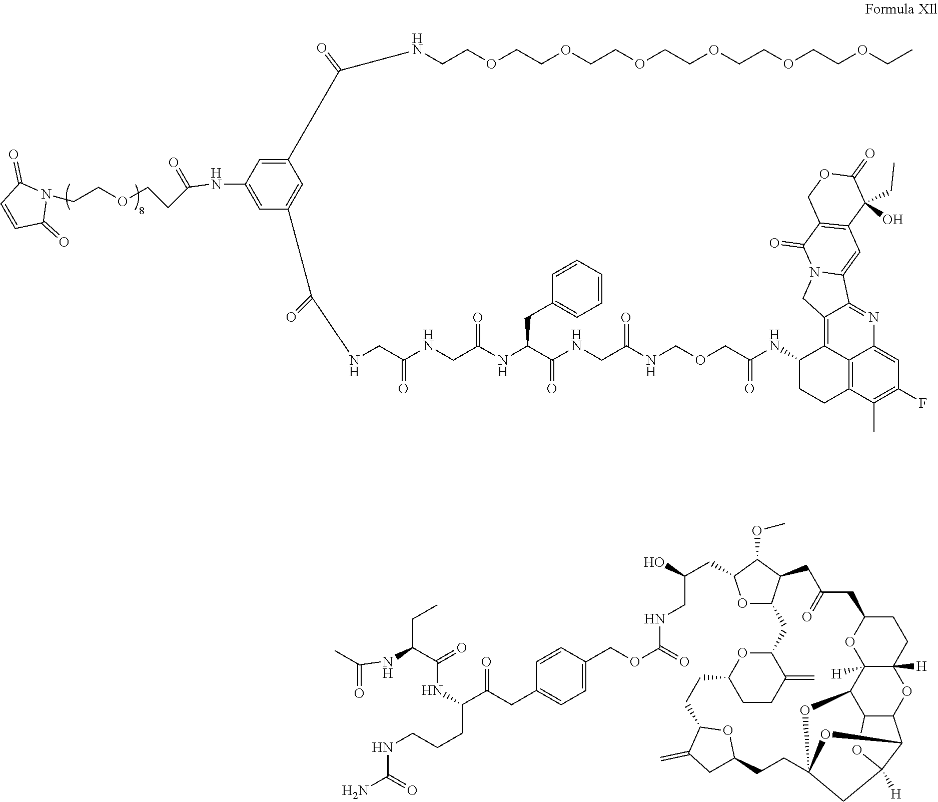

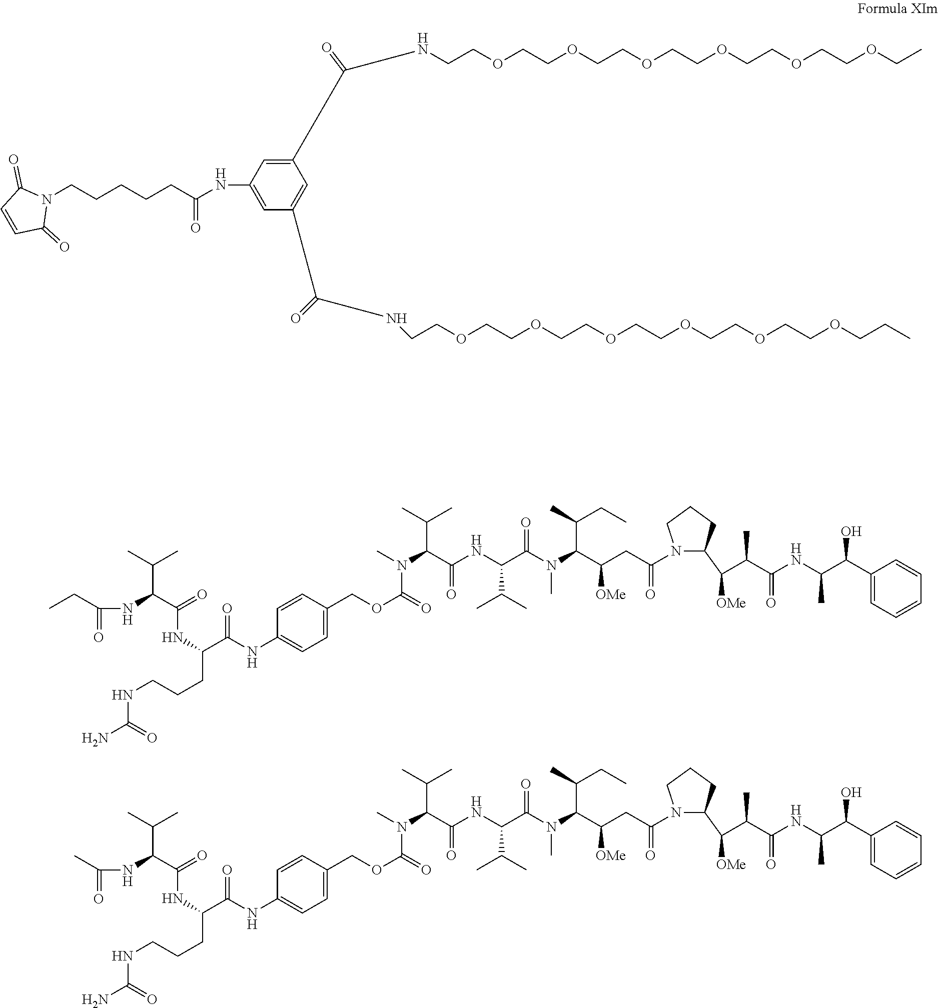

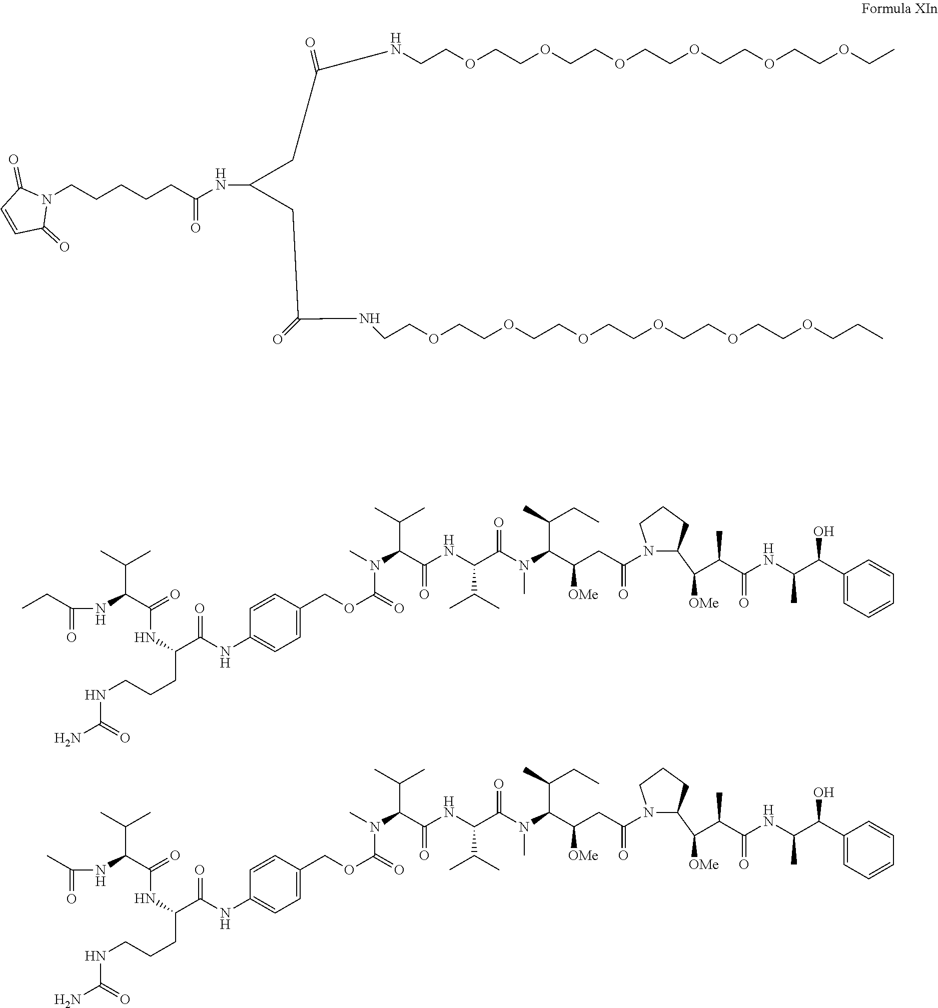

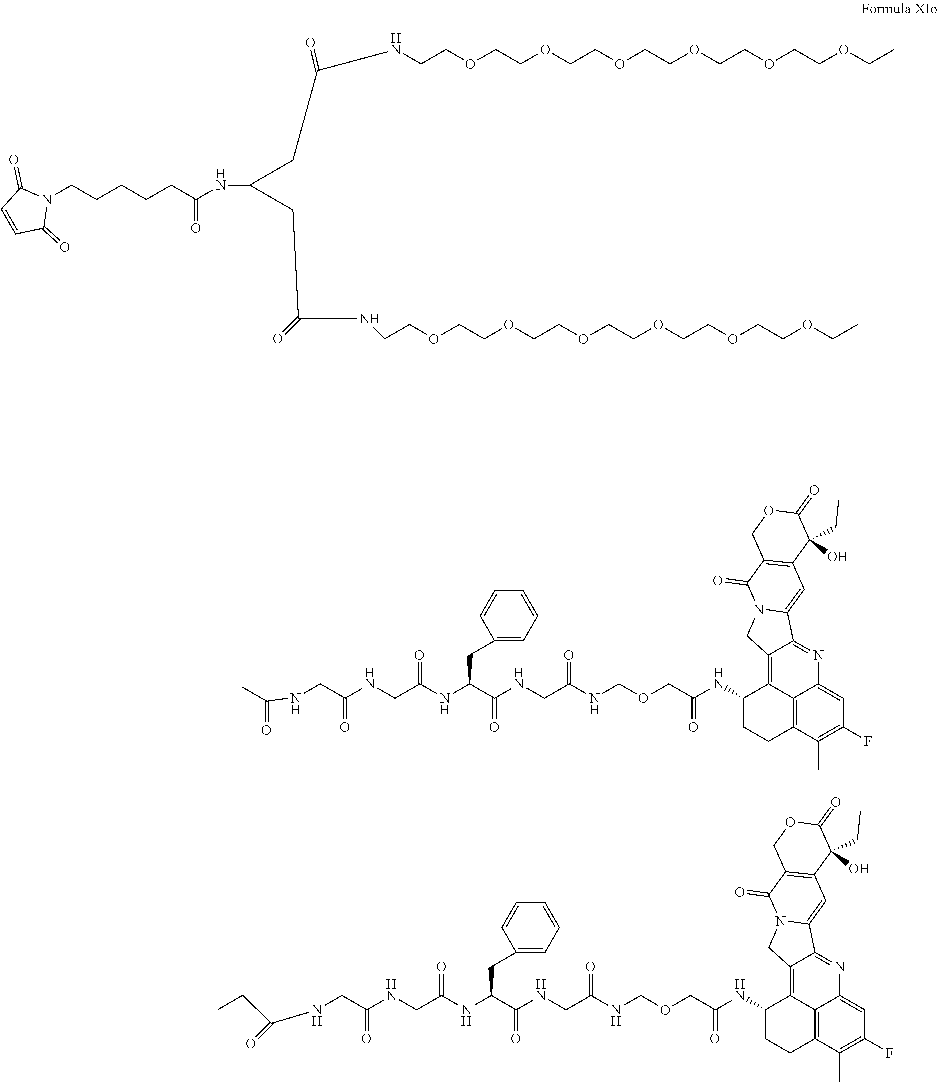

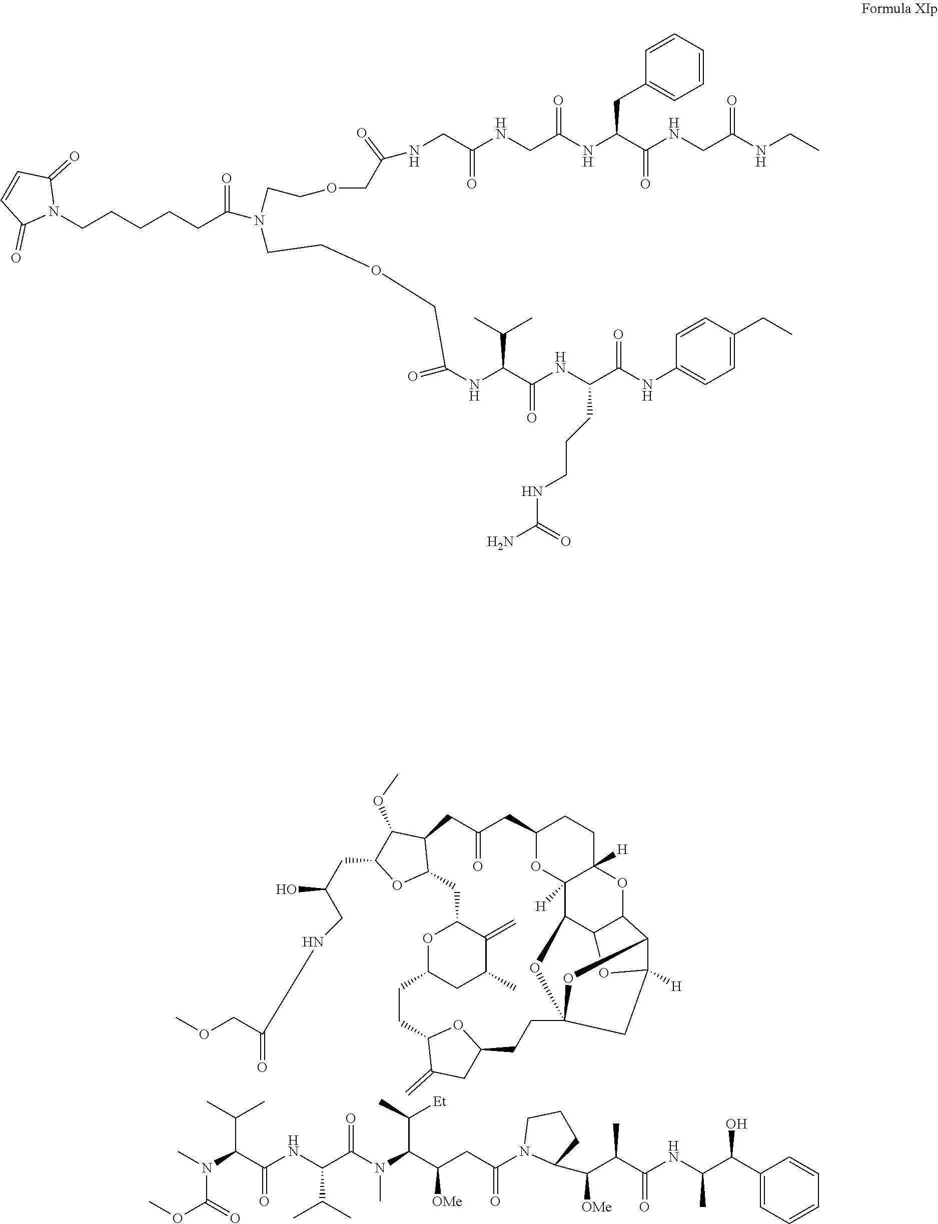

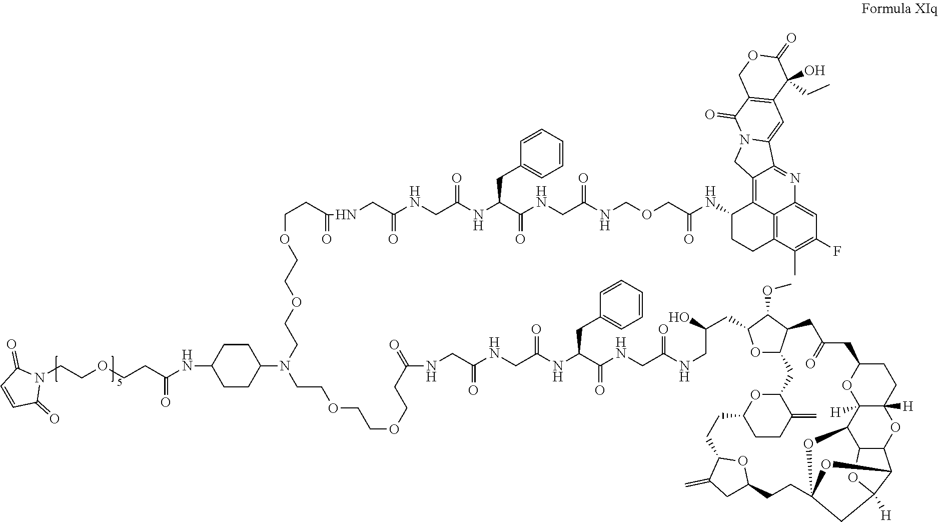

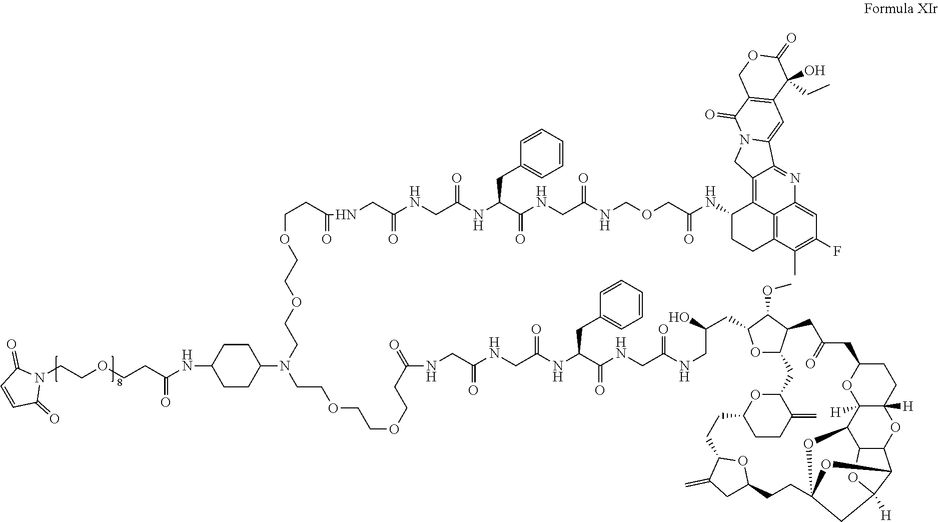

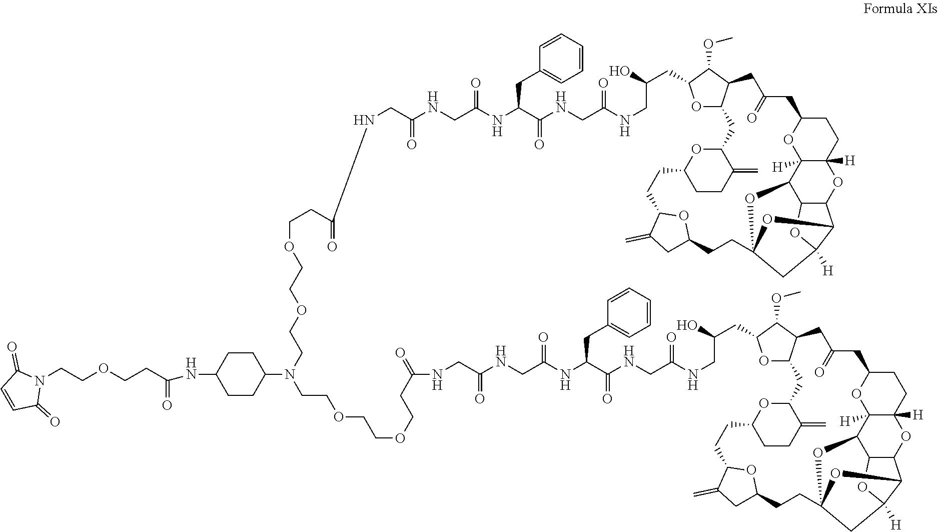 [SMILES: C=C1C[C@@H]2CC[C@@]34C[C@H]5OC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)CCOCCOCCN(CCOCCOCCC(=O)NCC(=O)NCC(=O)N[C@@H](Cc9ccccc9)C(=O)NCC(=O)NC[C@@H](O)C[C@H]9O[C@H]%10C[C@H]%11O[C@H](CCC%11=C)CC[C@@H]%11O[C@@H](CC[C@@]%12%13C[C@H]%14OC%15C(O[C@H]%16CC[C@H](CC(=O)C[C@@H]%10[C@H]9OC)O[C@@H]%16[C@@H]%15O%12)[C@H]%14O%13)CC%11=C)C9CCC(NC(=O)CCOCCN%10C(=O)C=CC%10=O)CC9)O[C@H]8C[C@H]8O[C@H](CCC8=C)CC[C@@H]1O2)O[C@@H]7[C@@H]6O3)[C@H]5O4]

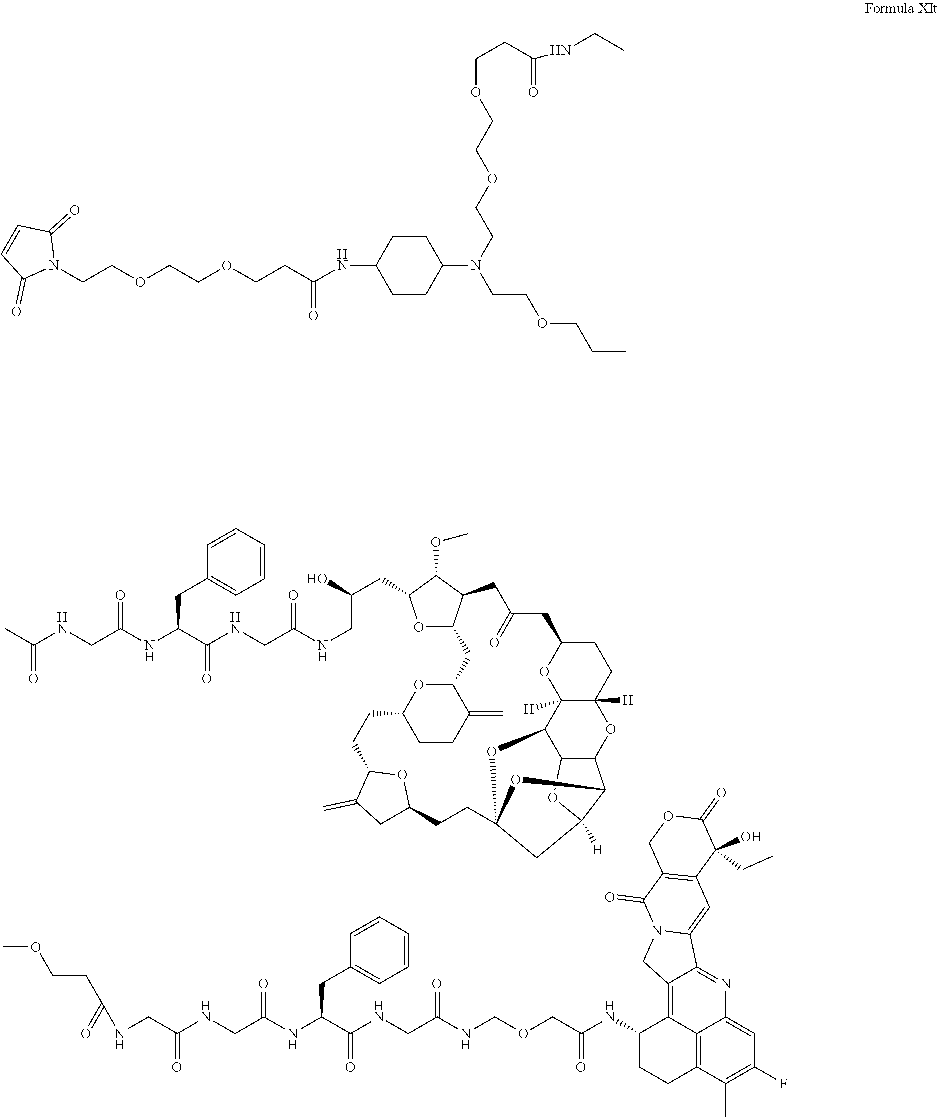

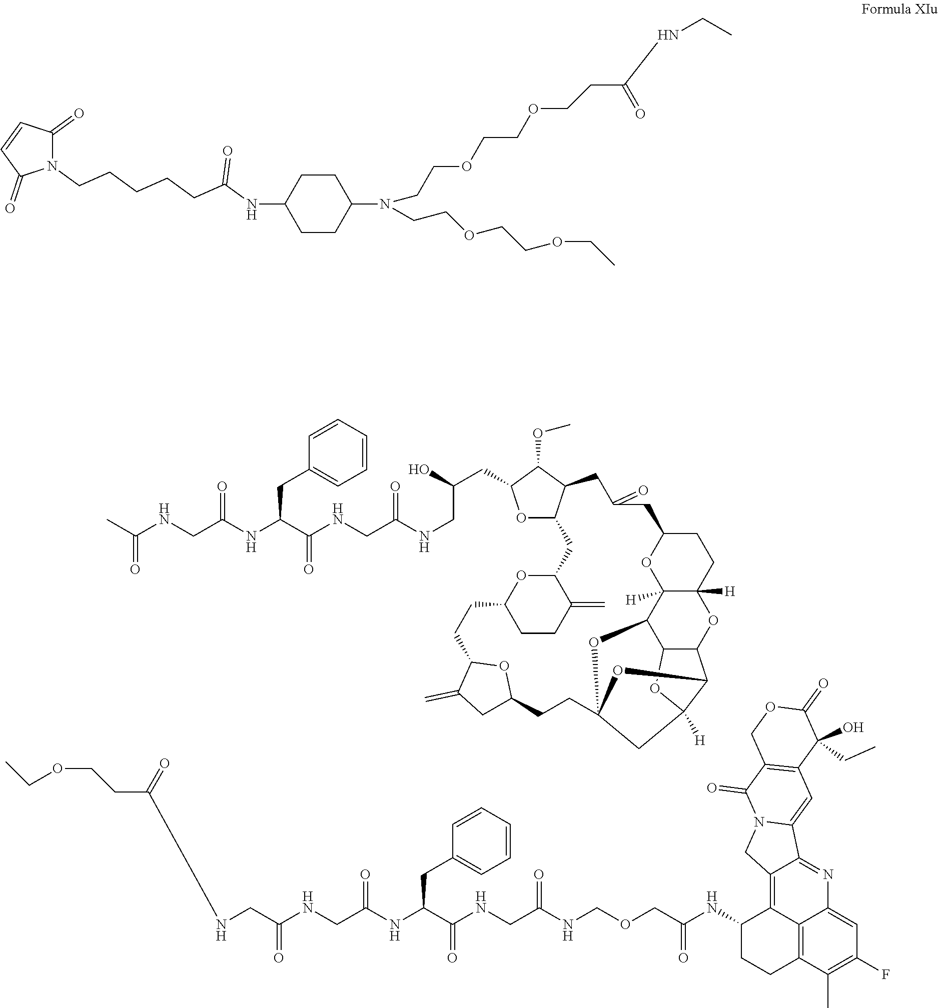

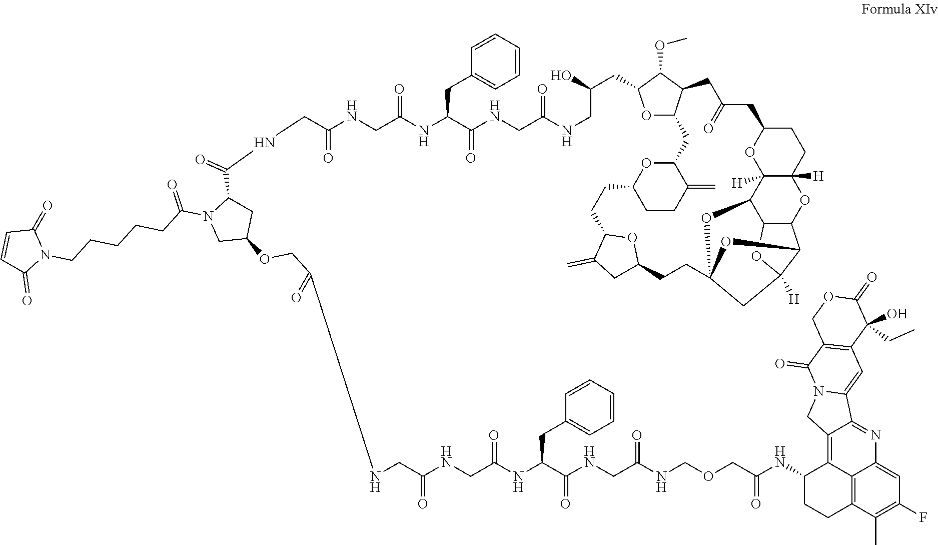 [SMILES: C=C1C[C@@H]2CC[C@@]34C[C@H]5OC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)[C@@H]9C[C@@H](OCC(=O)NCC(=O)NCC(=O)N[C@@H](Cc%10ccccc%10)C(=O)NCC(=O)NCOCC(=O)N[C@H]%10CCc%11c(C)c(F)cc%12nc%13c(c%10c%11%12)Cn%10c-%13cc%11c(c%10=O)COC(=O)[C@]%11(O)CC)CN9C(=O)CCCCCN9C(=O)C=CC9=O)O[C@H]8C[C@H]8O[C@H](CCC8=C)CC[C@@H]1O2)O[C@@H]7[C@@H]6O3)[C@H]5O4]

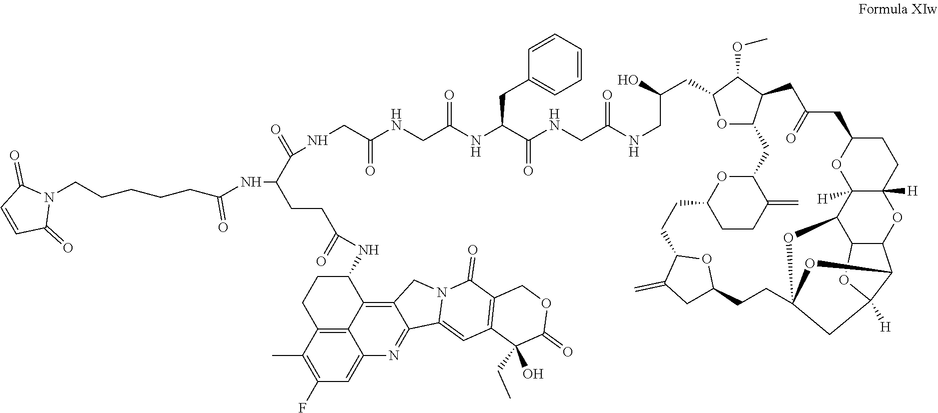 [SMILES: C=C1C[C@@H]2CC[C@@]34C[C@H]5OC6C(O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)C(CCC(=O)N[C@H]9CCc%10c(C)c(F)cc%11nc%12c(c9c%10%11)Cn9c-%12cc%10c(c9=O)COC(=O)[C@]%10(O)CC)NC(=O)CCCCCN9C(=O)C=CC9=O)O[C@H]8C[C@H]8O[C@H](CCC8=C)CC[C@@H]1O2)O[C@@H]7[C@@H]6O3)[C@H]5O4]

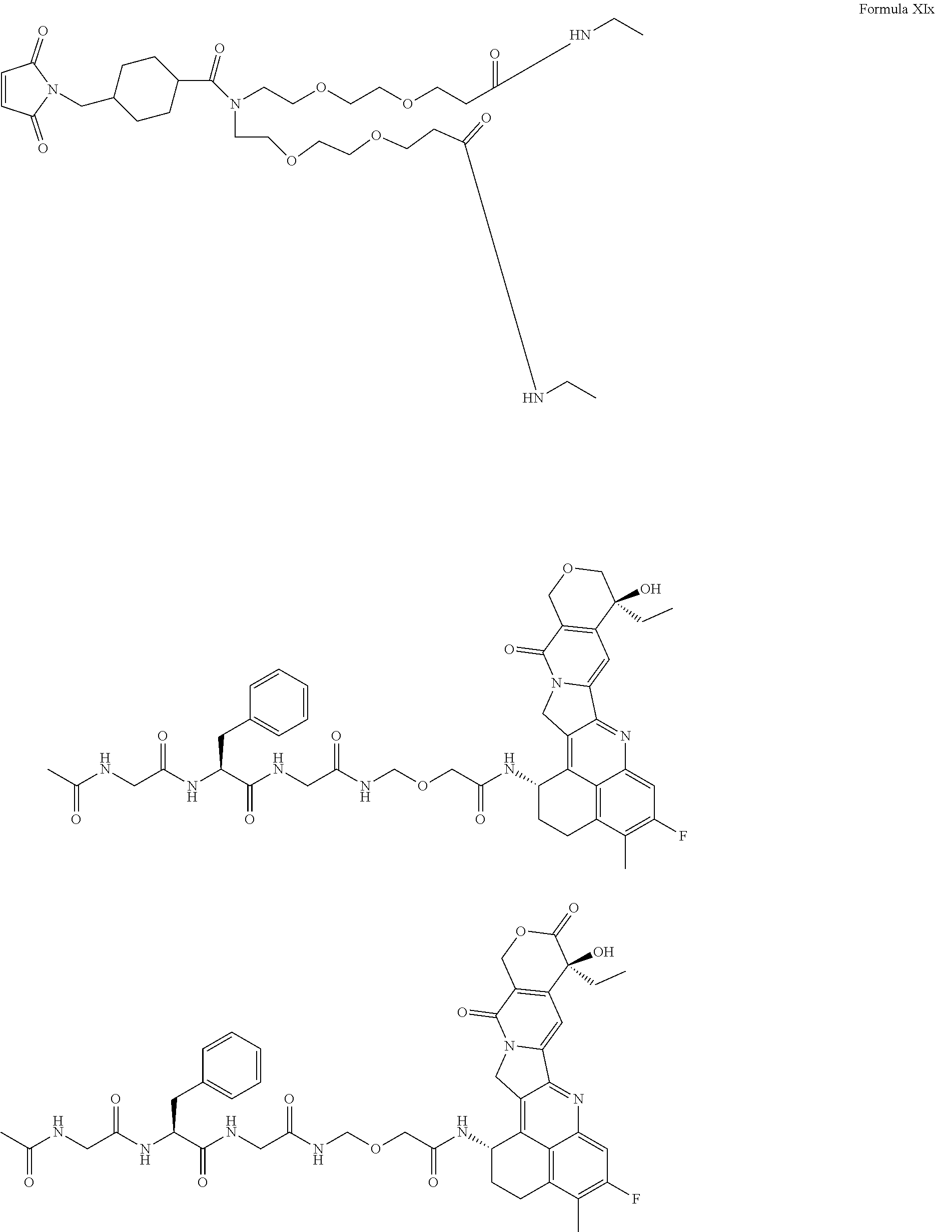

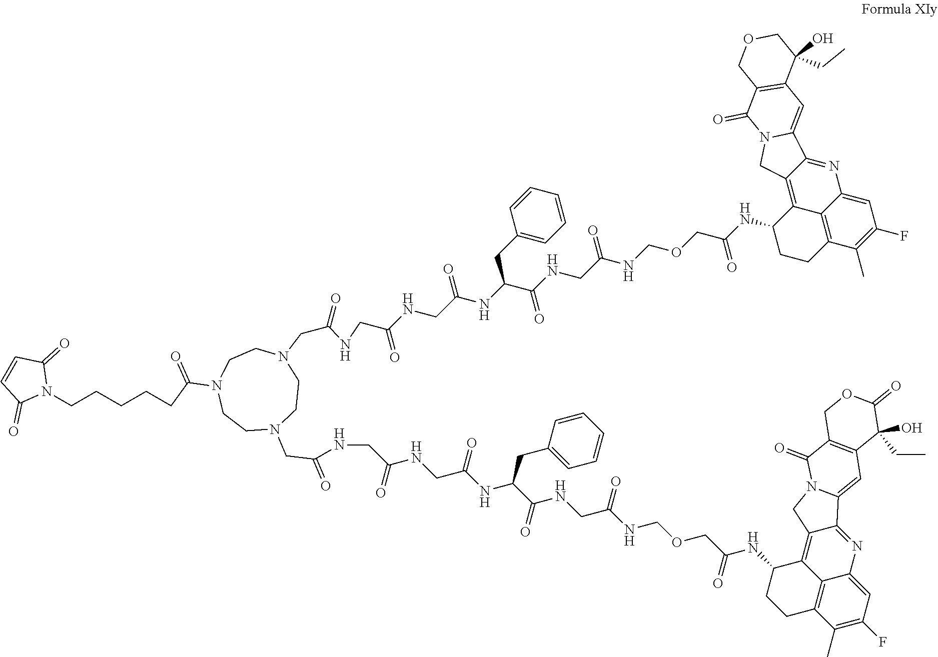 [SMILES: CC[C@@]1(O)COCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CN1CCN(CC(=O)NCC(=O)NCC(=O)N[C@@H](Cc2ccccc2)C(=O)NCC(=O)NCOCC(=O)N[C@H]2CCc4c(C)c(F)cc5nc6c(c2c45)Cn2c-6cc4c(c2=O)COC(=O)[C@]4(O)CC)CCN(C(=O)CCCCCN2C(=O)C=CC2=O)CC1)CC3]

11. A dual-drug targeting linker-drug conjugate molecule, or a stereoisomer thereof, or an optical isomer thereof, or a deuterated compound thereof, characterized in that the dual-drug targeting linker-drug conjugate is obtained by connecting the targeting linker and q dual-drug link assembly unit(s) according to claim 1; the targeting linker is a substance that can target and bind to the lesion site; the structure of the dual-drug targeting linker-drug coujugate is as represented by Formula I: 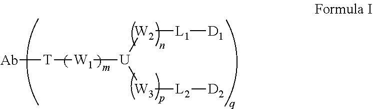
wherein, Ab is a targeting linker; 1≤q≤8; T, W₁, W₂, W₃, m, n, p, U, L₁, L₂, D₁, and D₂ are as defined in claim 1.

12. A dual-drug targeting linker-drug conjugate molecule according to claim 11, or a stereoisomer thereof, or an optical isomer thereof, or a deuterated compound thereof, characterized in that the targeting linker is an antibody, an antibody fragment, a protein, a peptide or an aptamer, and the antibody is preferably an antibody targeting cell surface receptors and tumor-related antigens.

13. A dual-drug targeting linker-drug conjugate molecule according to claim 11, or a stereoisomer thereof, or an optical isomer thereof, or a deuterated compound thereof, characterized in that the structure of said dual-drug targeting linker-drug conjugate is selected from one of the following structures:

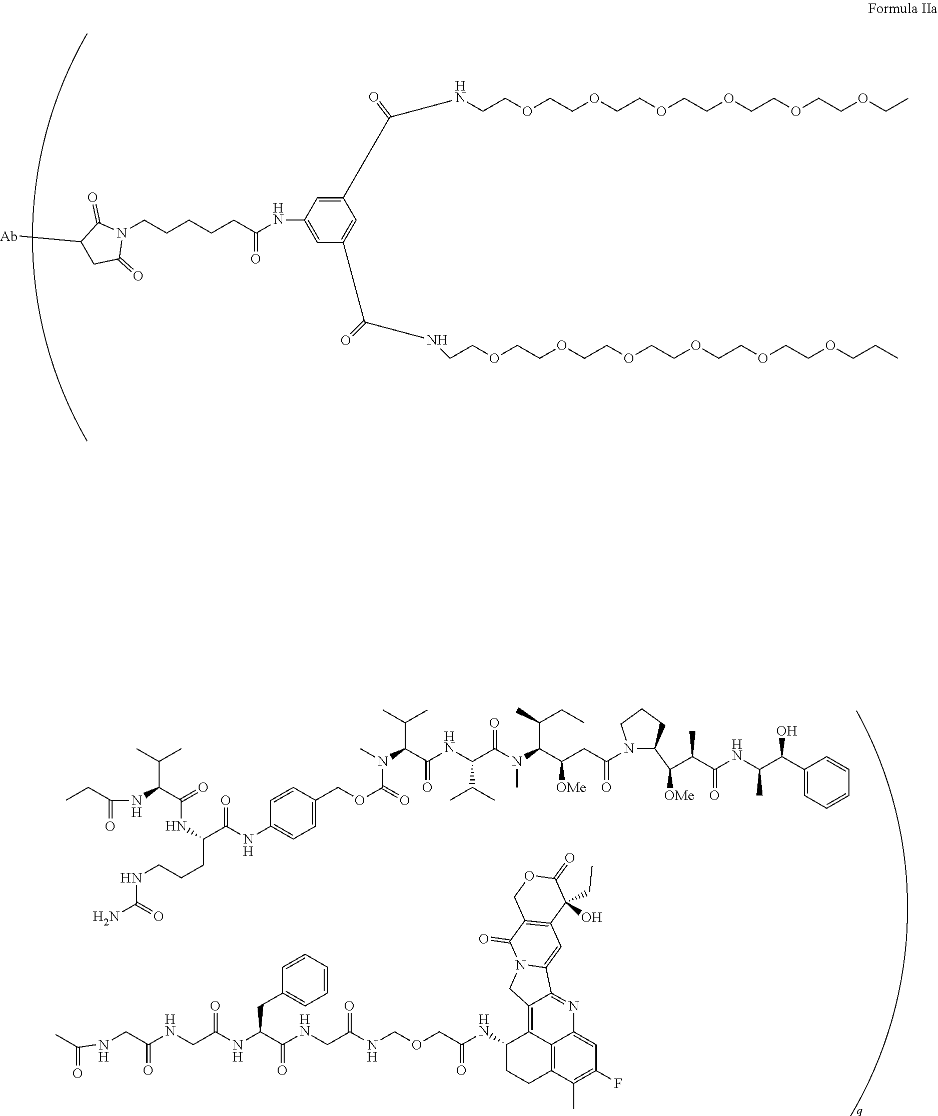

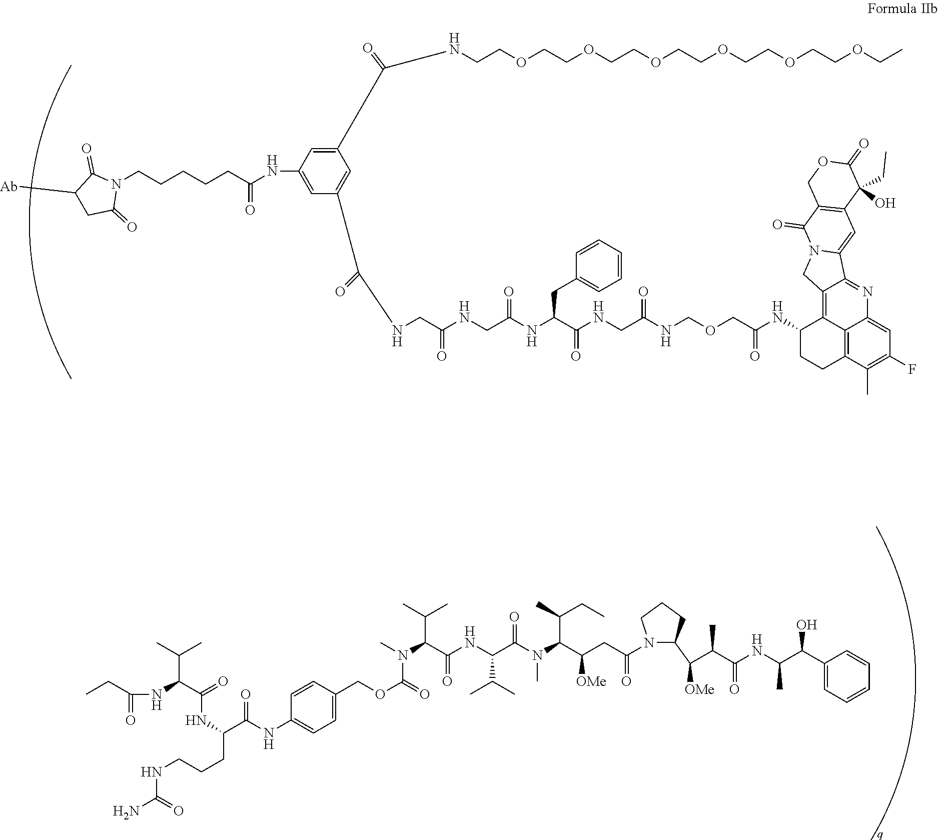

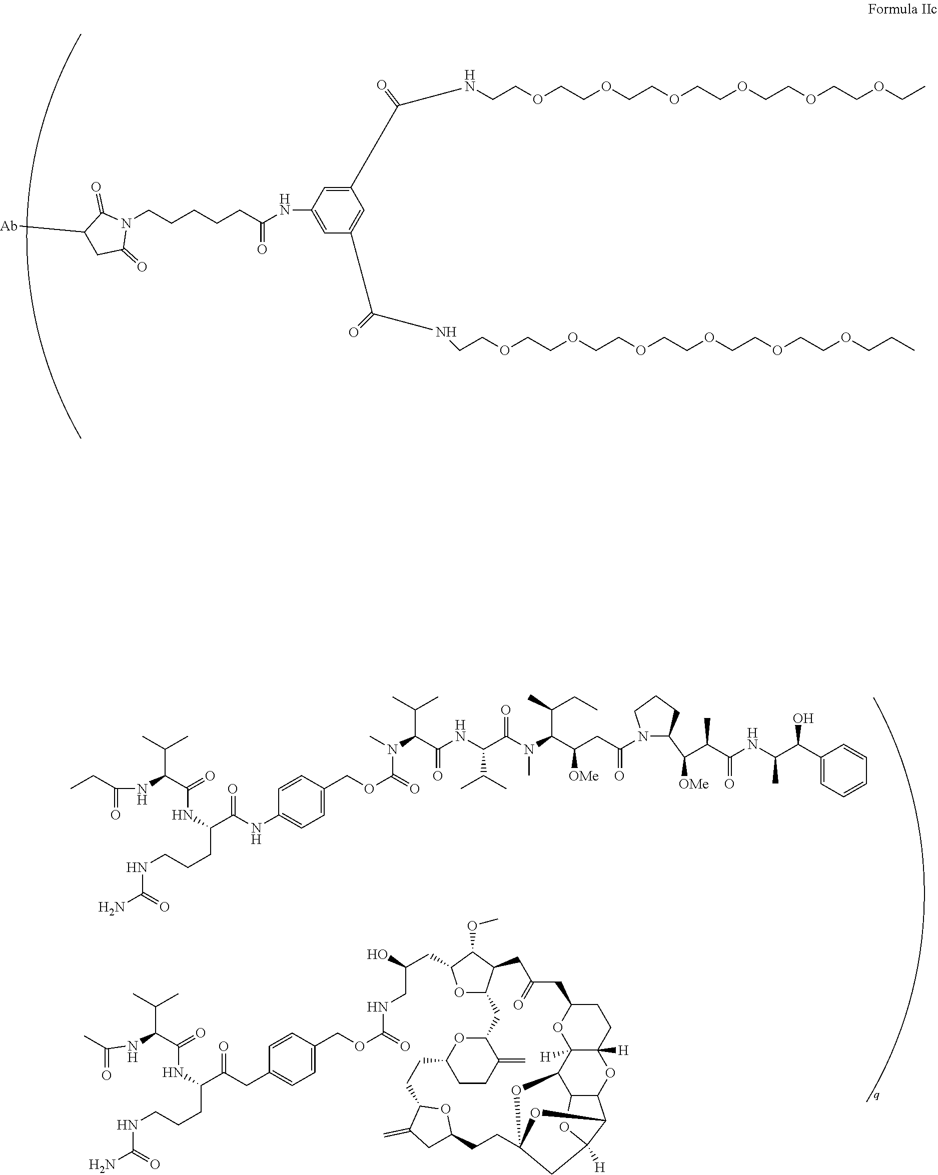

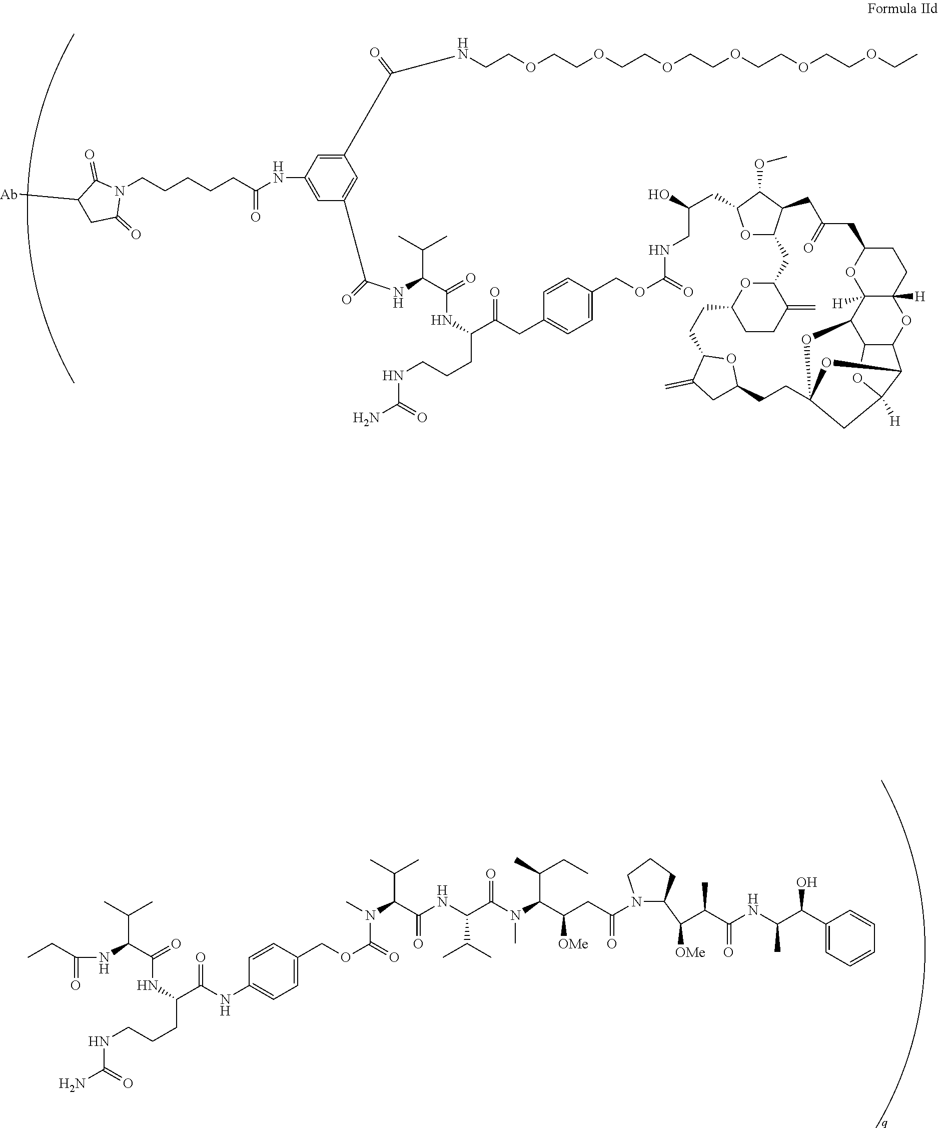

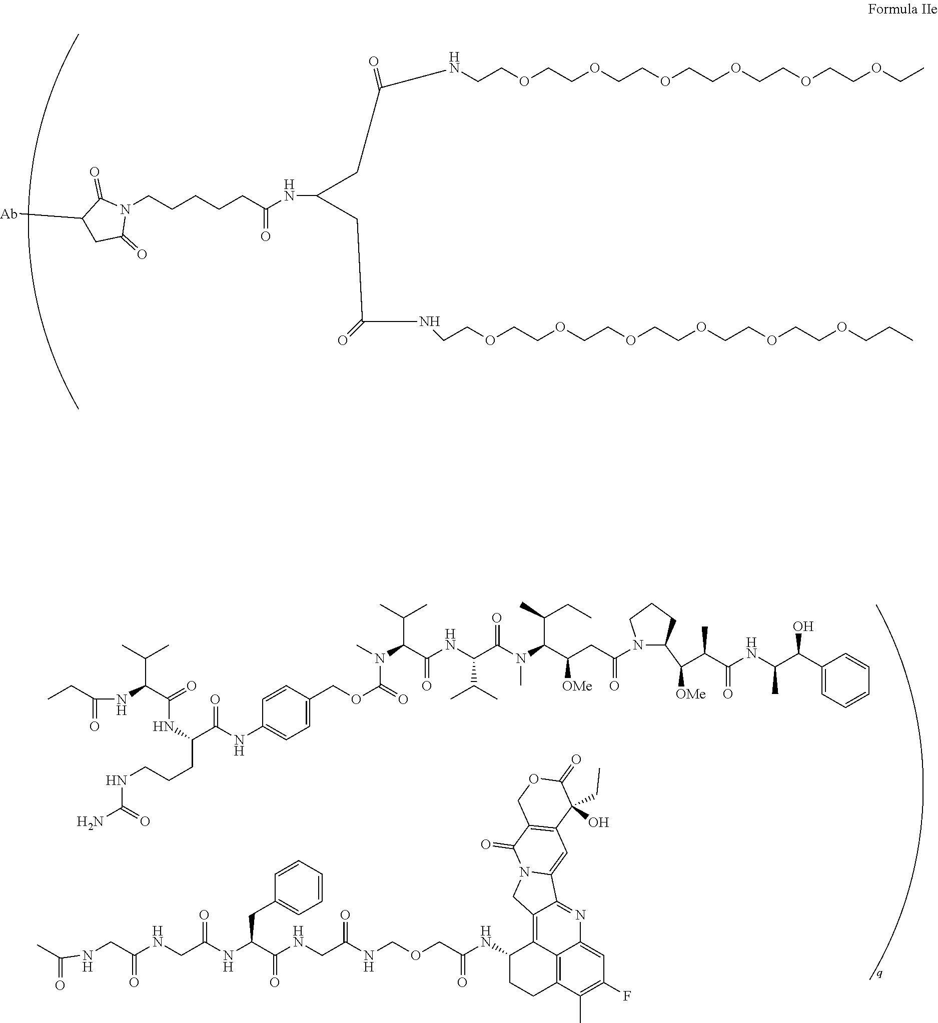

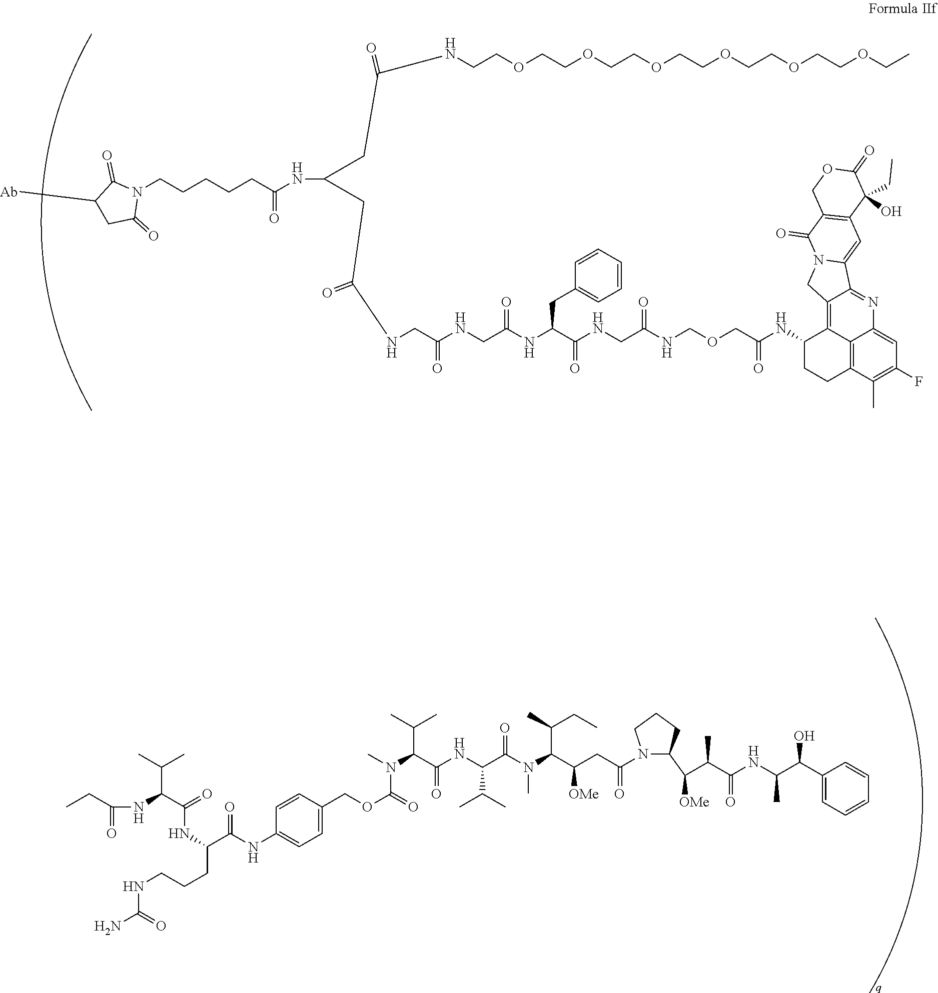

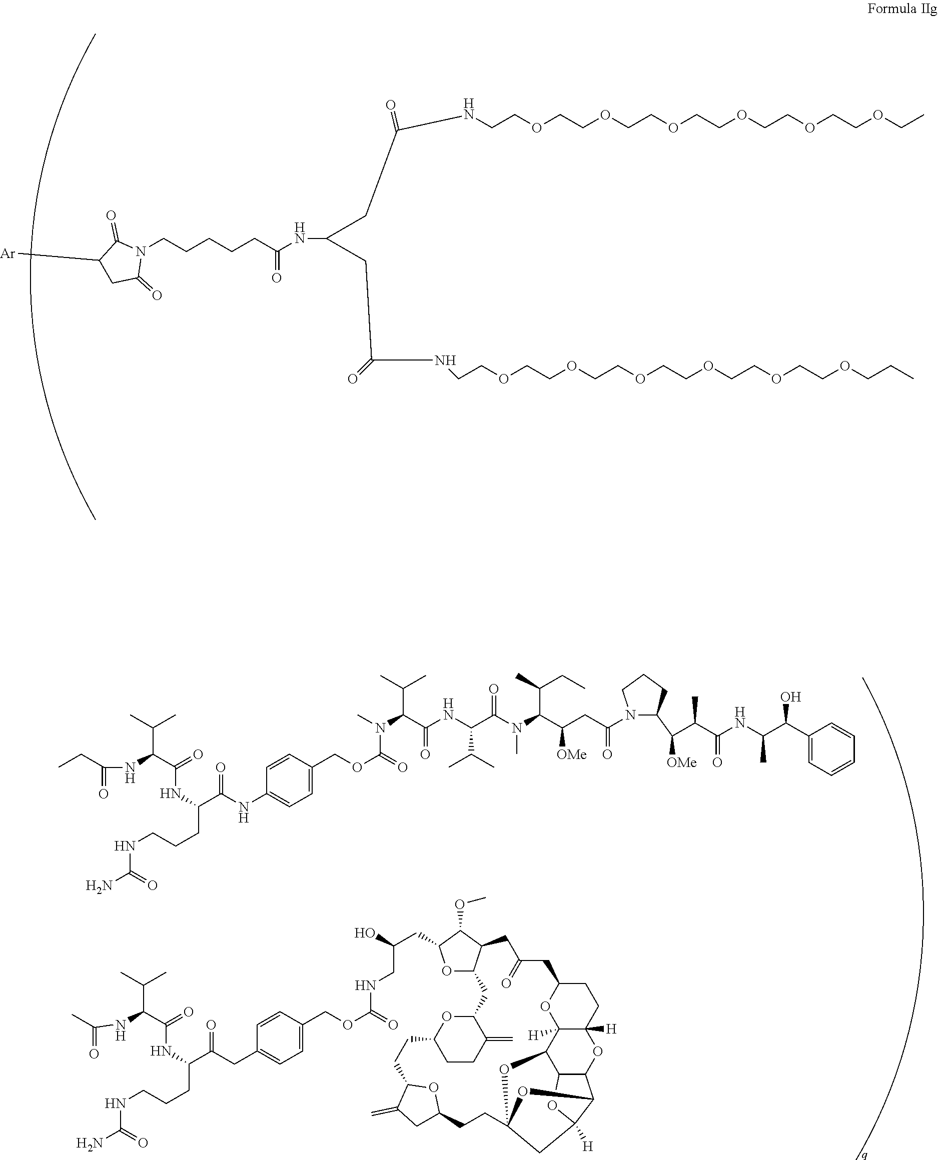

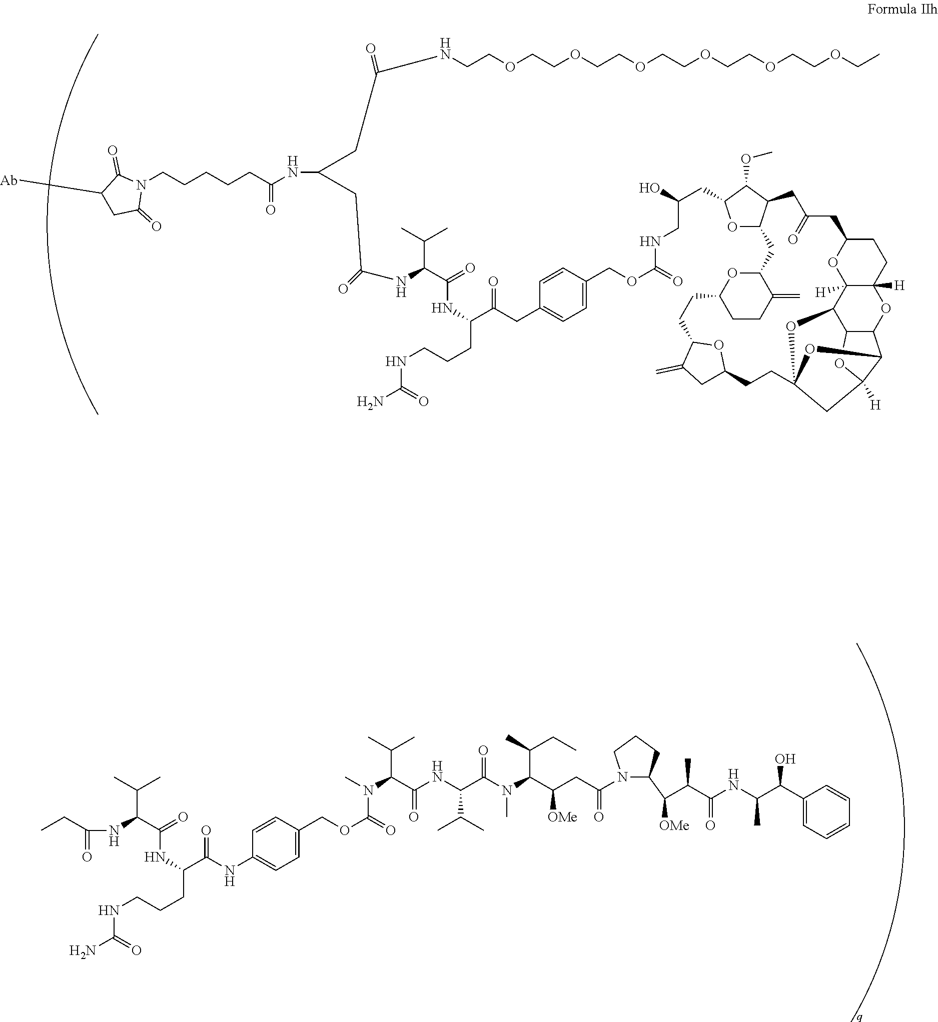

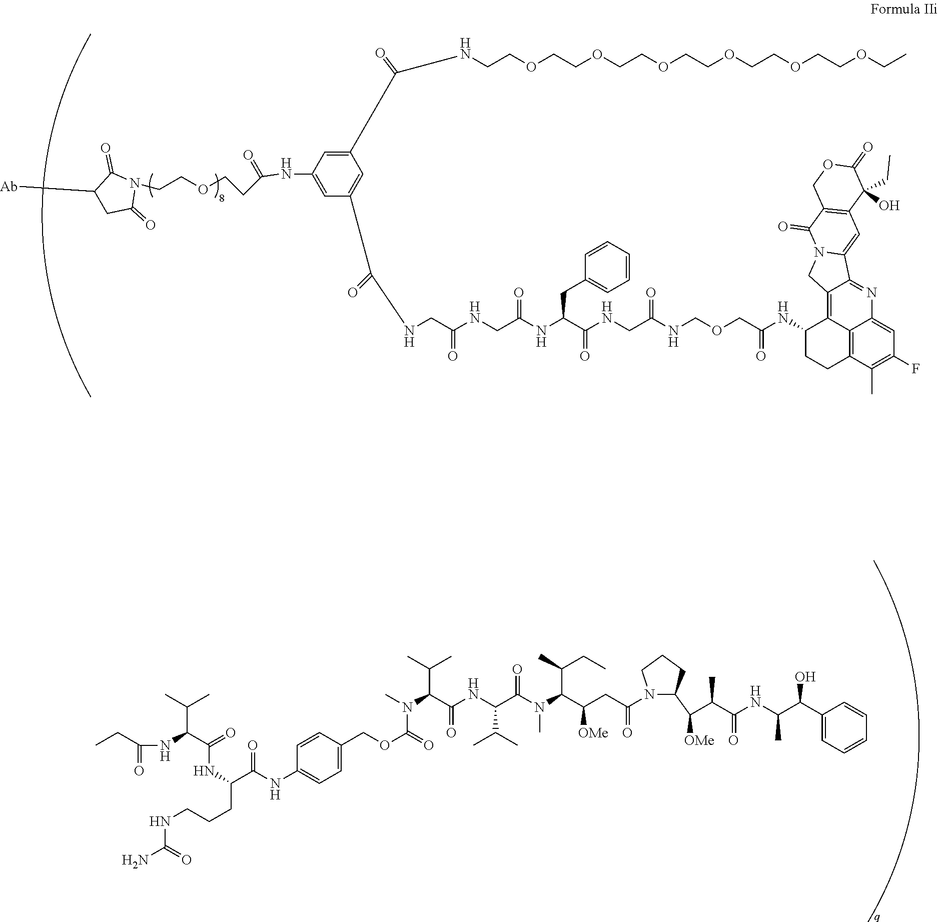

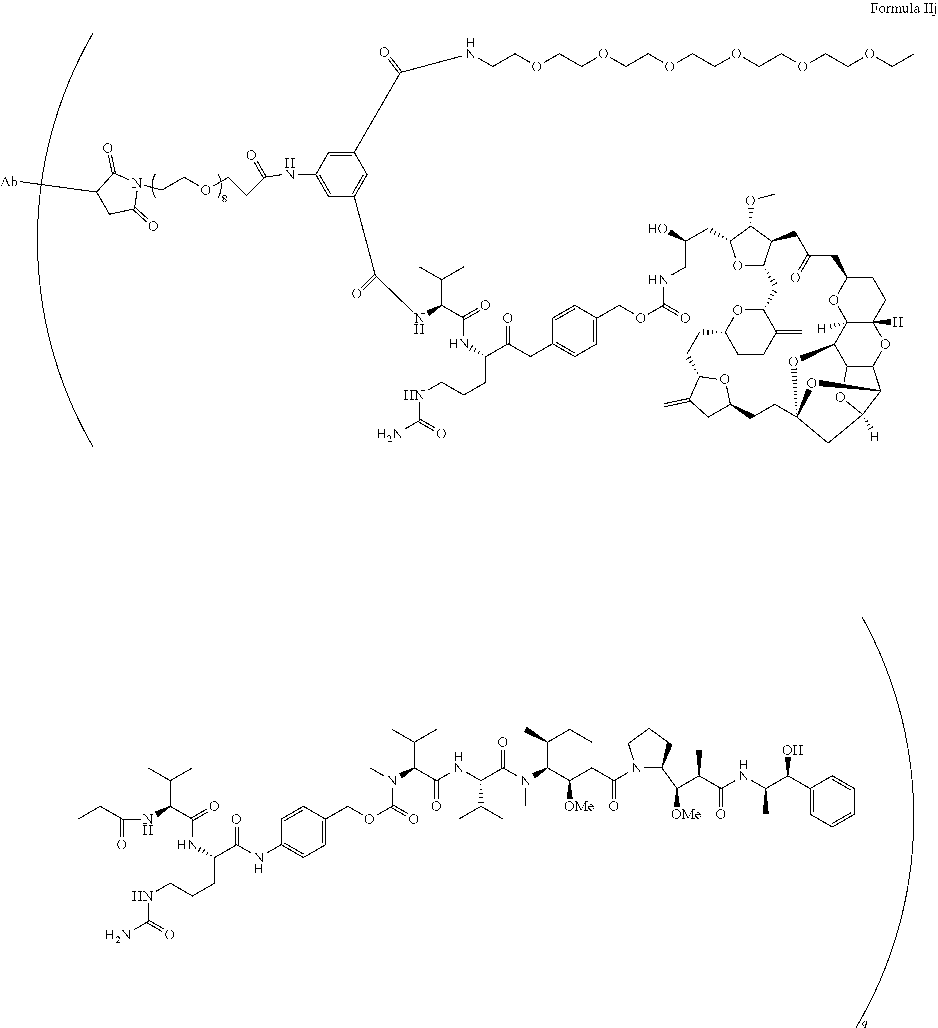

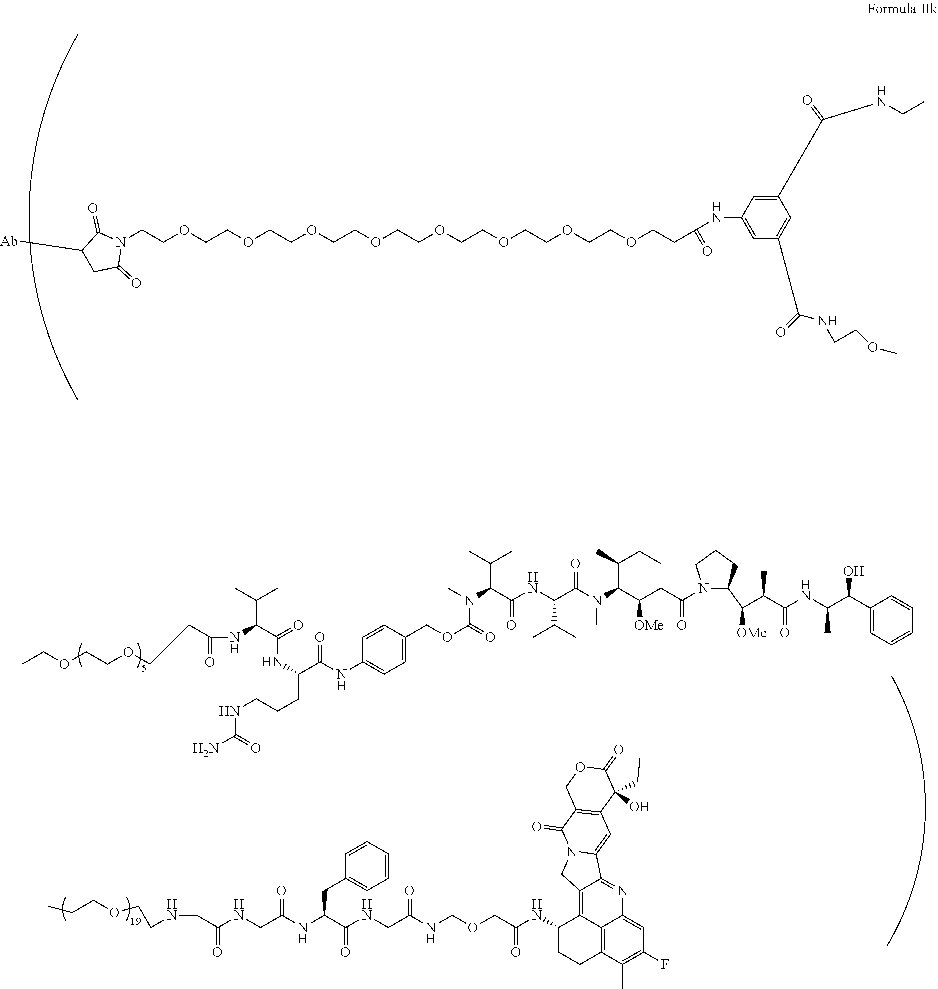

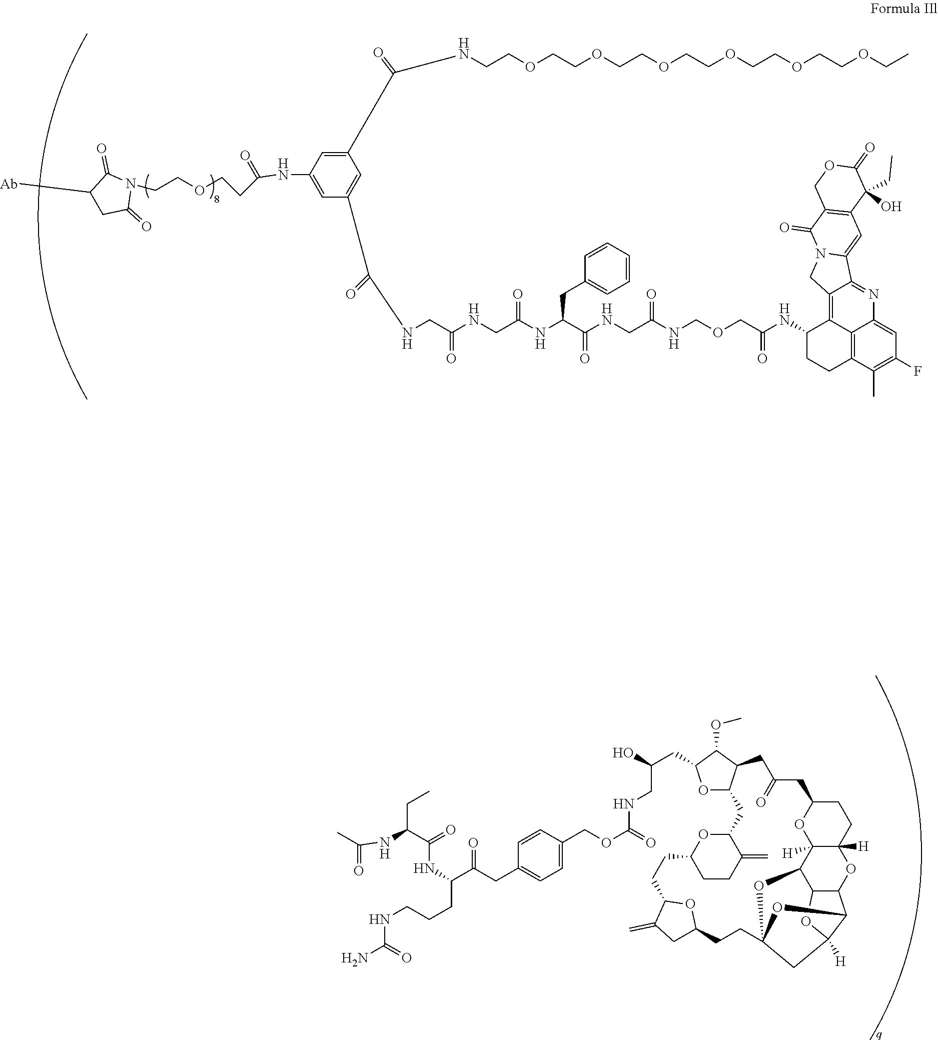

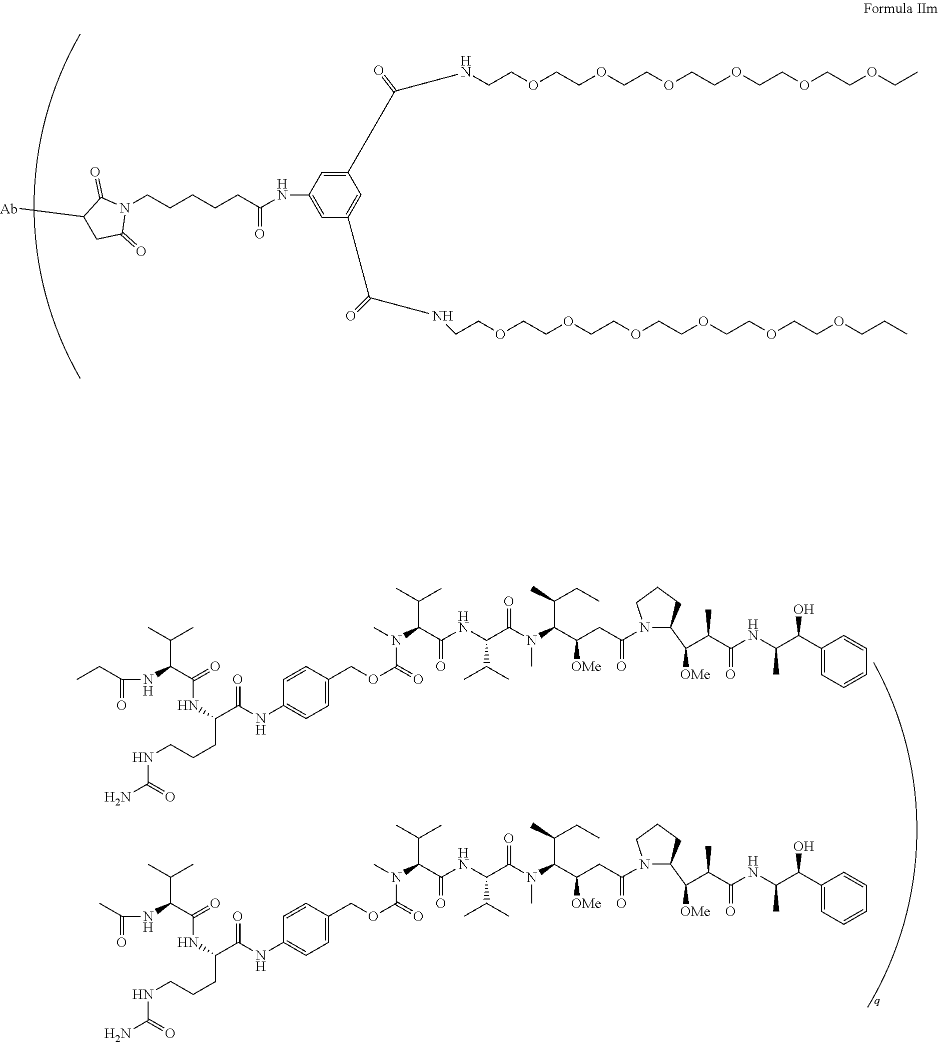

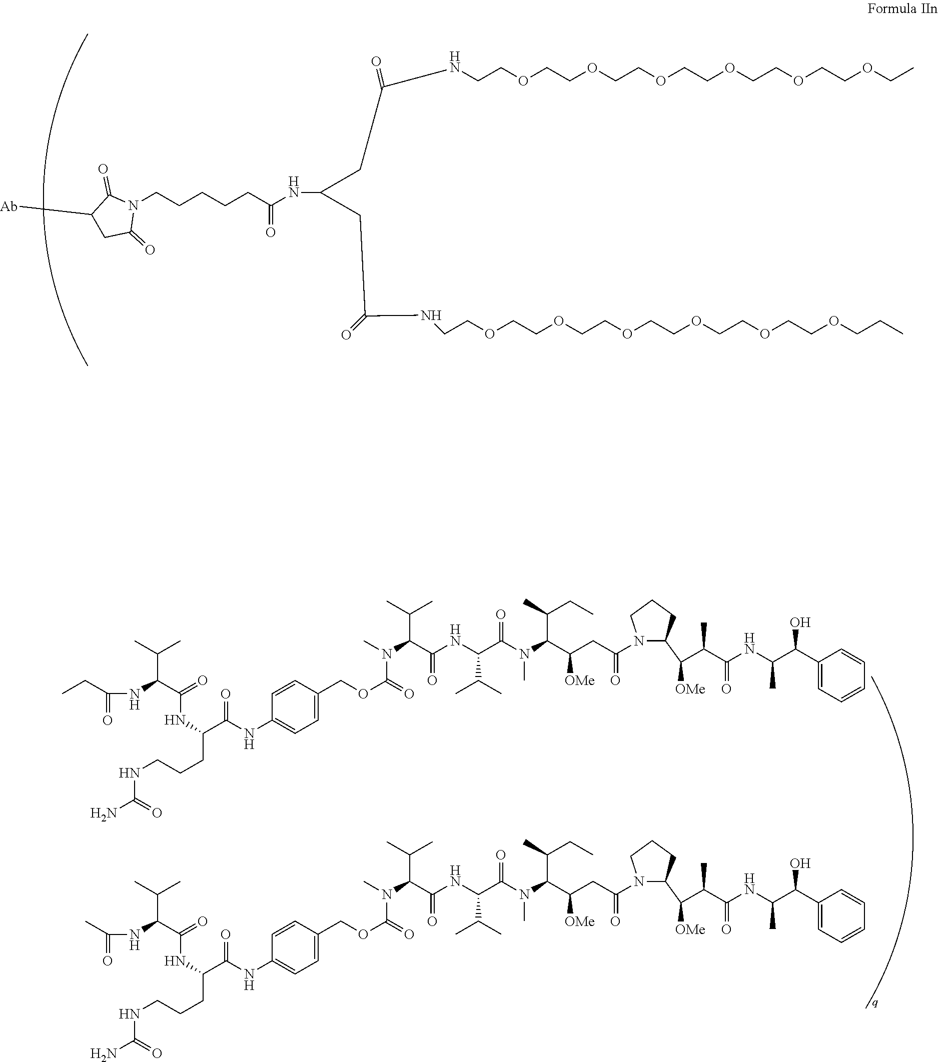

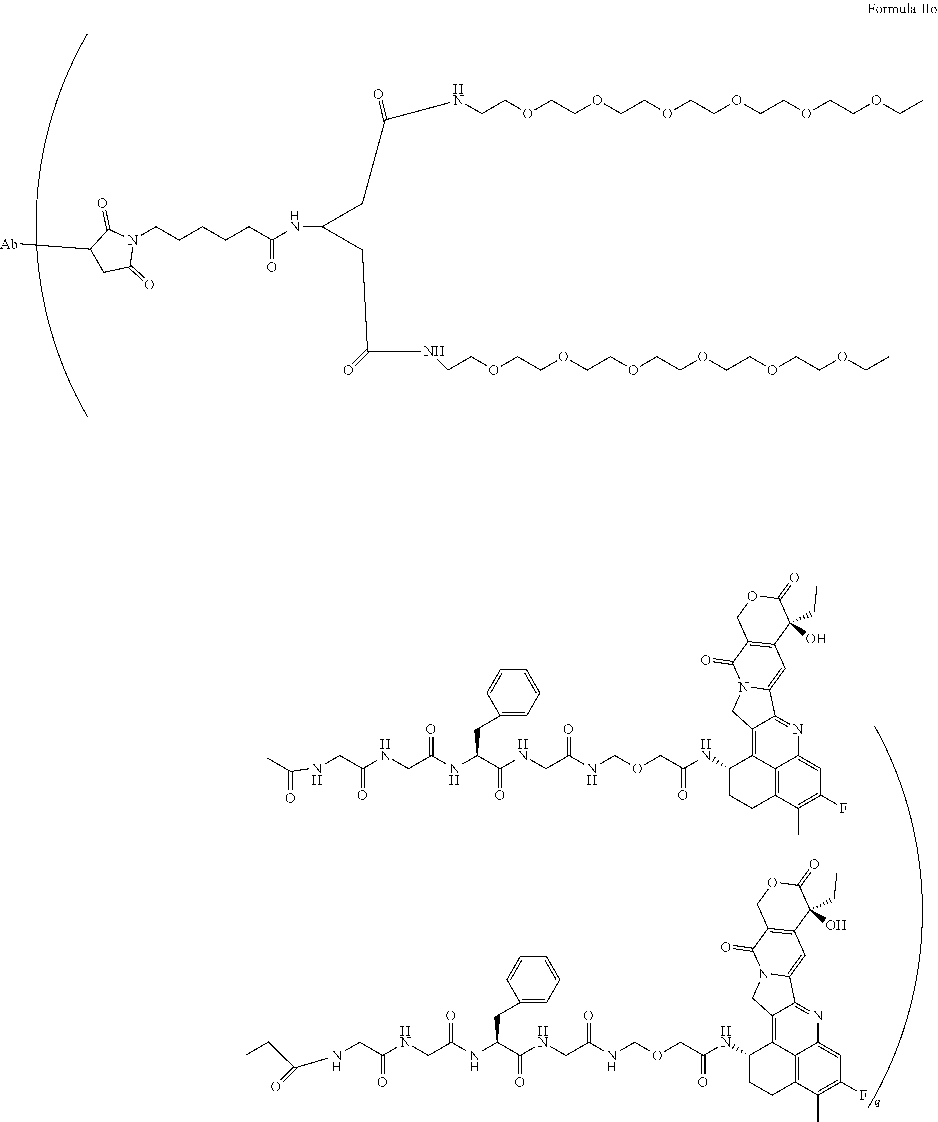

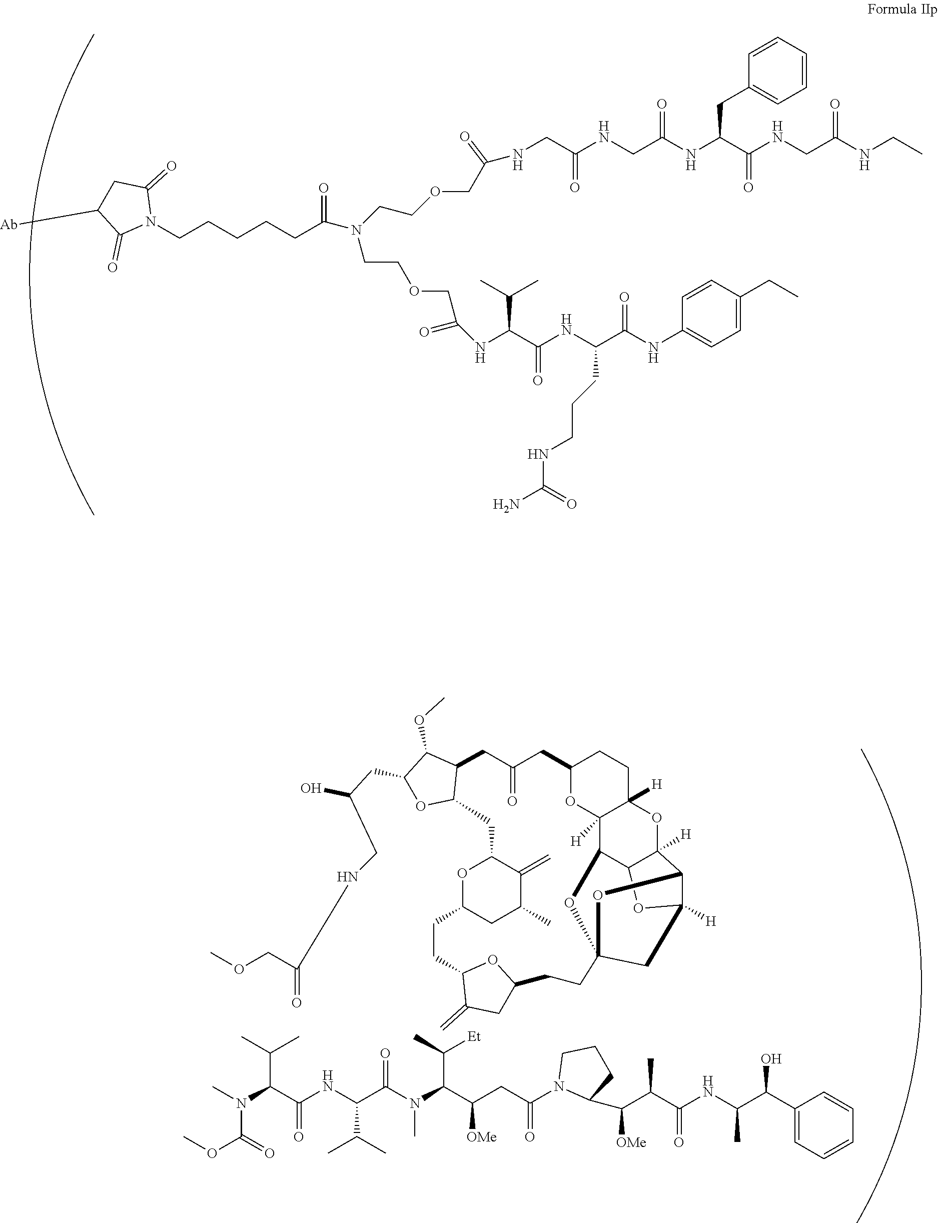

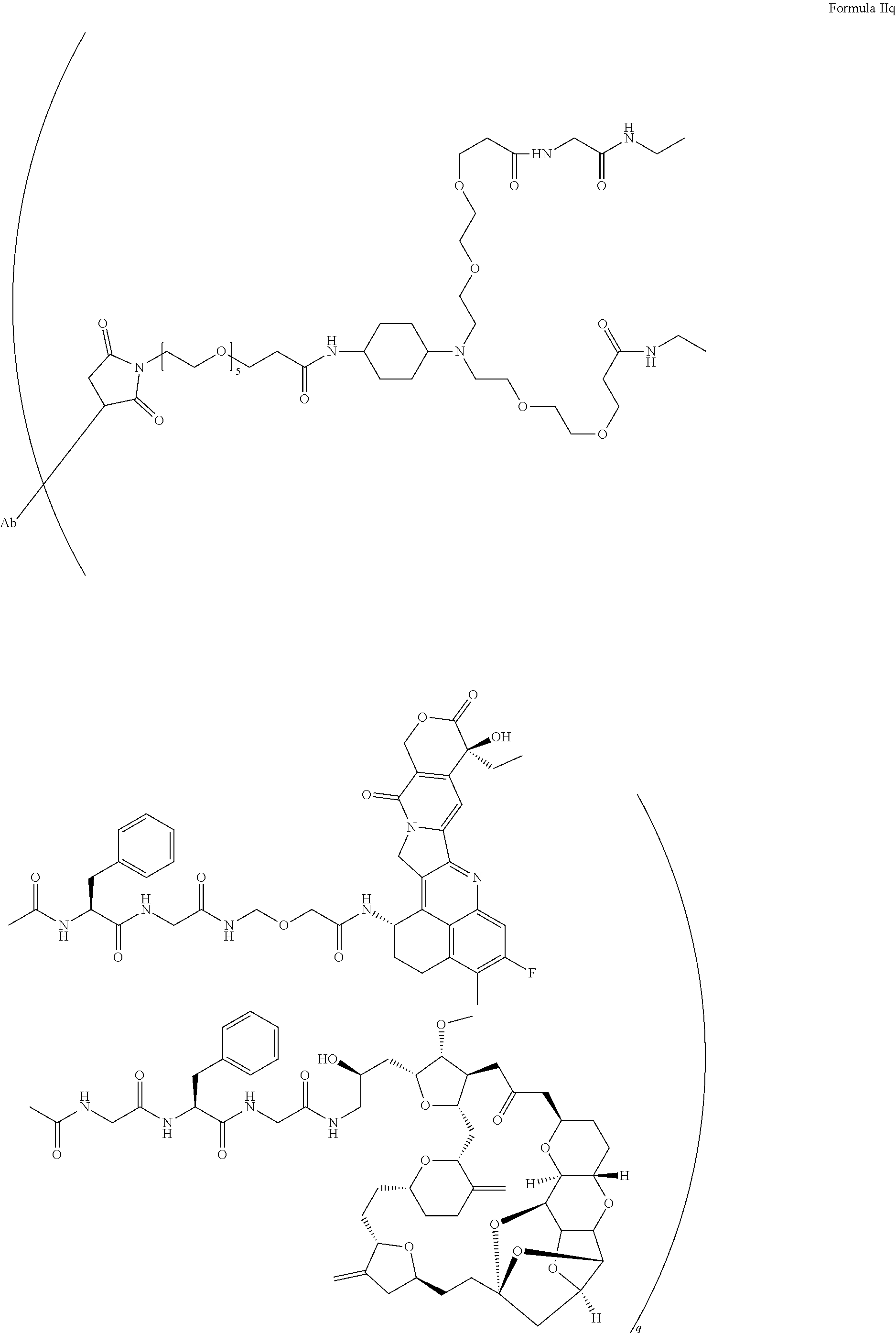

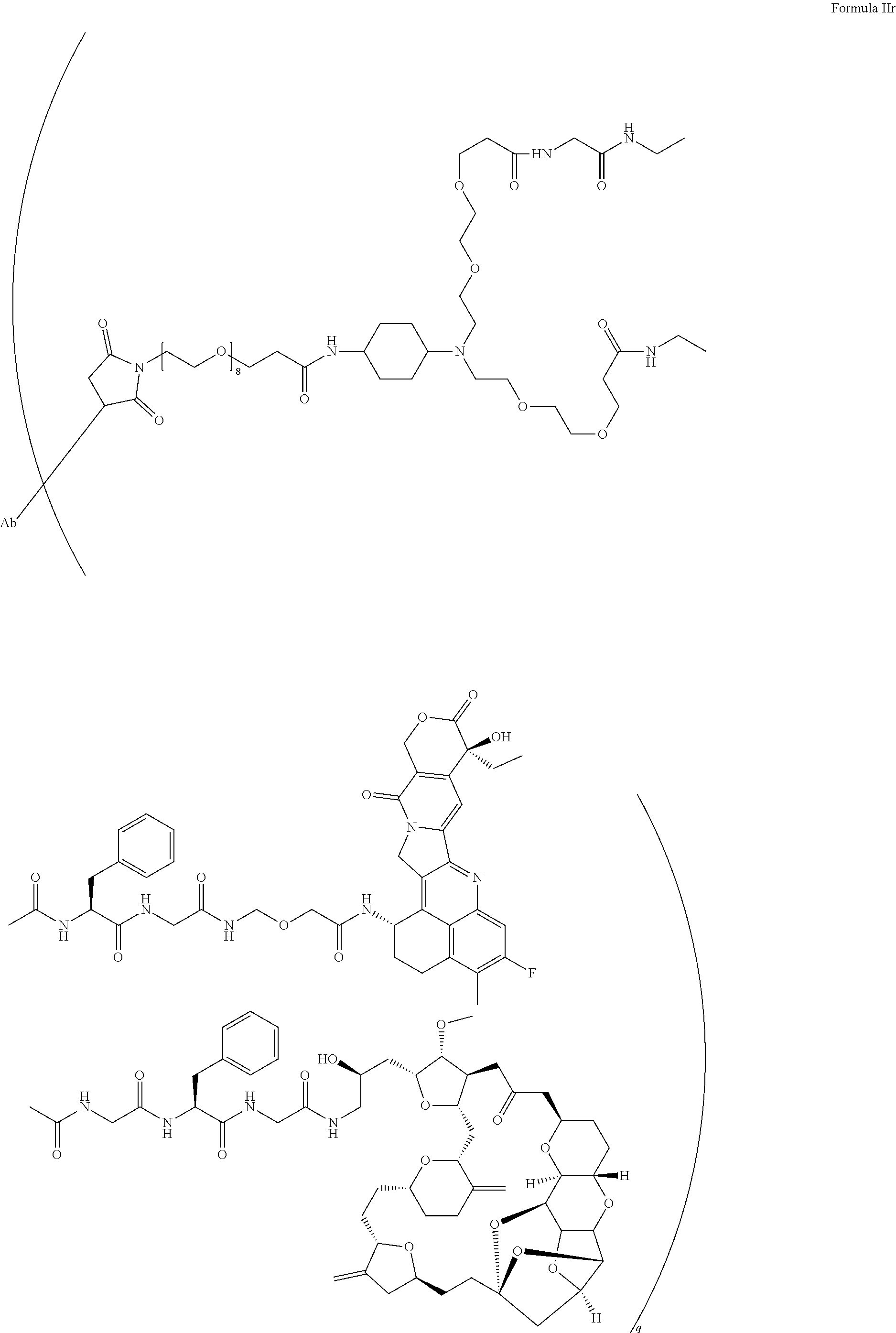

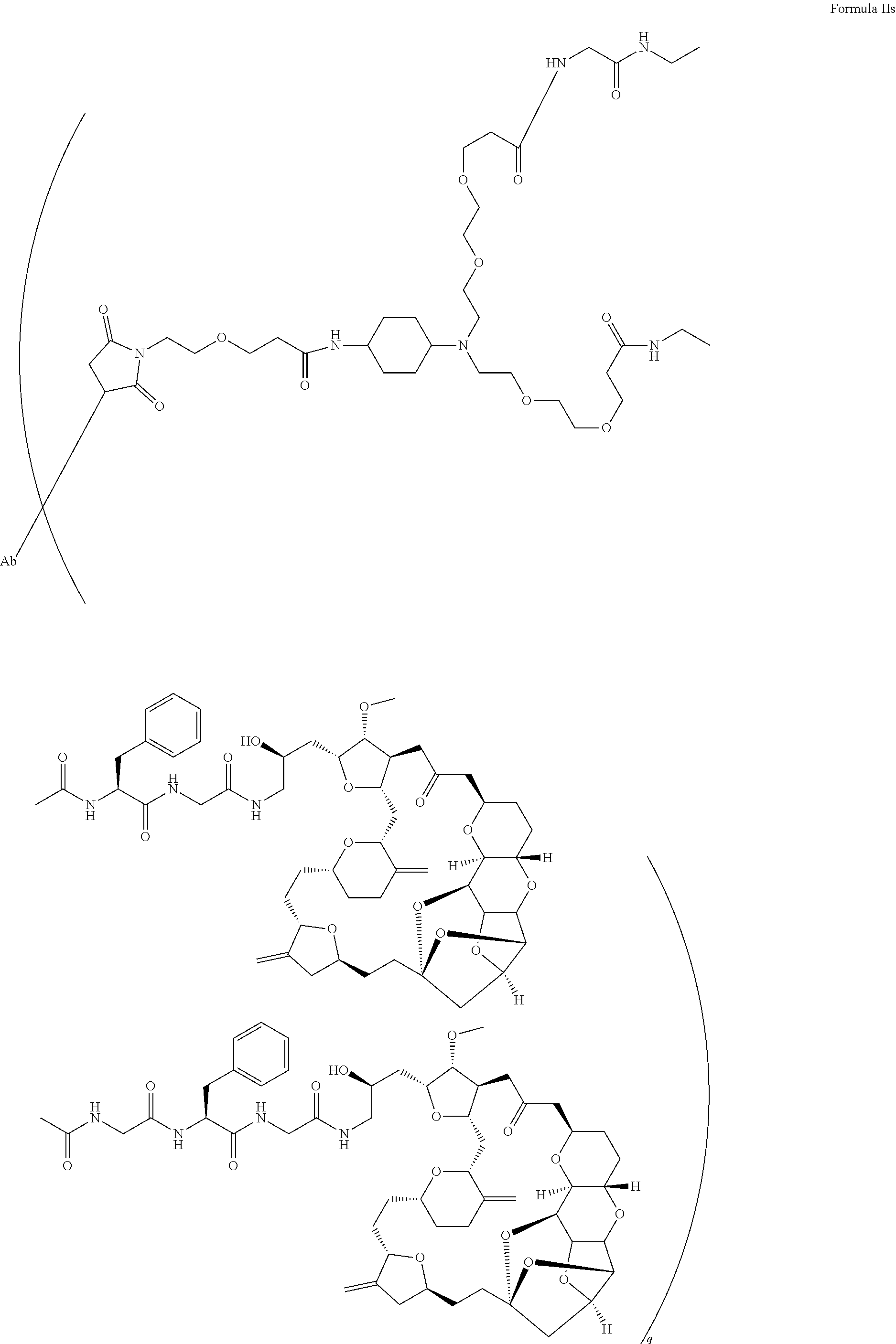

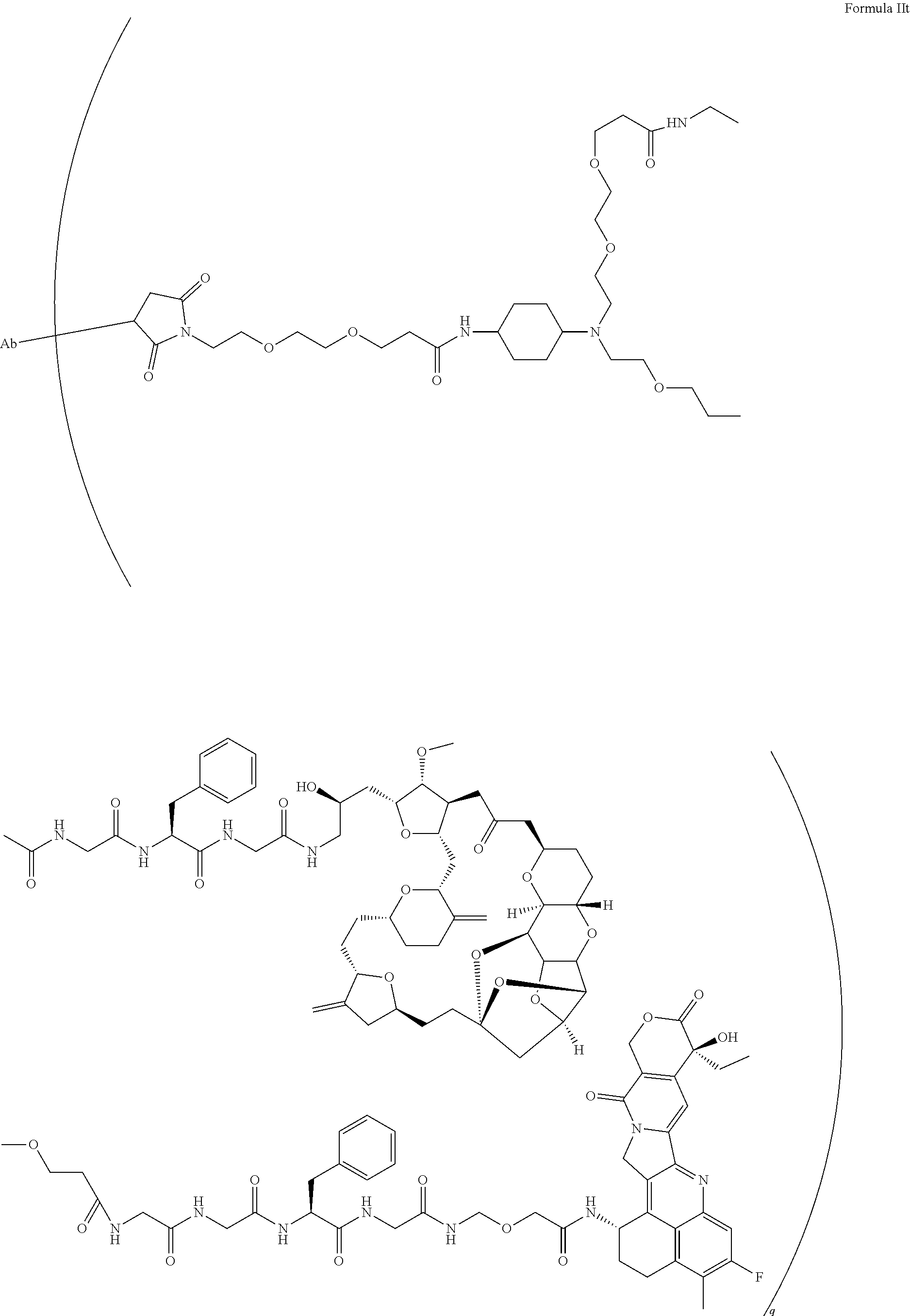

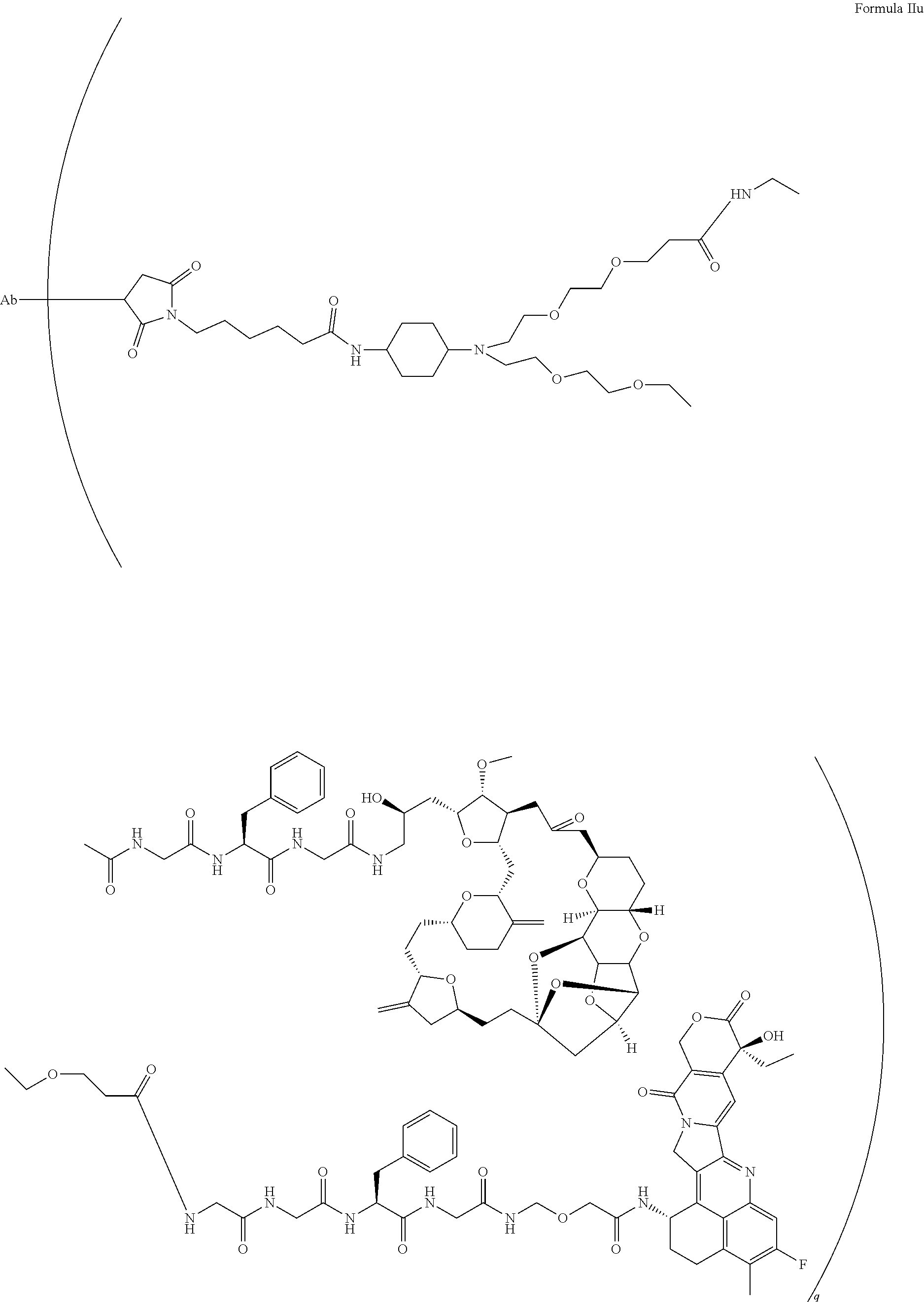

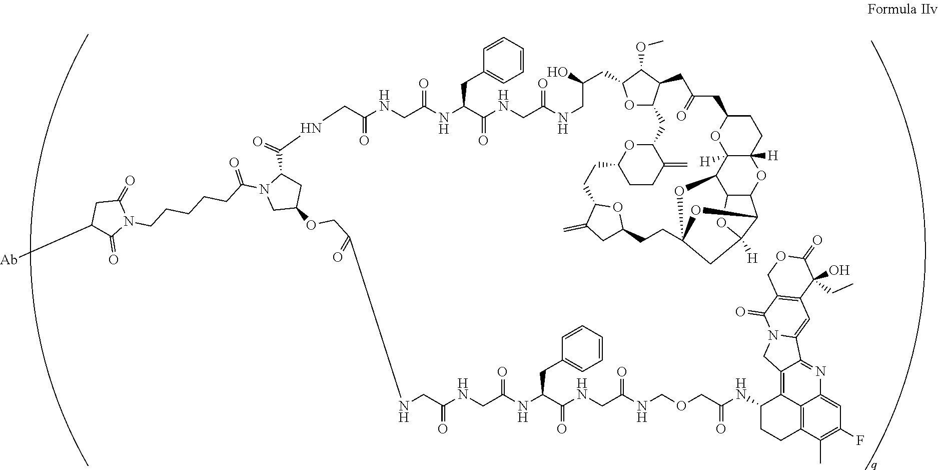

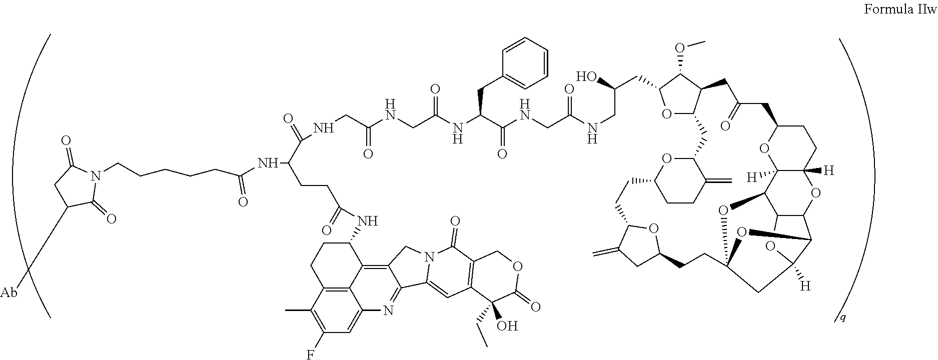

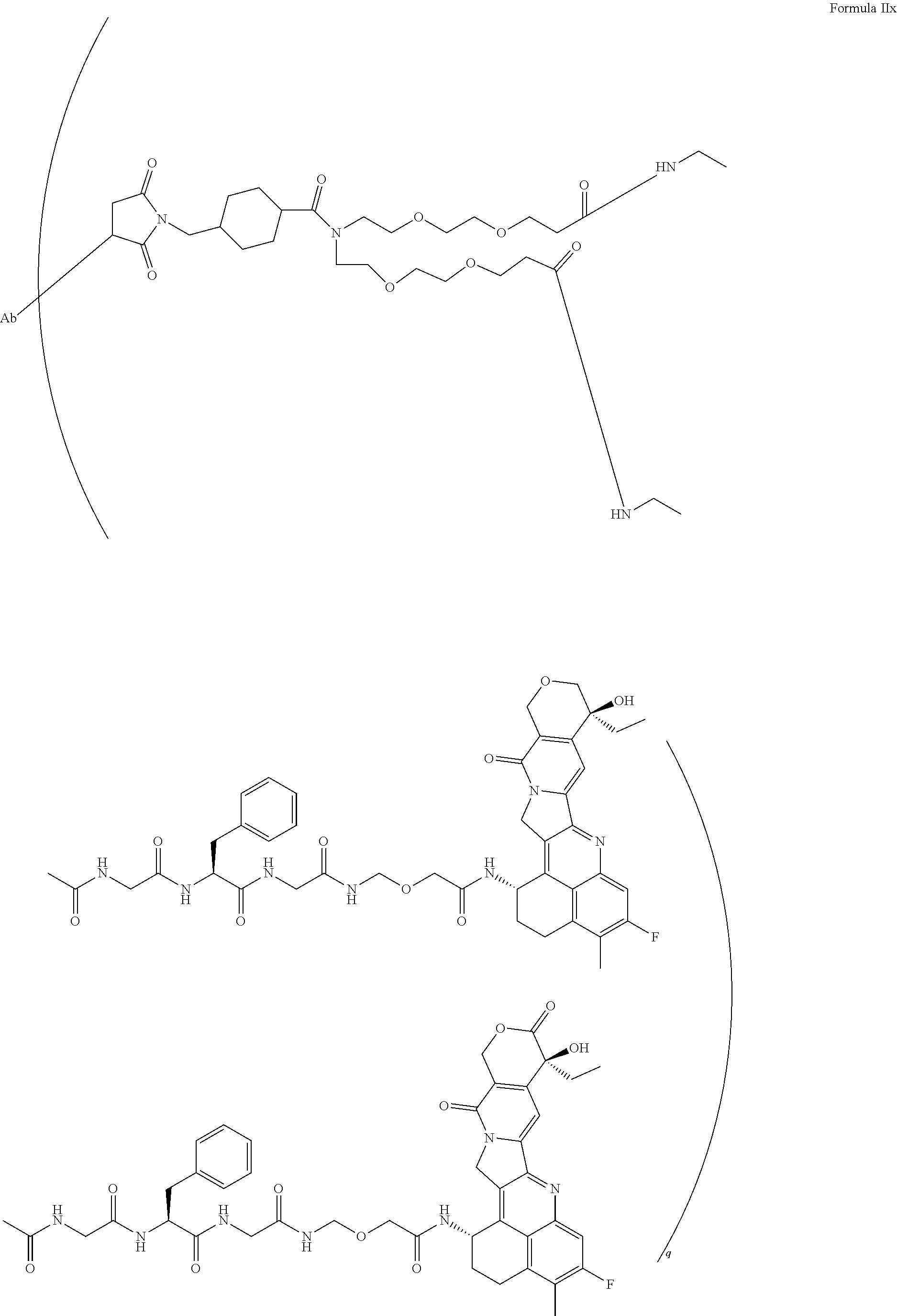

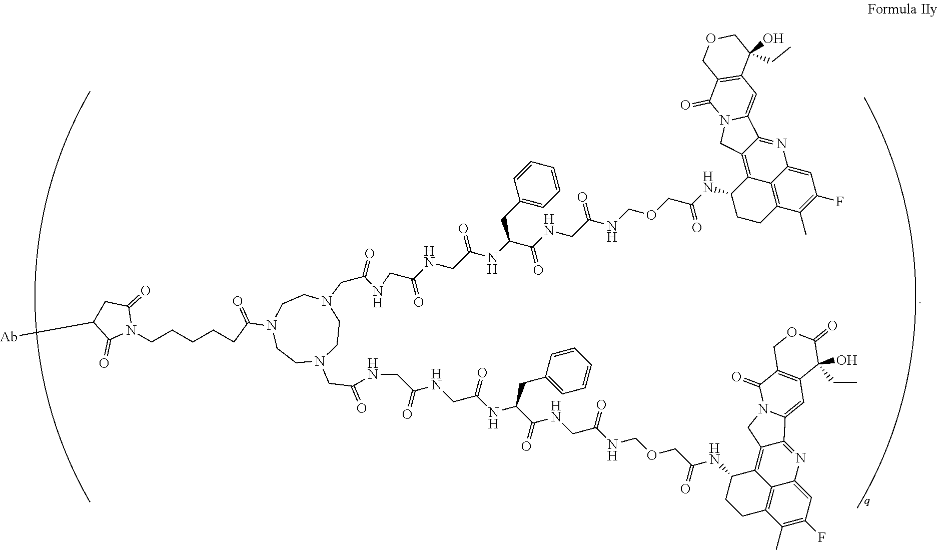

14. A dual-drug targeting linker-drug conjugate, or a stereoisomer thereof, or an optical isomer thereof, or a deuterated compound thereof, characterized in that the dual-drug targeting linker-drug conjugate is obtained by connecting the targeting linker and the dual-drug link assembly units according to claim 1; the targeting linker is a substance that can target and bind to the lesion site, and is preferably an antibody, an antibody fragment, a protein or an aptamer; the antibody is preferably an antibody targeting cell surface receptors and tumor-related antigens.

15. A dual-drug targeting linker-drug conjugate according to claim 14, or a stereoisomer thereof, or an optical isomer thereof, or a deuterated compound thereof, characterized in that the dual-drug targeting linker-drug conjugate is composed of two or more of dual-drug targeting linker-drug conjugates represented by formula XIa, with a DAR value of 3.5±1.0, preferably 3.5;
or, the dual-drug targeting linker-drug conjugate is composed of two or more of dual-drug targeting linker-drug conjugates represented by formula XIb, with a DAR value of 3.8±1.0, preferably 3.8;
or, the dual-drug targeting linker-drug conjugate is composed of two or more of dual-drug targeting linker-drug conjugates represented by formula XIc, with a DAR value of 3.9±1.0, preferably 3.9;
or, the dual-drug targeting linker-drug conjugate is composed of two or more of dual-drug targeting linker-drug conjugates represented by formula XId, with a DAR value of 4.1±1.0, preferably 4.1;
or, the dual-drug targeting linker-drug conjugate is composed of two or more of dual-drug targeting linker-drug conjugates represented by formula XIe, with a DAR value of 4.6±1.0, preferably 4.6;
or, the dual-drug targeting linker-drug conjugate is composed of two or more of dual-drug targeting linker-drug conjugates represented by formula XIf, with a DAR value of 3.7±1.0, preferably 3.7;
or, the dual-drug targeting linker-drug conjugate is composed of two or more of dual-drug targeting linker-drug conjugates represented by formula XIg, with a DAR value of 4.2±1.0, preferably 4.2;
or, the dual-drug targeting linker-drug conjugate is composed of two or more of dual-drug targeting linker-drug conjugates represented by formula XIh, with a DAR value of 4.1±1.0, preferably 4.1;
or, the dual-drug targeting linker-drug conjugate is composed of two or more of dual-drug targeting linker-drug conjugates represented by formula XIi, with a DAR value of 4.7±1.0, preferably 4.7;
or, the dual-drug targeting linker-drug conjugate is composed of two or more of dual-drug targeting linker-drug conjugates represented by formula XIj, with a DAR value of 2.8±1.0, preferably 2.8;
or, the dual-drug targeting linker-drug conjugate is composed of two or more of dual-drug targeting linker-drug conjugates represented by formula XIk, with a DAR value of 2.9±1.0, preferably 2.9;
or, the dual-drug targeting linker-drug conjugate is composed of two or more of dual-drug targeting linker-drug conjugates represented by formula XIl, with a DAR value of 4.5±1.0, preferably 4.5;
or, the dual-drug targeting linker-drug conjugate is composed of two or more of dual-drug targeting linker-drug conjugates represented by formula XIm, with a DAR value of 4.4±1.0, preferably 4.4;
or, the dual-drug targeting linker-drug conjugate is composed of two or more of dual-drug targeting linker-drug conjugates represented by formula XIn, with a DAR value of 3.7±1.0, preferably 3.7;
or, the dual-drug targeting linker-drug conjugate is composed of two or more of dual-drug targeting linker-drug conjugates represented by formula XIo, with a DAR value of 3.9±1.0, preferably 3.9;
or, the dual-drug targeting linker-drug conjugate is composed of two or more of dual-drug targeting linker-drug conjugates represented by formula XIp, with a DAR value of 3.5±1.0, preferably 3.5;
or, the dual-drug targeting linker-drug conjugate is composed of two or more of dual-drug targeting linker-drug conjugates represented by formula XIq, with a DAR value of 4.8±1.0, preferably 4.8;
or, the dual-drug targeting linker-drug conjugate is composed of two or more of dual-drug targeting linker-drug conjugates represented by formula XIr, with a DAR value of 4.9±1.0, preferably 4.9;
or, the dual-drug targeting linker-drug conjugate is composed of two or more of dual-drug targeting linker-drug conjugates represented by formula XIs, with a DAR value of 4.2±1.0, preferably 4.2;
or, the dual-drug targeting linker-drug conjugate is composed of two or more of dual-drug targeting linker-drug conjugates represented by formula XIt, with a DAR value of 5.3±1.0, preferably 5.3;
or, the dual-drug targeting linker-drug conjugate is composed of two or more of dual-drug targeting linker-drug conjugates represented by formula XIu, with a DAR value of 4.5±1.0, preferably 4.5;
or, the dual-drug targeting linker-drug conjugate is composed of two or more of dual-drug targeting linker-drug conjugates represented by formula XIv, with a DAR value of 4.9±1.0, preferably 4.9;
or, the dual-drug targeting linker-drug conjugate is composed of two or more of dual-drug targeting linker-drug conjugates represented by formula XIw, with a DAR value of 3.8±1.0, preferably 3.8;
or, the dual-drug targeting linker-drug conjugate is composed of two or more of dual-drug targeting linker-drug conjugates represented by formula XIx, with a DAR value of 4.9±1.0, preferably 4.9;
or, the dual-drug targeting linker-drug conjugate is composed of two or more of dual-drug targeting linker-drug conjugates represented by formula XIy, with a DAR value of 4.8±1.0, preferably 4.8;
wherein:

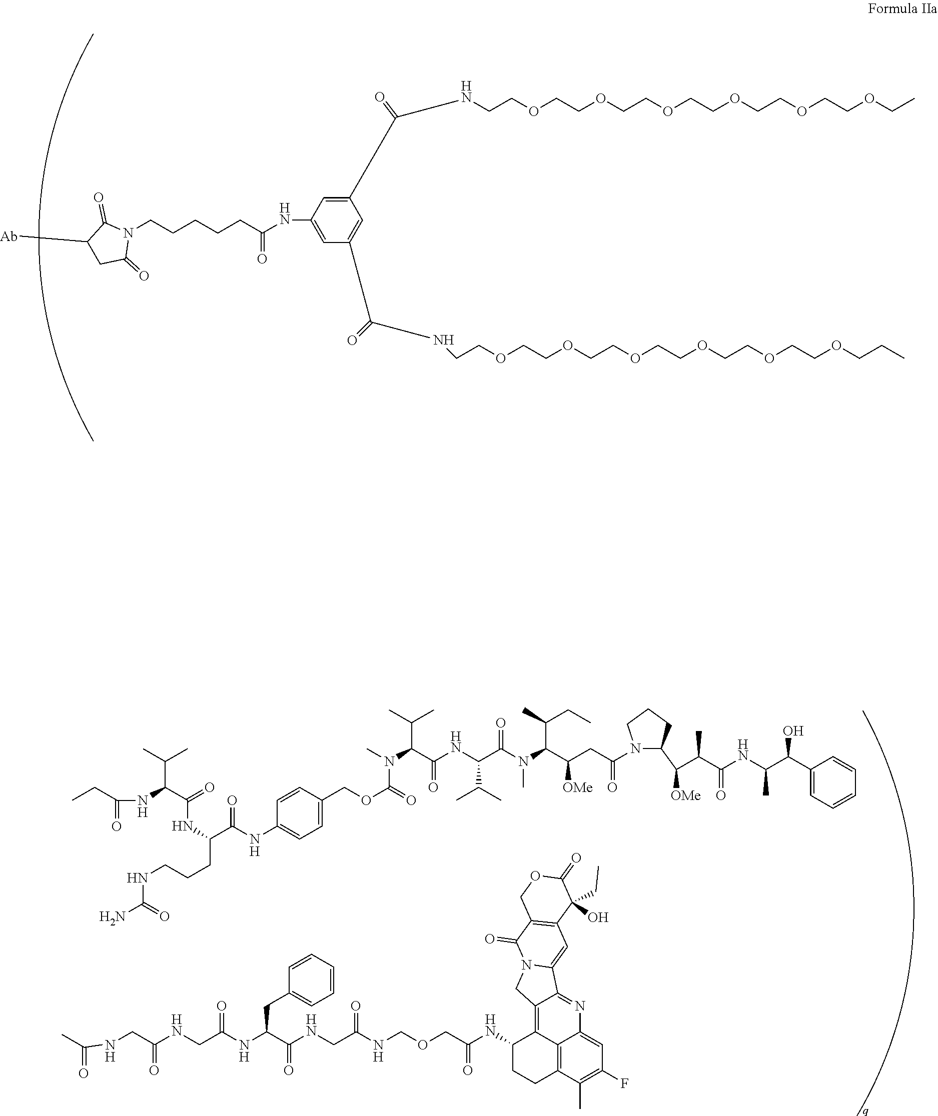

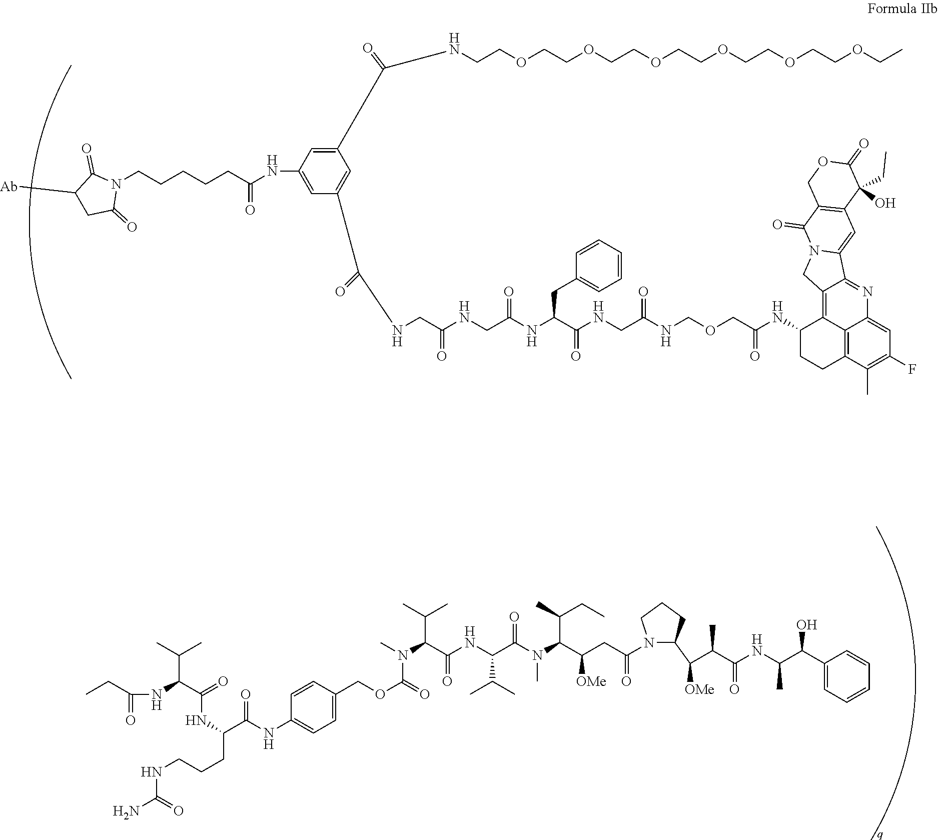

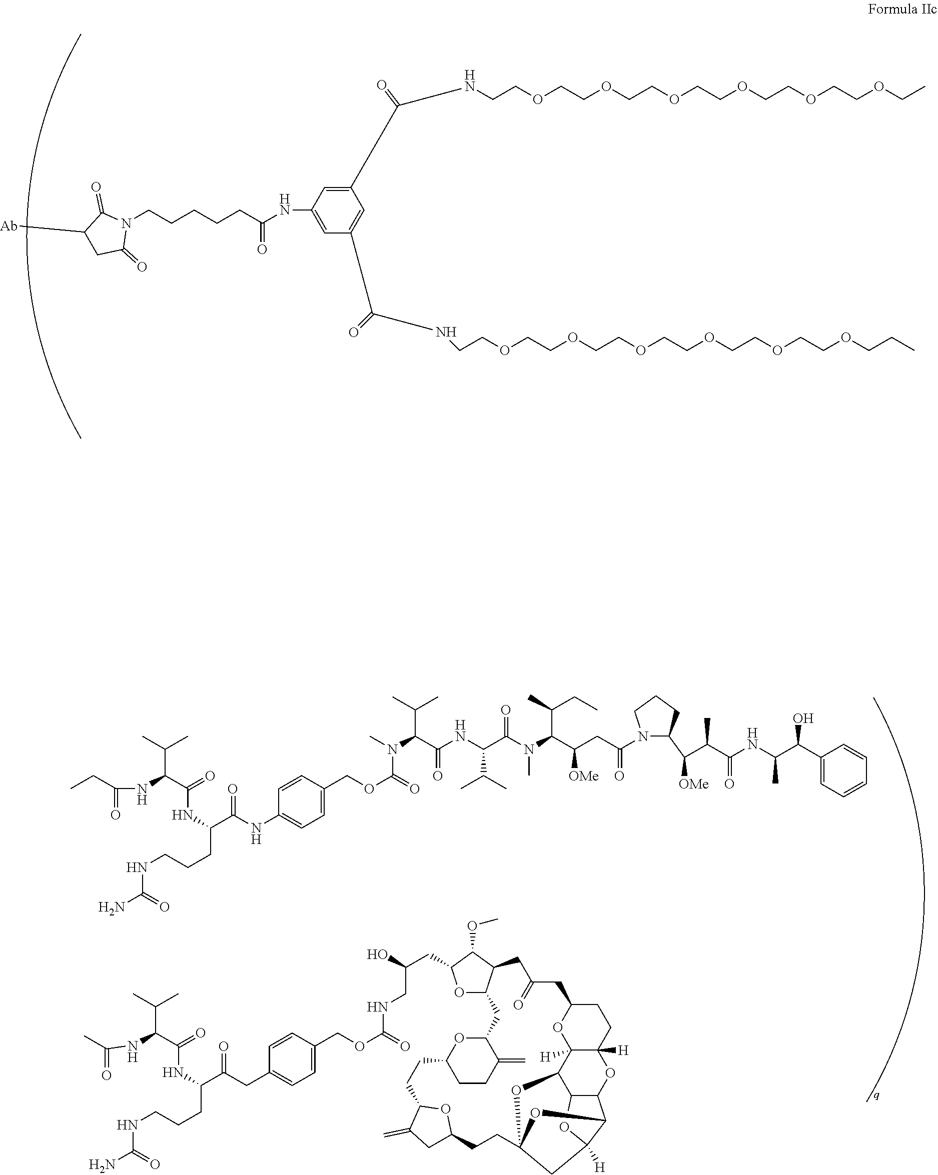

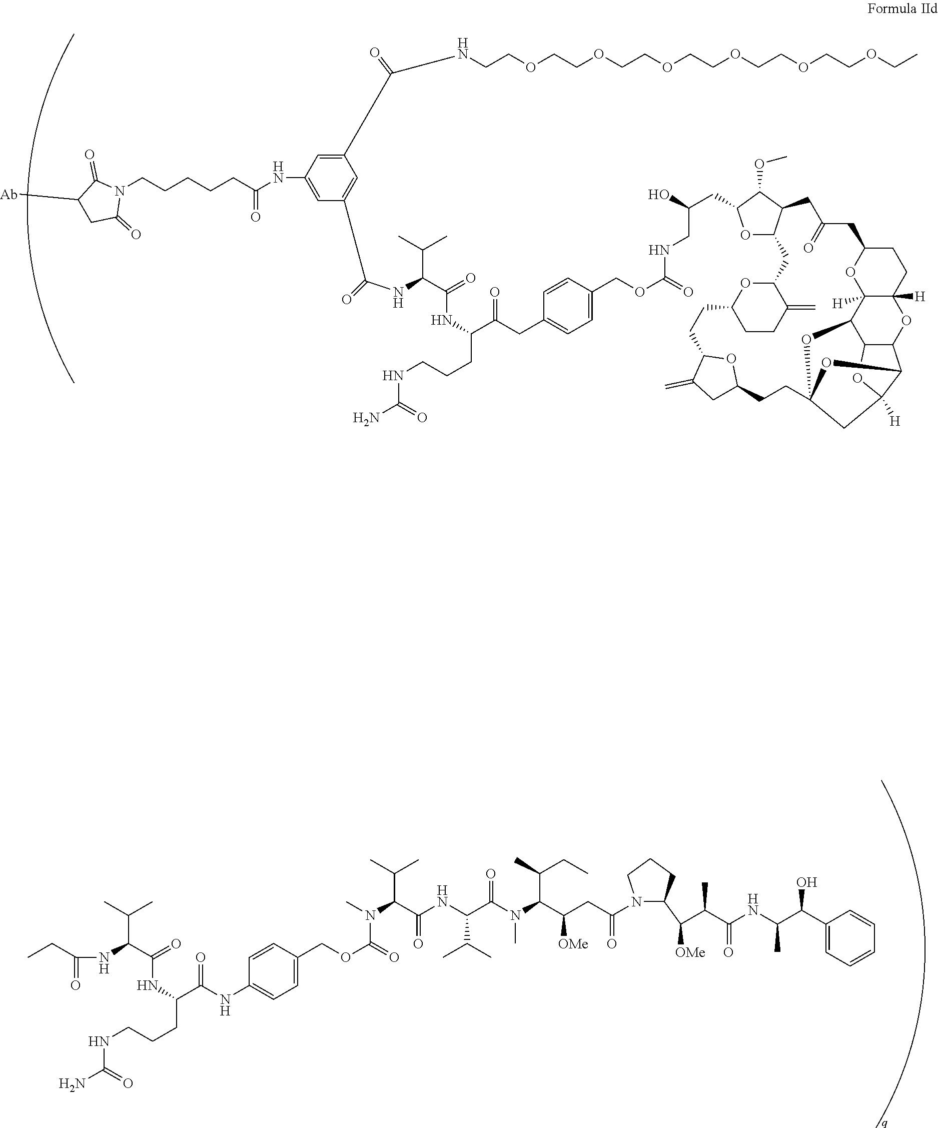

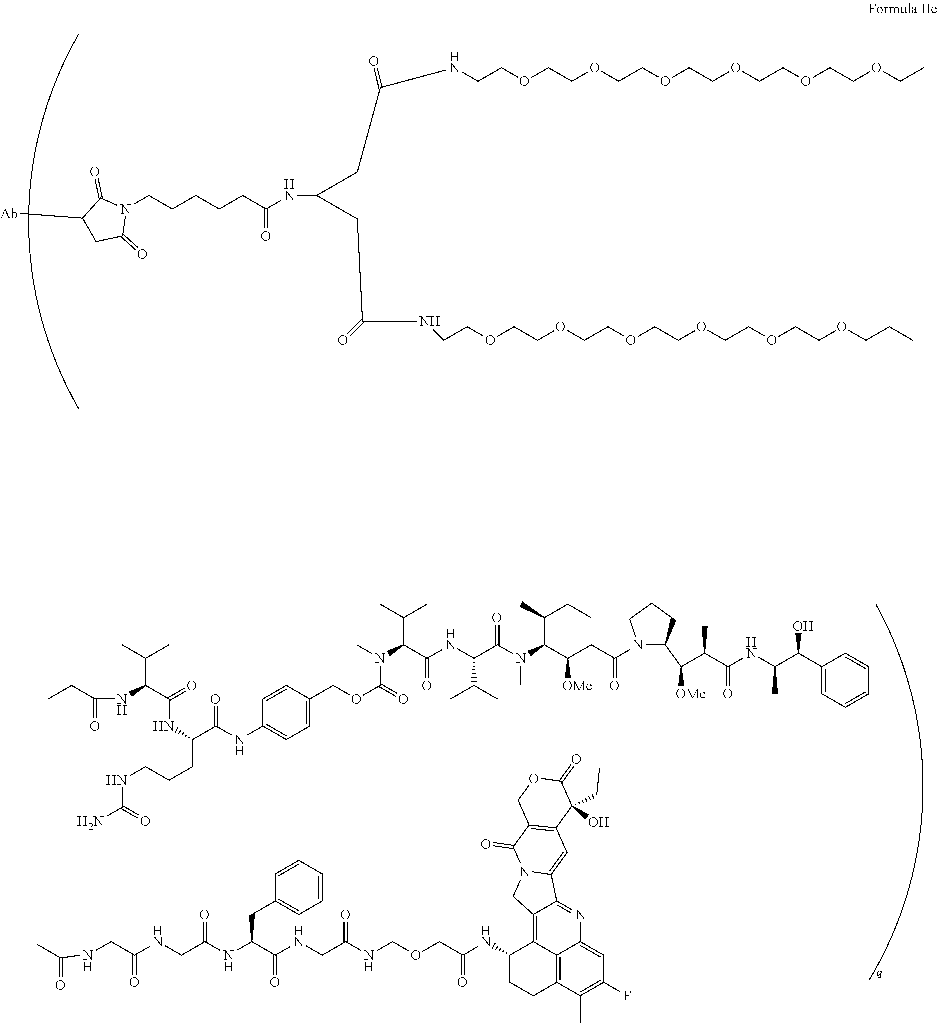

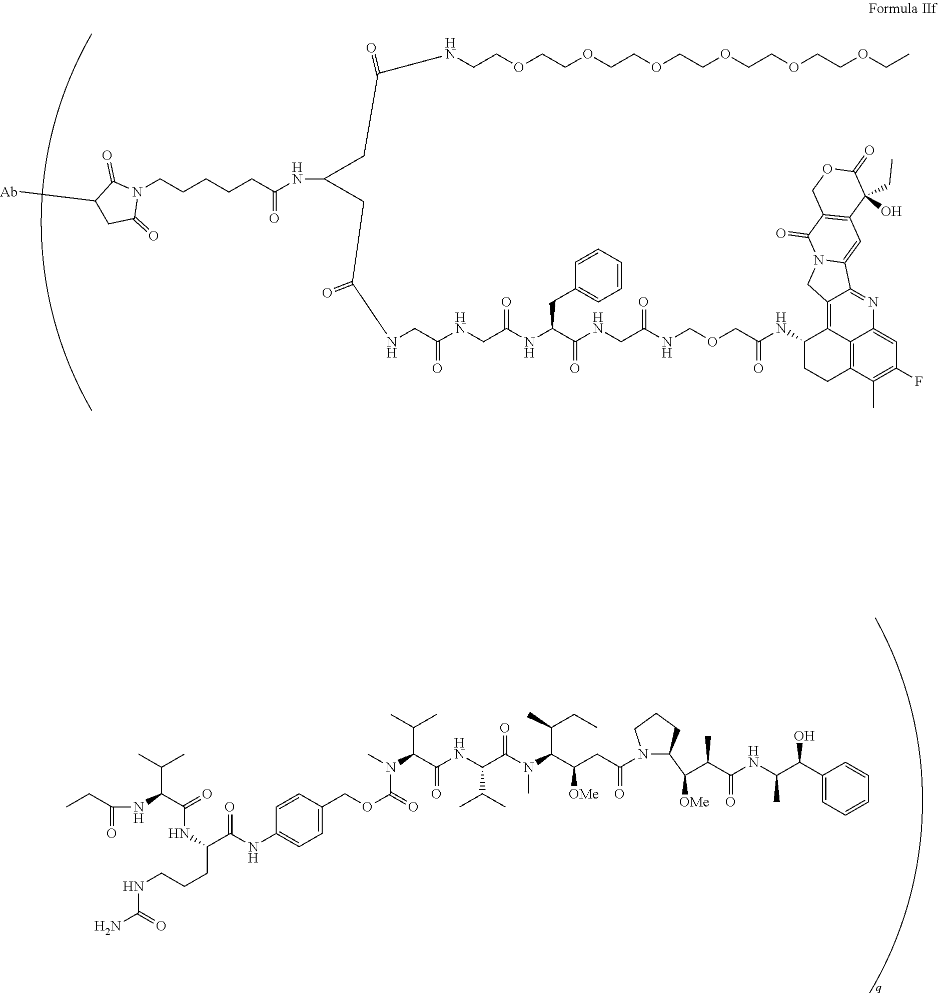

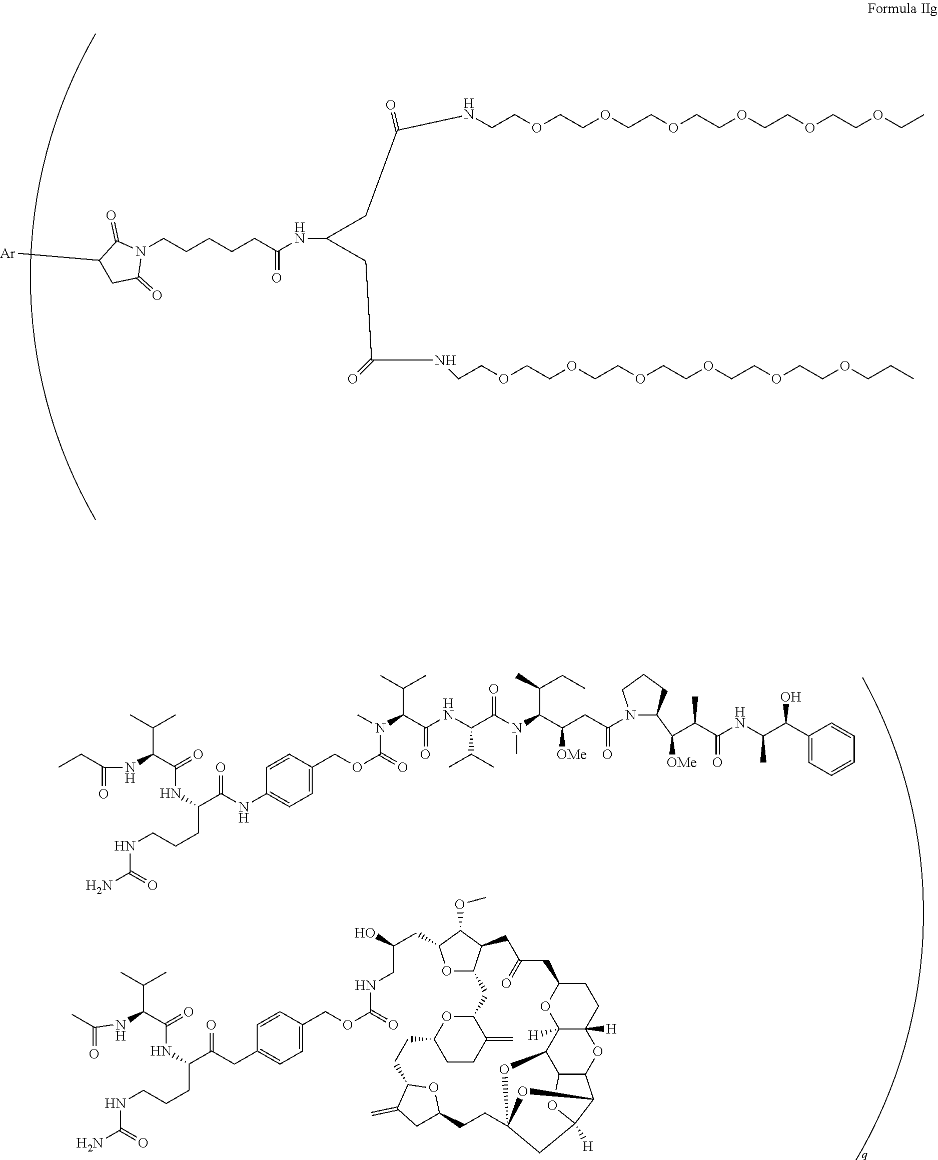

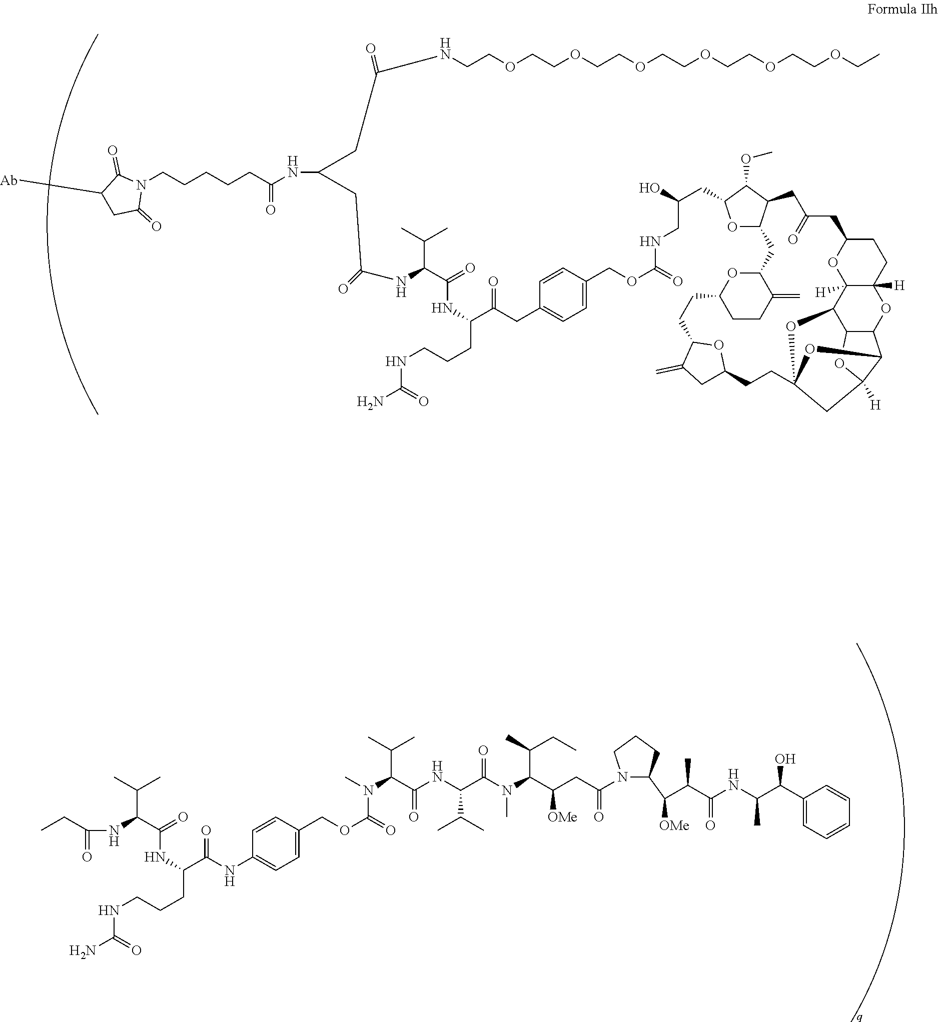

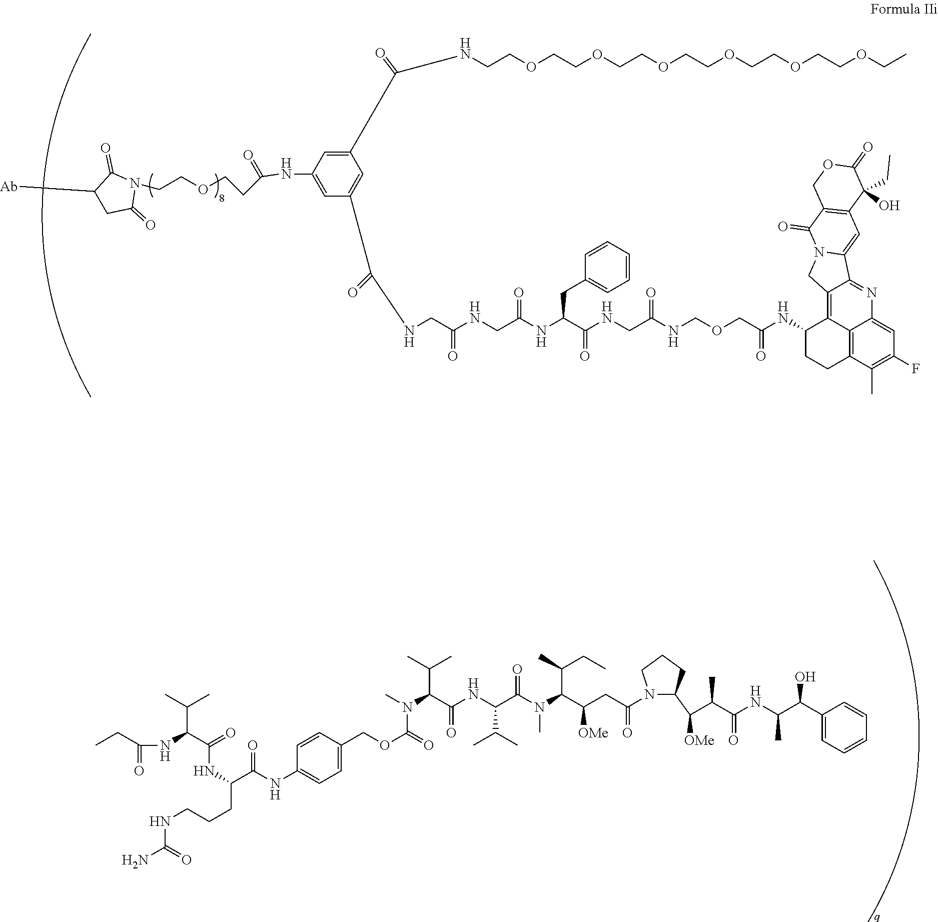

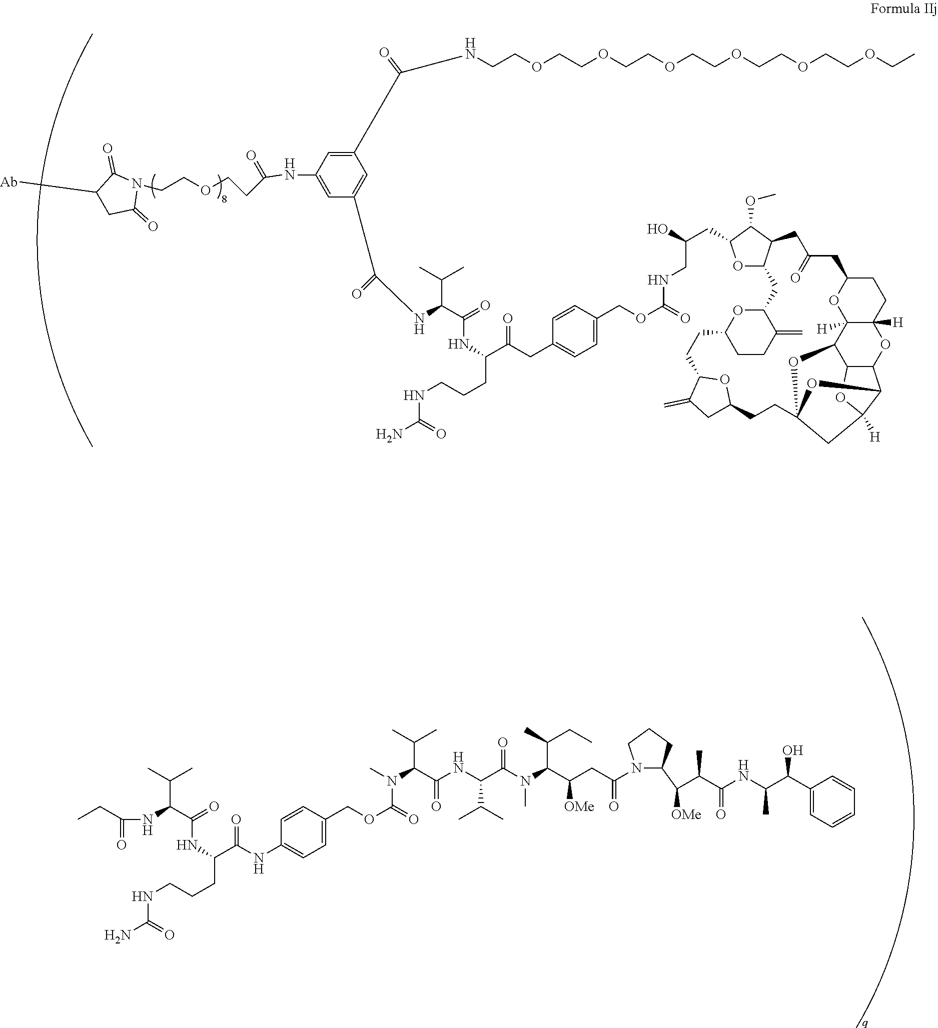

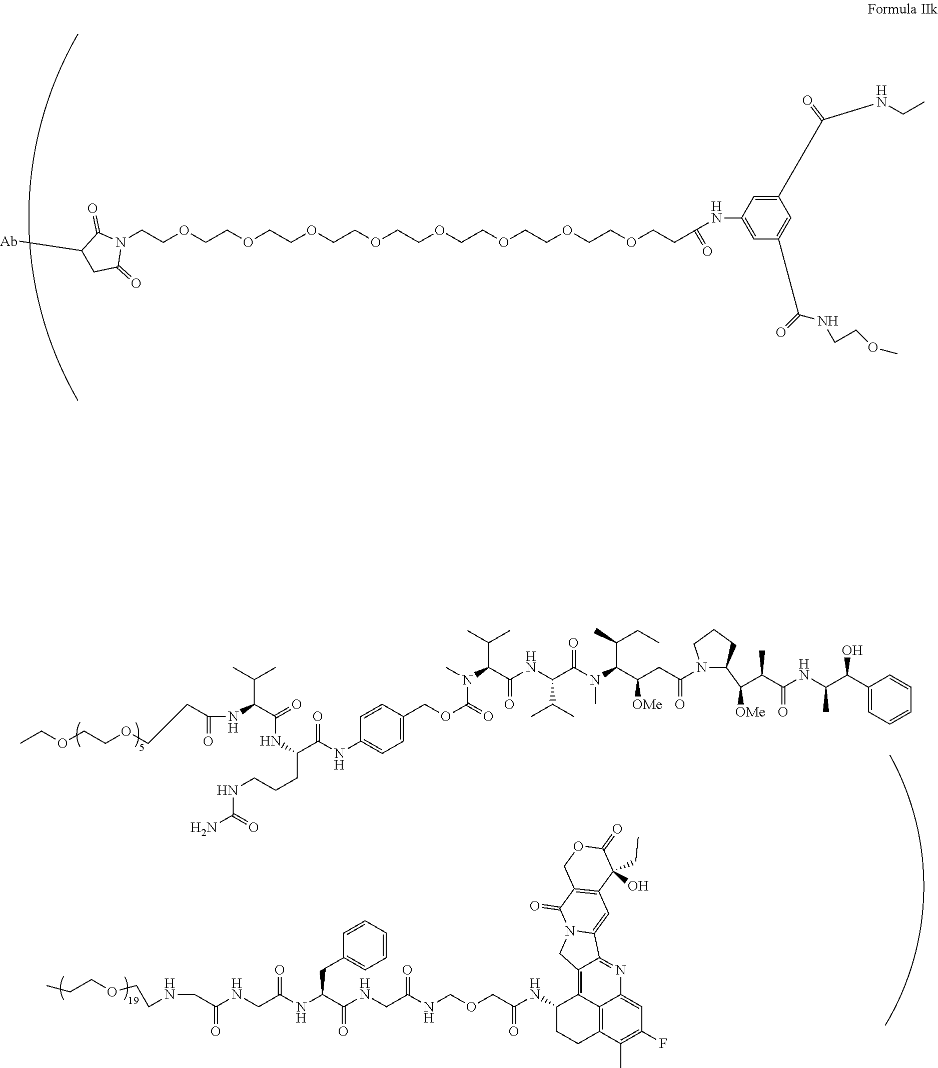

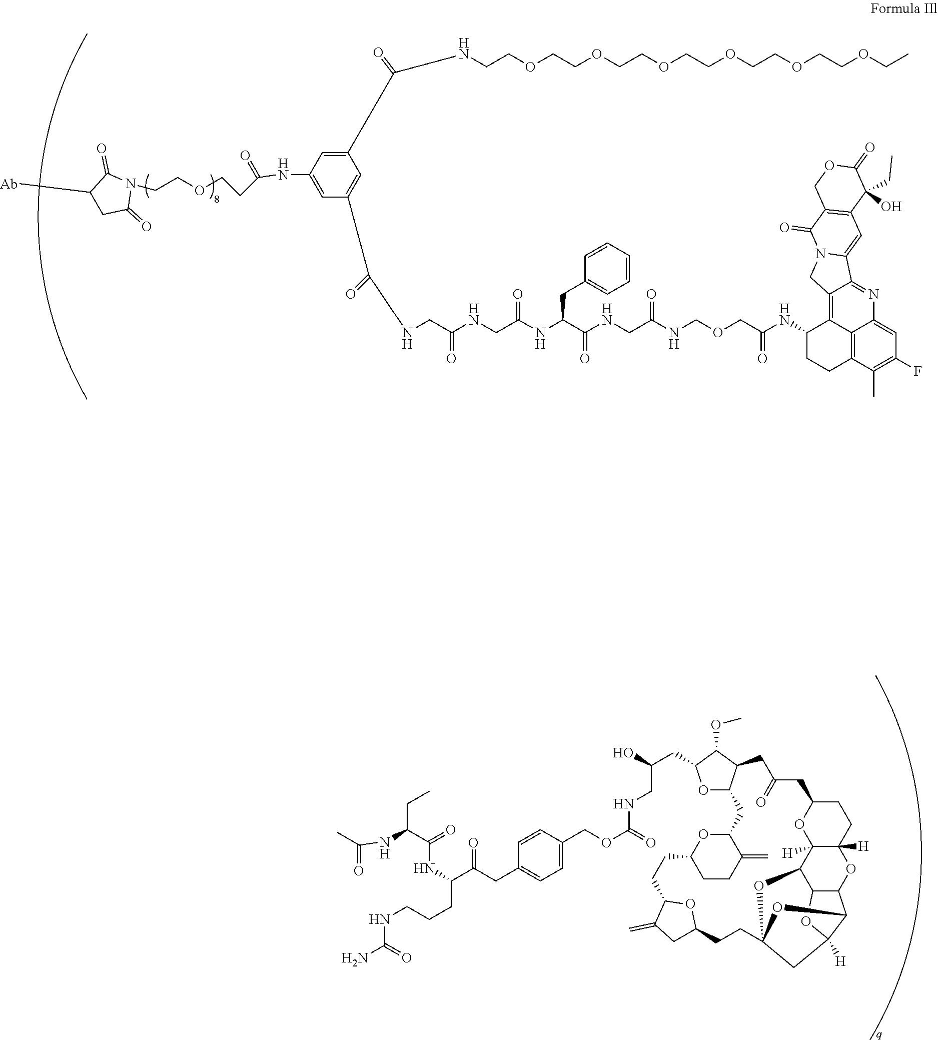

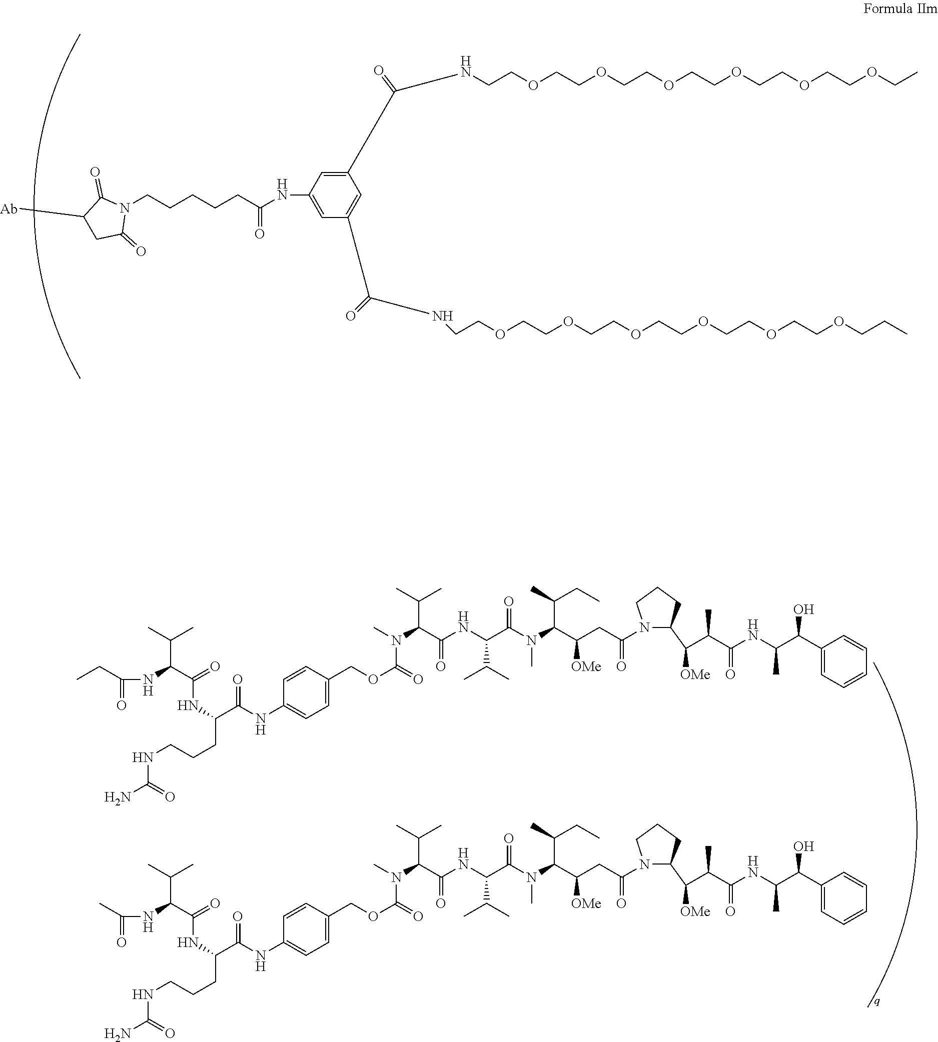

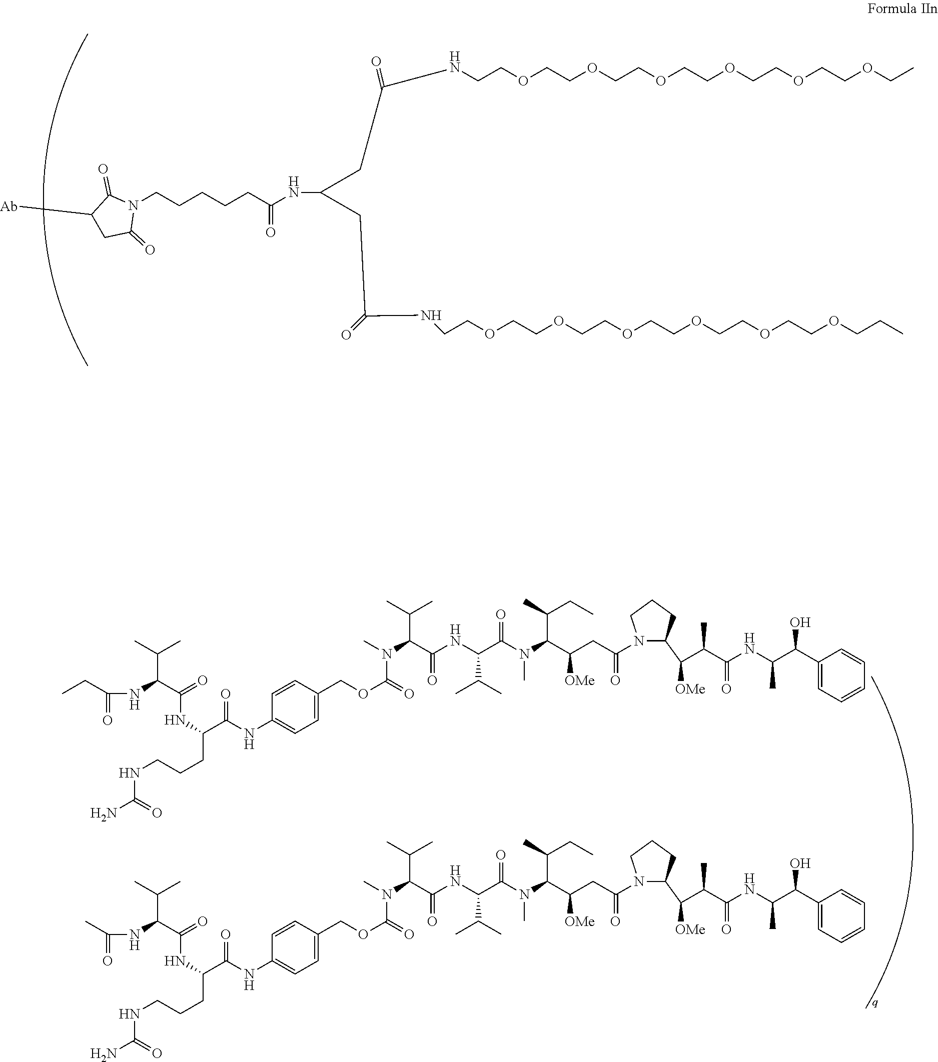

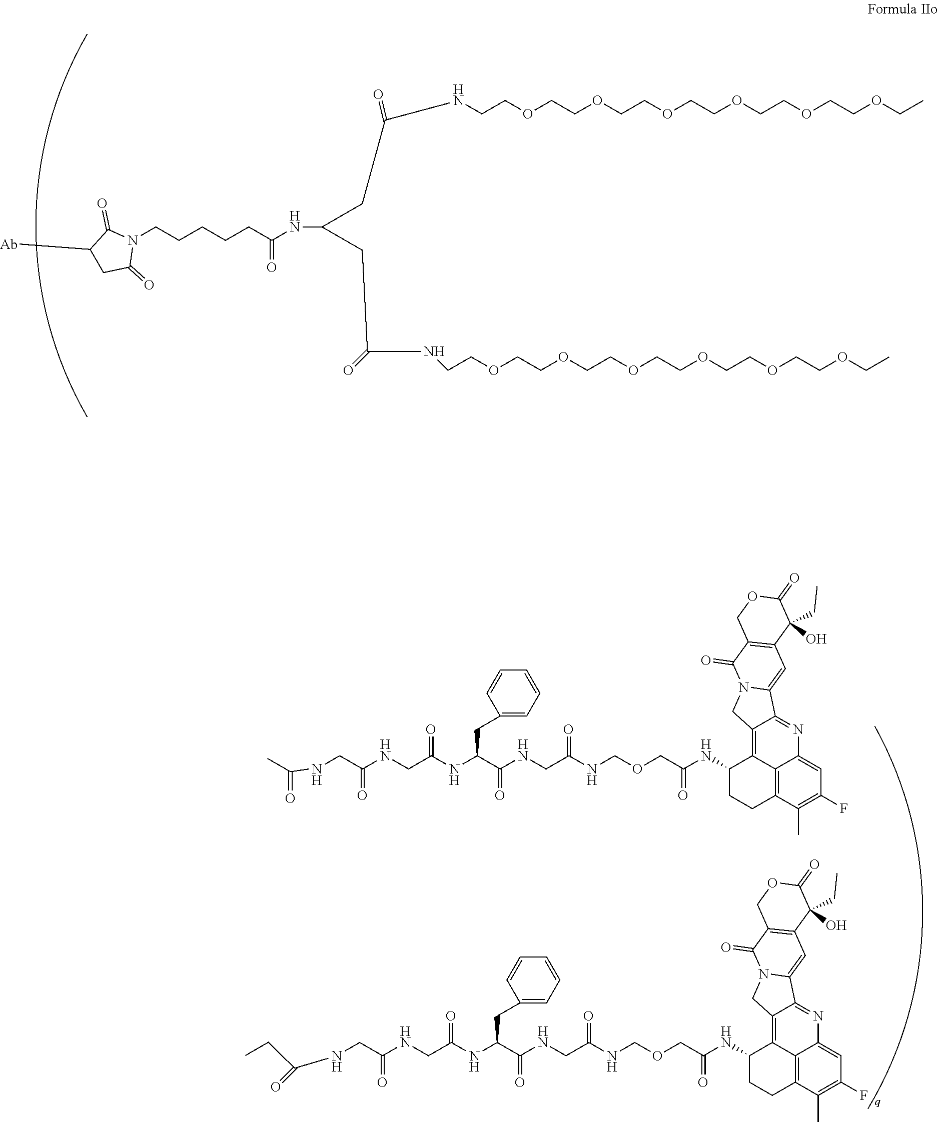

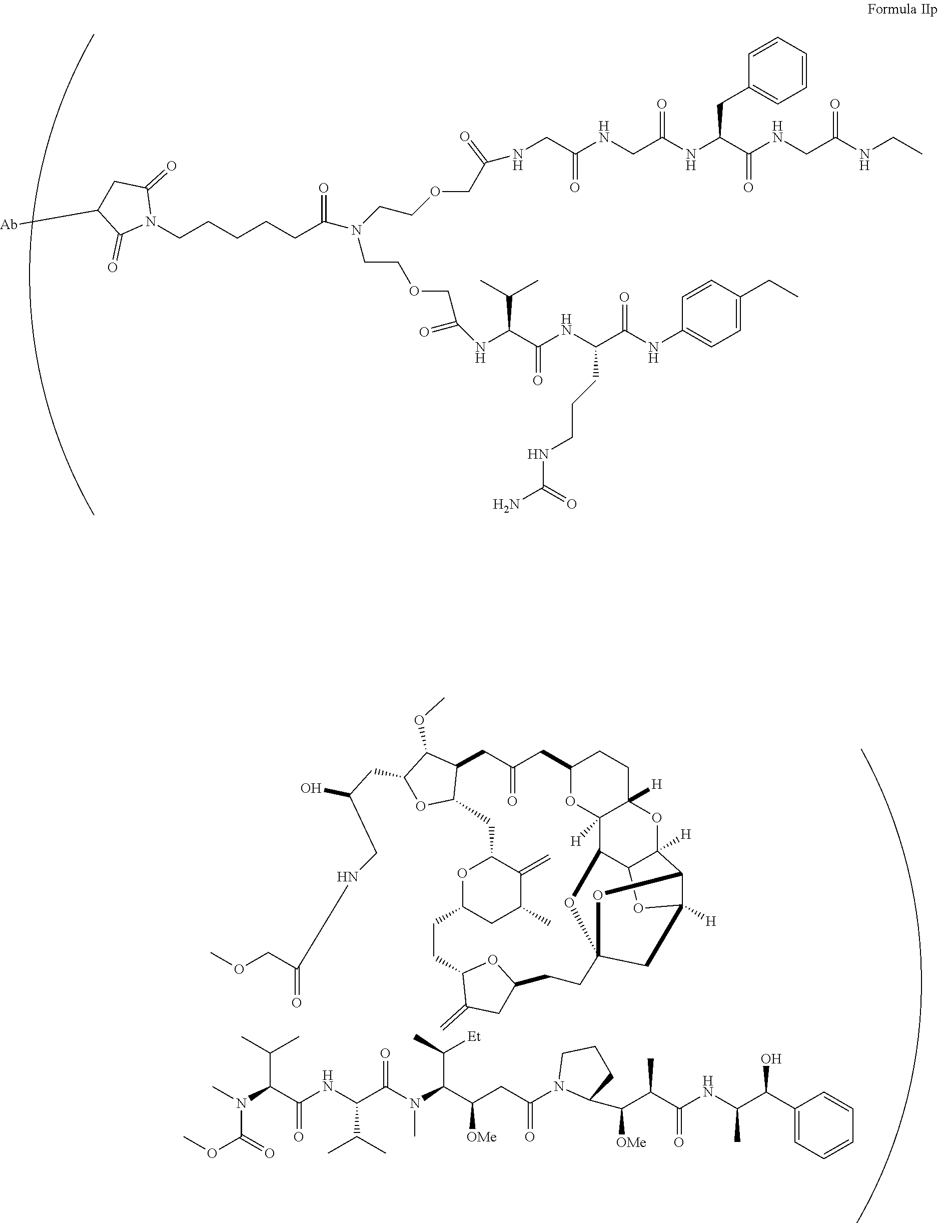

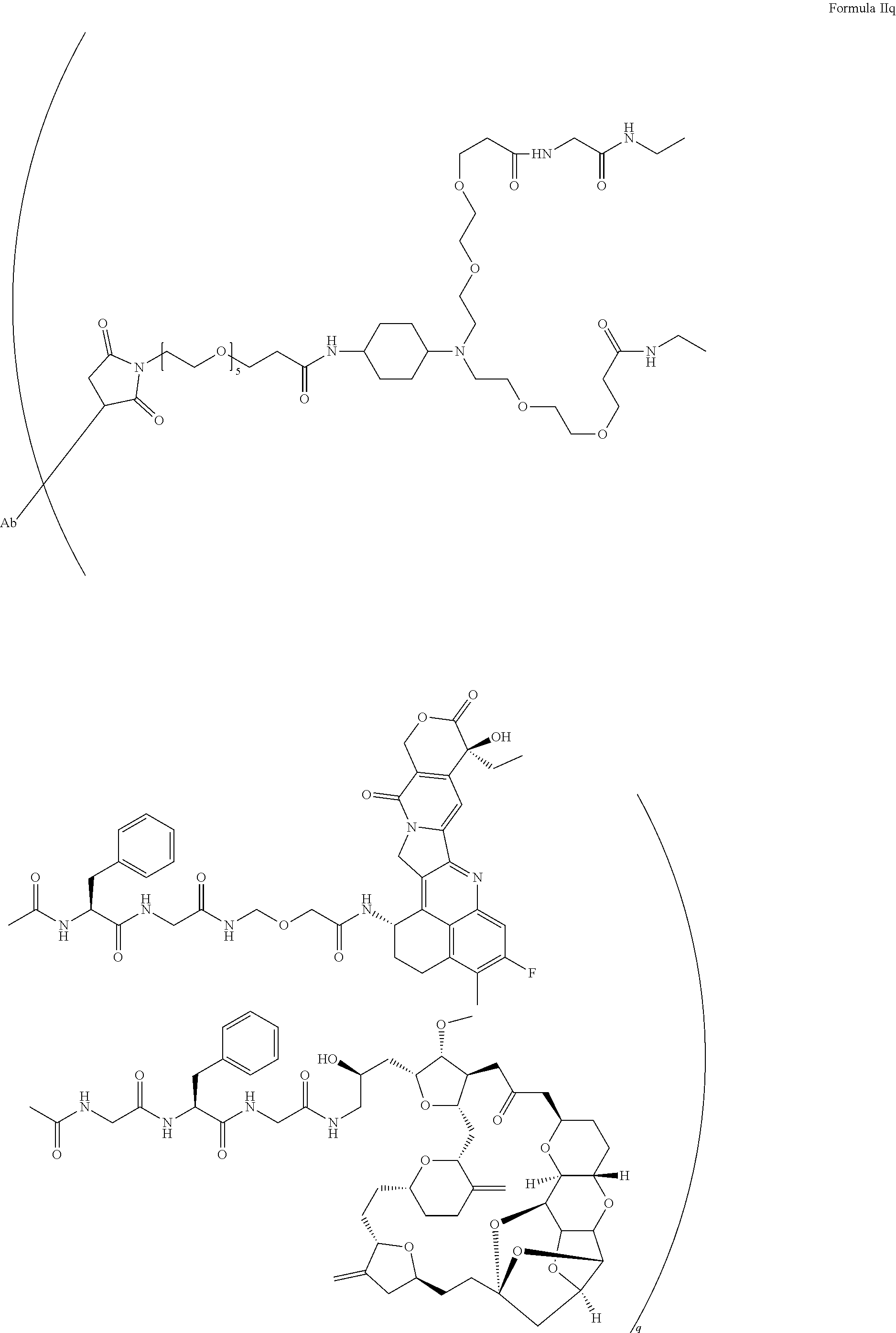

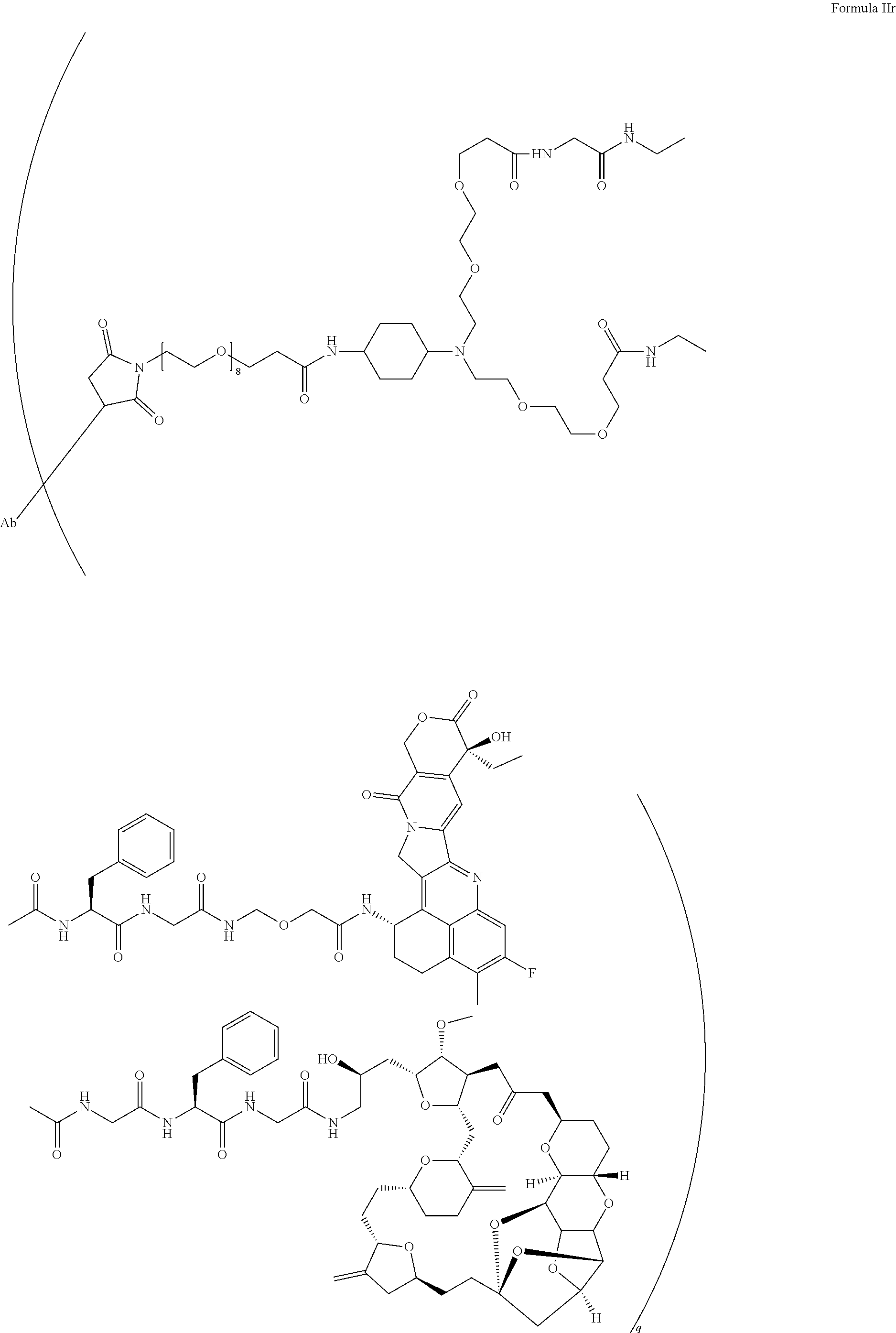

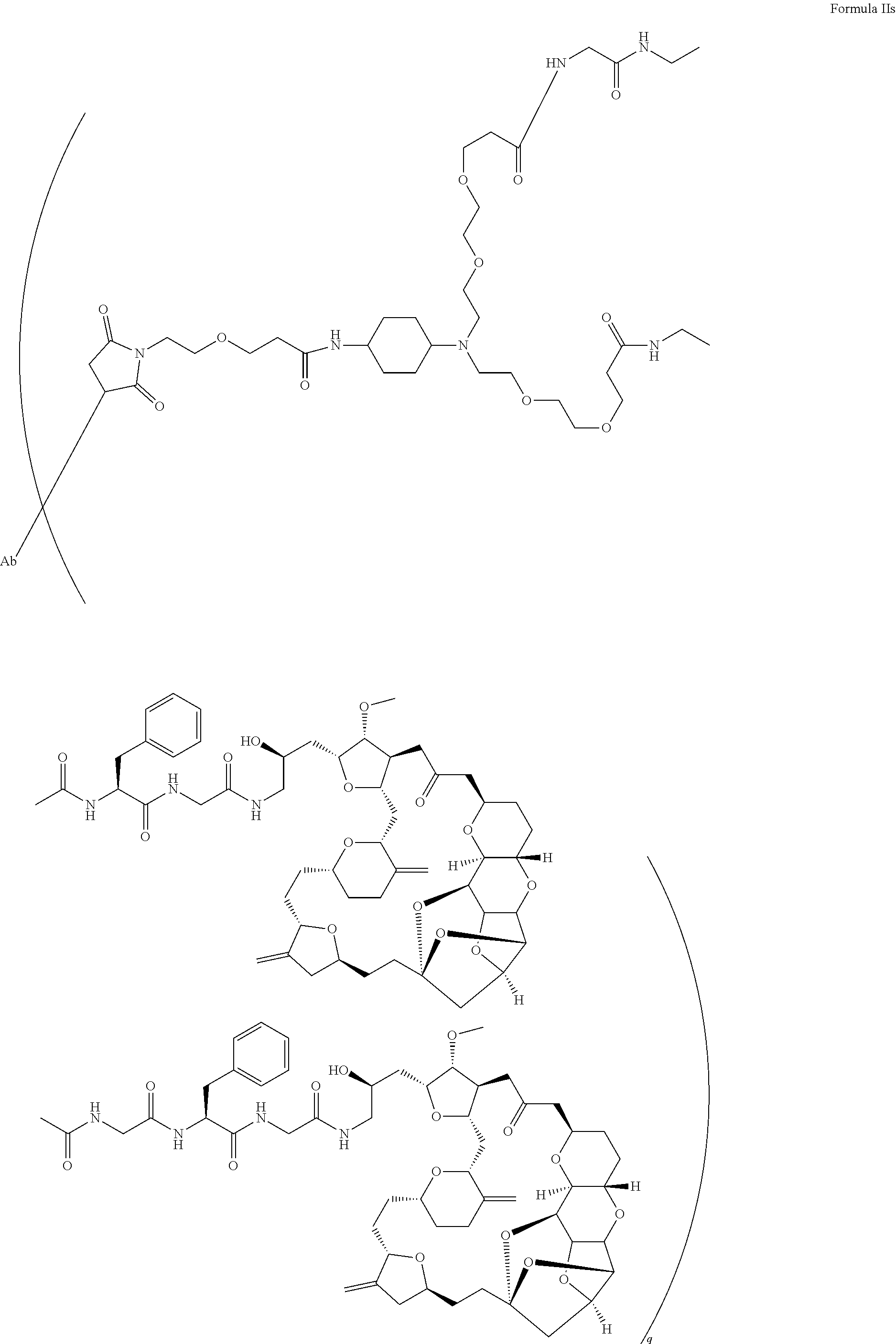

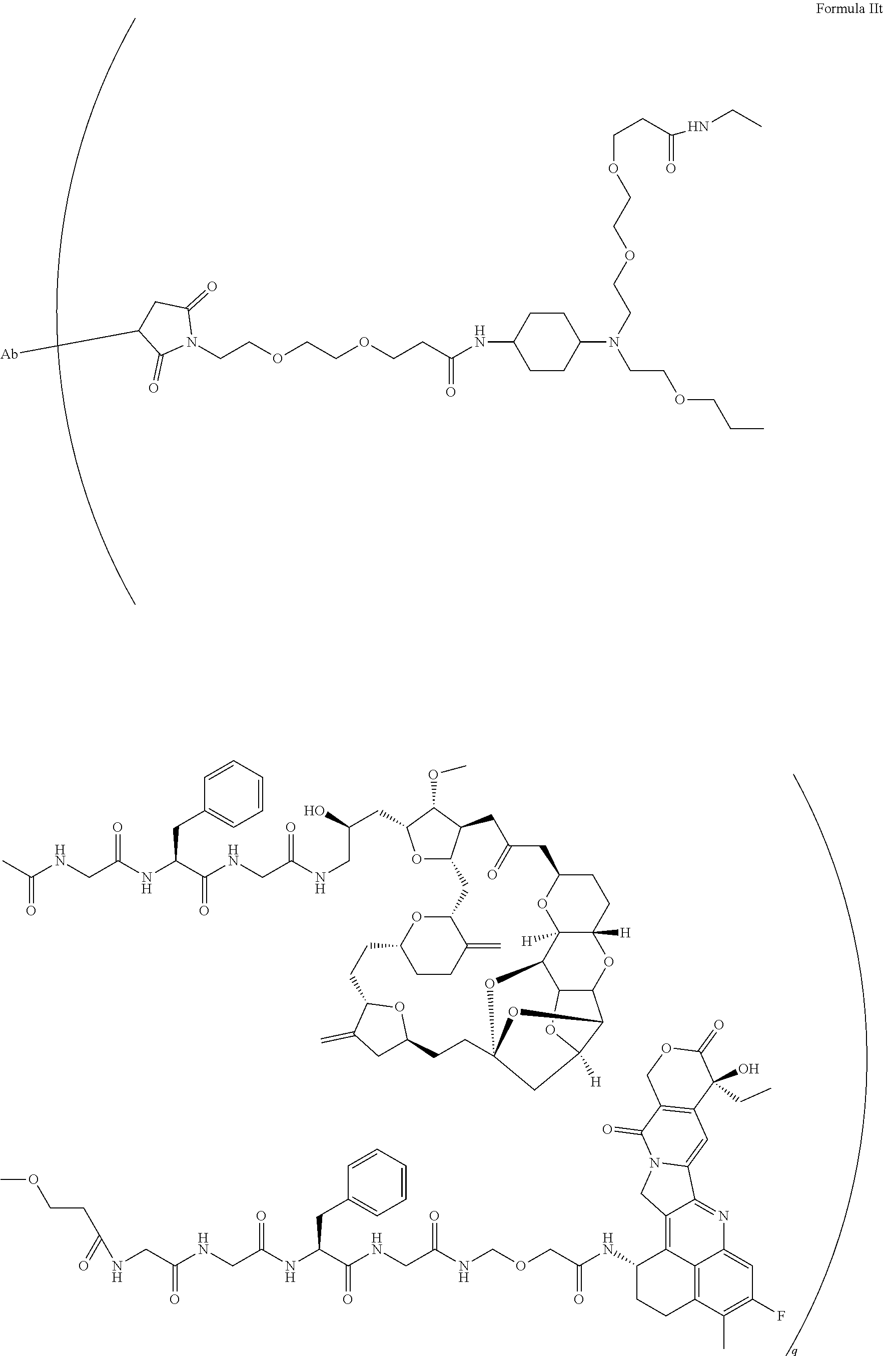

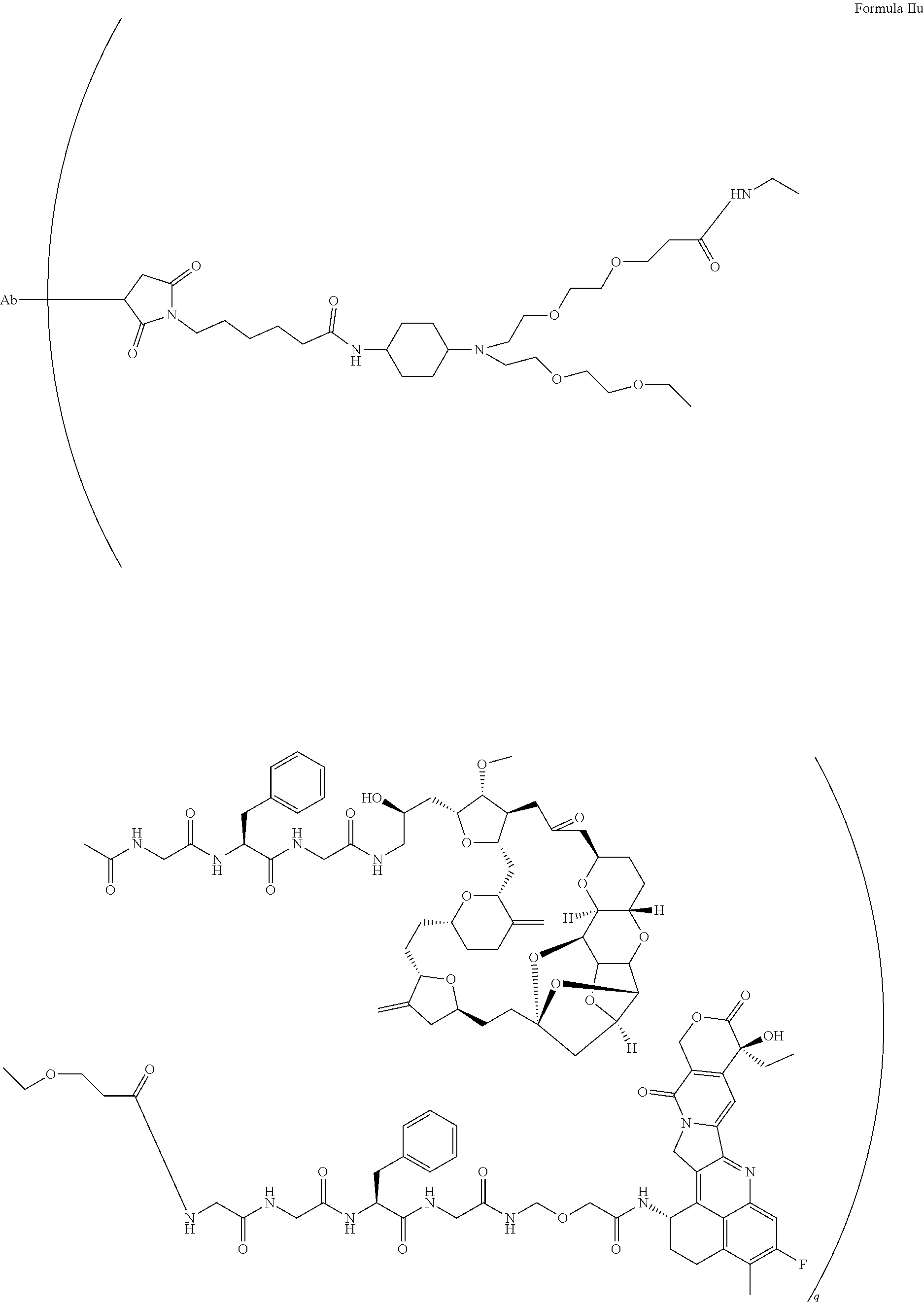

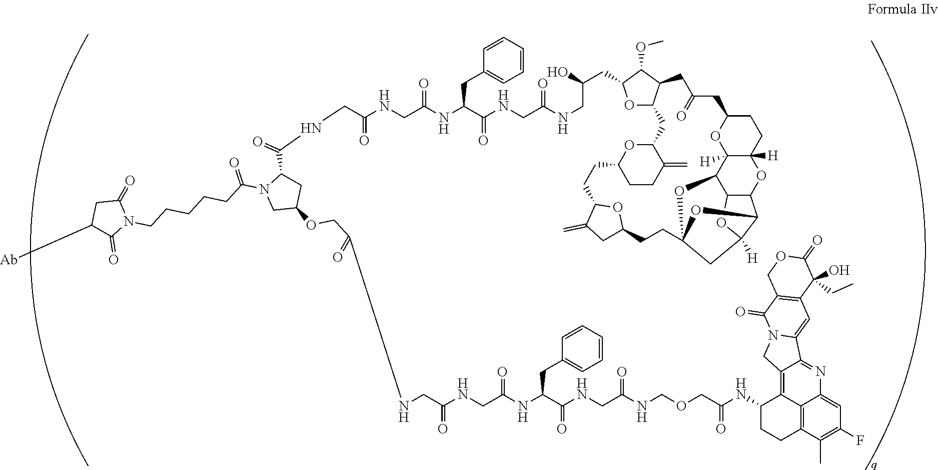

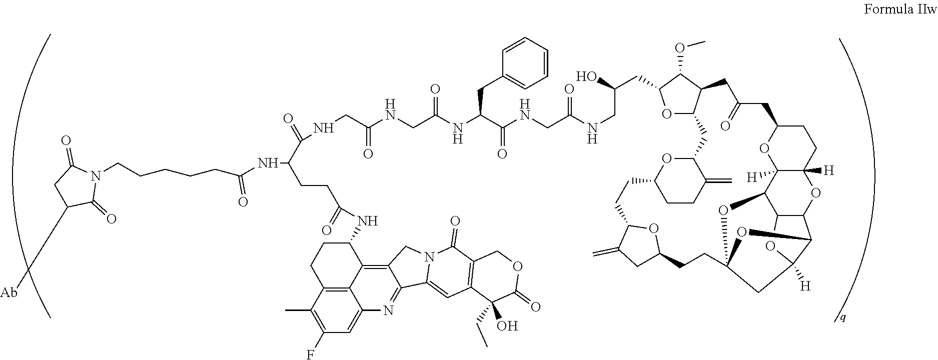

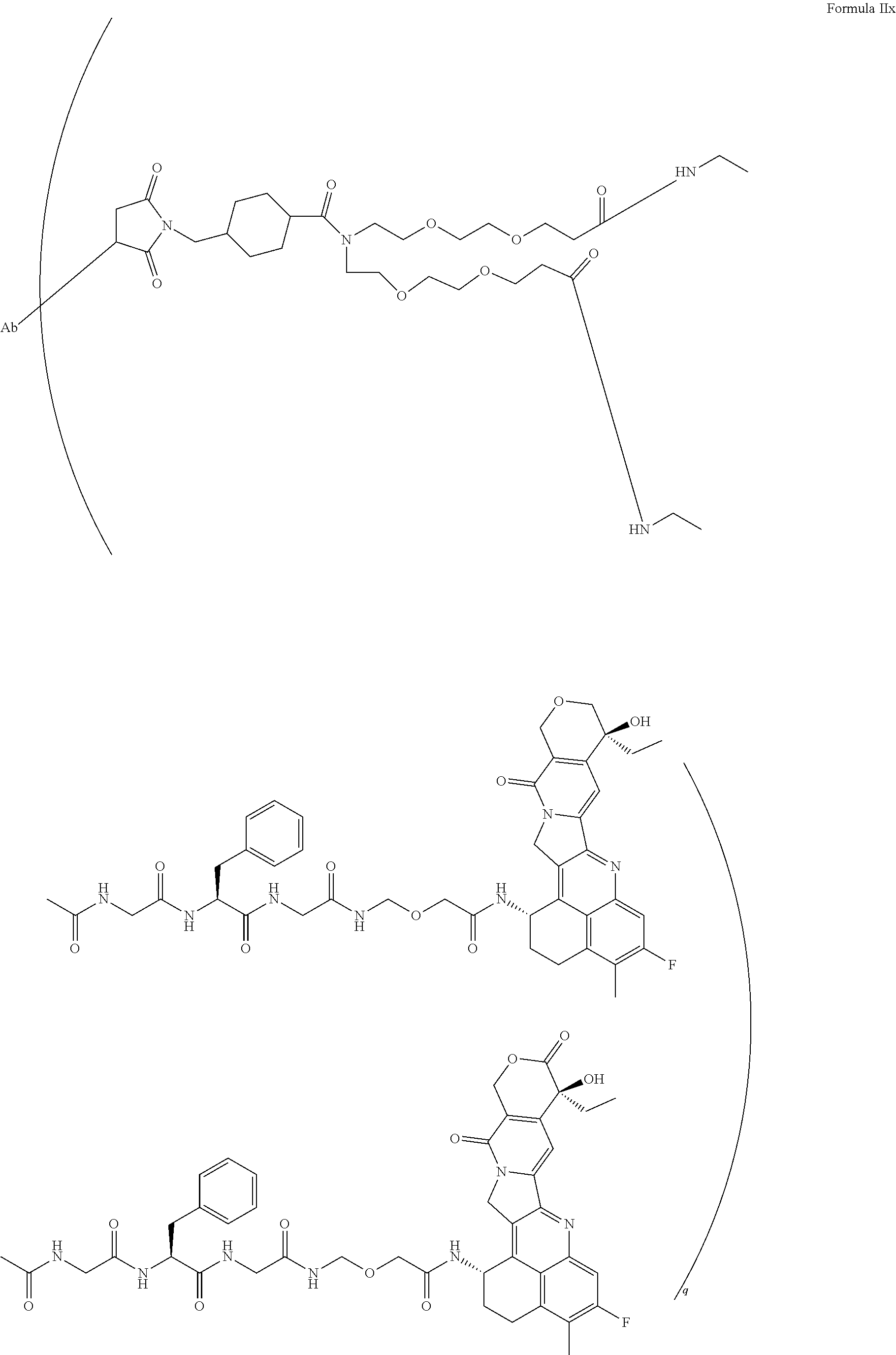

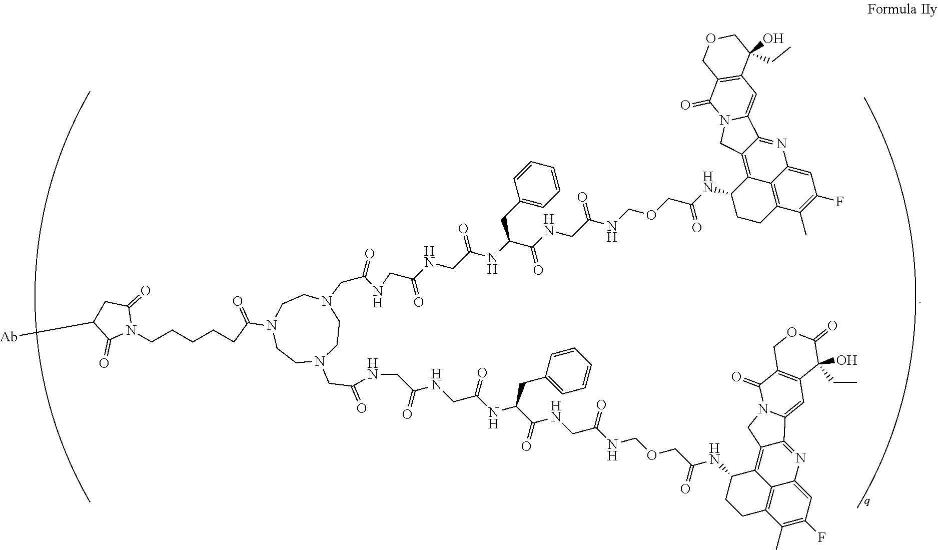

16. The preparation method for the dual-drug targeting linker-drug conjugate molecule according to claim 11, characterized in that the method comprises the following procedure: a targeting linker is conjugated with a dual-drug link assembly unit.

17. A medicament for preventing and/or treating tumors, characterized in that it is a composition made from the dual-drug targeting linker-drug conjugate molecule according to claim 11, as the active ingredient, in combination with pharmaceutically acceptable excipients.

18. The use of the dual-drug targeting linker-drug conjugate molecule according to claim 11 in the manufacture of medicaments for the prevention and/or treatment of tumors.

19. The use according to claim 18, characterized in that the tumor is HER2-positive.

20. The use according to claim 18, characterized in that the tumors are selected from the group consisting of lung cancer, urethra cancer, large intestine cancer, prostate adenocarcinoma, ovarian cancer, pancreatic cancer, breast cancer, bladder cancer, gastric cancer, gastrointestinal stromal tumor, cervical cancer, esophageal cancer, squamous cell cancer, peritoneal cancer, liver cancer, colon cancer, rectal cancer, colorectal cancer, uterine cancer, salivary gland cancer, kidney cancer, vulva cancer, thyroid cancer, penile cancer, leukemia, malignant lymphoma, plasmacytoma, myeloma or sarcoma.
